(19) United States
(12) Patent Application Publication
(10) Pub. No.: US 2025/0074887 A1
(43) Pub. Date: Mar. 6, 2025

(54) PHARMACEUTICAL COMPOSITIONS OF 6-(2-(2H-TETRAZOL-5-YL)ETHYL)DECAHYDROISOQUINOLINE-3-CARBOXYLIC ACID AND DERIVATIVES THEREOF

(71) Applicant: Sea Pharmaceuticals LLC, Cambridge, MA (US)

(72) Inventors: James Philip PEARSON, Cambridge, MA (US); Eduardo J. Martinez, Bryn Mawr, PA (US); Chris H. SENANAYAKE, Brookfield, CT (US); Yaping HONG, Princeton, NJ (US); Arindam CHAKRABORTY, Plainsboro, NJ (US)

(73) Assignee: Sea Pharmaceuticals LLC., Cambridge, MA (US)

(21) Appl. No.: 18/725,310

(22) PCT Filed: Dec. 29, 2022
(86) PCT No.: PCT/US2022/082532

§ 371 Date: Jun. 28, 2024

Related U.S. Application Data
(60) Provisional application No. 63/266,327, filed on Jan. 1, 2022.

(51) Int. Cl. C07D 401/06; A61K 31/4725; A61P 25/08

(52) U.S. Cl. CPC C07D 401/06; A61K 31/4725; A61P 25/08

(57) ABSTRACT

6-(2-(2H-tetrazol-5-yl)ethyl)decahydroisoquinoline-3-carboxylic acid and derivatives thereof, of formula

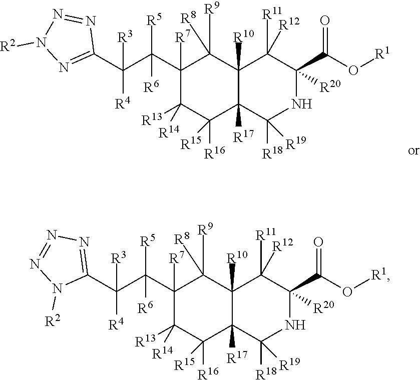

are disclosed, as are pharmaceutical compositions and methods for the treatment of epilepsy and seizure disorders employing those compositions.

### TECHNOLOGICAL FIELD

The present disclosure relates to pharmaceutical compositions of 6-(2-(2H-tetrazol-5-yl)ethyl)decahydroisoquinoline-3-carboxylic acid and derivatives thereof for use in the treatment of epilepsy and seizure disorders.

### BACKGROUND

The α-amino-3-hydroxy-5-methyl-4-isoxazolepropionic acid receptor (also known as AMPA receptor, AMPAR, or quisqualate receptor) is an ionotropic transmembrane receptor that is known as a glutamate-gated ion channel that mediates fast synaptic transmission in the central nervous system (CNS). AMPAR has been traditionally classified as a non-NMDA-type receptor, along with the kainate receptor (KAINR). The NMDA receptor (NMDAR) is a third glutamate-gated ion channel. AMPAR, KAINR and NMDAR are also referred to as ligand activated ion channels.

Glutamate is the major excitatory amino acid (EAA) neurotransmitter in the central nervous system. AMPA receptors (AMPARs) are large multi-subunit ion channels and are made of combinations of four AMPAR protein subunits GluA1, GluA2, GluA3, GluA4 (encoded by 4 separate genes). AMPARs are found in excitatory synapses of neurons and transduce fast excitatory neurotransmission. AMPARs transmit a glutamate signal into a depolarization of the postsynaptic neuron.

Glutamate released from the excitatory neuron diffuses across the synapse and binds to AMPARs in the postsynaptic neurons. AMPARs physically span the neuronal cell membrane and contain an ion pore or ion channel that is selectively inwardly permeable to the flow of primarily sodium ions (but also potassium and rarely to calcium) into the cell from the outside. In the absence of glutamate, the AMPAR ion pore is closed, and ions thus cannot flow into the neuron. When glutamate binds the AMPAR it opens allowing mainly sodium ions to pass through the pore, thus crossing the post-synaptic neuronal cell membrane, which results in depolarization. Thus, sodium carries the depolarizing current. AMPARs are critical to neuronal networks and to the physiological function of the brain and central nervous system. In summary AMPA receptors are neurotransmitter-gated (glutamate-gated) ion channels that open only in response to the chemical signal glutamate.

AMPA receptors play a key role in the generation and spread of epileptic seizures Scharfman H E. 2007. Curr. Neurol. Neurosci. Rep. 7:348-354. “The Neurobiology of Epilepsy”; Rogawski M A. 2011. Epilepsy Currents 11:56-63. “Revisiting AAMPA receptors as an antiepileptic drug target”. Neurosurgeons and neurologists have observed in clinical samples collected from human epilepsy (n=6 patients) brain (hippocampus) microdialysates that glutamate levels were increased prior to, and increased even further during, seizures in these patients (During M J and Spencer D D 1993. The Lancet. 341(8861):1607-10 “Extracellular Hippocampal Glutamate and Spontaneous Seizure in the Conscious Human Brain”).

Treatment with many different AMPAR antagonists in studies using animal models of seizures, convulsion, and epilepsy have consistently demonstrated pre-clinical efficacy of this class of compounds regardless of noncompetitive action or competitive mechanism of the AMPAR antagonist molecule. In early clinical trials, neurologists observed treatment with experimental therapeutic AMPAR antagonist talampanel, which showed reduction of seizures in epilepsy patients (Chappell A S, Sander J W, Brodie M J, Chadwick D, Lledo A, Zhang D, Bjerke J, Kiesler G M, Arroyo S. 2002. Neurology 58:1680-1682. “A Crossover, Add-On Trial of Talampanel in Patients With Refractory Partial Seizures”). A decade later, the AMPAR antagonist perampanel was the first FDA and EMEA approved AMPAR antagonist for the treatment of epilepsy (French J A, Krauss G L, Steinhoff B J, Squillacote D, Yang H, Kumar D, Laurenza A. 2013. Epilepsia 54:117-125. “Evaluation of adjunctive perampanel inpatients with refractory partial-onset seizures: Results of randomized global phase III study 305”; and Krauss G L, Perucca E, Ben-Menachem E, Kwan P, Shih J J, Squillacote D, Yang H, Gee M, Zhu J, Laurenza A. 2013. Epilepsia 54:126-134. “Perampanel, a selective, noncompetitive a-amino-3-hydroxy-5-methyl-4-isoxazolepropionic acid receptor antagonist, as adjunctive therapy for refractory partial-onset seizures: interim results from phase III, extension study 307”).

AMPAR's permeability to calcium and other cations, such as sodium and potassium, is governed by the GluA2 subunit. If an AMPAR lacks a GluA2 subunit, then it will be permeable to sodium, potassium, and calcium. The presence of a GluA2 subunit will almost always render the channel impermeable to calcium. This is determined by post-transcriptional modification (RNA editing) of the Q-to-R editing site of the GluA2 mRNA. Here, A→I (Adenosine to Inosine) editing (by Adenosine Deaminase Acting on RNA2) alters the GluA2 RNA coding for uncharged amino acid glutamine (Q) to instead code for the positively charged arginine (R) in the receptor's ion channel. The positively charged amino acid at the critical point makes it energetically unfavorable for calcium to enter the cell through the pore. Sodium is the major ion that the AMPAR glutamate-gated ion channel is permeable to.

Failure of the adenosine deaminase acting on RNA (ADAR) to edit GluA2 mRNA results in certain neurological disorders due to the permeability of the resulting altered AMPARs for calcium. Animals lacking ADAR die of seizures by day 21 after birth. However, in epilepsy normal AMPAR receptors are present, and they are permeable to sodium.

AMPAR antagonists represent a potential target for the treatment of epilepsy as they can reduce AMPAR mediated overactivation of neuronal networks in epilepsy (reviewed in Rogawski M A. 2011. Epilepsy Currents 11:56-63. “Revisiting AMPA receptors as an antiepileptic drug target”).

Kainate receptors (KAINRs) are another class of glutamate gated ion channels (in addition to NMDARs and AMPARs) that are expressed on some, but not all, neurons in the brain and central nervous system. Lerma J and Marques J M, Neuron 80:292-311 (2013) “Kainate Receptors in Health and Disease.” KAINRs were first identified by pharmacological studies demonstrating that kainate, a natural marine product from seaweed, acts as a potent agonist (glutamate mimic) and as a neurotoxin. KAINRs are composed of multiple protein subunits GluK1, GluK2, GluK3, GluK4 and GluK5 encoded by 5 different genes. Many accessory proteins are involved. KAINRs consist of tetramer combinations. Five KAINR subunits have been cloned from gene expression libraries of complementary DNA (cDNA) generated from mRNA, which was collected from brain tissue in humans or rodents by teams of molecular neurobiologists led by Peter H. Seeburg, Stephen F. Heinemann, and other scientists worldwide. The five KAINR protein subunits encoded by five different genes were renamed GluK1 (formerly called GluR5), GluK2 (formerly called GluR6), GluK3, GluK4, and GluK5. GluK1 cloning, its expression throughout the Central Nervous System of rodents, and its expression throughout animal development (e.g., from embryo through fetal and post-natal animal growth) and in adult animals, was first studied by Heineman. Bettler B. et al., Neuron 5:583-595 (1990) “Cloning of a novel glutamate receptor subunit GluR5: expression in the nervous system during development.” Native KAINR ion channels were studied ex vivo by testing effects of 3 different agonists, L-glutamate, kainate, or domoate (compared to other agents), in electrophysiology of whole cell patch-clamp recordings of neurons isolated from rat spinal DRGs (dorsal root ganglion) and were first characterized by Huettner J E. Neuron 5:255-266 (1990) “Glutamate Receptor Channels in Rat DRG Neurons: Activation by Kainate and Quisqualate and Blockaid of Desensitization by Con A.” Cloned mouse recombinant GRIK1 gene encoding the KAINR protein subunit GluK1 (formerly called GluR5) was studied in transfected HEK293 mammalian cells in whole cell patch clamp electrophysiology studies by Peter H. Seeburg. Sommer B. et al., The EMBO J. 11:1651-1656 (1992) “A glutamate receptor channel with high affinity for domoate and kainate.” The studies of mouse recombinant GluK1 (formerly called GluR5) recorded KAINR currents by applying kainate or its structural analog domoate to cells and measuring the resulting mouse recombinant KAINR ion channel activity in GluK1 transfected cells. Sommer et al. (1992). Human recombinant GluK1 was cloned from a cDNA library cloned from mRNA collected from Human brain tissue. See, e.g., U.S. Pat. No. 6,500,624; Korczak B. et al., Receptors and Channels 3:41-49 (1995) “cDNA cloning and functional properties of human glutamate receptor EAA3 (GluR5) in homomeric and heteromeric configuration.” Published data from human HEK293 cells stably transfected with GRIK1 gene (formerly called EAA3) expressing GluK1 (formerly called GluR5) show robust Kainate induced currents in whole cell patch clamp electrophysiology studies of these cells. See U.S. Pat. No. 6,500,624 and Korczak B. et al. (1995). Data confirming in vivo KAINR biology in humans has been observed by accident. In 1987 a public health outbreak occurred in Canada, wherein gastrointestinal and neurological symptoms occurred after >250 people reported symptoms, >100 reported strong neurological effects, >20 had memory loss, and 3 patients died after consuming contaminated shell fish (mussels) contaminated with a kainate analog called domoate [Perl et al., New England J. Med. 322:1775 (1990) “An outbreak of toxic encephalopathy caused by eating mussels contaminated with domoic acid” and Teitelbaum J S et al., NEJM 322:1781 (1990) “Neurological sequelae of domoic acid intoxication due to the ingestion of contaminated mussels.” ] Domoate or kainate each act as potent neurotoxin agonists of KAINRs. In the 1987 Canada outbreak, brain autopsies of the victims from the contaminated shellfish revealed human ingestion of domoate led to severe neurological damage (loss of neurons and tissue collapse) in the Hippocampus and the Amygdala regions of the brain. In rodents injected with kainate or domoate, seizures and severe excitotoxicity and neuron damage occurred. Rodents in seizure models pre-treated with KAINR antagonists are protected from seizures. Small molecule antagonists of KAINR or of both KAINR and AMPAR (dual antagonists) block seizures and block pain show promise and anti-convulsant, anti-seizure, and potentially anti-epilepsy drugs. See, e.g., Lerma J and Marques J M, Neuron 80:292-311 (2013) “Kainate Receptors in Health and Disease.” Some dual AMPAR and KAINR antagonists have been studied as potential analgesics or anti-hyperalgesic or anti-allodynia compounds.

U.S. Pat. No. 5,670,516 discloses that certain decahydroisoquinoline derivatives are AMPA receptor antagonists, and as such are useful in the treatment of many different neurological conditions, including convulsions and seizures. In addition, WO 01/02367 A3, published Jan. 11, 2001, discloses diester prodrug forms of the selective GluR₅ antagonist 3S,4aR,6S,8aR-6-(((4-carboxy)phenyl)methyl)-1,2,3,4,4a,5,6,7,8,8a-decahydroisoquinoline-3-carboxylic acid.

U.S. Pat. No. 7,247,644 discloses that monoesters of the monoacid, (3S,4aR,6R,8aR)-6-[2-(1(2)H-tetrazole-5-yl)ethyl-1,2,3,4,4a,5,6,7,8,8a-decahydroisoquinoline-3-carboxylic acid provide significantly improved bioavailability of the monoacid as compared to that provided by administration of the monoacid itself.

However, the bioavailability of these previous methods is still not sufficiently high to be considered for oral applications.

### BRIEF SUMMARY

In a first aspect, the present disclosure relates to a compound of formula Ia or formula Ib:

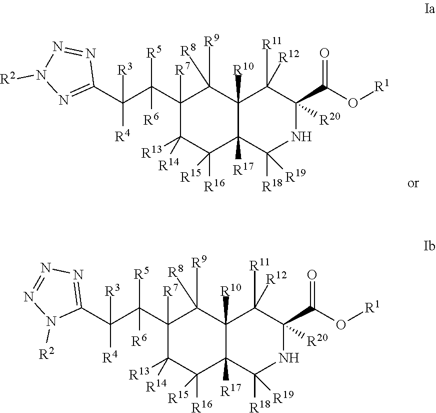

wherein: R¹ is selected from H, (C₁-C₂₀)hydrocarbyl, and —CH(R^{a})R^{b}; R^{a} is selected from H, —(C₁-C₆)alkyl, and —C(═O)—O—(C₁-C₆)alkyl; R^{b} is selected from —(C₁-C₁₀)hydrocarbyl-O—C(═O)—R^{c}, —(C₁-C₁₀)hydrocarbyl[-OC(═O)—R^{c}]₂, -direct bond-C(═O)—R or —(C₁-C₁₀)hydrocarbyl-C(═O)—R^{c}, —CH(NHR^{d})C(═O)—R^{c}, and —(C₁-C₁₀)hydrocarbyl-NR^{e}—C(═O)—R^{f}; R^{c} is selected from —O—(C₁-C₁₀)hydrocarbyl, —NH—(C₁-C₁₀)hydrocarbyl, —N—[(C₁-C₁₀)hydrocarbyl]₂, where the hydrocarbyl groups can be combined to form a (C₄-C₆) nitrogenous aliphatic heterocycle, —(C₁-C₁₀)hydrocarbyl, —NH₂, —(C₁-C₁₀)hydrocarbyl-NH—R^{e}, where the hydrocarbyl group can be combined with R^{e} to form a (C₄-C₆) nitrogenous aliphatic heterocycle, and —NR^{e}—(C₁-C₁₀)hydrocarbyl-C(═O)—O—(C₁-C₁₀)hydrocarbyl, where the hydrocarbyl group and R^{e} can be combined to form a (C₄-C₆) nitrogenous aliphatic heterocycle; R^{d} is selected from H, —(C₁-C₁₀)hydrocarbyl, and —C(═O)(C₁-C₁₀)hydrocarbyl; R^{e} is selected from H, and —(C₁-C₆)alkyl; R^{f} is selected from —O—(C₁-C₁₀)hydrocarbyl, —(C₁-C₁₀)hydrocarbyl, -direct bond-C(═O)—O—(C₁-C₁₀)hydrocarbyl or —(C₁-C₄)hydrocarbyl-C(═O)—O—(C₁-C₁₀)hydrocarbyl, -direct bond-C(═O)—NH₂ or —(C₁-C₄)hydrocarbyl-C(═O)—NH₂, -direct bond-C(═O)—NH—(C₁-C₁₀)hydrocarbyl or —(C₁-C₄)hydrocarbyl-C(═O)—NH—(C₁-C₁₀)hydrocarbyl, —(C₁-C₁₀)hydrocarbyl-NH—R^{e}, where the hydrocarbyl linker can be combined with R^{e} to form a (C₄-C₆) nitrogenous aliphatic heterocycle, and —(C₁-C₁₀)hydrocarbyl-NH—C(═O)(C₁-C₁₀)hydrocarbyl; R² is selected from H and optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂; R⁷ is selected from hydrogen, deuterium, and fluorine; R³-R⁶ and R⁸-R²⁰ are independently selected from hydrogen and deuterium; and with the provisos: (i) when R¹ is C₁-C₆ alkyl or aryl and R² is a nitrogen protecting group, wherein said nitrogen protecting group is selected from trityl, benzyl, t-butyl, t-butyldimethylsilyl, and triphenylsilyl, then either (a) R⁷ is fluorine or (b) at least one of R³-R²⁰ is deuterium; (ii) when R¹ is H, C₁-C₆ alkyl, substituted alkyl, cycloalkyl, or arylalkyl and R² is H, then either (a) R⁷ is fluorine or at (b) least one of R³-R²⁰ is deuterium; and (iii) when R⁷ is fluorine, then R² is not H.

In a second aspect, the present disclosure relates to a compound of formula IIa, IIb, IIIa, or IIIb:

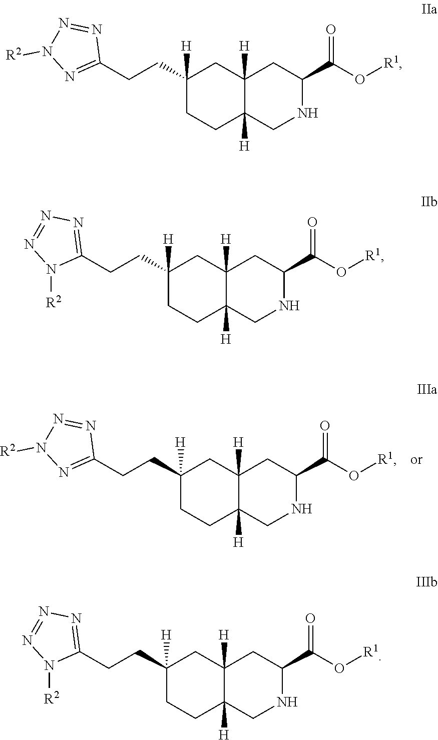

wherein: R¹ is selected from H and (C₁-C₂₀)hydrocarbyl; R² is optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂; and R³-R²⁰ are each H.

In a third aspect, the present disclosure relates to a compound of formula IVa, IVb, Va, or Vb:

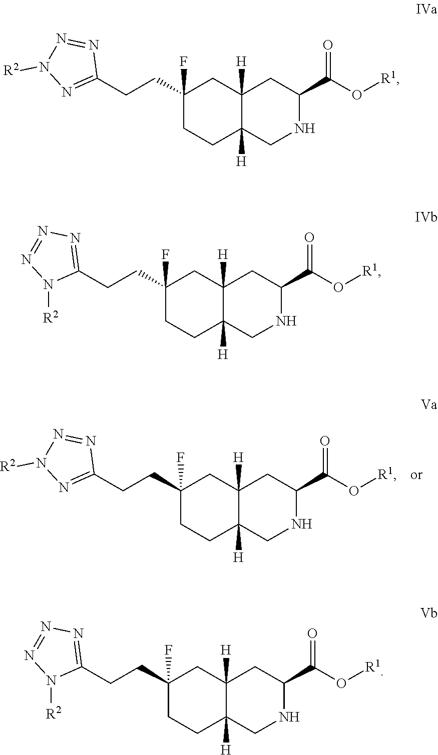

wherein: R¹ is selected from H and (C₁-C₂₀)hydrocarbyl; R³-R⁶ and R⁸-R²⁰ are each H; R⁷ is fluorine; and R² is optionally substituted (C₁-C₇)hydrocarbyl, wherein said optionally substituted (C₁-C₇)hydrocarbyl may be substituted with one or more of —F, —CN, —OH, —O—C(═O)(C₁-C₄)hydrocarbyl, —C(═O)—OH, —C(═O)—O—CH₃, —C(═O)—NH—CH₃, —NH(C₁-C₄)hydrocarbyl, —N[(C₁-C₄)hydrocarbyl)]₂, or —C(═O)—N[(C₁-C₃)hydrocarbyl]₂;

In a fourth aspect, the present disclosure relates to a compound of formula VIa or VIb:

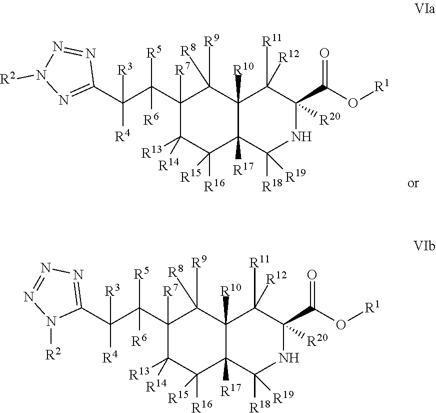

wherein: R¹ is selected from —CH(R^{a})R^{b} and aromatic (C₁-C₂₀)hydrocarbyl; R^{a} is selected from H, —(C₁-C₆)alkyl, and —C(═O)—O—(C₁-C₆)alkyl; R^{b} is selected from —(C₁-C₁₀)hydrocarbyl-O—C(═O)—R^{c}, —(C₁-C₁₀)hydrocarbyl[-OC(═O)—R^{c}]₂, -direct bond-C(═O)—R^{c} or —(C₁-C₁₀)hydrocarbyl-C(═O)—R^{c}, —CH(NHR^{d})C(═O)—R^{c}, and —(C₁-C₁₀)hydrocarbyl-NR^{e}—C(═O)—R; R^{c} is selected from —O—(C₁-C₁₀)hydrocarbyl, —NH—(C₁-C₁₀)hydrocarbyl, —N—[(C₁-C₁₀)hydrocarbyl]₂, where the hydrocarbyl groups can be combined to form a (C₄-C₆) nitrogenous aliphatic heterocycle, —(C₁-C₁₀)hydrocarbyl, —NH₂, —(C₁-C₁₀)hydrocarbyl-NH—R^{e}, where the hydrocarbyl group can be combined with R^{e} to form a (C₄-C₆) nitrogenous aliphatic heterocycle, and —NR^{e}—(C₁-C₁₀)hydrocarbyl-C(═O)—O—(C₁-C₁₀)hydrocarbyl, where the hydrocarbyl group and R^{e} can be combined to form a (C₄-C₆) nitrogenous aliphatic heterocycle; R^{d} is selected from H, —(C₁-C₁₀)hydrocarbyl, and —C(═O)(C₁-C₁₀)hydrocarbyl; R^{e} is selected from H, and —(C₁-C₆)alkyl; R^{f} is selected from —O—(C₁-C₁₀)hydrocarbyl, —(C₁-C₁₀)hydrocarbyl, -direct bond-C(═O)—O—(C₁-C₁₀)hydrocarbyl or —(C₁-C₄)hydrocarbyl-C(═O)—O—(C₁-C₁₀)hydrocarbyl, -direct bond-C(═O)—NH₂ or —(C₁-C₄)hydrocarbyl-C(═O)—NH₂, -direct bond-C(═O)—NH—(C₁-C₁₀)hydrocarbyl or —(C₁-C₄)hydrocarbyl-C(═O)—NH—(C₁-C₁₀)hydrocarbyl, —(C₁-C₁₀)hydrocarbyl-NH—R^{e}, where the hydrocarbyl linker can be combined with R^{e} to form a (C₄-C₆) nitrogenous aliphatic heterocycle, and —(C₁-C₁₀)hydrocarbyl-NH—C(═O)(C₁-C₁₀)hydrocarbyl; R² is selected from H and optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂; R³-R²⁰ are each independently selected from hydrogen and deuterium; and wherein at least one of R³-R²⁰ is deuterium.

In a fifth aspect, the present disclosure relates to a compound of formula VIIa or VIIb:

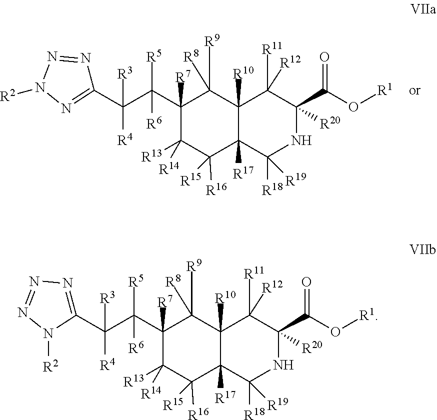

In a sixth aspect, the present disclosure relates to a compound of formula VIII:

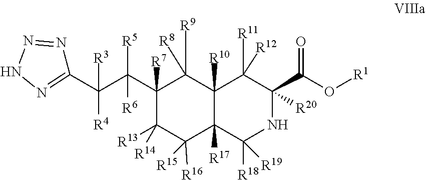

wherein: at least one of R³-R²⁰ is deuterium; and R¹ is chosen from H and (C₁-C₁₀) hydrocarbyl.

In a seventh aspect, the present disclosure relates to compounds of formulas I through VIII, and are shown to selectively inhibit (S)-α-amino-3-hydroxy-5-methyl-4-isoxazolepropionic acid (s-AMPA)-induced calcium permeability of AMPAR in vitro and prevent seizures in vivo in an animal model for epilepsy.

In an eighth aspect, the present disclosure relates to a pharmaceutical composition comprising a pharmaceutically acceptable carrier and a compound according to formula I, or a pharmaceutically acceptable salt thereof.

In a ninth aspect, the present disclosure relates to a method or medicament for treating epilepsy via the administration of said pharmaceutical compositions.

In an tenth aspect, the present disclosure relates to pharmaceutical compositions comprising a pharmaceutically acceptable carrier and a compound as described herein.

These and other objects, features, and advantages of the invention will become apparent from the following detailed description of the various aspects of the present invention.

### DETAILED DESCRIPTION

The present disclosure relates generally to 6-(2-(2H-tetrazol-5-yl)ethyl)decahydroisoquinoline-3-carboxylic acid and derivatives thereof, and to pharmaceutical compositions thereof, as well as methods for treating certain disorders.

Throughout this specification the terms and substituents retain their definitions.

For convenience and clarity, certain terms employed in the specification, examples, and claims are described herein.

The terms “hydrocarbyl,” “aliphatic hydrocarbyl,” and “aromatic hydrocarbyl,” as used herein and described below, include all possible structural features that are possible for the defined group, including linear, branched, cyclic, polycyclic, bridged, etc.

The term “alkyl,” as used herein and described below, includes all possible structural features that are possible for the defined group, including linear and branched and combinations thereof.

“Hydrocarbyl” (or “hydrocarbon”) refers to any group comprised of hydrogen and carbon as the only elemental constituents.

A first subset of hydrocarbyl is “aliphatic hydrocarbyl,” which refers to hydrocarbyl groups that are not aromatic. They include any variety of sp³-, sp²-, and sp-hybridized carbons that are not arranged to be aromatic as readily understood by a person with an ordinary knowledge of chemistry. Aliphatic hydrocarbyl groups encompass one or more alkane (sp³), alkene (sp²), alkyne (sp), and allene (sp² and sp) functional groups. Two or more alkene, alkyne, and/or allene functional groups may be conjugated in a hydrocarbyl group and the group is still defined as an aliphatic hydrocarbyl group as long as the conjugation does not constitute aromaticity.

Examples of aliphatic hydrocarbyl groups include, but are not limited to, methyl, ethyl, isopropyl, isobutyl, cyclopropyl, t-butyl, neopentyl, 3-methylbutyl, 3,3-dimethylbutyl, 2-propylpentyl, 2-butylhexyl, 2-pentylheptyl, 2-hexyloctyl, n-hexyl, n-octyl, 2-ethylbutyl, 1-methyl-2-ethylbutyl, decyl, dodecyl, tetradecyl, 9-hexadec-en-yl, 9-octadec-en-yl, 9,12-octadec-dien-yl, cyclohexyl, cyclohexylmethyl, 2-cyclohexylethyl, dicyclohexylmethyl, 2-butenyl, 2-butynyl, cyclopentyl, norbornyl, etc.

A second subset of hydrocarbyl is “aromatic hydrocarbyl” and “aryl,” which refers to hydrocarbyl groups that are aromatic. Aromatic hydrocarbyl groups include, but are not limited to, phenyl (C₆H₅), naphthyl (C₁₀H₇), anthracene (C₁₄H₉), etc.

A third subset of hydrocarbyl is “alkyl” (or alkane) which refers to hydrocarbyl groups consisting exclusively of sp³-hybridized carbon atoms. Alkyl groups are also a subset of aliphatic hydrocarbyl groups, except they are completely saturated hydrocarbyl groups that exclude the presence of sp²- and sp-hybridized carbon atoms. Examples of alkyl groups from the examples described above for aliphatic hydrocarbyl groups include methyl, ethyl, isopropyl, isobutyl, cyclopropyl, t-butyl, neopentyl, 3-methylbutyl, 3,3-dimethylbutyl, 2-propylpentyl, 2-butylhexyl, 2-pentylheptyl, 2-hexyloctyl, n-hexyl, n-octyl, 2-ethylbutyl, 1-methyl-2-ethylbutyl, decyl, dodecyl, tetradecyl, cyclohexyl, cyclohexylmethyl, 2-cyclohexylethyl, dicyclohexylmethyl, cyclopentyl, norbornyl, etc. Alkyl groups do not include, e.g., 9-hexadec-en-yl, 9-octadec-en-yl, 9,12-octadec-dien-yl, 2-butenyl, 2-butynyl, etc., which are also examples of aliphatic hydrocarbyl groups described above.

A fourth subset of hydrocarbyl is “cycloalkyl” and includes, but is not limited to, cyclic hydrocarbon groups of from 3 to 8 carbon atoms. Non-limiting examples of cycloalkyl groups include cyclopropyl, cyclobutyl, cyclopentyl, norbornyl, and the like.

Hydrocarbyl groups may be exclusively aliphatic hydrocarbyl, aromatic hydrocarbyl or alkyl in nature. Alternatively, the term hydrocarbyl encompasses combinations of one or more of these subset groups, typically as substituents. Thus, an aliphatic hydrocarbyl optionally substituted with, e.g., a phenyl group can be encompassed by the term “hydrocarbyl” or “aliphatic hydrocarbyl optionally substituted with phenyl.” “Alkyl optionally substituted with phenyl” indicates that, besides the phenyl group, all other carbon atoms are sp³-hybridized.

As defined above, aliphatic hydrocarbyl, aromatic hydrocarbyl (i.e., aryl), and alkyl, alone or in combinations, are proper limitations of hydrocarbyl provided carbon atom count is the same or less as the depended-upon hydrocarbyl carbon count. Likewise, alkyl is a further limitation of aliphatic hydrocarbyl provided carbon atom count is the same or less.

As used herein, the term “optionally substituted” may be used interchangeably with “unsubstituted or substituted”. The term “substituted” refers to the replacement of one or more hydrogen atoms in a specified group with a specified radical. For example, substituted alkyl, aryl, cycloalkyl, etc. refer to alkyl, aryl, or cycloalkyl wherein one or more H atoms in each residue are replaced with the specified substituent. In some embodiments, the substituent may be: halogen, haloalkyl, alkyl, acyl, alkoxyalkyl, hydroxy lower alkyl, carbonyl, phenyl, heteroaryl, benzenesulfonyl, hydroxy, lower alkoxy, haloalkoxy, oxaalkyl, carboxy, alkoxycarbonyl [—C(═O)O-alkyl], alkoxycarbonylamino [HNC(═O)O-alkyl], aminocarbonyl (also known as carboxamido) [—C(═O)NH₂], alkylaminocarbonyl [—C(═O)NH-alkyl], cyano, acetoxy, nitro, amino, alkylamino, dialkylamino, (alkyl)(aryl)aminoalkyl, alkylaminoalkyl (including cycloalkylaminoalkyl), dialkylaminoalkyl, dialkylaminoalkoxy, heterocyclylalkoxy, mercapto, alkylthio, sulfoxide, sulfone, sulfonylamino, alkylsulfinyl, alkylsulfonyl, acylaminoalkyl, acylaminoalkoxy, acylamino, amidino, aryl, benzyl, heterocyclyl, heterocyclylalkyl, phenoxy, benzyloxy, heteroaryloxy, hydroxyimino, alkoxyimino, oxaalkyl, aminosulfonyl, trityl, amidino, guanidino, ureido, benzyloxyphenyl, and benzyloxy. “Oxo” is also included among the substituents referred to in “optionally substituted”; it will be appreciated by persons of skill in the art that, because oxo is a divalent radical, there are circumstances in which it will not be appropriate as a substituent (e.g., on phenyl). In one embodiment, 1, 2, or 3 hydrogen atoms are replaced with a specified radical. In the case of alkyl and cycloalkyl, more than three hydrogen atoms can be replaced by fluorine; indeed, all available hydrogen atoms could be replaced by fluorine. In preferred embodiments, substituents are halogen, haloalkyl, alkyl, acyl, hydroxyalkyl, hydroxy, alkoxy, haloalkoxy, aminocarbonyl oxaalkyl, carboxy, cyano, acetoxy, nitro, amino, alkylamino, dialkylamino, alkylthio, alkylsulfinyl, alkylsulfonyl, alkylsulfonylamino arylsulfonyl, arylsulfonylamino, and benzyloxy. Most preferred are halogen, (C₁-C₄)alkyl, halo(C₁-C₄)alkyl, (C₁-C₄)alkoxy, halo(C₁-C₄)alkoxy, and aminocarbonyl.

As indicated above, definitions of R^{X} for hydrocarbyl, aliphatic hydrocarbyl, aromatic hydrocarbyl, and alkyl groups additionally include the number of carbons, designated as (Cₓ-C_{y}), where x is the minimum number of carbon atoms and y is the maximum number of carbon atoms. For example, “(C₁-C₂₀)hydrocarbyl” indicates a hydrocarbyl group of one to twenty carbons and “aliphatic (C₅-C₁₄)hydrocarbyl” indicates an aliphatic hydrocarbyl group of five to fourteen carbons. The carbon count of optional substituents containing carbon, e.g., phenyl, are separate from the (Cₓ-C_{y}) designation.

Unless otherwise specified, the term “carbocycle” is intended to include ring systems in which the ring atoms are all carbon but of any oxidation state. In a non-limiting example, (C₃-C₁₀) carbocycle refers to both non-aromatic and aromatic systems, including such systems as cyclopropane, benzene and cyclohexene. In another non-limiting example, (C₈-C₁₂) carbopolycycle refers to such systems as norbornane, decalin, indane, and naphthalene. Carbocycle, if not otherwise limited, refers to monocycles, bicycles and polycycles.

Heterocycle means an aliphatic or aromatic carbocycle residue in which from one to four carbons is replaced by a heteroatom selected from the group consisting of N, O, and S. The nitrogen and sulfur heteroatoms may optionally be oxidized, and the nitrogen heteroatom may optionally be quaternized. Unless otherwise specified, a heterocycle may be non-aromatic (heteroaliphatic) or aromatic (heteroaryl). Examples of heterocycles include, but are not limited to, pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, dihydrodioxole, tetrahydrofuran and the like. Examples of heterocyclyl residues include, but are not limited to, piperazinyl, piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl (also historically called thiophenyl), benzothienyl, thiamorpholinyl, oxadiazolyl, triazolyl and tetrahydroquinolinyl.

Alkoxy or alkoxyl refers to groups of from 1 to 20 carbon atoms, preferably 1 to 10 carbon atoms, more preferably 1 to 6 carbon atoms of a straight or branched configuration attached to the parent structure through an oxygen. Examples include, but are not limited to, methoxy, ethoxy, propoxy, isopropoxy and the like. Lower-alkoxy refers to groups containing one to four carbons. For the purpose of this application, alkoxy and lower alkoxy include methylenedioxy and ethylenedioxy.

Oxaalkyl refers to alkyl residues in which one or more carbons (and their associated hydrogens) have been replaced by oxygen. Examples include, but are not limited to, methoxypropoxy, 3,6,9-trioxadecyl and the like. The term oxaalkyl is intended as it is understood in the art (see Naming and Indexing of Chemical Substances for Chemical Abstracts, published by the American Chemical Society, 2002 edition, but without the restriction of 127(a)), i.e., it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively. Examples of azaalkyl include, but are not limited to, ethylaminoethyl and aminohexyl.

The term “halogen” means fluorine, chlorine, bromine, or iodine atoms.

The compounds described herein may contain, in a substituent R^{X}, double bonds and may also contain other centers of geometric asymmetry; unless specified otherwise, it is intended that the compounds include both E and Z geometric isomers. Likewise, all tautomeric forms are also intended to be included, for example tetrazole tautomers:

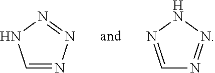 [SMILES: c1nnn[nH]1 ; c1nn[nH]n1]

The compounds described herein may contain one or more asymmetric centers and may thus give rise to enantiomers, diastereomers, and other stereoisomeric forms that may be defined, in terms of absolute stereochemistry, as (R)- or (S)-. The present disclosure is meant to include all such possible isomers, as well as their racemic and optically pure forms, and any mixture thereof. Optically active (R)- and (S)-isomers may be prepared using chiral synthons or chiral reagents or resolved using conventional techniques.

For clarity, the atom numbering convention for the decahydroisoquinoline ring system and its appendages is shown below:

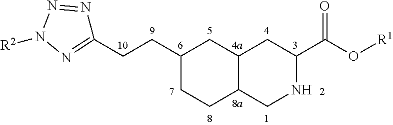

As used herein, and as would be understood by the person of skill in the art, the recitation of “a compound”—unless expressly further limited—is intended to include salts of that compound.

The term “pharmaceutically acceptable salt” refers to salts whose counter ion derives from pharmaceutically acceptable non-toxic acids and bases. Suitable pharmaceutically acceptable acids for salts of the amino-substituted compounds of the present invention include, for example, acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. Suitable pharmaceutically acceptable base addition salts for the carboxylate-substituted compounds of the present invention include, but are not limited to, metallic salts made from aluminum, calcium, lithium, magnesium, potassium, sodium and zinc or organic salts made from lysine, arginine, N,N-dibenzylethylenediamine, chloroprocaine, choline, diethanolamine, ethylenediamine, meglumine (N-methylglucamine), and procaine. Further pharmaceutically acceptable salts include, when appropriate, nontoxic ammonium cations and carboxylate, sulfonate and phosphonate anions attached to alkyl having from 1 to 20 carbon atoms.

It will be recognized that the compounds of this invention can exist in radiolabeled form, i.e., the compounds may contain one or more atoms containing an atomic mass or mass number different from the atomic mass or mass number usually found in nature. Alternatively, a plurality of molecules of a single structure may include at least one atom that occurs in an isotopic ratio that is different from the isotopic ratio found in nature. Radioisotopes of hydrogen, carbon, phosphorous, fluorine, chlorine and iodine include ²H, ³H, ¹¹C, ¹³C, ¹⁴C, ¹⁵N, ³⁵S, ¹⁸F, ³⁶Cl, ¹²⁵I, ¹²⁴I and ¹³¹I respectively. Compounds that contain those radioisotopes and/or other radioisotopes of other atoms are within the scope of this invention. Persons skilled in the art recognize that deuterium has been used to improve metabolic stability of compounds, and that principle can be applied to these compounds. Tritiated, i.e. ³H, and carbon-14, i.e., ¹⁴C, radioisotopes are particularly preferred for their ease in preparation and detectability. Compounds that contain isotopes ¹¹C, ¹³N, ¹⁵O, ¹²⁴I, and ¹⁸F are well suited for positron emission tomography. Radiolabeled compounds of formulae I and II of this invention and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

Although this invention is susceptible to embodiment in many different forms, preferred embodiments of the invention are shown. It should be understood, however, that the present disclosure is to be considered as an exemplification of the principles of this invention and is not intended to limit the invention to the embodiments illustrated. It may be found upon examination that certain members of the claimed genus are not patentable to the inventors in this application. In this event, subsequent exclusions of species from the compass of applicants' claims are to be considered artifacts of patent prosecution and not reflective of the inventors' concept or description of their invention; the invention encompasses all of the members of the genus I that are not already in the possession of the public.

The terms “subject” or “subject in need thereof” are used interchangeably herein. These terms refer to a patient who has been diagnosed with the underlying disorder to be treated. The subject may currently be experiencing symptoms associated with the disorder or may have experienced symptoms in the past. Additionally, a “subject in need thereof” may be a patient at risk of developing a particular disease, or to a patient reporting one or more of the physiological systems of a disease, even though a diagnosis of this disease may not have been made. As a non-limiting example, a “subject in need thereof”, for purposes of this application, may include a subject who is currently diagnosed with epilepsy or was diagnosed with epilepsy in the past, or who is at risk of seizures, regardless of current symptomatology.

As used herein, the terms “treatment” or “treating” are used interchangeably. These terms refer to an approach for obtaining beneficial or desired results including, but not limited to, therapeutic benefit. Therapeutic benefit includes eradication or amelioration of the underlying disorder being treated; it also includes the eradication or amelioration of one or more of the symptoms associated with the underlying disorder such that an improvement is observed in the patient, notwithstanding that the patient may still be afflicted with the underlying disorder.

The compounds described herein are useful for treating epilepsy. In the case of “treating epilepsy”, the term encompasses amelioration of the symptom of seizures and convulsions.

In one aspect, the present disclosure relates to a compound of formula Ia or Ib:

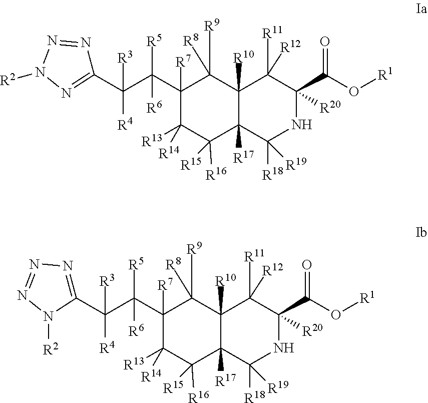

wherein:
R¹ is selected from H, (C₁-C₂₀)hydrocarbyl, and —CH(R^{a})R^{b};
R^{a} is selected from H, —(C₁-C₆)alkyl, and —C(═O)—O—(C₁-C₆)alkyl;
R^{b} is selected from —(C₁-C₁₀)hydrocarbyl-O—C(═O)—R^{c}, —(C₁-C₁₀)hydrocarbyl[-OC(═O)—R^{c}]₂, -direct bond-C(═O)—R^{c} or —(C₁-C₁₀)hydrocarbyl-C(═O)—R^{c}, —CH(NHR^{d})C(═O)—R^{e}, and —(C₁-C₁₀)hydrocarbyl-NR^{e}—C(═O)—R^{f};
R^{c} is selected from —O—(C₁-C₁₀)hydrocarbyl, —NH—(C₁-C₁₀)hydrocarbyl, —N—[(C₁-C₁₀)hydrocarbyl]₂, where the hydrocarbyl groups can be combined to form a (C₄-C₆) nitrogenous aliphatic heterocycle, —(C₁-C₁₀)hydrocarbyl, —NH₂, —(C₁-C₁₀)hydrocarbyl-NH—R^{e}, where the hydrocarbyl group can be combined with R^{e} to form a (C₄-C₆) nitrogenous aliphatic heterocycle, and —NR^{e}—(C₁-C₁₀)hydrocarbyl-C(═O)—O—(C₁-C₁₀)hydrocarbyl, where the hydrocarbyl group and R^{e} can be combined to form a (C₄-C₆) nitrogenous aliphatic heterocycle;
R^{d} is selected from H, —(C₁-C₁₀)hydrocarbyl, and —C(═O)(C₁-C₁₀)hydrocarbyl;
R^{e} is selected from H, and —(C₁-C₆)alkyl;
R^{f} is selected from —O—(C₁-C₁₀)hydrocarbyl, —(C₁-C₁₀)hydrocarbyl, -direct bond-C(═O)—O—(C₁-C₁₀)hydrocarbyl or —(C₁-C₄)hydrocarbyl-C(═O)—O—(C₁-C₁₀)hydrocarbyl, -direct bond-C(═O)—NH₂ or —(C₁-C₄)hydrocarbyl-C(═O)—NH₂, -direct bond-C(═O)—NH—(C₁-C₁₀)hydrocarbyl or —(C₁-C₄)hydrocarbyl-C(═O)—NH—(C₁-C₁₀)hydrocarbyl, —(C₁-C₁₀)hydrocarbyl-NH—R^{e}, where the hydrocarbyl linker can be combined with R^{e} to form a (C₄-C₆) nitrogenous aliphatic heterocycle, and —(C₁-C₁₀)hydrocarbyl-NH—C(═O)(C₁-C₁₀)hydrocarbyl;
R² is selected from H and optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂;
R⁷ is selected from hydrogen, deuterium, and fluorine;
R³-R⁶ and R⁸-R²⁰ are independently selected from hydrogen and deuterium; and with the provisos:
   (i) when R¹ is C₁-C₆ alkyl or aryl and R² is a nitrogen protecting group, wherein said nitrogen protecting group is selected from trityl, benzyl, t-butyl, t-butyldimethylsilyl, and triphenylsilyl, then either (a) R⁷ is fluorine or (b) at least one of R³-R²⁰ is deuterium;
   (ii) when R¹ is H, C₁-C₆ alkyl, substituted alkyl, cycloalkyl, or arylalkyl and R² is H, then either (a) R⁷ is fluorine or at (b) least one of R³-R²⁰ is deuterium; and
   (iii) when R⁷ is fluorine, then R² is not H.

In one aspect, the present disclosure relates to a compound of formula IIa, IIb, IIIa, or IIIb:

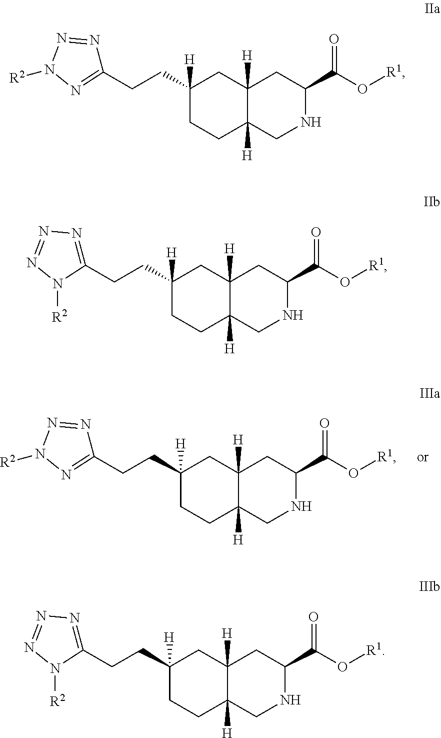

wherein:
R¹ is selected from H and (C₁-C₂₀)hydrocarbyl;
R² is optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂; and
R³-R²⁰ are each H.

In one aspect, the present disclosure relates to a compound of formula IVa, IVb, Va, or Vb:

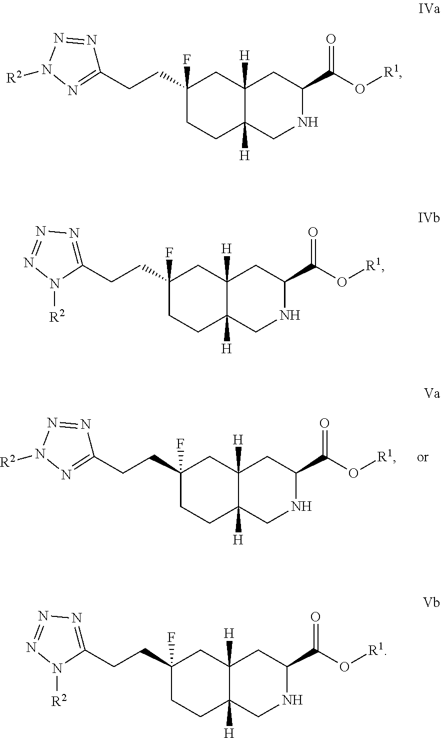

wherein:
R¹ is selected from H and (C₁-C₂₀)hydrocarbyl;
R³-R⁶ and R⁸-R²⁰ are each H;
R⁷ is fluorine; and
R² is optionally substituted (C₁-C₇)hydrocarbyl, wherein said optionally substituted (C₁-C₇)hydrocarbyl may be substituted with one or more of —F, —CN, —OH, —O—C(═O)(C₁-C₄)hydrocarbyl, —C(═O)—OH, —C(═O)—O—CH₃, —C(═O)—NH—CH₃, —NH(C₁-C₄)hydrocarbyl, —N[(C₁-C₄)hydrocarbyl)]₂, or —C(═O)—N[(C₁-C₃)hydrocarbyl]₂;

In some embodiments

In various embodiments of the present disclosure, R¹ is chosen from: (C₁-C₂₀)hydrocarbyl, (C₁-C₁₉)hydrocarbyl, (C₁-C₁₈)hydrocarbyl, (C₁-C₁₇)hydrocarbyl, (C₁-C₁₆)hydrocarbyl, (C₁-C₁₈)hydrocarbyl, (C₁-C₁₄)hydrocarbyl, (C₁-C₁₃)hydrocarbyl, (C₁-C₁₂)hydrocarbyl, (C₁-C₁₁)hydrocarbyl, (C₁-C₁₀)hydrocarbyl, (C₁-C₉)hydrocarbyl, (C₁-C₅)hydrocarbyl, (C₁-C₇)hydrocarbyl, (C₁-C₆)hydrocarbyl, (C₁-C₅)hydrocarbyl, (C₁-C₄)hydrocarbyl, and (C₁-C₃)hydrocarbyl.

In various embodiments of the present disclosure, R¹ is chosen from: (C₂-C₂₀)hydrocarbyl, (C₂-C₁₉)hydrocarbyl, (C₂-C₁₈)hydrocarbyl, (C₂-C₁₇)hydrocarbyl, (C₂-C₁₆)hydrocarbyl, (C₂-C₁₅)hydrocarbyl, (C₂-C₁₄)hydrocarbyl, (C₂-C₁₃)hydrocarbyl, (C₂-C₁₂)hydrocarbyl, (C₂-C₁₁)hydrocarbyl, (C₂-C₁₀)hydrocarbyl, (C₂-C₉)hydrocarbyl, (C₂-C₈)hydrocarbyl, (C₂-C₇)hydrocarbyl, (C₂-C₆)hydrocarbyl, (C₂-C₅)hydrocarbyl, (C₂-C₄)hydrocarbyl, and (C₂-C₃)hydrocarbyl.

In various embodiments of the present disclosure, R¹ is chosen from: (C₃-C₂₀)hydrocarbyl, (C₃-C₁₉)hydrocarbyl, (C₃-C₁₈)hydrocarbyl, (C₃-C₁₇)hydrocarbyl, (C₃-C₁₆)hydrocarbyl, (C₃-C₁₅)hydrocarbyl, (C₃-C₁₄)hydrocarbyl, (C₃-C₁₃)hydrocarbyl, (C₃-C₁₂)hydrocarbyl, (C₃-C₁₁)hydrocarbyl, (C₃-C₁₀)hydrocarbyl, (C₃-C₉)hydrocarbyl, (C₃-C₈)hydrocarbyl, (C₃-C₇)hydrocarbyl, (C₃-C₆)hydrocarbyl, (C₃-C₅)hydrocarbyl, and (C₃-C₄)hydrocarbyl.

In various embodiments of the present disclosure, R¹ is chosen from: (C₄-C₂₀)hydrocarbyl, (C₄-C₁₉)hydrocarbyl, (C₄-C₁₈)hydrocarbyl, (C₄-C₁₇)hydrocarbyl, (C₄-C₁₆)hydrocarbyl, (C₄-C₁₅)hydrocarbyl, (C₄-C₁₄)hydrocarbyl, (C₄-C₁₃)hydrocarbyl, (C₄-C₁₂)hydrocarbyl, (C₄-C₁₁)hydrocarbyl, (C₄-C₁₀)hydrocarbyl, (C₄-C₉)hydrocarbyl, (C₄-C₈)hydrocarbyl, (C₄-C₇)hydrocarbyl, (C₄-C₆)hydrocarbyl, and (C₄-C₅)hydrocarbyl.

In various embodiments of the present disclosure, R¹ is chosen from: (C₅-C₂₀)hydrocarbyl, (C₅-C₁₉)hydrocarbyl, (C₅-C₁₈)hydrocarbyl, (C₅-C₁₇)hydrocarbyl, (C₅-C₁₆)hydrocarbyl, (C₅-C₁₅)hydrocarbyl, (C₅-C₁₄)hydrocarbyl, (C₅-C₁₃)hydrocarbyl, (C₅-C₁₂)hydrocarbyl, (C₅-C₁₁)hydrocarbyl, (C₅-C₁₀)hydrocarbyl, (C₅-C₉)hydrocarbyl, (C₅-C₈)hydrocarbyl, (C₅-C₇)hydrocarbyl, and (C₅-C₆)hydrocarbyl.

In various embodiments of the present disclosure, R¹ is chosen from: (C₆-C₂₀)hydrocarbyl, (C₆-C₁₉)hydrocarbyl, (C₆-C₁₈)hydrocarbyl, (C₆-C₁₇)hydrocarbyl, (C₆-C₁₆)hydrocarbyl, (C₆-C₁₅)hydrocarbyl, (C₆-C₁₄)hydrocarbyl, (C₆-C₁₃)hydrocarbyl, (C₆-C₁₂)hydrocarbyl, (C₆-C₁₁)hydrocarbyl, (C₆-C₁₀)hydrocarbyl, (C₆-C₉)hydrocarbyl, (C₆-C₈)hydrocarbyl, and (C₆-C₇)hydrocarbyl.

In various embodiments of the present disclosure, R¹ is chosen from: (C₇-C₂₀)hydrocarbyl, (C₇-C₁₉)hydrocarbyl, (C₇-C₁₈)hydrocarbyl, (C₇-C₁₇)hydrocarbyl, (C₇-C₁₆)hydrocarbyl, (C₇-C₁₅)hydrocarbyl, (C₇-C₁₄)hydrocarbyl, (C₇-C₁₃)hydrocarbyl, (C₇-C₁₂)hydrocarbyl, (C₇-C₁₁)hydrocarbyl, (C₇-C₁₀)hydrocarbyl, (C₇-C₉)hydrocarbyl, and (C₇-C₈)hydrocarbyl.

In various embodiments of the present disclosure, R¹ is chosen from: (C₈-C₂₀)hydrocarbyl, (C₈-C₁₉)hydrocarbyl, (C₈-C₁₈)hydrocarbyl, (C₈-C₁₇)hydrocarbyl, (C₈-C₁₆)hydrocarbyl, (C₈-C₁₅)hydrocarbyl, (C₈-C₁₄)hydrocarbyl, (C₈-C₁₃)hydrocarbyl, (C₈-C₁₂)hydrocarbyl, (C₈-C₁₁)hydrocarbyl, (C₈-C₁₀)hydrocarbyl, and (C₈-C₉)hydrocarbyl.

In various embodiments of the present disclosure, R¹ is chosen from: (C₉-C₂₀)hydrocarbyl, (C₉-C₁₉)hydrocarbyl, (C₉-C₁₈)hydrocarbyl, (C₉-C₁₇)hydrocarbyl, (C₉-C₁₆)hydrocarbyl, (C₉-C₁₅)hydrocarbyl, (C₉-C₁₄)hydrocarbyl, (C₉-C₁₃)hydrocarbyl, (C₉-C₁₂)hydrocarbyl, (C₉-C₁₁)hydrocarbyl, and (C₉-C₁₀)hydrocarbyl.

In various embodiments of the present disclosure, R¹ is chosen from: (C₁₀-C₂₀)hydrocarbyl, (C₁₀-C₁₉)hydrocarbyl, (C₁₀-C₁₈)hydrocarbyl, (C₁₀-C₁₇)hydrocarbyl, (C₁₀-C₁₆)hydrocarbyl, (C₁₀-C₁₅)hydrocarbyl, (C₁₀-C₁₄)hydrocarbyl, (C₁₀-C₁₃)hydrocarbyl, (C₁₀-C₁₂)hydrocarbyl, and (C₁₀-C₁₁)hydrocarbyl.

In various embodiments of the present disclosure, R¹ is chosen from: (C₁₁-C₂₀)hydrocarbyl, (C₁₁-C₁₉)hydrocarbyl, (C₁₁-C₁₈)hydrocarbyl, (C₁₁-C₁₇)hydrocarbyl, (C₁₁-C₁₆)hydrocarbyl, (C₁₁-C₁₅)hydrocarbyl, (C₁₁-C₁₄)hydrocarbyl, (C₁₁-C₁₃)hydrocarbyl, and (C₁₁-C₁₂)hydrocarbyl.

In various embodiments of the present disclosure, R¹ is chosen from: (C₁₂-C₂₀)hydrocarbyl, (C₁₂-C₁₉)hydrocarbyl, (C₁₂-C₁₈)hydrocarbyl, (C₁₂-C₁₇)hydrocarbyl, (C₁₂-C₁₆)hydrocarbyl, (C₁₂-C₁₅)hydrocarbyl, (C₁₂-C₁₄)hydrocarbyl, and (C₁₂-C₁₃)hydrocarbyl.

In various embodiments of the present disclosure, R¹ is chosen from: (C₁₃-C₂₀)hydrocarbyl, (C₁₃-C₁₉)hydrocarbyl, (C₁₃-C₁₈)hydrocarbyl, (C₁₃-C₁₇)hydrocarbyl, (C₁₃-C₁₆)hydrocarbyl, (C₁₃-C₁₅)hydrocarbyl, and (C₁₃-C₁₄)hydrocarbyl.

In various embodiments of the present disclosure, R¹ is chosen from: (C₁₄-C₂₀)hydrocarbyl, (C₁₄-C₁₉)hydrocarbyl, (C₁₄-C₁₈)hydrocarbyl, (C₁₄-C₁₇)hydrocarbyl, (C₁₄-C₁₆)hydrocarbyl, and (C₁₄-C₁₅)hydrocarbyl.

In various embodiments of the present disclosure, R¹ is chosen from: (C₁₅-C₂₀)hydrocarbyl, (C₁₅-C₁₉)hydrocarbyl, (C₁₅-C₁₈)hydrocarbyl, (C₁₅-C₁₇)hydrocarbyl, and (C₁₅-C₁₆)hydrocarbyl.

In various embodiments of the present disclosure, R¹ is chosen from: (C₁₆-C₂₀)hydrocarbyl, (C₁₆-C₁₉)hydrocarbyl, (C₁₆-C₁₈)hydrocarbyl, and (C₁₆-C₁₇)hydrocarbyl.

In various embodiments of the present disclosure, R¹ is chosen from: (C₁₇-C₂₀)hydrocarbyl, (C₁₇-C₁₉)hydrocarbyl, and (C₁₇-C₁₈)hydrocarbyl.

In various embodiments of the present disclosure, R¹ is (C₁₈-C₂₀)hydrocarbyl or (C₁₈-C₁₉)hydrocarbyl.

In various embodiments of the present disclosure, R¹ is (C₁₉-C₂₀)hydrocarbyl.

In various embodiments of the present disclosure, R¹ is chosen from: (C₂₀)hydrocarbyl, (C₁₉)hydrocarbyl, (C₁₈)hydrocarbyl, (C₁₇)hydrocarbyl, (C₁₆)hydrocarbyl, (C₁₅)hydrocarbyl, (C₁₄)hydrocarbyl, (C₁₃)hydrocarbyl, (C₁₂)hydrocarbyl, (C₁₁)hydrocarbyl, (C₁₀)hydrocarbyl, (C₉)hydrocarbyl, (C₈)hydrocarbyl, (C₇)hydrocarbyl, (C₆)hydrocarbyl, (C₅)hydrocarbyl, (C₄)hydrocarbyl, and (C₃)hydrocarbyl.

In some embodiments of the present disclosure, R¹ is chosen from: aliphatic (C₁-C₂₀)hydrocarbyl optionally substituted with one or two phenyl groups with the proviso that R¹ contains twenty carbons or less, aliphatic (C₁-C₁₉)hydrocarbyl optionally substituted with one or two phenyl groups with the proviso that R¹ contains twenty carbons or less, aliphatic (C₁-C₁₈)hydrocarbyl optionally substituted with one or two phenyl groups with the proviso that R¹ contains twenty carbons or less, aliphatic (C₁-C₁₇)hydrocarbyl optionally substituted with one or two phenyl groups with the proviso that R¹ contains twenty carbons or less, aliphatic (C₁-C₁₆)hydrocarbyl optionally substituted with one or two phenyl groups with the proviso that R¹ contains twenty carbons or less, aliphatic (C₁-C₁₅)hydrocarbyl optionally substituted with one or two phenyl groups with the proviso that R¹ contains twenty carbons or less, aliphatic (C₁-C₁₄)hydrocarbyl optionally substituted with one or two phenyl groups with the proviso that R¹ contains twenty carbons or less, aliphatic (C₁-C₁₃)hydrocarbyl optionally substituted with one or two phenyl groups with the proviso that R¹ contains twenty carbons or less, aliphatic (C₁-C₁₂)hydrocarbyl optionally substituted with one or two phenyl groups with the proviso that R¹ contains twenty carbons or less, aliphatic (C₁-C₁₁)hydrocarbyl optionally substituted with one or two phenyl groups with the proviso that R¹ contains twenty carbons or less, aliphatic (C₁-C₁₀)hydrocarbyl optionally substituted with one or two phenyl groups with the proviso that R¹ contains twenty carbons or less, aliphatic (C₁-C₉)hydrocarbyl optionally substituted with one or two phenyl groups with the proviso that R¹ contains twenty carbons or less, aliphatic (C₁-C₈)hydrocarbyl optionally substituted with one or two phenyl groups, aliphatic (C₁-C₇)hydrocarbyl optionally substituted with one or two phenyl groups, aliphatic (C₁-C₆)hydrocarbyl optionally substituted with one or two phenyl groups, aliphatic (C₁-C₅)hydrocarbyl optionally substituted with one or two phenyl groups, aliphatic (C₁-C₄)hydrocarbyl optionally substituted with one or two phenyl groups, and aliphatic (C₁-C₃)hydrocarbyl optionally substituted with one or two phenyl groups.

In various embodiments of the present disclosure, R¹ is chosen from: aliphatic (C₂-C₂₀)hydrocarbyl optionally substituted with one or two phenyl groups with the proviso that R¹ contains twenty carbons or less, aliphatic (C₂-C₁₉)hydrocarbyl optionally substituted with one or two phenyl groups with the proviso that R¹ contains twenty carbons or less, aliphatic (C₂-C₁₈)hydrocarbyl optionally substituted with one or two phenyl groups with the proviso that R¹ contains twenty carbons or less, aliphatic (C₂-C₁₇)hydrocarbyl optionally substituted with one or two phenyl groups with the proviso that R¹ contains twenty carbons or less, aliphatic (C₂-C₁₆)hydrocarbyl optionally substituted with one or two phenyl groups with the proviso that R¹ contains twenty carbons or less, aliphatic (C₂-C₁₅)hydrocarbyl optionally substituted with one or two phenyl groups with the proviso that R¹ contains twenty carbons or less, aliphatic (C₂-C₁₄)hydrocarbyl optionally substituted with one or two phenyl groups with the proviso that R¹ contains twenty carbons or less, aliphatic (C₂-C₁₃)hydrocarbyl optionally substituted with one or two phenyl groups with the proviso that R¹ contains twenty carbons or less, aliphatic (C₂-C₁₂)hydrocarbyl optionally substituted with one or two phenyl groups with the proviso that R¹ contains twenty carbons or less, aliphatic (C₂-C₁₁)hydrocarbyl optionally substituted with one or two phenyl groups with the proviso that R¹ contains twenty carbons or less, aliphatic (C₂-C₁₀)hydrocarbyl optionally substituted with one or two phenyl groups with the proviso that R¹ contains twenty carbons or less, aliphatic (C₂-C₉)hydrocarbyl optionally substituted with one or two phenyl groups with the proviso that R¹ contains twenty carbons or less, aliphatic (C₂-C₈)hydrocarbyl optionally substituted with one or two phenyl groups, aliphatic (C₂-C₇)hydrocarbyl optionally substituted with one or two phenyl groups, aliphatic (C₂-C₆)hydrocarbyl optionally substituted with one or two phenyl groups, aliphatic (C₂-C₅)hydrocarbyl optionally substituted with one or two phenyl groups, aliphatic (C₂-C₄)hydrocarbyl optionally substituted with one or two phenyl groups, and aliphatic (C₂-C₃)hydrocarbyl optionally substituted with one or two phenyl groups.

In various embodiments of the present disclosure R¹ is chosen from: aliphatic (C₃-C₂₀)hydrocarbyl optionally substituted with one or two phenyl groups with the proviso that R¹ contains twenty carbons or less, aliphatic (C₃-C₁₉)hydrocarbyl optionally substituted with one or two phenyl groups with the proviso that R¹ contains twenty carbons or less, aliphatic (C₃-C₁₈)hydrocarbyl optionally substituted with one or two phenyl groups with the proviso that R¹ contains twenty carbons or less, aliphatic (C₃-C₁₇)hydrocarbyl optionally substituted with one or two phenyl groups with the proviso that R¹ contains twenty carbons or less, aliphatic (C₃-C₁₆)hydrocarbyl optionally substituted with one or two phenyl groups with the proviso that R¹ contains twenty carbons or less, aliphatic (C₃-C₁₅)hydrocarbyl optionally substituted with one or two phenyl groups with the proviso that R¹ contains twenty carbons or less, aliphatic (C₃-C₁₄)hydrocarbyl optionally substituted with one or two phenyl groups with the proviso that R¹ contains twenty carbons or less, aliphatic (C₃-C₁₃)hydrocarbyl optionally substituted with one or two phenyl groups with the proviso that R¹ contains twenty carbons or less, aliphatic (C₃-C₁₂)hydrocarbyl optionally substituted with one or two phenyl groups with the proviso that R¹ contains twenty carbons or less, aliphatic (C₃-C₁₁)hydrocarbyl optionally substituted with one or two phenyl groups with the proviso that R¹ contains twenty carbons or less, aliphatic (C₃-C₁₀)hydrocarbyl optionally substituted with one or two phenyl groups with the proviso that R¹ contains twenty carbons or less, aliphatic (C₃-C₉)hydrocarbyl optionally substituted with one or two phenyl groups with the proviso that R¹ contains twenty carbons or less, aliphatic (C₃-C₈)hydrocarbyl optionally substituted with one or two phenyl groups, aliphatic (C₃-C₇)hydrocarbyl optionally substituted with one or two phenyl groups, aliphatic (C₃-C₆)hydrocarbyl optionally substituted with one or two phenyl groups, aliphatic (C₃-C₅)hydrocarbyl optionally substituted with one or two phenyl groups, aliphatic (C₃-C₄)hydrocarbyl optionally substituted with one or two phenyl groups.

In various embodiments of the present disclosure R¹ is chosen from: aliphatic (C₄-C₂₀)hydrocarbyl optionally substituted with one or two phenyl groups with the proviso that R¹ contains twenty carbons or less, aliphatic (C₄-C₁₉)hydrocarbyl optionally substituted with one or two phenyl groups with the proviso that R¹ contains twenty carbons or less, aliphatic (C₄-C₁₈)hydrocarbyl optionally substituted with one or two phenyl groups with the proviso that R¹ contains twenty carbons or less, aliphatic (C₄-C₁₇)hydrocarbyl optionally substituted with one or two phenyl groups with the proviso that R¹ contains twenty carbons or less, aliphatic (C₄-C₁₆)hydrocarbyl optionally substituted with one or two phenyl groups with the proviso that R¹ contains twenty carbons or less, aliphatic (C₄-C₁₅)hydrocarbyl optionally substituted with one or two phenyl groups with the proviso that R¹ contains twenty carbons or less, aliphatic (C₄-C₁₄)hydrocarbyl optionally substituted with one or two phenyl groups with the proviso that R¹ contains twenty carbons or less, aliphatic (C₄-C₁₃)hydrocarbyl optionally substituted with one or two phenyl groups with the proviso that R¹ contains twenty carbons or less, aliphatic (C₄-C₁₂)hydrocarbyl optionally substituted with one or two phenyl groups with the proviso that R¹ contains twenty carbons or less, aliphatic (C₄-C₁₁)hydrocarbyl optionally substituted with one or two phenyl groups with the proviso that R¹ contains twenty carbons or less, aliphatic (C₄-C₁₀)hydrocarbyl optionally substituted with one or two phenyl groups with the proviso that R¹ contains twenty carbons or less, aliphatic (C₄-C₉)hydrocarbyl optionally substituted with one or two phenyl groups with the proviso that R¹ contains twenty carbons or less, aliphatic (C₄-C₈)hydrocarbyl optionally substituted with one or two phenyl groups, aliphatic (C₄-C₇)hydrocarbyl optionally substituted with one or two phenyl groups, aliphatic (C₄-C₆)hydrocarbyl optionally substituted with one or two phenyl groups, and aliphatic (C₄-C₅)hydrocarbyl optionally substituted with one or two phenyl groups.

In various embodiments of the present disclosure, R¹ is chosen from: aliphatic (C₅-C₂₀)hydrocarbyl optionally substituted with one or two phenyl groups with the proviso that R¹ contains twenty carbons or less, aliphatic (C₅-C₁₉)hydrocarbyl optionally substituted with one or two phenyl groups with the proviso that R¹ contains twenty carbons or less, aliphatic (C₅-C₁₈)hydrocarbyl optionally substituted with one or two phenyl groups with the proviso that R¹ contains twenty carbons or less, aliphatic (C₅-C₁₇)hydrocarbyl optionally substituted with one or two phenyl groups with the proviso that R¹ contains twenty carbons or less, aliphatic (C₅-C₁₆)hydrocarbyl optionally substituted with one or two phenyl groups with the proviso that R¹ contains twenty carbons or less, aliphatic (C₅-C₁₅)hydrocarbyl optionally substituted with one or two phenyl groups with the proviso that R¹ contains twenty carbons or less, aliphatic (C₅-C₁₄)hydrocarbyl optionally substituted with one or two phenyl groups with the proviso that R¹ contains twenty carbons or less, aliphatic (C₅-C₁₃)hydrocarbyl optionally substituted with one or two phenyl groups with the proviso that R¹ contains twenty carbons or less, aliphatic (C₅-C₁₂)hydrocarbyl optionally substituted with one or two phenyl groups with the proviso that R¹ contains twenty carbons or less, aliphatic (C₅-C₁₁)hydrocarbyl optionally substituted with one or two phenyl groups with the proviso that R¹ contains twenty carbons or less, aliphatic (C₅-C₁₀)hydrocarbyl optionally substituted with one or two phenyl groups with the proviso that R¹ contains twenty carbons or less, aliphatic (C₅-C₉)hydrocarbyl optionally substituted with one or two phenyl groups with the proviso that R¹ contains twenty carbons or less, aliphatic (C₅-C₈)hydrocarbyl optionally substituted with one or two phenyl groups, aliphatic (C₅-C₇)hydrocarbyl optionally substituted with one or two phenyl groups, and aliphatic (C₅-C₆)hydrocarbyl optionally substituted with one or two phenyl groups.

In various embodiments of the present disclosure, R¹ is chosen from: aliphatic (C₆-C₂₀)hydrocarbyl optionally substituted with one or two phenyl groups with the proviso that R¹ contains twenty carbons or less, aliphatic (C₆-C₁₉)hydrocarbyl optionally substituted with one or two phenyl groups with the proviso that R¹ contains twenty carbons or less, aliphatic (C₆-C₁₈)hydrocarbyl optionally substituted with one or two phenyl groups with the proviso that R¹ contains twenty carbons or less, aliphatic (C₆-C₁₇)hydrocarbyl optionally substituted with one or two phenyl groups with the proviso that R¹ contains twenty carbons or less, aliphatic (C₆-C₁₆)hydrocarbyl optionally substituted with one or two phenyl groups with the proviso that R¹ contains twenty carbons or less, aliphatic (C₆-C₁₅)hydrocarbyl optionally substituted with one or two phenyl groups with the proviso that R¹ contains twenty carbons or less, aliphatic (C₆-C₁₄)hydrocarbyl optionally substituted with one or two phenyl groups with the proviso that R¹ contains twenty carbons or less, aliphatic (C₆-C₁₃)hydrocarbyl optionally substituted with one or two phenyl groups with the proviso that R¹ contains twenty carbons or less, aliphatic (C₆-C₁₂)hydrocarbyl optionally substituted with one or two phenyl groups with the proviso that R¹ contains twenty carbons or less, aliphatic (C₆-C₁₁)hydrocarbyl optionally substituted with one or two phenyl groups with the proviso that R¹ contains twenty carbons or less, aliphatic (C₆-C₁₀)hydrocarbyl optionally substituted with one or two phenyl groups with the proviso that R¹ contains twenty carbons or less, aliphatic (C₆-C₉)hydrocarbyl optionally substituted with one or two phenyl groups with the proviso that R¹ contains twenty carbons or less, aliphatic (C₆-C₈)hydrocarbyl optionally substituted with one or two phenyl groups, and aliphatic (C₆-C₇)hydrocarbyl optionally substituted with one or two phenyl groups.

In various embodiments of the present disclosure, R¹ is chosen from: aliphatic (C₇-C₂₀)hydrocarbyl optionally substituted with one or two phenyl groups with the proviso that R¹ contains twenty carbons or less, aliphatic (C₇-C₁₉)hydrocarbyl optionally substituted with one or two phenyl groups with the proviso that R¹ contains twenty carbons or less, aliphatic (C₇-C₁₈)hydrocarbyl optionally substituted with one or two phenyl groups with the proviso that R¹ contains twenty carbons or less, aliphatic (C₇-C₁₇)hydrocarbyl optionally substituted with one or two phenyl groups with the proviso that R¹ contains twenty carbons or less, aliphatic (C₇-C₁₆)hydrocarbyl optionally substituted with one or two phenyl groups with the proviso that R¹ contains twenty carbons or less, aliphatic (C₇-C₁₅)hydrocarbyl optionally substituted with one or two phenyl groups with the proviso that R¹ contains twenty carbons or less, aliphatic (C₇-C₁₄)hydrocarbyl optionally substituted with one or two phenyl groups with the proviso that R¹ contains twenty carbons or less, aliphatic (C₇-C₁₃)hydrocarbyl optionally substituted with one or two phenyl groups with the proviso that R¹ contains twenty carbons or less, aliphatic (C₇-C₁₂)hydrocarbyl optionally substituted with one or two phenyl groups with the proviso that R¹ contains twenty carbons or less, aliphatic (C₇-C₁₁)hydrocarbyl optionally substituted with one or two phenyl groups with the proviso that R¹ contains twenty carbons or less, aliphatic (C₇-C₁₀)hydrocarbyl optionally substituted with one or two phenyl groups with the proviso that R¹ contains twenty carbons or less, aliphatic (C₇-C₉)hydrocarbyl optionally substituted with one or two phenyl groups with the proviso that R¹ contains twenty carbons or less, and aliphatic (C₇-C₈)hydrocarbyl optionally substituted with one or two phenyl groups.

In various embodiments of the present disclosure, R¹ is chosen from: aliphatic (C₈-C₂₀)hydrocarbyl optionally substituted with one or two phenyl groups with the proviso that R¹ contains twenty carbons or less, aliphatic (C₈-C₁₉)hydrocarbyl optionally substituted with one or two phenyl groups with the proviso that R¹ contains twenty carbons or less, aliphatic (C₈-C₁₈)hydrocarbyl optionally substituted with one or two phenyl groups with the proviso that R¹ contains twenty carbons or less, aliphatic (C₈-C₁₇)hydrocarbyl optionally substituted with one or two phenyl groups with the proviso that R¹ contains twenty carbons or less, aliphatic (C₈-C₁₆)hydrocarbyl optionally substituted with one or two phenyl groups with the proviso that R¹ contains twenty carbons or less, aliphatic (C₈-C₁₅)hydrocarbyl optionally substituted with one or two phenyl groups with the proviso that R¹ contains twenty carbons or less, aliphatic (C₈-C₁₄)hydrocarbyl optionally substituted with one or two phenyl groups with the proviso that R¹ contains twenty carbons or less, aliphatic (C₈-C₁₃)hydrocarbyl optionally substituted with one or two phenyl groups with the proviso that R¹ contains twenty carbons or less, aliphatic (C₈-C₁₂)hydrocarbyl optionally substituted with one or two phenyl groups with the proviso that R¹ contains twenty carbons or less, aliphatic (C₈-C₁₁)hydrocarbyl optionally substituted with one or two phenyl groups with the proviso that R¹ contains twenty carbons or less, aliphatic (C₈-C₁₀)hydrocarbyl optionally substituted with one or two phenyl groups with the proviso that R¹ contains twenty carbons or less, and aliphatic (C₈-C₉)hydrocarbyl optionally substituted with one or two phenyl groups with the proviso that R¹ contains twenty carbons or less.

In various embodiments of the present disclosure, R¹ is chosen from: aliphatic (C₉-C₂₀)hydrocarbyl optionally substituted with a phenyl group with the proviso that R¹ contains twenty carbons or less, aliphatic (C₉-C₁₉)hydrocarbyl optionally substituted with a phenyl group with the proviso that R contains twenty carbons or less, aliphatic (C₉-C₁₈)hydrocarbyl optionally substituted with a phenyl group with the proviso that R¹ contains twenty carbons or less, aliphatic (C₉-C₁₇)hydrocarbyl optionally substituted with a phenyl group with the proviso that R¹ contains twenty carbons or less, aliphatic (C₉-C₁₆)hydrocarbyl optionally substituted with a phenyl group with the proviso that R¹ contains twenty carbons or less, aliphatic (C₉-C₁₅)hydrocarbyl optionally substituted with a phenyl group with the proviso that R¹ contains twenty carbons or less, aliphatic (C₉-C₁₄)hydrocarbyl optionally substituted with a phenyl group, aliphatic (C₉-C₁₃)hydrocarbyl optionally substituted with a phenyl group, aliphatic (C₉-C₁₂)hydrocarbyl optionally substituted with a phenyl group, aliphatic (C₉-C₁₁)hydrocarbyl optionally substituted with a phenyl group, and aliphatic (C₉-C₁₀)hydrocarbyl optionally substituted with a phenyl group.

In various embodiments of the present disclosure, R¹ is chosen from: aliphatic (C₁₀-C₂₀)hydrocarbyl optionally substituted with a phenyl group with the proviso that R¹ contains twenty carbons or less, aliphatic (C₁₀-C₁₉)hydrocarbyl optionally substituted with a phenyl group with the proviso that R¹ contains twenty carbons or less, aliphatic (C₁₀-C₁₈)hydrocarbyl optionally substituted with a phenyl group with the proviso that R¹ contains twenty carbons or less, aliphatic (C₁₀-C₁₇)hydrocarbyl optionally substituted with a phenyl group with the proviso that R¹ contains twenty carbons or less, aliphatic (C₁₀-C₁₆)hydrocarbyl optionally substituted with a phenyl group with the proviso that R¹ contains twenty carbons or less, aliphatic (C₁₀-C₁₅)hydrocarbyl optionally substituted with a phenyl group with the proviso that R¹ contains twenty carbons or less, aliphatic (C₁₀-C₁₄)hydrocarbyl optionally substituted with a phenyl group, aliphatic (C₁₀-C₁₃)hydrocarbyl optionally substituted with a phenyl group, aliphatic (C₁₀-C₁₂)hydrocarbyl optionally substituted with a phenyl group, and aliphatic (C₁₀-C₁₁)hydrocarbyl optionally substituted with a phenyl group.

In various embodiments of the present disclosure, R¹ is chosen from: aliphatic (C₁₁-C₂₀)hydrocarbyl optionally substituted with a phenyl group with the proviso that R contains twenty carbons or less, aliphatic (C₁₁-C₁₉)hydrocarbyl optionally substituted with a phenyl group with the proviso that R¹ contains twenty carbons or less, aliphatic (C₁₁-C₁₈)hydrocarbyl optionally substituted with a phenyl group with the proviso that R¹ contains twenty carbons or less, aliphatic (C₁₁-C₁₇)hydrocarbyl optionally substituted with a phenyl group with the proviso that R contains twenty carbons or less, aliphatic (C₁₁-C₁₆)hydrocarbyl optionally substituted with a phenyl group with the proviso that R¹ contains twenty carbons or less, aliphatic (C₁₁-C₁₅)hydrocarbyl optionally substituted with a phenyl group with the proviso that R¹ contains twenty carbons or less, aliphatic (C₁₁-C₁₄)hydrocarbyl optionally substituted with a phenyl group, aliphatic (C₁₁-C₁₃)hydrocarbyl optionally substituted with a phenyl group, and aliphatic (C₁₁-C₁₂)hydrocarbyl optionally substituted with a phenyl group.

In various embodiments of the present disclosure, R¹ is chosen from: aliphatic (C₁₂-C₂₀)hydrocarbyl optionally substituted with a phenyl group with the proviso that R contains twenty carbons or less, aliphatic (C₁₂-C₁₉)hydrocarbyl optionally substituted with a phenyl group with the proviso that R¹ contains twenty carbons or less, aliphatic (C₁₂-C₁₈)hydrocarbyl optionally substituted with a phenyl group with the proviso that R¹ contains twenty carbons or less, aliphatic (C₁₂-C₁₇)hydrocarbyl optionally substituted with a phenyl group with the proviso that R contains twenty carbons or less, aliphatic (C₁₂-C₁₆)hydrocarbyl optionally substituted with a phenyl group with the proviso that R¹ contains twenty carbons or less, aliphatic (C₁₂-C₁₅)hydrocarbyl optionally substituted with a phenyl group with the proviso that R¹ contains twenty carbons or less, aliphatic (C₁₂-C₁₄)hydrocarbyl optionally substituted with a phenyl group, and aliphatic (C₁₂-C₁₃)hydrocarbyl optionally substituted with a phenyl group.

In various embodiments of the present disclosure, R¹ is chosen from: aliphatic (C₁₃-C₂₀)hydrocarbyl optionally substituted with a phenyl group with the proviso that R¹ contains twenty carbons or less, aliphatic (C₁₃-C₁₉)hydrocarbyl optionally substituted with a phenyl group with the proviso that R¹ contains twenty carbons or less, aliphatic (C₁₃-C₁₈)hydrocarbyl optionally substituted with a phenyl group with the proviso that R¹ contains twenty carbons or less, aliphatic (C₁₃-C₁₇)hydrocarbyl optionally substituted with a phenyl group with the proviso that R¹ contains twenty carbons or less, aliphatic (C₁₃-C₁₆)hydrocarbyl optionally substituted with a phenyl group with the proviso that R¹ contains twenty carbons or less, aliphatic (C₁₃-C₁₅)hydrocarbyl optionally substituted with a phenyl group with the proviso that R¹ contains twenty carbons or less, and aliphatic (C₁₃-C₁₄)hydrocarbyl optionally substituted with a phenyl group.

In various embodiments of the present disclosure, R¹ is chosen from: aliphatic (C₁₄-C₂₀)hydrocarbyl optionally substituted with a phenyl group with the proviso that R¹ contains twenty carbons or less, aliphatic (C₁₄-C₁₉)hydrocarbyl optionally substituted with a phenyl group with the proviso that R¹ contains twenty carbons or less, aliphatic (C₁₄-C₁₈)hydrocarbyl optionally substituted with a phenyl group with the proviso that R¹ contains twenty carbons or less, aliphatic (C₁₄-C₁₇)hydrocarbyl optionally substituted with a phenyl group with the proviso that R¹ contains twenty carbons or less, aliphatic (C₁₄-C₁₆)hydrocarbyl optionally substituted with a phenyl group with the proviso that R¹ contains twenty carbons or less, and aliphatic (C₁₄-C₁₅)hydrocarbyl optionally substituted with a phenyl group with the proviso that R¹ contains twenty carbons or less.

In various embodiments of the present disclosure, R¹ is chosen from: aliphatic (C₁₅-C₂₀)hydrocarbyl, aliphatic (C₁₅-C₁₉)hydrocarbyl, aliphatic (Cis-Cis)hydrocarbyl, aliphatic (C₁₅-C₁₇)hydrocarbyl, and aliphatic (C₁₅-C₁₆)hydrocarbyl.

In various embodiments of the present disclosure, R¹ is chosen from: aliphatic (C₁₆-C₂₀)hydrocarbyl, aliphatic (C₁₆-C₁₉)hydrocarbyl, aliphatic (C₁₆-C₁₈)hydrocarbyl, and aliphatic (C₁₆-C₁₇)hydrocarbyl.

In various embodiments of the present disclosure, R¹ is chosen from: aliphatic (C₁₇-C₂₀)hydrocarbyl, aliphatic (C₁₇-C₁₉)hydrocarbyl, and aliphatic (C₁₇-C₁₈)hydrocarbyl.

In various embodiments of the present disclosure, R¹ is aliphatic (C₁₈-C₂₀)hydrocarbyl or aliphatic (C₁₈-C₁₉)hydrocarbyl.

In various embodiments of the present disclosure, R¹ is aliphatic (C₁₉-C₂₀)hydrocarbyl.

In various embodiments of the present disclosure, R¹ is chosen from: aliphatic (C₂₀)hydrocarbyl, aliphatic (C₁₉)hydrocarbyl, aliphatic (C₁₈)hydrocarbyl, aliphatic (C₁₇)hydrocarbyl, aliphatic (C₁₆)hydrocarbyl, aliphatic (C₁₅)hydrocarbyl, aliphatic (C₁₄)hydrocarbyl optionally substituted with a phenyl group, aliphatic (C₁₃)hydrocarbyl optionally substituted with a phenyl group, aliphatic (C₁₂)hydrocarbyl optionally substituted with a phenyl group, aliphatic (C₁₁)hydrocarbyl optionally substituted with a phenyl group, aliphatic (C₁₀)hydrocarbyl optionally substituted with a phenyl group, aliphatic (C₉)hydrocarbyl optionally substituted with a phenyl group, aliphatic (C₈)hydrocarbyl optionally substituted with one or two phenyl groups, aliphatic (C₇)hydrocarbyl optionally substituted with one or two phenyl groups, aliphatic (C₆)hydrocarbyl optionally substituted with one or two phenyl groups, aliphatic (C₅)hydrocarbyl optionally substituted with one or two phenyl groups, aliphatic (C₄)hydrocarbyl optionally substituted with one or two phenyl groups, and aliphatic (C₃)hydrocarbyl optionally substituted with one or two phenyl groups.

In some embodiments of the present disclosure, R¹ is chosen from: (C₁-C₂₀)alkyl optionally substituted with one or two phenyl groups with the proviso that R¹ contains twenty carbons or less, (C₁-C₁₉)alkyl optionally substituted with one or two phenyl groups with the proviso that R¹ contains twenty carbons or less, (C₁-C₁₈)alkyl optionally substituted with one or two phenyl groups with the proviso that R¹ contains twenty carbons or less, (C₁-C₁₇)alkyl optionally substituted with one or two phenyl groups with the proviso that R¹ contains twenty carbons or less, (C₁-C₁₆)alkyl optionally substituted with one or two phenyl groups with the proviso that R¹ contains twenty carbons or less, (C₁-C₁₅)alkyl optionally substituted with one or two phenyl groups with the proviso that R¹ contains twenty carbons or less, (C₁-C₁₄)alkyl optionally substituted with one or two phenyl groups with the proviso that R¹ contains twenty carbons or less, (C₁-C₁₃)alkyl optionally substituted with one or two phenyl groups with the proviso that R¹ contains twenty carbons or less, (C₁-C₁₂)alkyl optionally substituted with one or two phenyl groups with the proviso that R¹ contains twenty carbons or less, (C₁-C₁₁)alkyl optionally substituted with one or two phenyl groups with the proviso that R¹ contains twenty carbons or less, (C₁-C₁₀)alkyl optionally substituted with one or two phenyl groups with the proviso that R¹ contains twenty carbons or less, (C₁-C₉)alkyl optionally substituted with one or two phenyl groups with the proviso that R¹ contains twenty carbons or less, (C₁-C₈)alkyl optionally substituted with one or two phenyl groups, (C₁-C₇)alkyl optionally substituted with one or two phenyl groups, (C₁-C₆)alkyl optionally substituted with one or two phenyl groups, (C₁-C₅)alkyl optionally substituted with one or two phenyl groups, (C₁-C₄)alkyl optionally substituted with one or two phenyl groups, and (C₁-C₃)alkyl optionally substituted with one or two phenyl groups.

In various embodiments of the present disclosure, R¹ is chosen from: (C₂-C₂₀)alkyl optionally substituted with one or two phenyl groups with the proviso that R¹ contains twenty carbons or less, (C₂-C₁₉)alkyl optionally substituted with one or two phenyl groups with the proviso that R¹ contains twenty carbons or less, (C₂-C₁₈)alkyl optionally substituted with one or two phenyl groups with the proviso that R¹ contains twenty carbons or less, (C₂-C₁₇)alkyl optionally substituted with one or two phenyl groups with the proviso that R¹ contains twenty carbons or less, (C₂-C₁₆)alkyl optionally substituted with one or two phenyl groups with the proviso that R¹ contains twenty carbons or less, (C₂-C₁₅)alkyl optionally substituted with one or two phenyl groups with the proviso that R¹ contains twenty carbons or less, (C₂-C₁₄)alkyl optionally substituted with one or two phenyl groups with the proviso that R¹ contains twenty carbons or less, (C₂-C₁₃)alkyl optionally substituted with one or two phenyl groups with the proviso that R¹ contains twenty carbons or less, (C₂-C₁₂)alkyl optionally substituted with one or two phenyl groups with the proviso that R¹ contains twenty carbons or less, (C₂-C₁₁)alkyl optionally substituted with one or two phenyl groups with the proviso that R¹ contains twenty carbons or less, (C₂-C₁₀)alkyl optionally substituted with one or two phenyl groups with the proviso that R¹ contains twenty carbons or less, (C₂-C₉)alkyl optionally substituted with one or two phenyl groups with the proviso that R¹ contains twenty carbons or less, (C₂-C₈)alkyl optionally substituted with one or two phenyl groups, (C₂-C₇)alkyl optionally substituted with one or two phenyl groups, (C₂-C₆)alkyl optionally substituted with one or two phenyl groups, (C₂-C₅)alkyl optionally substituted with one or two phenyl groups, (C₂-C₄)alkyl optionally substituted with one or two phenyl groups, and (C₂-C₃)alkyl optionally substituted with one or two phenyl groups.

In various embodiments of the present disclosure, R¹ is chosen from: (C₃-C₂₀)alkyl optionally substituted with one or two phenyl groups with the proviso that R¹ contains twenty carbons or less, (C₃-C₁₉)alkyl optionally substituted with one or two phenyl groups with the proviso that R¹ contains twenty carbons or less, (C₃-C₁₈)alkyl optionally substituted with one or two phenyl groups with the proviso that R¹ contains twenty carbons or less, (C₃-C₁₇)alkyl optionally substituted with one or two phenyl groups with the proviso that R¹ contains twenty carbons or less, (C₃-C₁₆)alkyl optionally substituted with one or two phenyl groups with the proviso that R¹ contains twenty carbons or less, (C₃-C₁₅)alkyl optionally substituted with one or two phenyl groups with the proviso that R¹ contains twenty carbons or less, (C₃-C₁₄)alkyl optionally substituted with one or two phenyl groups with the proviso that R¹ contains twenty carbons or less, (C₃-C₁₃)alkyl optionally substituted with one or two phenyl groups with the proviso that R¹ contains twenty carbons or less, (C₃-C₁₂)alkyl optionally substituted with one or two phenyl groups with the proviso that R¹ contains twenty carbons or less, (C₃-C₁₁)alkyl optionally substituted with one or two phenyl groups with the proviso that R¹ contains twenty carbons or less, (C₃-C₁₀)alkyl optionally substituted with one or two phenyl groups with the proviso that R¹ contains twenty carbons or less, (C₃-C₉)alkyl optionally substituted with one or two phenyl groups with the proviso that R¹ contains twenty carbons or less, (C₃-C₈)alkyl optionally substituted with one or two phenyl groups, (C₃-C₇)alkyl optionally substituted with one or two phenyl groups, (C₃-C₆)alkyl optionally substituted with one or two phenyl groups, (C₃-C₅)alkyl optionally substituted with one or two phenyl groups, (C₃-C₄)alkyl optionally substituted with one or two phenyl groups.

In various embodiments of the present disclosure, R¹ is chosen from: (C₄-C₂₀)alkyl optionally substituted with one or two phenyl groups with the proviso that R¹ contains twenty carbons or less, (C₄-C₁₉)alkyl optionally substituted with one or two phenyl groups with the proviso that R¹ contains twenty carbons or less, (C₄-C₁₈)alkyl optionally substituted with one or two phenyl groups with the proviso that R¹ contains twenty carbons or less, (C₄-C₁₇)alkyl optionally substituted with one or two phenyl groups with the proviso that R¹ contains twenty carbons or less, (C₄-C₁₆)alkyl optionally substituted with one or two phenyl groups with the proviso that R¹ contains twenty carbons or less, (C₄-C₁₅)alkyl optionally substituted with one or two phenyl groups with the proviso that R¹ contains twenty carbons or less, (C₄-C₁₄)alkyl optionally substituted with one or two phenyl groups with the proviso that R¹ contains twenty carbons or less, (C₄-C₁₃)alkyl optionally substituted with one or two phenyl groups with the proviso that R¹ contains twenty carbons or less, (C₄-C₁₂)alkyl optionally substituted with one or two phenyl groups with the proviso that R¹ contains twenty carbons or less, (C₄-C₁₁)alkyl optionally substituted with one or two phenyl groups with the proviso that R¹ contains twenty carbons or less, (C₄-C₁₀)alkyl optionally substituted with one or two phenyl groups with the proviso that R¹ contains twenty carbons or less, (C₄-C₉)alkyl optionally substituted with one or two phenyl groups with the proviso that R¹ contains twenty carbons or less, (C₄-C₈)alkyl optionally substituted with one or two phenyl groups, (C₄-C₇)alkyl optionally substituted with one or two phenyl groups, (C₄-C₆)alkyl optionally substituted with one or two phenyl groups, and (C₄-C₅)alkyl optionally substituted with one or two phenyl groups.

In various embodiments of the present disclosure, R¹ is chosen from: (C₅-C₂₀)alkyl optionally substituted with one or two phenyl groups with the proviso that R contains twenty carbons or less, (C₅-C₁₉)alkyl optionally substituted with one or two phenyl groups with the proviso that R¹ contains twenty carbons or less, (C₅-C₁₈)alkyl optionally substituted with one or two phenyl groups with the proviso that R¹ contains twenty carbons or less, (C₅-C₁₇)alkyl optionally substituted with one or two phenyl groups with the proviso that R¹ contains twenty carbons or less, (C₅-C₁₆)alkyl optionally substituted with one or two phenyl groups with the proviso that R¹ contains twenty carbons or less, (C₅-C₁₅)alkyl optionally substituted with one or two phenyl groups with the proviso that R¹ contains twenty carbons or less, (C₅-C₁₄)alkyl optionally substituted with one or two phenyl groups with the proviso that R¹ contains twenty carbons or less, (C₅-C₁₃)alkyl optionally substituted with one or two phenyl groups with the proviso that R¹ contains twenty carbons or less, (C₅-C₁₂)alkyl optionally substituted with one or two phenyl groups with the proviso that R¹ contains twenty carbons or less, (C₅-C₁₁)alkyl optionally substituted with one or two phenyl groups with the proviso that R¹ contains twenty carbons or less, (C₅-C₁₀)alkyl optionally substituted with one or two phenyl groups with the proviso that R¹ contains twenty carbons or less, (C₅-C₉)alkyl optionally substituted with one or two phenyl groups with the proviso that R¹ contains twenty carbons or less, (C₅-C₈)alkyl optionally substituted with one or two phenyl groups, (C₅-C₇)alkyl optionally substituted with one or two phenyl groups, and (C₅-C₆)alkyl optionally substituted with one or two phenyl groups.

In various embodiments of the present disclosure, R¹ is chosen from: (C₆-C₂₀)alkyl optionally substituted with one or two phenyl groups with the proviso that R¹ contains twenty carbons or less, (C₆-C₁₉)alkyl optionally substituted with one or two phenyl groups with the proviso that R¹ contains twenty carbons or less, (C₆-C₁₈)alkyl optionally substituted with one or two phenyl groups with the proviso that R¹ contains twenty carbons or less, (C₆-C₁₇)alkyl optionally substituted with one or two phenyl groups with the proviso that R¹ contains twenty carbons or less, (C₆-C₁₆)alkyl optionally substituted with one or two phenyl groups with the proviso that R¹ contains twenty carbons or less, (C₆-C₁₅)alkyl optionally substituted with one or two phenyl groups with the proviso that R¹ contains twenty carbons or less, (C₆-C₁₄)alkyl optionally substituted with one or two phenyl groups with the proviso that R¹ contains twenty carbons or less, (C₆-C₁₃)alkyl optionally substituted with one or two phenyl groups with the proviso that R¹ contains twenty carbons or less, (C₆-C₁₂)alkyl optionally substituted with one or two phenyl groups with the proviso that R¹ contains twenty carbons or less, (C₆-C₁₁)alkyl optionally substituted with one or two phenyl groups with the proviso that R¹ contains twenty carbons or less, (C₆-C₁₀)alkyl optionally substituted with one or two phenyl groups with the proviso that R¹ contains twenty carbons or less, (C₆-C₉)alkyl optionally substituted with one or two phenyl groups with the proviso that R¹ contains twenty carbons or less, (C₆-C₈)alkyl optionally substituted with one or two phenyl groups, and (C₆-C₇)alkyl optionally substituted with one or two phenyl groups.

In various embodiments of the present disclosure, R¹ is chosen from: (C₇-C₂₀)alkyl optionally substituted with one or two phenyl groups with the proviso that R¹ contains twenty carbons or less, (C₇-C₁₉)alkyl optionally substituted with one or two phenyl groups with the proviso that R¹ contains twenty carbons or less, (C₇-C₁₈)alkyl optionally substituted with one or two phenyl groups with the proviso that R¹ contains twenty carbons or less, (C₇-C₁₇)alkyl optionally substituted with one or two phenyl groups with the proviso that R¹ contains twenty carbons or less, (C₇-C₁₆)alkyl optionally substituted with one or two phenyl groups with the proviso that R¹ contains twenty carbons or less, (C₇-C₁₅)alkyl optionally substituted with one or two phenyl groups with the proviso that R¹ contains twenty carbons or less, (C₇-C₁₄)alkyl optionally substituted with one or two phenyl groups with the proviso that R¹ contains twenty carbons or less, (C₇-C₁₃)alkyl optionally substituted with one or two phenyl groups with the proviso that R¹ contains twenty carbons or less, (C₇-C₁₂)alkyl optionally substituted with one or two phenyl groups with the proviso that R¹ contains twenty carbons or less, (C₇-C₁₁)alkyl optionally substituted with one or two phenyl groups with the proviso that R¹ contains twenty carbons or less, (C₇-C₁₀)alkyl optionally substituted with one or two phenyl groups with the proviso that R¹ contains twenty carbons or less, (C₇-C₉)alkyl optionally substituted with one or two phenyl groups with the proviso that R¹ contains twenty carbons or less, and (C₇-C₈)alkyl optionally substituted with one or two phenyl groups.

In various embodiments of the present disclosure, R¹ is chosen from: (C₈-C₂₀)alkyl optionally substituted with one or two phenyl groups with the proviso that R¹ contains twenty carbons or less, (C₈-C₁₉)alkyl optionally substituted with one or two phenyl groups with the proviso that R¹ contains twenty carbons or less, (C₈-C₁₈)alkyl optionally substituted with one or two phenyl groups with the proviso that R¹ contains twenty carbons or less, (C₈-C₁₇)alkyl optionally substituted with one or two phenyl groups with the proviso that R¹ contains twenty carbons or less, (C₈-C₁₆)alkyl optionally substituted with one or two phenyl groups with the proviso that R¹ contains twenty carbons or less, (C₈-C₁₅)alkyl optionally substituted with one or two phenyl groups with the proviso that R¹ contains twenty carbons or less, (C₈-C₁₄)alkyl optionally substituted with one or two phenyl groups with the proviso that R¹ contains twenty carbons or less, (C₈-C₁₃)alkyl optionally substituted with one or two phenyl groups with the proviso that R¹ contains twenty carbons or less, (C₈-C₁₂)alkyl optionally substituted with one or two phenyl groups with the proviso that R¹ contains twenty carbons or less, (C₈-C₁₁)alkyl optionally substituted with one or two phenyl groups with the proviso that R¹ contains twenty carbons or less, (C₅-C₁₀)alkyl optionally substituted with one or two phenyl groups with the proviso that R¹ contains twenty carbons or less, and (C₈-C₉)alkyl optionally substituted with one or two phenyl groups with the proviso that R¹ contains twenty carbons or less.

In various embodiments of the present disclosure, R¹ is chosen from: (C₉-C₂₀)alkyl optionally substituted with a phenyl group with the proviso that R¹ contains twenty carbons or less, (C₉-C₁₉)alkyl optionally substituted with a phenyl group with the proviso that R¹ contains twenty carbons or less, (C₉-C₁₈)alkyl optionally substituted with a phenyl group with the proviso that R¹ contains twenty carbons or less, (C₉-C₁₇)alkyl optionally substituted with a phenyl group with the proviso that R¹ contains twenty carbons or less, (C₉-C₁₆)alkyl optionally substituted with a phenyl group with the proviso that R¹ contains twenty carbons or less, (C₉-C₁₅)alkyl optionally substituted with a phenyl group with the proviso that R¹ contains twenty carbons or less, (C₉-C₁₄)alkyl optionally substituted with a phenyl group, (C₉-C₁₃)alkyl optionally substituted with a phenyl group, (C₉-C₁₂)alkyl optionally substituted with a phenyl group, (C₉-C₁₁)alkyl optionally substituted with a phenyl group, and (C₉-C₁₀)alkyl optionally substituted with a phenyl group.

In various embodiments of the present disclosure, R¹ is chosen from: (C₁₀-C₂₀)alkyl optionally substituted with a phenyl group with the proviso that R¹ contains twenty carbons or less, (C₁₀-C₁₉)alkyl optionally substituted with a phenyl group with the proviso that R¹ contains twenty carbons or less, (C₁₀-C₁₈)alkyl optionally substituted with a phenyl group with the proviso that R¹ contains twenty carbons or less, (C₁₀-C₁₇)alkyl optionally substituted with a phenyl group with the proviso that R¹ contains twenty carbons or less, (C₁₀-C₁₆)alkyl optionally substituted with a phenyl group with the proviso that R¹ contains twenty carbons or less, (C₁₀-C₁₅)alkyl optionally substituted with a phenyl group with the proviso that R¹ contains twenty carbons or less, (C₁₀-C₁₄)alkyl optionally substituted with a phenyl group, (C₁₀-C₁₃)alkyl optionally substituted with a phenyl group, (C₁₀-C₁₂)alkyl optionally substituted with a phenyl group, and (C₁₀-C₁₁)alkyl optionally substituted with a phenyl group.

In various embodiments of the present disclosure, R¹ is chosen from: (C₁₁-C₂₀)alkyl optionally substituted with a phenyl group with the proviso that R contains twenty carbons or less, (C₁₁-C₁₉)alkyl optionally substituted with a phenyl group with the proviso that R¹ contains twenty carbons or less, (C₁₁-C₁₈)alkyl optionally substituted with a phenyl group with the proviso that R¹ contains twenty carbons or less, (C₁₁-C₁₇)alkyl optionally substituted with a phenyl group with the proviso that R¹ contains twenty carbons or less, (C₁₁-C₁₆)alkyl optionally substituted with a phenyl group with the proviso that R¹ contains twenty carbons or less, (C₁₁-C₁₅)alkyl optionally substituted with a phenyl group with the proviso that R¹ contains twenty carbons or less, (C₁₁-C₁₄)alkyl optionally substituted with a phenyl group, (C₁₁-C₁₃)alkyl optionally substituted with a phenyl group, and (C₁₁-C₁₂)alkyl optionally substituted with a phenyl group.

In various embodiments of the present disclosure, R¹ is chosen from: (C₁₂-C₂₀)alkyl optionally substituted with a phenyl group with the proviso that R¹ contains twenty carbons or less, (C₁₂-C₁₉)alkyl optionally substituted with a phenyl group with the proviso that R¹ contains twenty carbons or less, (C₁₂-C₁₈)alkyl optionally substituted with a phenyl group with the proviso that R¹ contains twenty carbons or less, (C₁₂-C₁₇)alkyl optionally substituted with a phenyl group with the proviso that R¹ contains twenty carbons or less, (C₁₂-C₁₆)alkyl optionally substituted with a phenyl group with the proviso that R¹ contains twenty carbons or less, (C₁₂-C₁₅)alkyl optionally substituted with a phenyl group with the proviso that R¹ contains twenty carbons or less, (C₁₂-C₁₄)alkyl optionally substituted with a phenyl group, and (C₁₂-C₁₃)alkyl optionally substituted with a phenyl group.

In various embodiments of the present disclosure, R¹ is chosen from: (C₁₃-C₂₀)alkyl optionally substituted with a phenyl group with the proviso that R¹ contains twenty carbons or less, (C₁₃-C₁₉)alkyl optionally substituted with a phenyl group with the proviso that R¹ contains twenty carbons or less, (C₁₃-C₁₈)alkyl optionally substituted with a phenyl group with the proviso that R¹ contains twenty carbons or less, (C₁₃-C₁₇)alkyl optionally substituted with a phenyl group with the proviso that R¹ contains twenty carbons or less, (C₁₃-C₁₆)alkyl optionally substituted with a phenyl group with the proviso that R¹ contains twenty carbons or less, (C₁₃-C₁₅)alkyl optionally substituted with a phenyl group with the proviso that R¹ contains twenty carbons or less, and (C₁₃-C₁₄)alkyl optionally substituted with a phenyl group.

In various embodiments of the present disclosure, R¹ is chosen from: (C₁₄-C₂₀)alkyl optionally substituted with a phenyl group with the proviso that R¹ contains twenty carbons or less, (C₁₄-C₁₉)alkyl optionally substituted with a phenyl group with the proviso that R¹ contains twenty carbons or less, (C₁₄-C₁₈)alkyl optionally substituted with a phenyl group with the proviso that R¹ contains twenty carbons or less, (C₁₄-C₁₇)alkyl optionally substituted with a phenyl group with the proviso that R¹ contains twenty carbons or less, (C₁₄-C₁₆)alkyl optionally substituted with a phenyl group with the proviso that R¹ contains twenty carbons or less, and (C₁₄-C₁₅)alkyl optionally substituted with a phenyl group with the proviso that R¹ contains twenty carbons or less.

In various embodiments of the present disclosure, R¹ is chosen from: (C₁₅-C₂₀)alkyl, (C₁₅-C₁₉)alkyl, (C₁₅-C₁₈)alkyl, (C₁₅-C₁₇)alkyl, and (C₁₅-C₁₆)alkyl.

In various embodiments of the present disclosure, R¹ is chosen from: (C₁₆-C₂₀)alkyl, (C₁₆-C₁₉)alkyl, (C₁₆-C₁₈)alkyl, and (C₁₆-C₁₇)alkyl.

In various embodiments of the present disclosure, R¹ is chosen from: (C₁₇-C₂₀)alkyl, (C₁₇-C₁₉)alkyl, and (C₁₇-C₁₈)alkyl.

In various embodiments of the present disclosure, R¹ is (C₁₈-C₂₀)alkyl or (C₁₈-C₁₉)alkyl.

In various embodiments of the present disclosure, R¹ is (C₁₉-C₂₀)alkyl.

In various embodiments of the present disclosure, R¹ is chosen from: (C₂₀)alkyl, (C₁₉)alkyl, (C₁₈)alkyl, (C₁₇)alkyl, (C₁₆)alkyl, (C₁₅)alkyl, (C₁₄)alkyl optionally substituted with a phenyl group, (C₁₃)alkyl optionally substituted with a phenyl group, (C₁₂)alkyl optionally substituted with a phenyl group, (C₁₁)alkyl optionally substituted with a phenyl group, (C₁₀)alkyl optionally substituted with a phenyl group, (C₉)alkyl optionally substituted with a phenyl group, (C₈)alkyl optionally substituted with one or two phenyl groups, (C₇)alkyl optionally substituted with one or two phenyl groups, (C₆)alkyl optionally substituted with one or two phenyl groups, (C₅)alkyl optionally substituted with one or two phenyl groups, (C₄)alkyl optionally substituted with one or two phenyl groups, (C₃)alkyl optionally substituted with one or two phenyl groups, ethyl optionally substituted with one or two phenyl groups, and methyl optionally substituted with one or two phenyl groups.

In various embodiments of the present disclosure, R¹ is CᵣHₛ. In some of these embodiments, q is 1 and r is 3, (i.e., methyl). In some of these embodiments, q is 2 and r is 5, (i.e., ethyl). In some of these embodiments, q is 3 and r is chosen from: 3, 5, and 7. In some of these embodiments, q is 4 and r is chosen from: 5, 7, and 9. In some of these embodiments, q is 5 and r is chosen from: 7, 9, and 11. In some of these embodiments, q is 6 and r is chosen from: 5, 7, 9, 11, and 13. In some of these embodiments, q is 7 and r is chosen from: 7, 9, 11, 13, and 15. In some of these embodiments, q is 8 and r is chosen from: 5, 7, 9, 11, 13, 15, and 17. In some of these embodiments, q is 9 and r is chosen from: 7, 9, 11, 13, 15, 17, and 19. In some of these embodiments, q is 10 and r is chosen from: 7, 9, 11, 13, 15, 17, 19, and 21. In some of these embodiments, q is 11 and r is chosen from: 9, 11, 13, 15, 17, 19, 21, and 23. In some of these embodiments, q is 12 and r is chosen from: 7, 9, 11, 13, 15, 17, 19, 21, 23, and 25. In some of these embodiments, q is 13 and r is chosen from: 9, 11, 13, 15, 17, 19, 21, 23, 25, and 27. In some of these embodiments, q is 14 and r is chosen from: 9, 11, 13, 15, 17, 19, 21, 23, 25, 27, and 29. In some of these embodiments, q is 15 and r is chosen from: 11, 13, 15, 17, 19, 21, 23, 25, 27, 29, and 31. In some of these embodiments, q is 16 and r is chosen from: 9, 11, 13, 15, 17, 19, 21, 23, 25, 27, 29, 31, and 33. In some of these embodiments, q is 17 and r is chosen from: 11, 15, 17, 19, 21, 23, 25, 27, 29, 31, 33, and 35. In some of these embodiments, q is 18 and r is chosen from: 11, 13, 15, 17, 19, 21, 23, 25, 27, 29, 31, 33, 35, and 37. In some of these embodiments, q is 19 and r is chosen from: 13, 15, 17, 19, 21, 23, 25, 27, 29, 31, 33, 35, 37, and 39. In some of these embodiments, q is 20 and r is chosen from: 11, 13, 15, 17, 19, 21, 23, 25, 27, 29, 31, 33, 35, 37, 39, and 41.

In some embodiments, R¹ is n-propyl, isopropyl, cyclopropyl, n-butyl, 1-methylpropyl, 2-ethylbutyl, 1-methyl-2-ethylbutyl, 2-methylpropyl, tert-butyl, 2-methylcyclopropyl, 1-methylcyclopropyl, cyclobutyl, cyclopropylmethyl (i.e.,

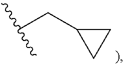 [SMILES: *CC1CC1]

n-pentyl, 1-methylbutyl, 2-methylbutyl, 3-methylbutyl, 1-ethylpropyl, 1,2-dimethylpropyl, 1,1-dimethylpropyl, 2,2-dimethylpropyl, cyclobutylmethyl (i.e.,

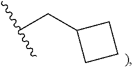 [SMILES: *CC1CCC1]

2-(cyclopropyl)ethyl (i.e.,

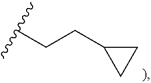 [SMILES: *CCC1CC1]

cyclopentyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,3-dimethylbutyl, 2,2-dimethylbutyl, 3,3-dimethylbutyl, 1,1,2-trimethylpropyl, 1,2,2-trimethylpropyl, 3-(cyclopropyl)propyl (i.e.,

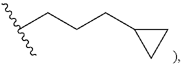 [SMILES: *CCCC1CC1]

2-(cyclobutyl)ethyl (i.e.,

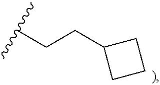 [SMILES: *CCC1CCC1]

cyclopentylmethyl (i.e.,

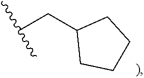 [SMILES: *CC1CCCC1]

cyclohexyl, cyclohexylmethyl, 2-cyclohexylethyl, dicyclohexylmethyl, n-octyl, benzyl, diphenylmethyl, decyl, dodecyl, tetradecyl, hexadecyl, hexadec-9-enyl, octadecyl, octadec-9-enyl, octadec-9,12-dienyl, 2-propylpentyl, 2-butylhexyl, 2-pentylheptyl, or 2-hexyloctyl.

In some embodiments, R² is selected from H and optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂. In some embodiments, R² is H.

In some embodiments, R² is optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂. In some embodiments, R² is optionally substituted benzyl, and wherein said optionally substituted benzyl is optionally substituted with one or more of —C(═O)—OH, —C(═O)—O—CH₃, or —C(═O)—NH—CH₃.

In some embodiments, R² is optionally substituted (C₁-C₃)hydrocarbyl, and wherein said optionally substituted (C₁-C₃)hydrocarbyl is substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂.

In some embodiments, R² is methyl optionally substituted with one or more of —F, —O—C(═O)(C₁-C₄)hydrocarbyl, —C(═O)—OH, —C(═O)—O—CH₃, or —C(═O)—NH—CH₃. In some embodiments, R² is methyl, difluoro methyl, CH₂—O—C(═O)(C₁-C₄)hydrocarbyl, CH₂—O—C(═O)—CH₃, CH₂—C(═O)—OH, CH₂—C(═O)—O—CH₃, or CH₂—C(═O)—NH—CH₃.

In some embodiments, R² is ethyl optionally substituted with one or more of —OH, —O—C(═O)(C₁-C₄)hydrocarbyl, —NH(C₁-C₄)hydrocarbyl, or —N[(C₁-C₄)hydrocarbyl)]₂. In some embodiments, R² is ethyl, ethanol, (CH₂)₂—O—C(═O)—CH₃, (CH₂)₂—N[(C₁-C₄)hydrocarbyl)]₂.

In some embodiments, R² is propyl optionally substituted with one or more of —CN.

In one aspect, the present disclosure relates to a compound of formula VIa or VIb:

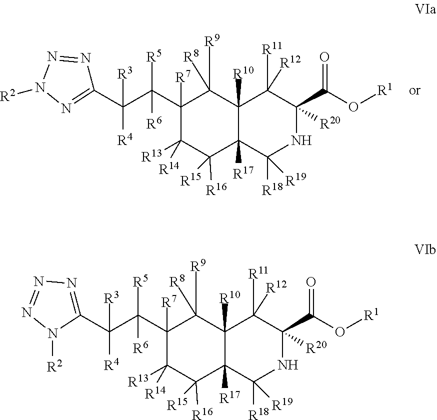

wherein:
R¹ is selected from —CH(R^{a})R^{b} and aromatic (C₁-C₂₀)hydrocarbyl;
R^{a} is selected from H, —(C₁-C₆)alkyl, and —C(═O)—O—(C₁-C₆)alkyl;
R^{b} is selected from —(C₁-C₁₀)hydrocarbyl-O—C(═O)—R^{c}, —(C₁-C₁₀)hydrocarbyl[-OC(═O)—R^{c}]₂, -direct bond-C(═O)—R^{c} or —(C₁-C₁₀)hydrocarbyl-C(═O)—R^{c}, —CH(NHR^{d})C(═O)—R^{c}, and —(C₁-C₁₀)hydrocarbyl-NR^{e}—C(═O)—R^{f};
R^{c} is selected from —O—(C₁-C₁₀)hydrocarbyl, —NH—(C₁-C₁₀)hydrocarbyl, —N—[(C₁-C₁₀)hydrocarbyl]₂, where the hydrocarbyl groups can be combined to form a (C₄-C₆) nitrogenous aliphatic heterocycle, —(C₁-C₁₀)hydrocarbyl, —NH₂, —(C₁-C₁₀)hydrocarbyl-NH—R^{e}, where the hydrocarbyl group can be combined with R^{e} to form a (C₄-C₆) nitrogenous aliphatic heterocycle, and —NR^{e}—(C₁-C₁₀)hydrocarbyl-C(═O)—O—(C₁-C₁₀)hydrocarbyl, where the hydrocarbyl group and R^{e} can be combined to form a (C₄-C₆) nitrogenous aliphatic heterocycle;
R^{d} is selected from H, —(C₁-C₁₀)hydrocarbyl, and —C(═O)(C₁-C₁₀)hydrocarbyl;
R^{e} is selected from H, and —(C₁-C₆)alkyl;
R^{f} is selected from —O—(C₁-C₁₀)hydrocarbyl, —(C₁-C₁₀)hydrocarbyl, -direct bond-C(═O)—O—(C₁-C₁₀)hydrocarbyl or —(C₁-C₄)hydrocarbyl-C(═O)—O—(C₁-C₁₀)hydrocarbyl, -direct bond-C(═O)—NH₂ or —(C₁-C₄)hydrocarbyl-C(═O)—NH₂, -direct bond-C(═O)—NH—(C₁-C₁₀)hydrocarbyl or —(C₁-C₄)hydrocarbyl-C(═O)—NH—(C₁-C₁₀)hydrocarbyl, —(C₁-C₁₀)hydrocarbyl-NH—R^{e}, where the hydrocarbyl linker can be combined with R^{e} to form a (C₄-C₆) nitrogenous aliphatic heterocycle, and —(C₁-C₁₀)hydrocarbyl-NH—C(═O)(C₁-C₁₀)hydrocarbyl;
R² is selected from H and optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂;
R³-R²⁰ are each independently selected from hydrogen and deuterium; and
wherein at least one of R³-R²⁰ is deuterium.

In various aspects, the present disclosure relates to compounds of formula VIIa or VIIb:

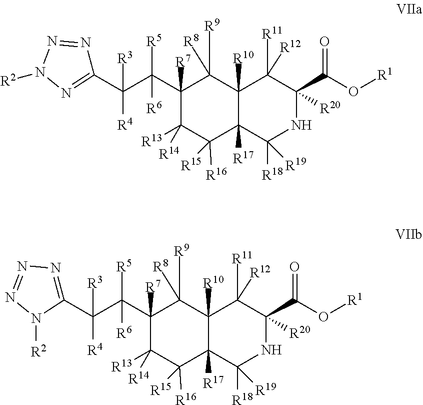

wherein: R¹ is selected from —CH(R^{a})R^{b} and aromatic (C₁-C₂₀)hydrocarbyl; R^{a} is selected from H, —(C₁-C₆)alkyl, and —C(═O)—O—(C₁-C₆)alkyl; R^{b} is selected from —(C₁-C₁₀)hydrocarbyl-O—C(═O)—R^{c}, —(C₁-C₁₀)hydrocarbyl[-OC(═O)—R^{c}]₂, -direct bond-C(═O)—R^{c} or —(C₁-C₁₀)hydrocarbyl-C(═O)—R^{c}, —CH(NHR^{d})C(═O)—R^{c}, and —(C₁-C₁₀)hydrocarbyl-NR^{e}—C(═O)—R; R^{c} is selected from —O—(C₁-C₁₀)hydrocarbyl, —NH—(C₁-C₁₀)hydrocarbyl, —N—[(C₁-C₁₀)hydrocarbyl]₂, where the hydrocarbyl groups can be combined to form a (C₄-C₆) nitrogenous aliphatic heterocycle, —(C₁-C₁₀)hydrocarbyl, —NH₂, —(C₁-C₁₀)hydrocarbyl-NH—R^{e}, where the hydrocarbyl group can be combined with R^{e} to form a (C₄-C₆) nitrogenous aliphatic heterocycle, and —NR^{e}—(C₁-C₁₀)hydrocarbyl-C(═O)—O—(C₁-C₁₀)hydrocarbyl, where the hydrocarbyl group and R^{e} can be combined to form a (C₄-C₆) nitrogenous aliphatic heterocycle; R^{d} is selected from H, —(C₁-C₁₀)hydrocarbyl, and —C(═O)(C₁-C₁₀)hydrocarbyl; R^{e} is selected from H, and —(C₁-C₆)alkyl; R^{f} is selected from —O—(C₁-C₁₀)hydrocarbyl, —(C₁-C₁₀)hydrocarbyl, -direct bond-C(═O)—O—(C₁-C₁₀)hydrocarbyl or —(C₁-C₄)hydrocarbyl-C(═O)—O—(C₁-C₁₀)hydrocarbyl, -direct bond-C(═O)—NH₂ or —(C₁-C₄)hydrocarbyl-C(═O)—NH₂, -direct bond-C(═O)—NH—(C₁-C₁₀)hydrocarbyl or —(C₁-C₄)hydrocarbyl-C(═O)—NH—(C₁-C₁₀)hydrocarbyl, —(C₁-C₁₀)hydrocarbyl-NH—R^{e}, where the hydrocarbyl linker can be combined with R^{e} to form a (C₄-C₆) nitrogenous aliphatic heterocycle, and —(C₁-C₁₀)hydrocarbyl-NH—C(═O)(C₁-C₁₀)hydrocarbyl; R² is selected from H and optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂; R³-R²⁰ are each independently selected from hydrogen and deuterium; and wherein at least one of R³-R²⁰ is deuterium.

In some embodiments, R¹ is —CH(R^{a})R^{b}. In these embodiments, R^{b} can include —(C₁-C₁₀)hydrocarbyl-O—C(═O)—R^{c}, —(C₁-C₅)alkyl-O—C(═O)—R^{c}, -direct bond-(CH(CH₃))—O—C(═O)—R^{c} or —(C₁-C₄)alkyl-(CH(CH₃))—O—C(═O)—R^{c}, and R^{c} can include —O—(C₁-C₆)alkyl, —O-phenyl, —NH₂, —NH—(C₁-C₆)alkyl, —N—[(C₁-C₆)alkyl]₂, where the alkyl groups can be combined to form a (C₄-C₆) nitrogenous aliphatic heterocycle, —(C₁-C₇)alkyl, -phenyl, or —(C₁-C₆)alkyl-NH—R^{e}. When R^{c} is —(C₁-C₆)alkyl-NH—R^{e}, R^{e} may be (C₁-C₆)alkyl, including but not limited to methyl or ethyl. Additionally, the alkyl linker and R^{e} can combined to form a (C₄-C₆) nitrogenous aliphatic heterocycle, which may include azetidin, pyrrolidine, or piperidine. In any of these embodiments, R^{a} may include hydrogen or methyl.

In some embodiments, R^{b} can be —(C₁-C₁₀)hydrocarbyl[-OC(═O)—R^{c}]₂. In these embodiments, R^{b} may be —(C₁-C₄)alkyl[-OC(═O)—R^{c}]₂ and R^{c} may include —(C₁-C₇)alkyl or -phenyl. In any of these embodiments, R^{a} may include hydrogen or methyl.

In some embodiments, R^{b} can include —(C₁-C₁₀)hydrocarbyl-NR^{e}—C(═O)—R, for instance —(C₁-C₅)alkyl-NR^{e}—C(═O)—R^{f}, -direct bond-(CH(CH₃))—NR^{e}—C(═O)—R^{f} or —(C₁-C₄)alkyl-(CH(CH₃))—NR^{e}—C(═O)—R^{f}. In these embodiments, R^{e} can include H, methyl, or ethyl. Additionally, R^{f} can include —O—(C₁-C₆)alkyl, —O-phenyl, —(C₁-C₆)alkyl, -phenyl, -direct bond-C(═O)—O—(C₁-C₆)alkyl, —(C₁-C₄)hydrocarbyl-C(═O)—O—(C₁-C₆)alkyl, or -direct bond-C(═O)—NH₂ or —(C₁-C₄)hydrocarbyl-C(═O)—NH₂ (such as —CH═CH—C(═O)—NH₂), -direct bond-C(═O)—NH—(C₁-C₆)alkyl or —(C₁-C₄)hydrocarbyl-C(═O)—NH—(C₁-C₆)alkyl (such as —CH═CH—C(═O)—NH—(C₁-C₆)alkyl), —(C₁-C₆)alkyl-NH—R^{e}, where the alkyl linker can be combined with R^{e} to form a (C₄-C₆) nitrogenous aliphatic heterocycle, such as azetidine, pyrrolidine, or piperidine, or —(C₁-C₆)alkyl-NH—C(═O)(C₁-C₁₀)hydrocarbyl (such as (—(C₁-C₆)alkyl-NH—C(═O)(C₁-C₆)alkyl or —(C₁-C₆)alkyl-NH—C(═O)phenyl). In any of these embodiments, R^{a} may include hydrogen or methyl.

In some embodiments, R^{b} can include -direct bond-C(═O)—R^{c} or —(C₁-C₅)alkyl-C(═O)—R^{c}, wherein R^{c} can include —O—(C₁-C₆)alkyl, —NH₂, —NH—(C₁-C₆)alkyl, or —N—[(C₁-C₆)alkyl]₂, where the alkyl groups can be combined to form a (C₄-C₆) nitrogenous aliphatic heterocycle, with the nitrogenous aliphatic heterocycle including azetidine, pyrrolidine, or piperidine. Additionally, R^{c} may be —NR^{e}—(C₁-C₆)alkyl-C(═O)—O—(C₁-C₆)alkyl, with R^{e} being hydrogen or methyl, or wherein the alkyl group is combined with R^{e} to form a (C₄-C₆) nitrogenous aliphatic heterocycle including azetidine, pyrrolidine, or piperidine. In any of these embodiments, R^{a} may include hydrogen, methyl, or —C(═O)—O—(C₁-C₄)alkyl.

In some embodiments, R^{b} can include —CH(NHR^{d})C(═O)—R^{c}. In these embodiments, R^{c} can include —O—(C₁-C₆)alkyl while R^{d} can include —(C₁-C₆)alkyl, —C(═O)(C₁-C₆)alkyl, and —C(═O)phenyl. In any of these embodiments, R^{a} may include hydrogen or methyl.

In some embodiments, R¹ may include an aromatic (C₁-C₂₀)hydrocarbyl. The aromatic (C₁-C₂₀)hydrocarbyl may include substituted or unsubstituted benzoate ester, such as ethyl meta-benzoate, ethyl ortho-benzoate, and ethyl para-benzoate. Additionally, the aromatic (C₁-C₂₀)hydrocarbyl may include a tyrosine derivative, α-tocopherol, and anilides (such as acetaminophen).

In some embodiments, each of R³-R²⁰ can include hydrogen. In an alternative embodiment, at least one of R³-R²⁰ is deuterium, wherein any number of R³-R²⁰ may include deuterium. For instance, in some embodiments, R³⁻⁴ are deuterium. In another embodiment, R²⁰ is deuterium. Additionally, in some embodiments, R¹⁸⁻²⁰ is deuterium.

In some embodiments, a compound can include the formula VIII:

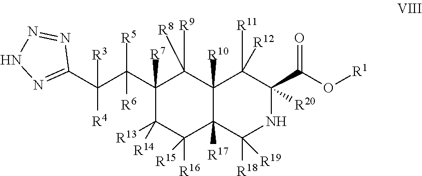

wherein at least one of R³-R²⁰ is deuterium; and R¹ is chosen from hydrogen and —(C₁-C₁₀) hydrocarbyl. Some such embodiments are illustrated in Table 1. These molecules are not meant to be limiting.

In various embodiments where at least one of R³-R²⁰ is deuterium, R² is selected from H and optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂. In some embodiments, R² is H.

In some of these embodiments, R² is optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂. In some embodiments, R² is optionally substituted benzyl, and wherein said optionally substituted benzyl is optionally substituted with one or more of —C(═O)—OH, —C(═O)—O—CH₃, or —C(═O)—NH—CH₃.

In some of these embodiments, R² is optionally substituted (C₁-C₃)hydrocarbyl, and wherein said optionally substituted (C₁-C₃)hydrocarbyl is substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂.

In some of these embodiments, R² is methyl optionally substituted with one or more of —F, —O—C(═O)(C₁-C₄)hydrocarbyl, —C(═O)—OH, —C(═O)—O—CH₃, or —C(═O)—NH—CH₃. In some embodiments, R² is methyl, difluoro methyl, CH₂—O—C(═O)(C₁-C₄)hydrocarbyl, CH₂—O—C(═O)—CH₃, CH₂—C(═O)—OH, CH₂—C(═O)—O—CH₃, or CH₂—C(═O)—NH—CH₃.

In some of these embodiments, R² is ethyl optionally substituted with one or more of —OH, —O—C(═O)(C₁-C₄)hydrocarbyl, —NH(C₁-C₄)hydrocarbyl, or —N[(C₁-C₄)hydrocarbyl)]₂. In some embodiments, R² is ethyl, ethanol, (CH₂)₂—O—C(═O)—CH₃, (CH₂)₂—N[(C₁-C₄)hydrocarbyl)]₂.

In some of these embodiments, R² is propyl optionally substituted with one or more of —CN.

In various embodiments, a pharmaceutical composition comprises a pharmaceutically acceptable carrier and a compound of formula Ia, Ib, IIa, IIb, IIIa, IIIb, IVa, IVb, Va, Vb, VIa, VIb, VIIa, VIIb, or VIII or any other disclosed herein, or a pharmaceutically acceptable salt thereof.

In various embodiments, a method of treating a seizure disorder, status epilepticus, partial-onset seizures, primary generalized tonic-clonic seizures, or convulsions, comprises administering to a subject a therapeutically or prophylactically effective amount of the pharmaceutical composition described above or a compound of formula Ia, Ib, IIa, IIb, IIIa, IIIb, IVa, IVb, Va, Vb, VIa, VIb, VIIa, VIIb, or VIII, or any other disclosed herein, or a pharmaceutically acceptable salt thereof. In a non-limiting example, the seizure disorder is epilepsy. In another non-limiting example, the seizure disorder is a heritable genetic seizure disorder. In some embodiments, the seizure disorder may result from a brain tumor, traumatic brain injury (either concussive or penetrating), or head injury. In some embodiments, the seizure-disorder may result from brain injury during or after viral infection, bacterial infection, parasitic infection, prion disease or idiopathic causes. In some embodiments, the seizure-disorder may result from hemorrhagic stroke or ischemic stroke as post-stroke seizures or recurrent epilepsy after stroke. In some embodiments, the seizure-disorder may result from systemic autoimmune disorders (autoimmune epilepsy), autoimmune encephalitis, Rasmussen's encephalitis (RE), febrile-seizures, febrile-related epilepsies, fever induced refractory epilepsy syndrome (FIRES), or neuroinflammation.

In some embodiments, disclosed are pharmaceutically acceptable salts of the compounds disclosed herein. Preferred embodiments of pharmaceutically acceptable acid addition salts for the compounds of the present invention include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like.

The present disclosure relates to methods and uses for the treatment of pain, convulsions, seizures, epilepsy and status epilepticus.

### Chemical Synthesis of Compounds

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known but are not mentioned here. The starting materials are either commercially available, synthesized as described in the examples or may be obtained by the methods well known to persons of skill in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given; other process conditions can also be used unless otherwise stated. Optimum reaction conditions can vary with the particular reactants or solvent used. Those skilled in the art will recognize that the nature and order of the synthetic steps presented can be varied for the purpose of optimizing the formation of the compounds described comprehensive list of abbreviations utilized by organic chemists appears in the first issue of each volume of the Journal of Organic Chemistry. The list, which is typically presented in a table entitled “Standard List of Abbreviations”, provides definitions for abbreviations not found in the following list of abbreviations:
° C. degrees Celsius
ACN acetonitrile
Cl chloride
Cs₂CO₃ cesium carbonate
DCC N,N′-dicyclohexylcarbodiimide
DCM dichloromethane or methylene chloride
DIPEA diisopropylethylamine
DMAP 4-dimethylaminopyridine or N,N-dimethylaminopyridine
DMF N,N-dimethylformamide
EDC N-(3-dimethylaminopropyl)-N′-ethylcarbodiimide hydrochloride
eq. equivalent(s)
EtOAc ethyl acetate
EtOH ethanol
ESI electrospray ionization
g gram(s)
HATU 1-[bis(dimethylamino)methylene]-1H-1,2,3-triazolo[4,5-b]pyridinium 3-oxid hexafluorophosphate
HBTU 2-(1H-benzotriazole-1-yl)-1,1,3,3-tetramethyluronium hexafluorophosphate
HCl hydrochloric acid
HPLC high performance liquid chromatography
h hour(s)
I iodide
LC/MS liquid chromatography mass spectrometry
LDA lithium diisopropylamide
LiHMDS lithium bis(trimethylsilyl)amide
K₂CO₃ potassium carbonate
KHMDS potassium bis(trimethylsilyl)amide
KMnO₄ potassium permanganate
M molarity concentration
μm micron
MeOH methanol
mg milligram(s)
min minute(s)
mL milliliter(s)
mmol millimole(s)
N normal concentration
Na₂CO₃ sodium carbonate
NaH sodium hydride
NaHCO₃ sodium hydrogen carbonate
NaHMDS sodium bis(trimethylsilyl)amide
NaOH sodium hydroxide
NMR nuclear magnetic resonance
OMs mesylate
OTf triflate
OTs tosylate
Pd/C palladium on carbon
Ph phenyl
psi pounds per square inch
PyBOP (benzotriazol-1-yloxy)tripyrrolidinophosphonium hexafluorophosphate
Ru/C ruthenium on carbon
TBTU 2-(1H-benzotriazole-1-yl)-1,1,3,3-tetramethylaminium tetrafluoroborate
TEA triethylamine
TFA trifluoroacetic acid
THE tetrahydrofuran
TMS-I iodotrimethylsilane
TLC thin layer chromatography

Terminology related to “protecting”, “deprotecting” and “protected” functionalities occurs throughout this application. Such terminology is well understood by persons of skill in the art and is used in the context of processes that involve sequential treatment with a series of reagents. In that context, a protecting group refers to a group, which is used to mask a functionality during a process step in which it would otherwise react, but in which reaction is undesirable. The protecting group prevents reaction at that step but may be subsequently removed to expose the original functionality. The removal or “deprotection” occurs after the completion of the reaction or reactions in which the functionality would interfere. Thus, when a sequence of reagents is specified, as it is in the processes described herein, the person of ordinary skill can readily envision those groups that would be suitable as “protecting groups”. Suitable groups for that purpose are discussed in standard textbooks in the field of chemistry, such as Protective Groups in Organic Synthesis by T. W. Greene and P. G. M. Wuts [John Wiley & Sons, New York, 1999].

### List of Protecting Groups and their Abbreviations

Acetyl (Ac)
Acylals
Carboallyloxy (Alloc)
Benzoyl (Bz)
Benzyl (Bn, Bnl)
Benzyl esters
Carbamate
Carbobenzyloxy (Cbz)
Dimethoxytrityl, [bis-(4-methoxyphenyl)phenylmethyl](DMT)
Dithianes
Ethoxyethyl ethers (EE)
Fluorenylmethyloxycarbonyl (Fmoc)
Methoxymethyl ether (MOM)
Methoxytrityl [(4-methoxyphenyl)diphenylmethyl], MMT)
Methyl Ethers
Methyl (Me)
Methyl esters
Methylthiomethyl ether
Orthoesters
Oxazoline
Pivaloyl (Piv)
Phthalimido
p-Methoxybenzyl carbonyl (Moz or MeOZ)
p-Methoxybenzyl (PMB)
p-Methoxyphenyl (PMP)
Propargyl alcohols
Silyl groups (e.g., trimethylsilyl (TMS), tert-butyldimethylsilyl (TBDMS), tri-iso-propylsilyloxymethyl(TOM) and triisopropylsilyl (TIPS))
Silyl esters
tert-Butyl esters
tert-Butyloxycarbonyl (Boc or tBoc)
Tetrahydropyranyl (THP)
Tosyl (Ts or Tos)
Trichloroethyl chloroformate (Troc)
Trimethylsilylethoxymethyl (SEM)
Trityl (triphenylmethyl, Tr)
β-Methoxyethoxymethyl ether (MEM)
(4-Nitrophenyl)sulfonyl or (4-nitrophenyl)(dioxido)-lambda(6)-sulfanyl) (Nosyl)
2-Cyanoethyl
2-Nitrophenylsulfenyl (Nps)
3,4-Dimethoxybenzyl (DMPM)
2,2,4,6,7-Pentamethyldihydrobenzofuran-5-sulfonyl (Pbf)

### I. Tetrazol and Carboxyl-Ester Derivatives

### A. Generic Chemical Synthesis

Compounds of the present invention are prepared using methods illustrated in the general synthetic schemes and experimental procedures detailed below. These general synthetic schemes and experimental procedures are presented for purposes of illustration and are not intended to be limiting. Starting materials used to prepare compounds of the present invention are commercially available or can be prepared using routine methods known in the art. Where present, names of compounds were generated using ChemAxon's Instant JChem v6.1 for Desktop and IUPAC Naming Plugin.

Intermediate compounds I, V, and VIII are made using protocols previously described in the literature (B. Huff “Excitatory amino acid receptor antagonists.” U.S. Pat. No. 5,284,957, 1994; A. M. Brian Arnold, et al. “Process for preparing isoquinoline compounds.” U.S. Pat. No. 5,648,492, 1997; Paul L. Ornstein, et al. “(3SR,4aRS,6RS,8aRS)-6-[2-(1H-tetrazol-5-yl)ethyl]decahydroisoquinoline-3-carboxylic Acid: A Structurally Novel, Systemically Active, Competitive AMPA Receptor Antagonist.” J. Med. Chem., 1993, 36, 2046-2048; Paul L. Ornstein, et al. “Syntheses of Oxodecahydroisoquinoline-3-carboxylates. Useful Intermediates for the Preparation of Conformationally Defined Excitatory Amino Acid Antagonists.” J. Org. Chem., 1991, 56, 4388-4392).

Scheme 1 illustrates a general synthetic scheme to make the desired compounds IV from ketone I. In step 1, methyl carbamate-protected carboxylic acid I is treated with iodotrimethylsilane at room temperature, or alternatively, with 6 N hydrochloric acid at 90° C., to remove the methyl carbamate-protecting group and afford ketone amino acid II. Compound II is reacted, under basic conditions (e.g. 2 N aqueous NaOH, triethylamine, diisopropylethylamine, etc.), with benzyl chloroformate (Cbz), di-tert-butyl dicarbonate (Boc), or similar reagent to afford carbamate-protected carboxylic acids III. Compounds III are esterified to make ketone carbamate esters IV. Several methods are possible: 1) compounds III are esterified with alcohols (HO-R1) using a coupling reagent (e.g. N,N′-dicyclohexylcarbodiimide (DCC), EDC, HBTU, HATU, PyBOP, etc.) under basic conditions (e.g. triethylamine, diisopropylethylamine, pyridine, N,N-4-dimethylaminopyridine, etc.), 2) compounds III are alkylated with an activated alkane (X-R1, where X=OTf, OTs, OMs, I, Br, and Cl) under basic conditions (e.g. NaOH, NaH, NaCO₃, K₂CO₃, Cs₂CO₃, NaHCO₃, etc.), and 3) compounds III are esterified with alcohols (HO-R1) using Mitsunobu conditions (e.g. diethyl azodicarboxylate and triphenylphosine, or similar reagents).

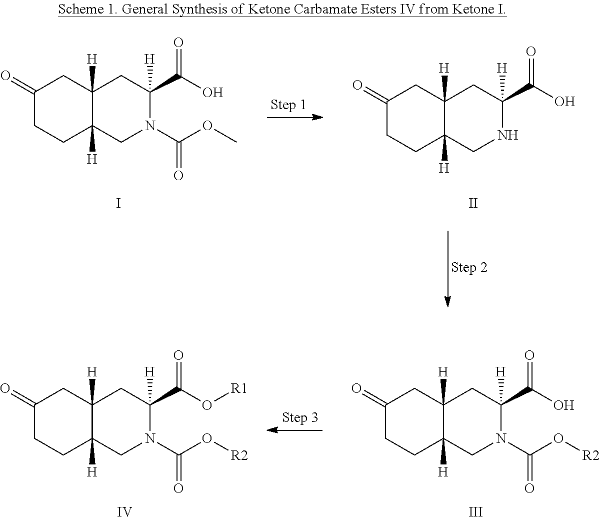

Scheme 2 illustrates a general synthetic scheme to make the desired compounds III from ketone ester V. In step 1, methyl carbamate-protected ethyl ester V is treated with iodotrimethylsilane at room temperature to remove the methyl carbamate-protecting group and afford amino ester VI. Ethyl ester VI is reacted, under basic conditions (e.g. 2 N aqueous NaOH, triethylamine, diisopropylethylamine, etc.), with benzyl chloroformate (Cbz), di-tert-butyl dicarbonate (Boc), or similar protecting group that is stable to basic conditions but that can be removed more easily than methyl carbamate to afford carbamate-protected ethyl ester VII. Compounds VII are then hydrolyzed under standard aqueous basic conditions (e.g. 2 N aqueous NaOH in alcoholic solvent or similar aqueous miscible organic solvent) to afford carbamate-protected acids III. Compounds III are then esterified as described in Scheme 1 to make ketone carbamate esters IV.

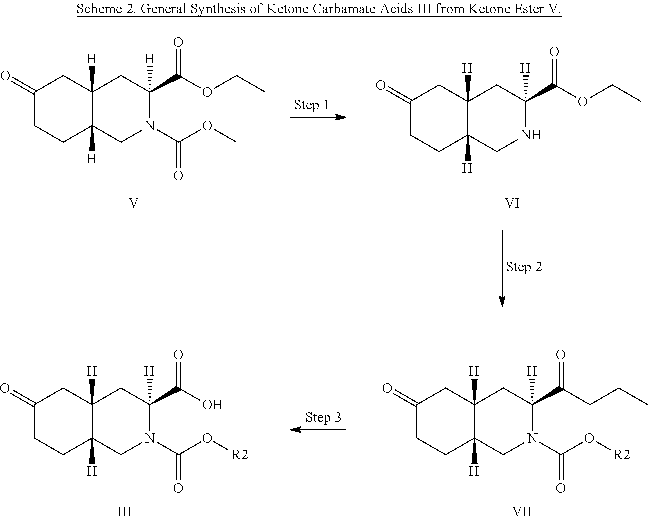

Scheme 3 illustrates a general synthetic scheme to make the desired compounds IX from protected ketone carbamate esters IV. The tetrazole Wittig reagent VIII (made as previously described in B. Huff U.S. Pat. No. 5,284,957) is deprotonated under strong basic conditions (e.g. LiHMDS, NaHMDS, LDA, etc.) and reacted with ketones IV to make olefin compounds IX.

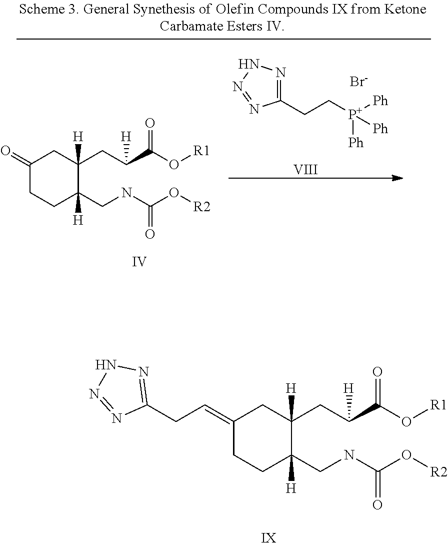

Scheme 4A illustrates a general synthetic scheme to make the desired compounds XIII from protected olefins IX. The protected olefins IX are hydrogenated in the presence of catalyst PtO₂ (Sigma-Aldrich No. 206032) and hydrogen gas (Roberts Oxygen Company, Inc. No. 27700324-00).

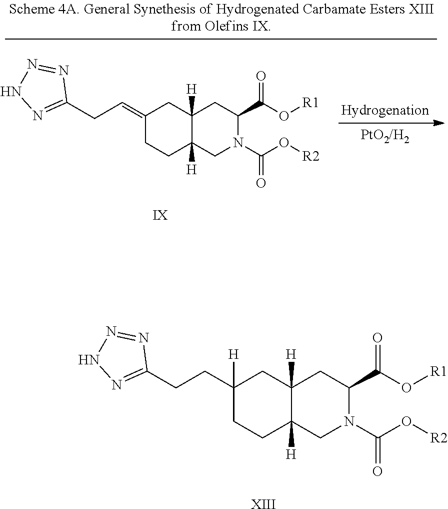

Scheme 4B illustrates a general synthetic scheme to make the desired compounds X from protected olefins IX. Free radical hydrofluorination of IX provides a mixture of fluorinated carbamate esters X (J. Am. Chem. Soc., 2012, 134, 33, 13588-13591 “Fe(III)/NaBH4-Mediated Free Radical Hydrofluorination of Unactivated Alkenes.” Timothy J. Barker and Dale L. Boger). Isomers may be separated by crystallization or chromatography to afford isomerically pure fluorinated carbamate esters X. In addition, alternative hydrofluorination conditions can be used, including but not limited to protocols using KHSO₄-13HF complex (J. Am. Chem. Soc., 2017, 139, 18202-18205 “Widely Applicable Hydrofluorination of Alkenes via Bifunctional Activation of Hydrogen Fluoride.” Zhichao Lu, Xiaojun Zeng, Gerald B. Hammond, and Bo Xu), fluorine cobalt complexes (Org. Lett., 2013, 15, 20, 5158 “Cobalt-Catalyzed Hydrofluorination of Unactivated Olefins: A Radical Approach of Fluorine Transfer.” Hiroki Shigehisa, Eriko Nishi, Mayu Fujisawa, and Kou Hiroya), Superacid HF/SbF5 (Chem. Commun., 2007, 31, 98. “A novel, facile route to beta-fluoroamines by hydrofluorination using superacid HF/SbF5.” Sébastien Thibaudeau, Agnès Martin-Mingot, Marie-Paule Jouannetaud, Omar Karamb, and Fabien Zuninob) and HF-Pyridine (Org. Synth., 1978, 58, 75 “Fluorinations With Pyridinium Polyhydrogen Fluoride.” George A. Olah and Michael Watkins.

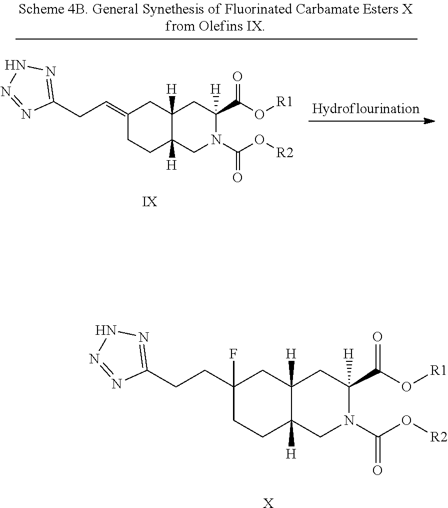

Scheme 5 illustrates a general synthetic scheme to make fluorinated ester amine prodrugs IX from fluorinated carbamate esters X. The carbamate-protecting group is removed using established published protocols (e.g., hydrogenation using hydrogen gas and a Pd/C catalyst for Cbz, TFA conditions or 4 N HCl for Boc, etc.). Isomers are separated by crystallization or chromatography to afford isomerically pure fluorinated ester amines XI.

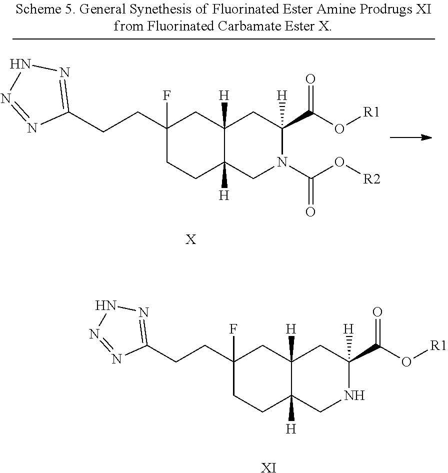

Scheme 6 illustrates a general synthetic scheme to make the desired fluorinated amino acid compound 1 from fluorinated amino esters XI. The esters are hydrolyzed under standard aqueous basic conditions (e.g. 2 N aqueous NaOH or 2 N aqueous NaOH in alcoholic solvent or similar aqueous miscible organic solvent) to afford fluorinated amino acid XII.

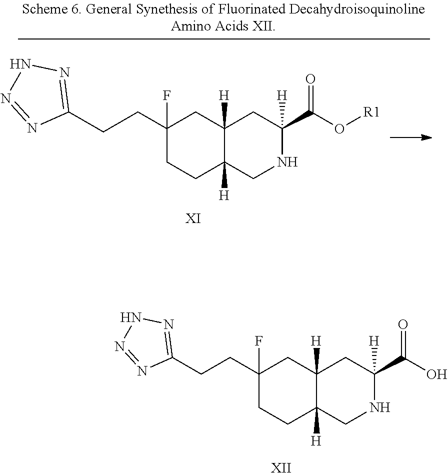

Final purification and isolation affords fluorinated decahydroisoquinoline amino acid 1 and 2.

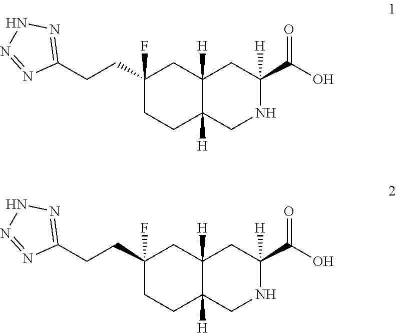 [SMILES: O=C(O)[C@@H]1C[C@H]2C[C@@](F)(CCc3nn[nH]n3)CC[C@H]2CN1 ; O=C(O)[C@@H]1C[C@H]2C[C@](F)(CCc3nn[nH]n3)CC[C@H]2CN1]

### B. Experimental Chemical Synthesis

All chemical reagents were purchased commercially and were used without further purification. Reactions were done under air/nitrogen atmosphere according to the requirements. Column chromatography was performed on silica gel 60 (230-400 mesh) and analytical TLC was performed on plates coated with silica gel. TLC plates were stained with ceric ammonium molybdate (CAM), p-anisaldehyde (Anis), potassium permanganate (KMnO₄), or ninhydrin staining solutions. Routine ¹H NMR spectra were recorded using a Bruker 300 MHz or Varian 300 MHz instrument using deuterium oxide, chloroform-d, or methanol-d₄ as solvents. HPLC spectra were recorded using Agilent Series 1100 HPLC using a Zorbax SB-C18 (4.6×150 mm) column with gradient elution from 5% B to 95% B (Mobile Phase A: 0.05% HClO₄ in water; Mobile Phase B: acetonitrile) over 8.5 min and UV-detection at 205 nm or a Waters Sunfire C18 (4.6×75 mm, 3.5 m, Part No. 186002552) column with gradient elution from 5% B to 95% B (Mobile Phase A: 0.1% TFA in water; Mobile Phase B: 0.1% TFA in acetonitrile) over 8.6 min and UV-detection at all wave length. Mass spectrometry was done using an Advion Expression CMS (ESI) or Agilent (Hewlett Packard Series 1100 MSD) with MassLynx interface (ESI: positive or negative ion mode) or a Waters 29996, Micromass ZQ (ESI: positive or negative ion mode). Preparative reverse phase chromatography was conducted using a Gilson System with a Waters Sunfire C18 OBD preparative column (30×150 mm column, 10 m, Part No. 186002670). In some cases, normal phase silica gel column chromatography was performed using a CombiFlash Teledyne ISCO system.

### Preparation of Ethyl (3S,4aS,8aR)-6-oxo-decahydroisoquinoline-3-carboxylate (I-01)

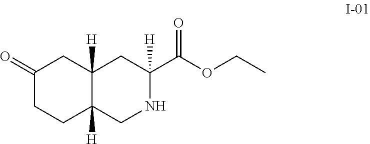 [SMILES: CCOC(=O)[C@@H]1C[C@H]2CC(=O)CC[C@H]2CN1]

To a solution of 3-ethyl 2-methyl (3S,4aS,8aR)-6-oxo-decahydroisoquinoline-2,3-dicarboxylate (35.42 g, 125 mmol) in methylene chloride (600 mL) under nitrogen, was added iodotrimethylsilane (100 g, 500 mmol) in one portion at room temperature. The reaction mixture was stirred overnight and quenched with ethanol (250 mL). The solution was concentrated under vacuum and dried for 3 h under reduced pressure to afford the desired crude amino ester I-01 as a golden yellow solid (43 g crude) which was used directly without purification in the next step. ¹H NMR (300.13 MHz, CD₃OD) δ 4.31 (q, J=5.3 Hz, 2H), 4.17 (d, J=9.6 Hz, 1H), 3.31-3.21 (m, 1H), 3.14 (dd, J=9.6, 3.2 Hz, 1H), 2.21 (d, J=9.5 Hz, 1H), 2.13-2.08 (m, 2H), 2.00 (dt, J=10.1, 3.1 Hz, 1H), 1.88-1.69 (m, 4H), 1.58 (d, J=9.9 Hz, 1H), 1.40-1.35 (m, 1H), 1.32 (t, J=5.3 Hz, 3H) ppm.

Scale-Up Batch: To a solution of 3-ethyl 2-methyl (3S,4aS,8aR)-6-oxo-decahydroisoquinoline-2,3-dicarboxylate (74.4 g, 262.6 mmol) in methylene chloride (1200 mL) under nitrogen, was added iodotrimethylsilane (200 g, 1.0 mol) in one portion at room temperature. The reaction mixture was stirred overnight and quenched with ethanol (280 mL). The solution was concentrated under vacuum and dried for 3 h under reduced pressure to afford the desired crude amino ester I-01 as a golden yellow solid (90.5 g crude) which was used directly without further purification.

### Synthesis of Ketone Carbamate Esters IV

### Preparation of 2-benzyl 3-ethyl (3S,4aS,8aR)-6-oxo-decahydroisoquinoline-2,3-dicarboxylate (I-02)

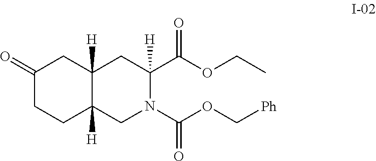 [SMILES: CCOC(=O)[C@@H]1C[C@H]2CC(=O)CC[C@H]2CN1C(=O)OCc1ccccc1]

To a slurry of I-01 (crude, 6.25 mmol) in methylene chloride (50 mL) was added triethylamine (3.5 mL, 25.1 mmol) at 5-10° C. and the mixture was stirred for 10 min under nitrogen atmosphere. Benzyl chloroformate (1.12 mL, 7.62 mmol) was added slowly at 5-10° C. The mixture was warmed to room temperature and stirred for 2-3 h (the reaction was monitored by TLC and KMnO₄ staining). The mixture was adjusted to pH 3-4 using 3 N HCl and diluted with ethyl acetate (50 mL). The layers were separated and the combined organic layers were washed with brine (15 mL), dried over sodium sulfate, and concentrated under vacuum. The resulting residue was purified by flash column chromatography (silica gel, 0% to 40% ethyl acetate/hexane) to afford I-02 as a light yellow oil (2.12 g, 94% yield). ¹H NMR (300.13 MHz, CDCl₃) δ 7.36-7.27 (m, 5H), 5.14 (d, J=24.6, 14.1 Hz, 2H), 4.92 (dd, J=41.3, 8.3 Hz, 1H), 4.22-4.14 (m, 2H), 4.02 (dd, J=22.5, 13.5 Hz, 1H), 3.27 (ddd, J=34.5, 13.5, 3.0 Hz, 1H), 2.59 (dd, J=16.5, 6.0 Hz, 1H), 2.38-1.65 (m, 9H), 1.26 (t, J=7.2 Hz, 1.5H), 1.21 (t, J=7.2 Hz, 1.5H) ppm.

### Preparation of 2-tert-butyl 3-ethyl (3S,4aS,8aR)-6-oxo-decahydroisoquinoline-2,3-dicarboxylate (I-03)

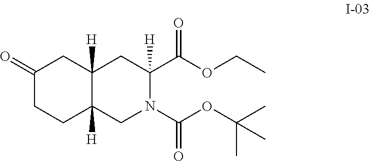 [SMILES: CCOC(=O)[C@@H]1C[C@H]2CC(=O)CC[C@H]2CN1C(=O)OC(C)(C)C]

Crude I-01 (125 mmol) from a previous reaction was dissolved in methylene chloride (600 mL) and triethylamine (60.7 mL, 435 mmol) was added. After stirring for 15 minutes, a solution of di-tert-butyl-dicarbonate (32.7 g, 150 mmol) in methylene chloride (100 mL) was added. The resulting mixture was stirred overnight at room temperature and then concentrated under vacuum. The resulting solid was suspended in ethyl acetate (300 mL) and filtered. The filtrate was washed with 1 N HCl (60 mL) and brine (100 mL). The organic phase was dried over sodium sulfate, filtered, and concentrated under vacuum. The resulting residue was purified by flash column chromatography (silica gel, 0% to 25% ethyl acetate/hexane) to afford I-03 as a colorless oil (30.0 g, 74% yield). ¹H NMR (300.13 MHz, CDCl₃) δ 4.82 (dd, J=65.0, 3.9 Hz, 1H), 4.17-4.14 (m, 2H), 3.90 (dd, J=27.9, 10.2 Hz, 1H), 3.15 (dd, J=39.7, 10.1 Hz, 1H), 2.56 (dd, J=10.7, 4.3 Hz, 1H), 2.41-2.30 (m, 2H), 2.16-1.97 (m, 5H), 1.95-1.78 (m, 1H), 1.75-1.65 (m, 1H), 1.42 (s, 4.5H), 1.40 (s, 4.5H), 1.23 (t, J=4.3 Hz, 3H) ppm.

Scale-Up Batch: Crude I-01 (393.9 mmol) from a previous reaction was dissolved in methylene chloride (1.8 L) and triethylamine (219.3 mL, 1573 mmol) was added. After stirring for 15 minutes, a solution of di-tert-butyl-dicarbonate (343.3 g, 1573 mmol) in methylene chloride (300 mL) was added. The resulting mixture was stirred overnight at room temperature and then concentrated under vacuum. The resulting solid was suspended in ethyl acetate (900 mL) and filtered. The filtrate was washed with 1 N HCl (180 mL) and brine (100 mL). The organic phase was dried over sodium sulfate, filtered, and concentrated under vacuum. The resulting colorless oil (151 g crude, quantitative yield) which was used directly without further purification.

### Preparation of (3S,4aS,8aR)-2-[(tert-butoxy)carbonyl]-6-oxo-decahydroisoquinoline-3-carboxylic acid (I-04)

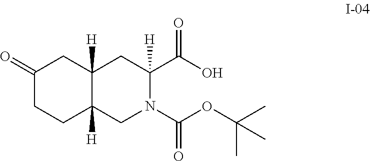 [SMILES: CC(C)(C)OC(=O)N1C[C@@H]2CCC(=O)C[C@@H]2C[C@H]1C(=O)O]

To a solution of I-03 (35.2 g, 108 mmol) in THE (100 mL) was added 2 N NaOH (486 mL, 972 mmol) at room temperature under nitrogen atmosphere. The solution was stirred at room temperature for 24 h and then concentrated under vacuum to remove most of the THF. The aqueous layer was extracted with MTBE (3×150 mL) to remove organic impurities, acidified with 1 N HCl to pH ˜2, and extracted with ethyl acetate (4×300 mL). The combined organic layers were washed with brine (250 mL), dried over sodium sulfate, and concentrated under vacuum to afford I-04 as a white foaming solid (24.9 g, 78% yield). ¹H NMR (300.13 MHz, CDCl₃) δ 4.92 (d, J=58.3 Hz, 1H), 3.95 (dd, J=29.7, 10.1 Hz, 1H), 3.21 (dd, J=32.9, 10.2 Hz, 1H), 2.61 (d, J=10.2 Hz, 1H), 2.42-1.71 (m, 9H), 1.46 (s, 4.5H), 1.44 (s, 4.5H) ppm.

Scale-Up Batch: To a solution of I-03 (Crude 151 g, 393.9 mmol) in THE (360 mL) was added 2 N NaOH (1772 mL, 3545 mmol) at room temperature under nitrogen atmosphere. The solution was stirred at room temperature for 24 h and then concentrated under vacuum to remove most of the THF. The aqueous layer was extracted with MTBE (3×250 mL) to remove organic impurities, acidified with 1 N HCl to pH ˜2, and extracted with ethyl acetate (4×600 mL). The combined organic layers were washed with brine (500 mL), dried over sodium sulfate, and concentrated under vacuum to afford I-04 as a white foaming solid (122 g crude, quantitative yield).

### Preparation of (3S,4aS,8aR)-2-[(tert-butoxy)carbonyl]-6-oxo-decahydroisoquinoline-3-carboxylic acid (I-05)

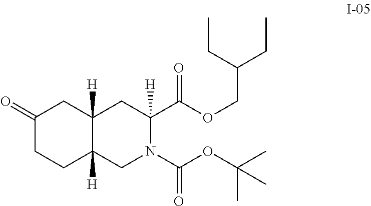 [SMILES: CCC(CC)COC(=O)[C@@H]1C[C@H]2CC(=O)CC[C@H]2CN1C(=O)OC(C)(C)C]

To a solution of I-04 (24.9 g, 83.7 mmol) in DMF (110 mL) was added solid NaHCO₃ (49.4 g, 588 mmol) and 3-(iodomethyl)pentane (23.33 g, 110 mmol) at room temperature under nitrogen atmosphere and the mixture was stirred at 35-40° C. for 4 h. The reaction was monitored by HPLC and after completion of the reaction the mixture was filtered, and the solid was washed with acetonitrile (400 mL). The combined organic layers were concentrated and the crude residue obtained was re-dissolved in ethyl acetate (500 mL). The solution was washed with water (300 mL), brine (300 mL), dried over sodium sulfate, and concentrated under vacuum. The residue was purified by flash column chromatography (silica gel, 0% to 40% ethyl acetate/hexane) to afford I-05 as a red semi-solid (15.2 g, 49% yield pure and 3.5 g, 11% yield less pure). ¹H NMR (300.13 MHz, CDCl₃) δ 4.86 (d, J=63.5 Hz, 1H), 4.16-3.97 (m, 2.5H), 3.88 (d, J=10.1 Hz, 0.5H), 3.15 (dd, J=31.3, 9.8 Hz, 1H), 2.58 (d, J=8.4 Hz, 1H), 2.35 (m, 2H), 2.18-1.48 (m, 8H), 1.44 (m, 9H), 1.40-1.30 (m, 4H), 0.87 (m, 6H) ppm.

Scale-Up Batch: To a solution of crude I-04 (426.8 mmol) in DMF (500 mL) was added solid NaHCO₃ (231.6 g, 2.76 mol) and 3-(iodomethyl)pentane (159.7 g, 754.5 mmol) at room temperature under nitrogen atmosphere and the mixture was stirred at 35-40° C. for 3 days. The reaction was monitored by HPLC and after completion of the reaction the mixture was filtered, and the solid was washed with acetonitrile (1.5 L). The combined organic layers were concentrated and the crude residue obtained was re-dissolved in ethyl acetate (2 L). The solution was washed with water (500 mL), brine (500 mL), dried over sodium sulfate, and concentrated under vacuum to obtain crude product I-05 as a red semi-solid (92.6 g, 57% yield) which was used directly without further purification.

### Preparation of 2-tert-butyl 3-nonyl (3S,4aS,8aR)-6-oxo-decahydroisoquinoline-2,3-dicarboxylate (I-06)

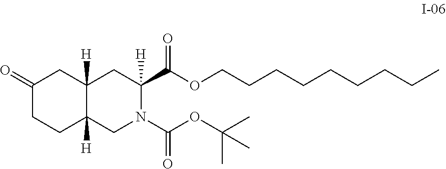 [SMILES: CCCCCCCCCOC(=O)[C@@H]1C[C@H]2CC(=O)CC[C@H]2CN1C(=O)OC(C)(C)C]

To a solution of I-04 (0.5 mmol) in DMF (2.0 mL) is added NaHCO₃ (3.5 mmol) and 1-iodononane (1.3 mmol) at room temperature under nitrogen atmosphere and the mixture is stirred at room temperature for 24 h. The reaction is monitored by HPLC and after completion of the reaction is poured into water (30 mL) and extracted ethyl acetate (2×30 mL). The combined organic layers are dried over sodium sulfate, and concentrated under vacuum. The resulting residue is purified by flash column chromatography (silica gel, ethyl acetate/hexane) to afford I-06.

### Preparation of 3-(2R)-butan-2-yl 2-tert-butyl (3S,4aS,8aR)-6-oxo-decahydroisoquinoline-2,3-dicarboxylate (I-07)

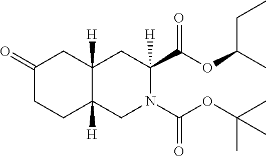 [SMILES: CC[C@@H](C)OC(=O)[C@@H]1C[C@H]2CC(=O)CC[C@H]2CN1C(=O)OC(C)(C)C]

To a mixture of I-04 (0.5 mmol) and (2R)-butanol (0.6 mmol) in dichloromethane (1.0 mL) is added DCC (0.6 mmol) and catalytic DMAP under nitrogen atmosphere. The mixture is stirred at room temperature for 18 h and monitored by TLC or HPLC. Upon completion, acetonitrile (10 mL) is added and the mixture is stirred for 5-10 min. The solid precipitate is removed by filtration through a sintered glass-funnel and the solid is washed with acetonitrile (10 mL). The filtrate is concentrated under vacuum and the residue is purified by flash column chromatography (silica gel, ethyl acetate/hexane) to afford I-07.

### Synthesis of Olefin Compounds IX

### Preparation of 2-benzyl 3-ethyl (3S,4aR,6E,8aR)-6-[2-(1H-1,2,3,4-tetrazol-5-yl)ethylidene]-decahydroisoquinoline-2,3-dicarboxylate (I-08)

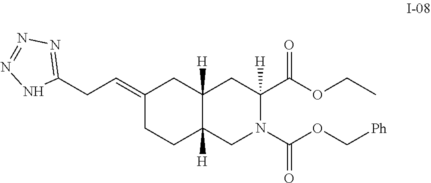 [SMILES: CCOC(=O)[C@@H]1C[C@H]2C/C(=C/Cc3nnn[nH]3)CC[C@H]2CN1C(=O)OCc1ccccc1]

To a solution of triphenyl[2-(1H-1,2,3,4-tetrazol-5-yl)ethyl]phosphanium bromide salt (VIII, 9.09 g, 20.7 mmol) and ketone (I-02, 6.2 g, 17.25 mmol) in anhydrous DMF (50 mL) was added 2.0 M NaHMDS (24.15 mL, 48.3 mmol) in THE at 0° C. to −10° C. under nitrogen atmosphere. The internal temperature of the reaction was maintained at 0° C. during addition. The mixture was stirred at this temperature for 30 min and then allowed to warm to room temperature. After stirring for 18 h at room temperature, the mixture was slowly quenched with an ice-cold brine solution (120 mL) and extracted with MTBE (7×250 mL) to partially remove triphenylphosphine oxide. The pH of the aqueous layer was adjusted to pH 2 using 3 N HCl and extracted with ethyl acetate (4×250 mL). The combined organic layers were washed with water (2×200 mL), brine (200 mL), dried over sodium sulfate, and concentrated under vacuum. The residue was purified by flash column chromatography (silica gel, 10% to 60% ethyl acetate/hexane) to afford I-08 as a pure white foaming solid (2.0 g, 26% yield) and as a less pure yellow oil (2.0 g, 26% yield). ¹H NMR (299.96 MHz, CDCl₃) δ 7.38-7.27 (m, 5H), 5.50 (t, J=6.7 Hz, 0.5H), 5.32 (dt, J=21.5, 6.4 Hz, 0.5H), 5.23-5.14 (m, 1.5H), 5.06 (d, J=12.9 Hz, 0.5H), 4.89 (d, J=27.0 Hz, 1H), 4.17 (q, J=6.8 Hz, 2H), 3.95-3.87 (m, 1H), 3.76-3.69 (m, 2H), 3.25 (d, J=14.3 Hz, 0.5H), 3.15 (d, J=12.9 Hz, 0.5H), 2.65 (d, J=12.6 Hz, 0.5H), 2.47 (d, J=13.8 Hz, 0.5H), 2.34 (d, J=13.2 Hz, 0.5H), 2.24 (d, J=11.4 Hz, 0.5H), 2.08-1.65 (m, 6H), 1.62-1.47 (m, 2H), 1.20 (d, J=7.8 Hz, 3H) ppm.

### Preparation of 2-tert-butyl 3-ethyl (3S,4aR,8aR)-6-[2-(1H-1,2,3,4-tetrazol-5-yl)ethylidene]-decahydroisoquinoline-2,3-dicarboxylate (I-09)

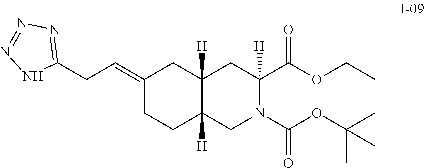 [SMILES: CCOC(=O)[C@@H]1C[C@H]2C/C(=C/Cc3nnn[nH]3)CC[C@H]2CN1C(=O)OC(C)(C)C]

To a solution of triphenyl[2-(1H-1,2,3,4-tetrazol-5-yl)ethyl]phosphanium bromide salt (VIII, 6.16 g, 14.0 mmol) and ketone (I-03, 3.8 g, 11.7 mmol) in anhydrous DMF (35 mL) was added 2.0 M NaHMDS (16.4 mL, 32.8 mmol) in THE at 0° C. to −10° C. under nitrogen atmosphere. The internal temperature of the reaction was maintained at 0° C. during addition. The mixture was stirred at this temperature for 30 min and then allowed to warm to room temperature After stirring for 18 h at room temperature, the mixture was slowly quenched with an ice-cold brine solution (50 mL) and extracted with MTBE (8×60 mL) to partially remove triphenylphosphine oxide. The pH of the aqueous layer was adjusted to pH 2 using 3 N HCl and extracted with ethyl acetate (4×250 mL). The combined organic layers were washed with water (2×200 mL), brine (200 mL), dried over sodium sulfate, and concentrated under vacuum to afford I-09 as a red oil (6.1 g crude). ¹H NMR (300.13 MHz, CDCl₃) δ 5.37 (m, 1H), 4.82 (dd, J=48.5, 3.5 Hz, 0.5H), 4.77 (d, J=45.5 Hz, 0.5H), 4.21-4.18 (m, 2H), 3.87-3.78 (m, 1H), 3.25-3.12 (m, 3H), 2.49-2.47 (m, 1.5H), 2.34 (d, J=13.1 Hz, 0.5H), 2.1.0-1.60 (m, 8H), 1.54-1.46 (m, 9H), 1.30-1.22 (m, 3H) ppm.

### Preparation of 2-tert-butyl 3-(2-ethylbutyl) (3S,4aR,8aR)-6-[2-(1H-1,2,3,4-tetrazol-5-yl)ethylidene]-decahydroisoquinoline-2,3-dicarboxylate (I-10)

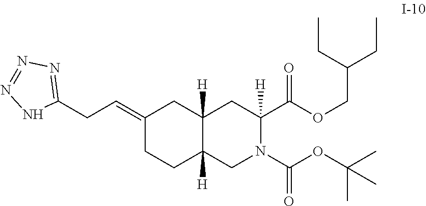 [SMILES: CCC(CC)COC(=O)[C@@H]1C[C@H]2C/C(=C/Cc3nnn[nH]3)CC[C@H]2CN1C(=O)OC(C)(C)C]

To a solution of tetrazole Wittig salt (VIII, 23.37 g, 53.2 mmol) and ketone (I-04, 16.9 g, 44.3 mmol) in anhydrous DMF (150 mL) was added 2.0 M NaHMDS (62 mL, 124 mmol) in THE at 0° C. to −10° C. under nitrogen atmosphere. The internal temperature of the reaction was maintained at 0° C. during addition. The mixture was stirred at this temperature for 30 min and then allowed to warm to room temperature After stirring for 2 h at room temperature, the mixture was slowly quenched with 10% brine solution (200 mL) and extracted with MTBE (4×300 mL) to partially remove triphenylphosphine oxide. The pH of the aqueous layer was adjusted to pH 2 using 3 N HCl and extracted with ethyl acetate (4×300 mL). The combined organic layers were washed with water (2×150 mL), brine (200 mL), dried over sodium sulfate, and concentrated under vacuum. The residue was purified by flash column chromatography (silica gel, 0% to 50% ethyl acetate/hexane) to afford I-10 as foaming solid (13.1 g, 64% yield pure) and as a pale yellow semi-solid (2.6 g, 13% yield, less pure). ¹H NMR (299.96 MHz, CDCl₃) δ 5.55 (t, J=7.0 Hz, 0.4H), 5.38 (dt, J=20.3, 16.8 Hz, 0.6H), 4.84 (d, J=46.8 Hz, 1H), 4.09-4.00 (m, 2H), 3.89-3.73 (m, 3H), 3.14-3.03 (m, 1H), 2.69 (t, J=10.6 Hz, 0.6H), 2.49 (dd, J=13.6, 6.1 Hz, 0.4H), 2.34 (d, J=13.5 Hz, 0.6H), 2.31-2.21 (m, 0.4H), 2.09 (d, J=14.7 Hz, 1H), 2.01 (t, J=11.7 Hz, 1H), 1.90-1.75 (m, 4H), 1.65-1.40 (m, 3H), 1.45 (s, 9H), 1.40-1.26 (m, 5H), 0.91-0.85 (m, 6H) ppm.

Scale-Up Batch: To a solution of tetrazole Wittig salt (VIII, 121.8 g, 277.3 mmol) and crude ketone (I-04, 88 g, 230.7 mmol) in anhydrous DMF (670 mL) was added 2.0 M NaHMDS (323.3 mL, 646.6 mmol) in THE at 0° C. to −10° C. under nitrogen atmosphere. The internal temperature of the reaction was maintained at 0° C. during addition. The mixture was stirred at this temperature for 30 min and then allowed to warm to room temperature After stirring for 2 h at room temperature, the mixture was slowly quenched with 10% brine solution (500 mL) and extracted with MTBE (2×400 mL) to partially remove triphenylphosphine oxide. The pH of the aqueous layer was adjusted to pH 2 using 3 N HCl and extracted with ethyl acetate (4×1 L). The combined organic layers were washed with water (2×1L), brine (500 mL), dried over sodium sulfate, and concentrated under vacuum. The residue was purified by flash column chromatography (silica gel, 0% to 50% ethyl acetate/hexane) to afford I-10 as foaming solid (51.6 g, 49% yield).

### Synthesis of Fluorinated Carbamate Esters X

### Preparation of 2-benzyl 3-ethyl (3S,4aS,8aR)-6-fluoro-6-[2-(1H-1,2,3,4-tetrazol-5-yl)ethyl]-decahydroisoquinoline-2,3-dicarboxylate (I-11)

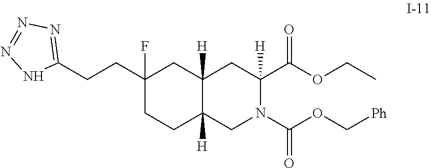 [SMILES: CCOC(=O)[C@@H]1C[C@H]2CC(F)(CCc3nnn[nH]3)CC[C@H]2CN1C(=O)OCc1ccccc1]

A 3-necked, 500 mL flask was prepared with a nitrogen inlet, temperature probe, and cooling bath. Water (85 mL) was charged into the flask, followed by iron (III) nitrate nonahydrate (Fe(NO₃)₃·9 H₂O (1.46 g, 3.04 mmol) and the mixture was stirred until dissolved (Solution A). Acetonitrile (85 mL) was charged into a 500 mL round-bottomed flask, followed by I-08 (667 mg, 1.517 mmol) and Selectfluor® (1.61 g, 4.55 mmol), and the mixture was stirred until dissolved (Solution B). Solution B was charged into Solution A while stirring at 22-25° C. A clear yellow solution was observed and the pH of the solution was measured to be pH 2. The reaction was degassed using nitrogen bubbling for 10 min and the mixture was cooled to −10° C. Sodium borohydride (591.2 mg) was charged in 4 installments as a solid over 5-10 min. The mixture was stirred at −10° C. for 2 h, then the mixture was warmed to 22-25° C. and stirring was continued for 5 h monitoring by HPLC. The mixture was concentrated to remove acetonitrile under vacuum using a rotary evaporator. To this mixture was added 1 N HCl (110 mL) while stirring and maintaining the temperature below 25° C., to adjust to pH 2. The solution was extracted with ethyl acetate (4×100 mL). The organic layers were washed with water (2×100 mL) and brine (100 mL), the solution was dried over sodium sulfate and concentrated under vacuum using a rotary evaporator to afford crude product (690 mg, 100%). The resulting residue was purified by flash column chromatography (silica gel, 0% to 6% methanol/dichloromethane) to afford I-11 as a white forming solid (590 mg, 85% yield). ¹H NMR (299.96 MHz, CDCl₃) δ 7.35-7.26 (m, 5H), 5.25-5.09 (m, 2H), 4.93 (dd, J=24.9, 5.4 Hz, 0.6H), 4.62 (m, 0.4H), 4.21-4.10 (m, 2H), 4.00 (t, J=11.2 Hz, 0.6H), 3.61 (dd, J=12.9, 6.6 Hz, 0.4H), 3.43 (dd, J=13.5, 3.6 Hz, 0.4H), 3.27 (t, J=14.5 Hz, 0.6H), 3.17-3.05 (m, 2H), 2.39-1.36 (m, 12H), 1.27-1.21 (m, 3H) ppm.

### Preparation of 2-tert-butyl 3-ethyl (3S,4aS,8aR)-6-fluoro-6-[2-(1H-1,2,3,4-tetrazol-5-yl)ethyl]-decahydroisoquinoline-2,3-dicarboxylate (I-12)

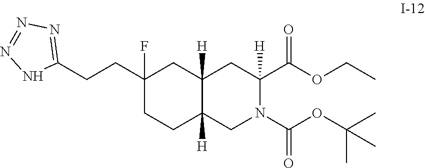 [SMILES: CCOC(=O)[C@@H]1C[C@H]2CC(F)(CCc3nnn[nH]3)CC[C@H]2CN1C(=O)OC(C)(C)C]

A 3-necked, 3-L flask was prepared with a nitrogen inlet, temperature probe, and cooling bath. Water (1.0 L) was charged into the flask, followed by iron (III) nitrate nonahydrate (Fe(NO₃)₃·9 H₂O, 44.17 g, 109.34 mmol) and the mixture was stirred until dissolved (Solution A). Acetonitrile (1.0 L) was charged into a 2-L round-bottomed flask, followed by I-09 (14.3 g, 35.27 mmol) and Selectfluor® (38.73 g, 109 mmol), and the mixture was stirred until dissolved (Solution B). Solution B was charged into Solution A while stirring at 22-25° C. A clear yellow solution was observed and the pH of the solution was measured to be pH 2. The reaction was degassed using nitrogen bubbling for 30 min and the mixture was cooled to −10° C. Sodium borohydride (13.34 g, 353 mmol) was charged in ten installments as a solid over 10-15 min. The mixture was stirred at −10° C. for 2 h, then the mixture was warmed to 22-25° C. and stirring was continued for 5 h while monitoring by HPLC. The mixture was concentrated under vacuum to remove acetonitrile using a rotary evaporator. To this mixture was added 1 N HCl (500 mL) while stirring and maintaining the temperature below 25° C., the mixture was adjusted to pH 2. The solution was extracted with ethyl acetate (4×500 mL). The combined organic layers were washed with water (2×500 mL) and brine (100 mL), dried over sodium sulfate, and concentrated under vacuum using a rotary evaporator to afford crude product (13.5 g, 90%). The resulting residue was purified by flash column chromatography (silica gel, 0% to 70% ethyl acetate/heptane) to afford I-12 as white foaming solid (11.36 g, 76% yield) as a mixture of diastereomers. ¹H NMR (299.962 MHz, CDCl₃) δ 4.86 (dd, J=51.3, 5.1 Hz, 0.5H), 4.49 (t, J=5.1 Hz, 0.5H), 4.25-4.14 (m, 2H), 3.90 (t, J=15.0 Hz, 0.5H), 3.47 (dd, J=13.3, 7.3 Hz, 0.5H), 3.37 (dd, J=13.2, 4.8 Hz, 0.5H), 3.22-3.14 (m, 2H), 3.14-3.04 (m, 0.5H), 2.34-1.53 (m, 12H), 1.49-1.46 (m, 9H), 1.27 (t, J=6.8 Hz, 3H) ppm.

### Preparation of tert-butyl (3S,4aR,6R,8aR)-3-(2-ethylbutoxycarbonyl)-6-[2-(2H-tetraazol-5-yl)ethyl]perhydro-2-isoquinolinecarboxylate (I-13A)

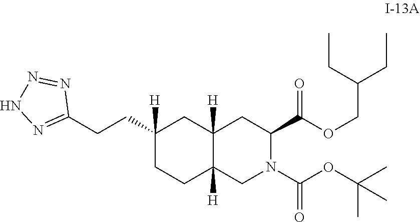 [SMILES: CCC(CC)COC(=O)[C@@H]1C[C@H]2C[C@@H](CCc3nn[nH]n3)CC[C@H]2CN1C(=O)OC(C)(C)C]

To a solution of IX (6.5 g, 14.08 mmol) in THF (60 mL; Sigma-Aldrich No. 186562) was added catalyst PtO2 (600 mg, 2.7 mmol). The mixture was degassed using vacuum and refilled with hydrogen gas. This process was repeated three times. The solution was then stirred at 25-30° C. for 16-18 h. After completion of reaction the mixture was filtered through celite and celite cake was washed with THF (10 mL). Combined THF solution was concentrated at 35-40° C. under vacuum to I-13A as light brown semi-solid (5.1 g, 78%) and used for next step without further purification. Molecular Formula: C₂₄H₄₁N₅O₄. Mol. Wt. 463.61. 1H NMR (CDCl₃, 300 MHz) spectrum showed the correct compound was made. TLC (60% ethyl acetate in hexane). Rf Starting Material: 0.4. Rf Product reaction mixture: 0.3.

### Preparation of 2-tert-butyl 3-(2-ethylbutyl) (3S,4aS,8aR)-6-fluoro-6-[2-(1H-1,2,3,4-tetrazol-5-yl)ethyl]-decahydroisoquinoline-2,3-dicarboxylate (I-13B)

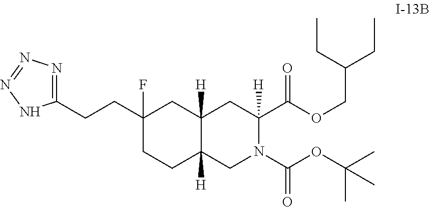 [SMILES: CCC(CC)COC(=O)[C@@H]1C[C@H]2CC(F)(CCc3nnn[nH]3)CC[C@H]2CN1C(=O)OC(C)(C)C]

A 3-necked, 2-L flask was prepared with a nitrogen inlet, temperature probe, and cooling bath. Water (400 mL) was charged into the flask, followed by iron (III) nitrate nonahydrate (Fe(NO₃)₃·9 H₂O, 34.2 g, 84.63 mmol) and the mixture was stirred until dissolved (Solution A). Acetonitrile (400 mL) was charged into a 1-L round-bottomed flask, followed by I-10 (12.6 g, 27.3 mmol) and Selectfluor® (30 g, 84.63 mmol), and the mixture was stirred until dissolved (Solution B). Solution B was charged into Solution A while stirring at 22-25° C. A clear yellow solution was observed and the pH of the solution was measured to be pH 2. The reaction was degassed using nitrogen bubbling for 30 min and the mixture was cooled to −10° C. Sodium borohydride (10.33 g, 273 mmol) was charged in ten installments as a solid over 10-15 min. The mixture was stirred at −10° C. for 2 h, then the mixture was warmed to 22-25° C. and stirring was continued for 5 h monitoring by HPLC. The mixture was concentrated under vacuum to remove acetonitrile using a rotary evaporator. To this mixture was added 1 N HCl (˜300 mL) while stirring and maintaining the temperature below 25° C., the mixture was adjusted to pH 2. The solution was extracted with ethyl acetate (3×350 mL). The combined organic layers were washed with water (400 mL) and brine (300 mL), dried over sodium sulfate, and concentrated under vacuum using a rotary evaporator to afford crude product. The resulting residue was purified by flash column chromatography (silica gel, 0% to 50% ethyl acetate/heptane) to afford I-13B as white foaming solid (8.03 g, 61% yield) as a mixture of diastereomers. ¹H NMR (299.96 MHz, CDCl₃) δ 5.55 (t, J=7.0 Hz, 0.4H), 5.38 (dt, J=20.3, 16.8 Hz, 0.6H), 4.84 (d, J=46.8 Hz, 1H), 4.09-4.00 (m, 2H), 3.89-3.73 (m, 3H), 3.14-3.03 (m, 1H), 2.69 (t, J=10.6 Hz, 0.6H), 2.49 (dd, J=13.6, 6.1 Hz, 0.4H), 2.34 (d, J=13.5 Hz, 0.6H), 2.31-2.21 (m, 0.4H), 2.09 (d, J=14.7 Hz, 1H), 2.01 (t, J=11.7 Hz, 1H), 1.90-1.75 (m, 4H), 1.65-1.40 (m, 3H), 1.45 (s, 9H), 1.40-1.26 (m, 5H), 0.91-0.85 (m, 6H) ppm.

Scale-Up Batch: A 3-necked, 5-L flask was prepared with a nitrogen inlet, temperature probe, and cooling bath. Water (1.5 L) was charged into the flask, followed by iron (III) nitrate nonahydrate (Fe(NO₃)₃·9 H₂O, 72.3 g, 178.96 mmol) and the mixture was stirred until dissolved (Solution A). Acetonitrile (1.5 L) was charged into a 3-L round-bottomed flask, followed by I-10 (26.6 g, 57.9 mmol) and Selectfluor® (63.4 g, 178.96 mmol), and the mixture was stirred until dissolved (Solution B). Solution B was charged into Solution A while stirring at 22-25° C. A clear yellow solution was observed and the pH of the solution was measured to be pH 2. The reaction was degassed using nitrogen bubbling for 30 min and the mixture was cooled to −10° C. Sodium borohydride (21.8 g, 576.2 mmol) was charged in ten installments as a solid over 10-15 min. The mixture was stirred at −10° C. for 2 h, then the mixture was warmed to 22-25° C. and stirring was continued for 5 h monitoring by HPLC. The mixture was concentrated under vacuum to remove acetonitrile using a rotary evaporator. To this mixture was added 1 N HCl (˜640 mL) while stirring and maintaining the temperature below 25° C., the mixture was adjusted to pH 2. The solution was extracted with ethyl acetate (3×500 mL). The combined organic layers were washed with water (500 mL) and brine (500 mL), dried over sodium sulfate, and concentrated under vacuum using a rotary evaporator to afford crude product. The resulting residue was purified by flash column chromatography (silica gel, 0% to 50% ethyl acetate/heptane) to afford I-13B as white foaming solid (21.7 g, 61% yield) as a mixture of diastereomers.

### Preparation of (3S,4aS,8aR)-2-[(benzyloxy)carbonyl]-6-fluoro-6-[2-(2H-1,2,3,4-tetrazol-5-yl)ethyl]-decahydroisoquinoline-3-carboxylic acid (I-14)

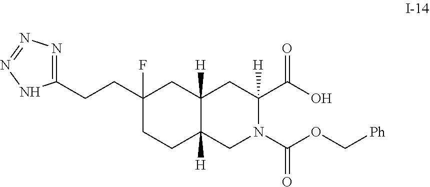 [SMILES: O=C(O)[C@@H]1C[C@H]2CC(F)(CCc3nnn[nH]3)CC[C@H]2CN1C(=O)OCc1ccccc1]

Compound I-11 (0.59 g, 1.28 mmol) was dissolved in 9:1 ethanol-water (2.0 mL) and 2N NaOH (5.3 mL) was added. The mixture was stirred at room temperature for 4 h. The reaction was diluted with water (50 mL) and extracted with MTBE (50 mL) to remove the organic impurities. The aqueous layer was acidified with 6N HCl to pH ˜2. The mixture was extracted with ethyl acetate (3×50 mL) and washed with brine (50 mL). The organic layers were combined, dried over sodium sulfate, and concentrated under vacuum using a rotary evaporator to afford crude product I-14 as a white solid (435 mg, 79% yield). The crude product was used without further purification directly in a subsequent step.

### Preparation of (3S,4aS,8aR)-2-[(tert-butoxy)carbonyl]-6-fluoro-6-[2-(1H-1,2,3,4-tetrazol-5-yl)ethyl]-decahydroisoquinoline-3-carboxylic acid (I-15)

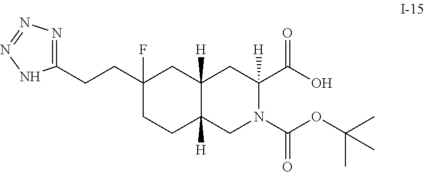 [SMILES: CC(C)(C)OC(=O)N1C[C@@H]2CCC(F)(CCc3nnn[nH]3)C[C@@H]2C[C@H]1C(=O)O]

Compound I-12 (185 mg, 0.435 mmol) was dissolved in 9:1 ethanol-water (1.0 mL) and 2N NaOH (2.5 mL) was added. The mixture was stirred at room temperature for 4 h. The reaction was diluted with water (50 mL) and extracted with MTBE (50 mL) to remove the organic impurities. The aqueous layer was acidified with 6N HCl to pH ˜2. The mixture was extracted with ethyl acetate (3×50 mL) and washed with brine (50 mL). The organic layers were combined, dried over sodium sulfate, and concentrated under vacuum using a rotary evaporator to afford crude product (183 mg), which was purified by silica gel column chromatography (using an ISCO gradient system 0% to 10% methanol-dichloromethane) to afford I-15 as an oil (143 mg, 83% yield). This material was used directly in a subsequent step.

### Preparation of 2-benzyl 3-cyclohexyl (3S,4aS,8aR)-6-fluoro-6-[2-(1H-1,2,3,4-tetrazol-5-yl)ethyl]-decahydroisoquinoline-2,3-dicarboxylate (I-16)

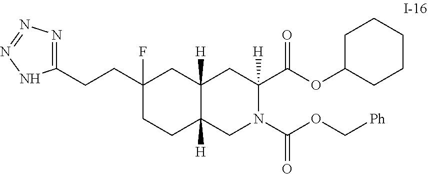 [SMILES: O=C(OC1CCCCC1)[C@@H]1C[C@H]2CC(F)(CCc3nnn[nH]3)CC[C@H]2CN1C(=O)OCc1ccccc1]

Compound I-14 (51.5 mg, 0.12 mmol) and cyclohexanol (14.4 mg, 0.144 mmol) were dissolved in dichloromethane (1 mL). Diisopropylcarbodiimide (20 mg, 0.16 mmol) and catalytic 4-dimethylaminopyridine (5 mg, 0.04 mmol) were added to the mixture under nitrogen atmosphere and the reaction was stirred at room temperature for 16-18 h. The reaction was monitored by HPLC, and upon completion, acetonitrile (1 mL) was added and the mixture was stirred for 5-10 min. The solid precipitate was removed by filtration through a sintered glass-funnel and the solid was washed with acetonitrile (5 mL). The filtrate was concentrated under vacuum and the residue was purified by silica gel flash column chromatography (0% to 5% methanol/dichloromethane) to afford I-16 as a colorless oil (40.4 mg, 66% yield). ¹H NMR (299.96 MHz, CDCl₃) δ 7.35-7.27 (m, 5H), 5.23-5.07 (m, 2H), 4.95-4.50 (m, 2H), 3.97 (t, J=12.3 Hz, 0.4H), 3.89-3.73 (m, 0.4H), 3.61 (dd, J=13.6, 6.7 Hz, 0.4H), 3.40 (dd, J=12.9, 4.2 Hz, 0.3H), 3.32-3.20 (m, 0.5H), 3.16-3.05 (m, 2H), 2.34-1.65 (m, 13H), 1.51-1.25 (m, 8H), 1.16 (d, J=6.3 Hz, 1H) ppm.

### Preparation of 2-benzyl 3-octyl (3S,4aS,8aR)-6-fluoro-6-[2-(1H-1,2,3,4-tetrazol-5-yl)ethyl]-decahydroisoquinoline-2,3-dicarboxylate (I-17)

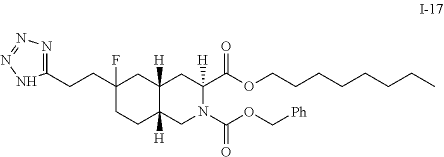 [SMILES: CCCCCCCCOC(=O)[C@@H]1C[C@H]2CC(F)(CCc3nnn[nH]3)CC[C@H]2CN1C(=O)OCc1ccccc1]

Compound I-14 (54.8 mg, 0.127 mmol) and 1-octanol (20 mg, 0.153 mmol) were dissolved in dichloromethane (1.0 mL). Dicyclohexylcarbodiimide (35 mg, 0.168 mmol) and catalytic 4-dimethylaminopyridine (5 mg, 0.037 mmol) were added to the mixture under nitrogen atmosphere and the reaction was stirred at room temperature for 16-18 h. The reaction was monitored by HPLC, and upon completion, the solid precipitate was removed by filtration through a sintered glass-funnel and the solid was washed with acetonitrile (5 mL). The filtrate was concentrated under vacuum and the residue was purified by silica gel flash column chromatography (0% to 5% methanol/dichloromethane) to afford I-17 as a colorless oil (40.2 mg, 58% yield). ¹H NMR (299.96 MHz, CDCl₃) δ 7.40-7.25 (m, 5H), 5.25-5.10 (m, 2H), 4.94 (d, J=25.5 Hz, 0.65H), 4.65 (s, 0.35H), 4.10-3.95 (m, 3H), 3.70-3.60 (m, 1H), 3.45-3.20 (m, 1H), 3.19-3.05 (m, 2H), 2.35-1.56 (m, 24H), 0.87 (m, 3H) ppm.

Synthesis of hydrogenated dual tetrazol and carboxylester derivative compounds.

The hydrogenated mono carboxylester derivative compounds illustrated herein may also be synthesized as hydrogenated dual tetrazol and carboxylester derivatives through various methods which are known to a person skilled in the art.

Non-limiting examples of the synthesis of hydrogenated dual tetrazol and carboxylester derivative compounds are shown below.

### Preparation of 2-ethylbutyl (3S,4aR,6R,8aR)-6-{2-[2-(acetoxymethyl)-2H-tetraazol-5-yl]ethyl}perhydro-3-isoquinolinecarboxylate (310) and 2-ethylbutyl (3S,4aR,6R,8aR)-6-{2-[1-(acetoxymethyl)-2H-tetraazol-5-yl]ethyl}perhydro-3-isoquinolinecarboxylate (311)

### Step 1. Synthesis of tert-butyl (3S,4aR,6R,8aR)-6-{2-[2-(acetoxymethyl)-2H-tetraazol-5-yl]ethyl}-3-(2-ethylbutoxycarbonyl)perhydro-2-isoquinolinecarboxylate and tert-butyl (3S,4aR,6R,8aR)-6-{2-[1-(acetoxymethyl)-1H-tetraazol-5-yl]ethyl}-3-(2-ethylbutoxycarbonyl)perhydro-2-isoquinolinecarboxylate

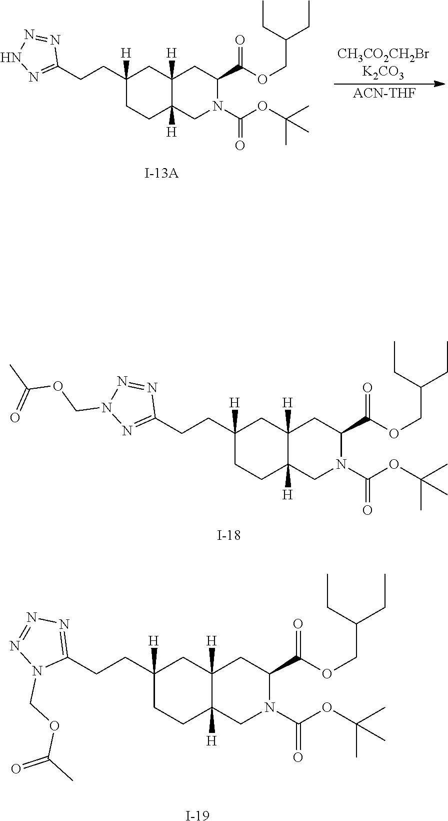

To a solution of I-13A (4.63 g, 10 mmol) in ACN-THF (90 mL, 4:5; Sigma-Aldrich Nos. 360457 and 186562, respectively) was added K₂CO₃ (2.77 g, 20 mmol; Sigma-Aldrich No. 900501), Bromomethyl acetate (CH₃CO₂CH₂Br, Mol. Wt. 152.97; Alfa Aesar No. H56755) 1.63 g (11 mmol) at room temperature under nitrogen atmosphere. The solution was stirred at 25-27° C. for 16-18 h and then diluted with EtOAc (100 mL). After filtration through celite the solution was concentrated at 35-40° C. under vacuum. The residue was purified by flash column chromatography (silica gel, 10-60% EtOAc/Hexanes; Sigma-Aldrich Nos. 717185 (silica gel), 319902 (EtOAc), and 178918 (Hexanes)) to afford the product which was eluted and collected as two peaks (4.8 g, 89%) (3.2 g) and (1.6 g). Mass Spec analysis of the 2 respective peaks from silica gel chromatography showed the expected M+1 ion for I-18 and the expected M+1 for I-19. Molecular Formula: C₂₇H₄₅N₅O₆ Mol. Wt. 535.68. 1H NMR of product before Silica gel column flash chromatography (CDCl₃, 300 MHz) confirmed a mixture of tetrazole isomers was made.

1H NMR of products after Silica gel column flash chromatography (CDCl₃, 300 MHz) confirmed each of the respective tetrazole isomers was separated and isolated.

TLC (50% ethyl acetate in hexane). Rf starting material I-13A (crude): (Rf 0.2). Rf products I-18 and I-19 (crude): top spot (Rf 0.6), bottom spot (Rf 0.4).

### Step 2. Synthesis of 2-ethylbutyl (3S,4aR,6R,8aR)-6-{2-[2-(acetoxymethyl)-2H-tetraazol-5-yl]ethyl}perhydro-3-isoquinolinecarboxylate (310) and 2-ethylbutyl (3S,4aR,6R,8aR)-6-{2-[l-(acetoxymethyl)-2H-tetraazol-5-yl]ethyl}perhydro-3-isoquinolinecarboxylate (311)

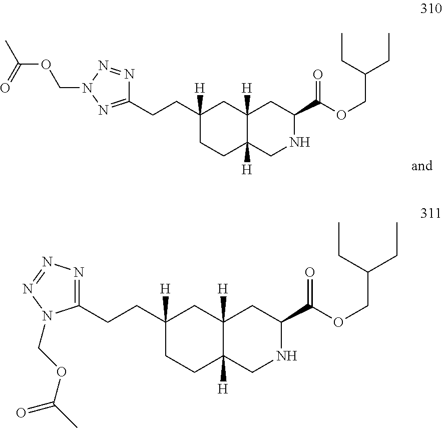 [SMILES: CCC(CC)COC(=O)[C@@H]1C[C@H]2C[C@@H](CCc3nnn(COC(C)=O)n3)CC[C@H]2CN1 ; CCC(CC)COC(=O)[C@@H]1C[C@H]2C[C@@H](CCc3nnnn3COC(C)=O)CC[C@H]2CN1]

A solution of I-18 (3.5 g, 6.53 mmol) in TFA-DCM (15 mL, 1:1; TFA from Oakwood Products No. 001271 and DCM from Sigma-Aldrich No. D65100-18L-CS) was stirred at 25-27° C. for 16-18 h and concentrated at 35-40° C. under vacuum. The residue was re-dissolved in EtOAc (75 mL) and washed with saturated aq. NaHCO₃ solution (20 mL; Sigma-Aldrich No. S6014), dried over sodium sulfate, and concentrated under vacuum at 35-40° C. to obtain 310 as light-brown fluffy solid (2.4 g, 84.5% yield).

To a solution of I-19 (1.6 g, 2.99 mmol) in TFA-DCM (10 mL, 1:1) was stirred at 25-27° C. for 16-18 h and concentrated at 35-40° C. under vacuum. The residue was re-dissolved in EtOAc (55 mL) and washed with saturated aq. NaHCO₃ solution (10 mL), dried over sodium sulfate, and concentrated under vacuum at 35-40° C. to obtain 311 as brown oil (1.1 g, 84.6% yield).

### TLC (70% Ethyl Acetate in Hexane)

TLC (KMnO4 stained). Rf Starting Material: 0.7. Rf Reaction Mixture: 0.1.

TLC (Ninhydrin stained). Rf Starting Material: does not stain by Ninhydrin (Boc-protected DHIQ-ring Nitrogen). Rf 310: 0.3. Rf 311: 0.4.

### Chemical Analysis of 310 and 311

1H-NMR spectrum for isomer 310. Molecular Formula: C₂₂H₃₇N₅O₄. ¹H NMR (CD₃OD, 300 MHz) δ 6.54 (s, 2H), 4.20 4.20 (ddd, J=25.2, 10.8, 5.7 Hz, 2H), 4.20-4.10 (m, 2H), 3.35-3.26 (m, 1H), 3.11 (dd, J=12.8, 4.6 Hz, 1H), 2.16 (s, 3H), 2.16-1.98 (m, 4H), 1.94-1.84 (m, 3H), 1.74-1.51 (m, 8H), 1.45-1.35 (m, 4H), 0.92 (t, J=7.5 Hz, 6H) ppm.

1H-NMR spectrum for isomer 311. Molecular Formula: C₂₂H₃₇N₅O₄. ¹H NMR (CD₃OD, 300 MHz) δ 6.55 (s, 2H), 4.30 (ddd, J=33.8, 14.4, 7.5 Hz, 2H), 4.26-4.19 (m, 1H), 3.34 (t, J=20.6 Hz, 2H), 3.11 (dd, J=17.0, 6.2 Hz, 1H), 3.02 (dd, J=17.3, 9.4 Hz, 1H), 2.19 (s, 3H), 2.27-2.01 (m, 4H), 1.79-1.64 (m, 11H), 1.52-1.43 (m, 8H), 1.09 (t, J=7.2 Hz, 6H) ppm

The 1H NMR of 310 and 1H NMR of 311 confirmed each of the 2 tetrazole isomers were synthesized.

### Standard MS Analysis of 310 and 311: Observed Mass Ion (Electrical Ionization) M+1 436.2

High Resolution LCMS analysis 310 and 311 (reverse phase HPLC and Electrospray Ionization “E.S.I.” Mass Spectrometry): samples dissolved in DMSO then diluted in mobile phase A: 20% acetonitrile in 10 mM ammonium formate, pH 7.0. Gradient beginning with mobile phase A to increasing to 100% acetonitrile over 30 minutes. Observed chromatographic peak eluted with retention time 11.3 minutes with observed Mass (M+1 ESI) 436.2914.

### Synthesis of Fluorinated Dual Tetrazol and Carboxylester Derivative Compounds

The fluorinated mono carboxylester derivative compounds illustrated herein may also be synthesized as fluorinated dual tetrazol and carboxylester derivatives through various methods which are known to a person skilled in the art.

Non-limiting examples of the synthesis of fluorinated dual tetrazol and carboxylester derivative compounds are shown below.

### Synthesis of 2-ethylbutyl (3S,4aS,6S,8aR)-6-{2-[1-(acetoxymethyl)-1H-tetraazol-5-yl]ethyl}-6-fluoroperhydro-3-isoquinolinecarboxylate (312) and 2-ethylbutyl (3S,4aS,6S,8aR)-6-{2-[2-(acetoxymethyl)-2H-tetraazol-5-yl]ethyl}-6-fluoroperhydro-3-isoquinolinecarboxylate (313)

Compounds 312 and 313 can be made using a method similar to that used to make Compounds 310 and 311 (which are hydrogenated dual tetrazol and carboxylester derivatives), which is described above.

### Step 1. Preparation of tert-Butyl (3S,4aS,6S,8aR)-6-{2-[1-(acetoxymethyl)-1H-tetraazol-5-yl]ethyl}-3-(2-ethylbutoxycarbonyl)-6-fluoroperhydro-2-isoquinolinecarboxylate (I-20) and tert-Butyl (3S,4aS,6S,8aR)-6-{2-[2-(acetoxymethyl)-2H-tetraazol-5-yl]ethyl}-3-(2-ethylbutoxycarbonyl)-6-fluoroperhydro-2-isoquinolinecarboxylate (I-21)

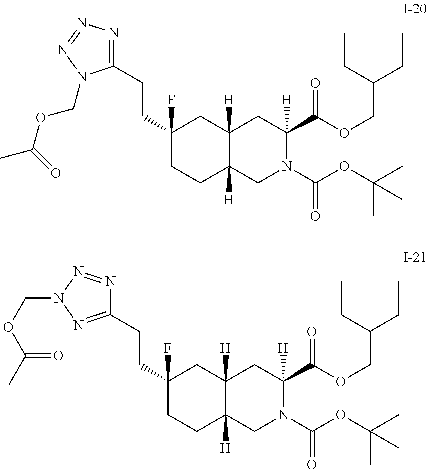 [SMILES: CCC(CC)COC(=O)[C@@H]1C[C@H]2C[C@@](F)(CCc3nnnn3COC(C)=O)CC[C@H]2CN1C(=O)OC(C)(C)C ; CCC(CC)COC(=O)[C@@H]1C[C@H]2C[C@@](F)(CCc3nnn(COC(C)=O)n3)CC[C@H]2CN1C(=O)OC(C)(C)C]

To a solution of I-13B in ACN-THF is added K₂CO₃, Bromomethyl acetate at room temperature under nitrogen atmosphere. The solution is stirred at room temperature for 16-18 h and diluted with EtOAc. The solution is filtered through celite and concentrated at 35-40 C under vacuum. The residue is purified by flash column chromatography. Mass Spec analysis can be performed on the peaks from the chromatography to observe the expected M+1 ions of Compounds I-20 and I-21. 1H NMR of the product before chromatography can be used to confirm that the mixture was made, whereas 1H NMR of the product after chromatography can be used to confirm that the isomers were separated.

### Step 2. Preparation of 2-ethylbutyl (3S,4aS,6S,8aR)-6-{2-[1-(acetoxymethyl)-1H-tetraazol-5-yl]ethyl}-6-fluoroperhydro-3-isoquinolinecarboxylate (312) and 2-ethylbutyl (3S,4aS,6S,8aR)-6-{2-[2-(acetoxymethyl)-2H-tetraazol-5-yl]ethyl}-6-fluoroperhydro-3-isoquinolinecarboxylate (313)

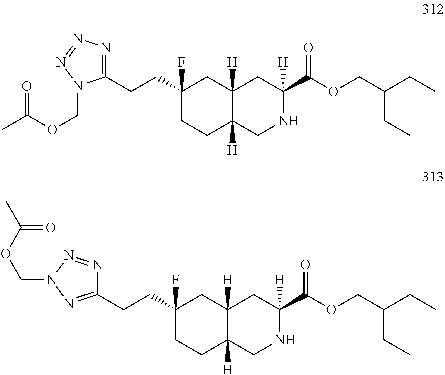 [SMILES: CCC(CC)COC(=O)[C@@H]1C[C@H]2C[C@@](F)(CCc3nnnn3COC(C)=O)CC[C@H]2CN1 ; CCC(CC)COC(=O)[C@@H]1C[C@H]2C[C@@](F)(CCc3nnn(COC(C)=O)n3)CC[C@H]2CN1]

A solution of I-20 in TFA-DCM is stirred at 25-27 C for 16-18 h and concentrated at 35-40 C under vacuum. The residue is re-dissolved in EtOAc and washed with saturated aq. NaHCO₃ solution, dried over sodium sulfate, and concentrated under vacuum at 35-40 C to obtain 312.

A solution of I-21 in TFA-DCM is stirred at 25-27 C for 16-18 h and concentrated at 35-40 C under vacuum. The residue is re-dissolved in EtOAc and washed with saturated aq. NaHCO₃ solution, dried over sodium sulfate, and concentrated under vacuum at 35-40 C to obtain 313.

Alternatively, enantiopure Compound 4 may be used as the starting material, wherein Boc is reinstalled onto the DHIQ nitrogen of Compound 4 to form I-13B.

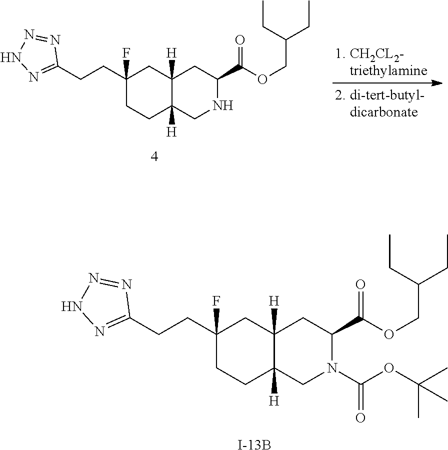

Subsequently, steps 1 and 2 described above are performed to yield 2-ethylbutyl (3S,4aS,6S,8aR)-6-{2-[1-(acetoxymethyl)-1H-tetraazol-5-yl]ethyl}-6-fluoroperhydro-3-isoquinolinecarboxylate (312) and 2-ethylbutyl (3S,4aS,6S,8aR)-6-{2-[2-(acetoxymethyl)-2H-tetraazol-5-yl]ethyl}-6-fluoroperhydro-3-isoquinolinecarboxylate (313).

### Synthesis of Fluorinated Ester Amine Prodrugs XI

### Preparation of Ethyl (3S,4aS,6S,8aR)-6-fluoro-6-[2-(1H-1,2,3,4-tetrazol-5-yl)ethyl]-decahydroisoquinoline-3-carboxylate (3)

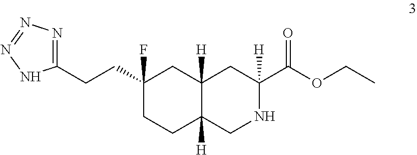 [SMILES: CCOC(=O)[C@@H]1C[C@H]2C[C@@](F)(CCc3nnn[nH]3)CC[C@H]2CN1]

Compound I-12 (4.14 g, 9.73 mmol) was dissolved in 7:1 dioxane-anisole (57 mL, 0.17 M) and treated with 4 N HCl in dioxane (10 eq, 24 mL). After 1 h at room temperature, the reaction was checked by HPLC, which showed the reaction was not complete. Additional 4 N HCl (5 eq, 12 mL) was added and continue was stirring at room temperature. After 1 h, HPLC showed the reaction was not complete and so more 4 N HCl (5 eq, 12 mL) was added. After 30 min, HPLC again showed incomplete reaction and so more 4 N HCl (2 eq, 5 mL) was added. The reaction was complete after 4 h. Nitrogen gas was bubbled into the reaction to purge the solution of excess HCl and then the solvent was partially evaporated under vacuum to half the volume. Hexanes was added to precipitate the HCl salt and the supernatant was discarded. The residue was triturated with dioxane (2 mL), the hexanes (2 mL) was added to fully precipitate the HCl salt of the desired product. The residue was dried under vacuum to afford crude product 3 (3.48 g, 99 3% yield) as a mixture of fluorinated diastereomer isomers. The residue was purified by SFC chromatography (Analytical SFC Method—Column: 4.6×100 mm Chromegabond Ethyl Pyridine (ES Industries, West Berlin, NJ); Solvent A: CO₂, Solvent B: Methanol with 0.1% triethylamine; Gradient Method: 5%-65% B over 4 minutes, hold at 65% B for one minute and return to initial conditions at 4 mL/min; System Pressure: 125 bar; Column Temperature: 40° C.; Sample Diluent: Methanol; Retention Time (3): 2.07 min; Retention Time (C6-F-Isomer): 1.06 min. Preparative SFC Method—Column: 3.0×25.0 cm 2-Ethylpyridine (Princeton Chromatography Inc., Princeton, NJ); Solvent A: CO₂, Solvent B: Methanol with 0.5% triethylamine; Isocratic Method: 25% Solvent B at 100 g/min; System Pressure: 100 bar; Column Temperature: 25° C.; Sample Diluent: Methanol with 0.5% triethylamine) to afford 3 as a semi-solid (414 mg, 96.2% isomeric purity). The material was further purified via Gilson reverse phase chromatography (5% to 50% acetonitrile with 0.1% TFA-water with 0.1% TFA and then 5% to 95% acetonitrile-water) to provide pure compound for testing. ¹H NMR (299.96 MHz, CD₃OD) δ 4.31 (q, J=7.1 Hz, 2H), 4.04 (dd, J=12.9, 3.9 Hz, 1H), 3.20 (t, J=13.0 Hz, 1H), 3.09 (dd, J=12.7, 4.3 Hz, 1H), 3.00 (t, J=8.1 Hz, 2H), 2.33-2.42 (m, 1H), 2.15-1.90 (m, 7H), 1.84-1.65 (m, 3H), 1.57-1.45 (m, 1H), 1.32 (t, J=7.2 Hz, 3H) ppm. ¹⁹F NMR (282.22 MHz, CD₃OD) δ −160.70 (m, uncorrected, TFA reference−76.97) ppm. Mass Analysis (ES+)=326.24 [M+H](Formula: C₁₅H₂₄FN₅O₂, Exact Mass: 325.19).

### Preparation of 2-ethylbutyl (3S,4aS,6S,8aR)-6-fluoro-6-[2-(1H-1,2,3,4-tetrazol-5-yl)ethyl]-decahydroisoquinoline-3-carboxylate (4)

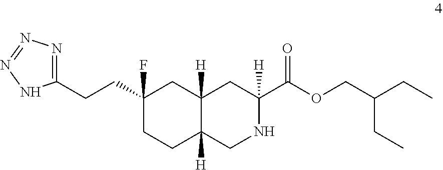 [SMILES: CCC(CC)COC(=O)[C@@H]1C[C@H]2C[C@@](F)(CCc3nnn[nH]3)CC[C@H]2CN1]

To a solution of I-13B (2.2 g, 4.57 mmol) in anhydrous THE (15 mL) was added 4 N HCl in dioxane (11.4 mL, 45.7 mmol) at room temperature while stirring. After stirring for 4 h at room temperature, the reaction was shown to be complete by HPLC. Nitrogen gas was bubbled into the reaction mixture to purge out the excess HCl and the solvent was partially evaporated under vacuum to half the volume. The mixture was diluted with 1:1 MTBE-Heptane (15 mL) resulting in separation of an oily layer, which was triturated and the supernatant discarded. The oily residue was triturated again with 1:1 MTBE-Heptane (15 mL) to further remove lipophilic impurities and the supernatant was discarded. The oily residue was placed under vacuum to remove residual solvent and to afford crude product as a white solid and a mixture of fluorinated diastereomers which was further dried under high vacuum to constant weight (1.6 g crude, 84% yield). The residue was purified by SFC chromatography (Analytical SFC Method—Column: 4.6×100 mm Chiralpak IC SFC (Chiral Technologies, West Chester, PA); Solvent A: CO₂, Solvent B: Ethanol with 0.1% triethylamine; Gradient Method: 5%-65% Solvent B over 4 minutes at 4 mL/min; System Pressure: 125 bar; Column Temperature: 40° C.; Sample Diluent: Ethanol; SFC Retention Time (4): 3.40 min; Preparative SFC Method—Column: 2.1×25.0 cm Chiralpak IC (Chiral Technologies, West Chester, PA); Solvent A: CO₂, Solvent B: Ethanol with 0.25% triethylamine; Isocratic Method: 40% Solvent B at 70 g/min; System Pressure: 100 bar; Column Temperature: 25° C.; Sample Diluent: Ethanol with 0.25% triethylamine) to afford 4 as a thick colorless oil (96.9% isomeric purity). The material was further purified via Gilson reverse phase chromatography (15% to 60% acetonitrile with 0.1% TFA-water with 0.1% TFA and then 10% to 98% acetonitrile-water) to provide pure compound for testing. ¹H NMR (299.96 MHz, CD₃OD) δ 4.21 (ddd, J=20.6, 10.9, 5.6 Hz, 2H), 4.07 (dd, J=12.6, 4.2 Hz, 1H), 3.19 (t, J=13.0 Hz, 1H), 4.07 (dd, J=12.6, 4.2 Hz, 1H), 2.98 (t, J=7.9 Hz, 2H), 2.33-2.42 (m, 1H), 2.13-1.90 (m, 6H), 1.84-1.65 (m, 3H), 1.60-1.30 (m, 7H), 0.93 (t, J=7.2 Hz, 6H) ppm. ¹⁹F NMR (282.22 MHz, CD₃OD) δ −160.46 (m, uncorrected, TFA reference−76.95) ppm. Mass Analysis (ES+)=282.23 [M+H](Formula: C₁₉H₃₂FN₅O₂, Exact Mass: 381.25).

Scale-Up Batch: To a solution of I-13B (21.7 g, 45.06 mmol) in anhydrous THE (15 mL) was added 4 N HCl in dioxane (112.5 mL, 450.0 mmol) at room temperature while stirring. After stirring for 4 h at room temperature, the reaction was shown to be complete by HPLC. Nitrogen gas was bubbled into the reaction mixture to purge out the excess HCl and the solvent was partially evaporated under vacuum to half the volume. The mixture was diluted with 1:1 MTBE-Heptane (150 mL) resulting in separation of an oily layer which was triturated and the supernatant discarded. The oily residue was triturated again with 1:1 MTBE-Heptane (150 mL) to further remove lipophilic impurities and the supernatant was discarded. The oily residue was placed under vacuum to remove residual solvent and to afford crude product as a white solid and a mixture of fluorinated diastereomers which was further dried under high vacuum to constant weight (14 g crude, 75% yield). The residue was purified by SFC chromatography (Analytical SFC Method—Column: 4.6×100 mm Chiralpak IC SFC (Chiral Technologies, West Chester, PA); Solvent A: CO₂, Solvent B: Ethanol with 0.1% ammonium hydroxide; Gradient Method: 5%-65% Solvent B over 4 minutes at 4 mL/min; System Pressure: 125 bar; Column Temperature: 40° C.; Sample Diluent: Ethanol; SFC Retention Time (4): 2.90 min; Preparative SFC Method 1—Column: 2.1×25.0 cm Chiralpak IC (Chiral Technologies, West Chester, PA); Solvent A: CO₂, Solvent B: Ethanol with 0.25% ammonium hydroxide; Isocratic Method: 35% Solvent B at 70 g/min; System Pressure: 100 bar; Column Temperature: 25° C.; Sample Diluent: Ethanol with 0.25% ammonium hydroxide; Preparative SFC Method 2—Column: 2.0×25.0 cm PVA-Sil (YMC, Allentown, PA); Solvent A: CO₂, Solvent B: Ethanol with 0.25% ammonium hydroxide; Isocratic Method: 50% Solvent B at 80 g/min; System Pressure: 100 bar; Column Temperature: 25° C.; Sample Diluent: Ethanol with 0.25% ammonium hydroxide). The desired diastereomer fraction was then further purified via reverse phase chromatography (Column: 19×50 mm XBridge OBD Prep C18 5 μm, 5% acetonitrile and 95% water with 0.1% ammonium hydroxide for 2 minutes and then 5% to 95% acetonitrile-water with 0.1% ammonium hydroxide for 3 minutes; Flow Rate: 25 mL/min; Column Temperature: 40° C.; Sample Diluent: 2:1:1 Ethanol:Acetonitrile:Water). The desired fractions were concentrated via rotary evaporation at 35° C. The dried material was reconstituted in 1:1 acetonitrile:water and the solution was concentrated via rotary evaporation to remove the acetonitrile and then frozen and lyophilized to afford 4 as a white solid (2.42 g, 90% purity, 97.8% ee).

### Preparation of 2-ethylbutyl (3S,4aS,6R,8aR)-6-fluoro-6-[2-(1H-1,2,3,4-tetrazol-5-yl)ethyl]-decahydroisoquinoline-3-carboxylate (5)

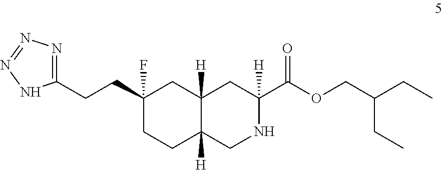 [SMILES: CCC(CC)COC(=O)[C@@H]1C[C@H]2C[C@](F)(CCc3nnn[nH]3)CC[C@H]2CN1]

SFC chromatography to provide compound 4 also provided the C6-F-Isomer 5 (SFC Retention Time: 3.04 min) as a thick colorless oil (97.9% isomeric purity). The material was further purified via Gilson reverse phase chromatography (15% to 60% acetonitrile with 0.1% TFA-water with 0.1% TFA and then 10% to 98% acetonitrile-water) to provide pure compound for testing. ¹H NMR (299.96 MHz, CD₃OD) δ 4.30-4.25 (m, 2H), 4.18 (dd, J=10.9, 5.5 Hz, 1H), 3.35-3.27 (m, 2H), 3.09 (t, J=8.1 Hz, 2H), 2.28-1.94 (m, 7H), 1.86-1.69 (m, 5H), 1.59 (p, J=6.1 Hz, 1H), 1.46-1.36 (m, 4H), 0.94 (t, J=7.3 Hz, 6H) ppm.

### Preparation of cyclohexyl (3S,4aS,6S,8aR)-6-fluoro-6-[2-(1H-1,2,3,4-tetrazol-5-yl)ethyl]-decahydroisoquinoline-3-carboxylate (6)

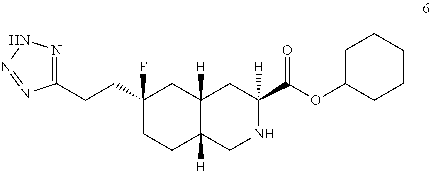 [SMILES: O=C(OC1CCCCC1)[C@@H]1C[C@H]2C[C@@](F)(CCc3nn[nH]n3)CC[C@H]2CN1]

Compound I-16 (100 mg, 0.2 mmol) was dissolved in THE (20 mL) and 10% Pd/C (100 mg) was added. This mixture was degassed by three hydrogen purge cycles (vacuum, release, and pressurization with hydrogen to 52 psi). The mixture was stirred under hydrogen overnight and then monitored by HPLC. The mixture was filtered through Celite®, washed with THE (20 mL), and the filtrate was concentrated under vacuum. The residue was dissolved in THE (20 mL) and fresh 10% Pd/C (100 mg) catalyst was added, followed by hydrogen purge cycles as before. The mixture was stirred under hydrogen overnight and monitored by HPLC. The mixture was filtered through Celite®, washed with THE (100 mL), and the filtrate was concentrated under vacuum. The crude product was purified via Gilson reverse phase chromatography (20% to 60% acetonitrile with 0.1% TFA-water with 0.1% TFA) to afford 6 as the TFA salt (17.0 mg, 2^{nd} peak) and the C6-F-Isomer TFA Salt (21.7 mg, 1^{st} peak). The desired isomer was further purified via Gilson reverse phase chromatography (10% to 98% acetonitrile-water) to provide pure compound 6 for testing. ¹H NMR (299.96 MHz, CD₃OD) δ 4.89-4.94 (m, 1H), 4.01 (dd, J=12.9, 3.9 Hz, 1H), 3.17 (t, J=12.7 Hz, 1H), 3.06 (dd, J=13.0, 4.6 Hz, 1H), 2.98 (t, J=7.9 Hz, 2H), 2.33-2.42 (m, 1H), 1.97-2.15 (m, 5H), 1.83-1.96 (m, 3H), 1.64-1.82 (m, 5H), 1.25-1.62 (m, 8H) ppm. ¹⁹F NMR (282.22 MHz, CD₃OD) δ −160.20 (m, uncorrected, TFA reference −76.97) ppm. Mass Analysis (ES+)=380.19 [M+H](Formula: C₁₉H₃₀FN₅O₂, Exact Mass: 379.24).

### Preparation of octyl (3S,4aS,6S,8aR)-6-fluoro-6-[2-(1H-1,2,3,4-tetrazol-5-yl)ethyl]-decahydroisoquinoline-3-carboxylate (7)

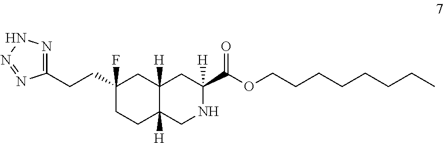 [SMILES: CCCCCCCCOC(=O)[C@@H]1C[C@H]2C[C@@](F)(CCc3nn[nH]n3)CC[C@H]2CN1]

Compound I-17 (40 mg, 0.074 mmol) was dissolved in THE (10 mL) and 10% Pd/C (100 mg) was added. This mixture was degassed by three hydrogen purge cycles (vacuum, release, and pressurization with hydrogen to 52 psi). The mixture was stirred under hydrogen overnight and then monitored by HPLC. The mixture was filtered through Celite®, washed with THE (20 mL), and the filtrate was concentrated under vacuum. The residue was dissolved in THE (20 mL) and fresh 10% Pd/C (100 mg) catalyst was added, followed by hydrogen purge cycles as before. The mixture was stirred under hydrogen overnight and monitored by HPLC. The mixture was filtered through Celite®, washed with THE (100 mL), and the filtrate was concentrated under vacuum. The crude product was purified via Gilson (30% to 60% acetonitrile with 0.1% TFA -water with 0.1% TFA) to afford 7 as the TFA salt (2.9 mg, 2^{nd} peak) and the C6-F-Isomer TFA salt (4.5 mg, 1^{st} peak). ¹H NMR (299.96 MHz, CD₃OD) δ 4.26 (dt, J=6.6, 1.8 Hz, 2H), 4.10 (dd, J=12.7, 4.0 Hz, 1H), 3.23 (d, J=13.2 Hz, 1H), 3.17-3.09 (m, 3H), 2.43-2.36 (m, 1H), 2.20-1.93 (m, 1H), 1.89-1.52 (m, 1H), 1.45-1.30 (m, 1H), 0.91 (t, J=6.9 Hz, 3H) ppm. Mass Analysis (ES+)=410.17 [M+H](Formula: C₂₁H₃₆FN₅O₂, Exact Mass: 409.29).

Using the above protocols, additional ketone carbamate esters IV can be made and then reacted with the tetrazole Wittig VIII, fluorinated, and deprotected to generate novel fluorinated ester amine prodrugs (XI) as shown in Table 1.

TABLE 1
Fluorinated Ester Amine Prodrugs (XI).

| Compound | |
|---|---|
| No. | Structure |
| 3 | 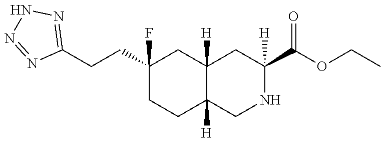 [SMILES: CCOC(=O)[C@@H]1C[C@H]2C[C@@](F)(CCc3nn[nH]n3)CC[C@H]2CN1] |
| 4 | 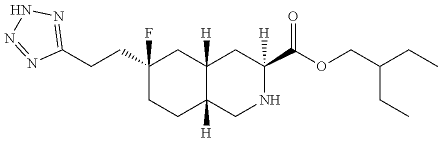 [SMILES: CCC(CC)COC(=O)[C@@H]1C[C@H]2C[C@@](F)(CCc3nn[nH]n3)CC[C@H]2CN1] |
| 5 | 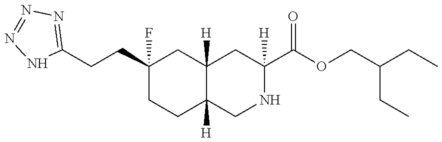 [SMILES: CCC(CC)COC(=O)[C@@H]1C[C@H]2C[C@](F)(CCc3nnn[nH]3)CC[C@H]2CN1] |
| 6 | 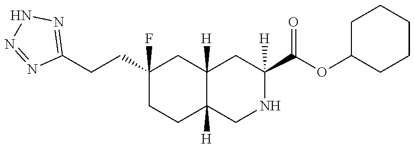 [SMILES: O=C(OC1CCCCC1)[C@@H]1C[C@H]2C[C@@](F)(CCc3nn[nH]n3)CC[C@H]2CN1] |
| 7 | 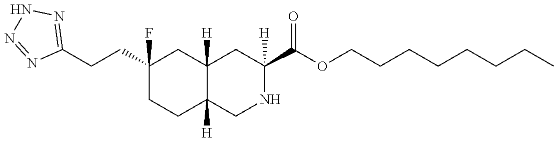 [SMILES: CCCCCCCCOC(=O)[C@@H]1C[C@H]2C[C@@](F)(CCc3nn[nH]n3)CC[C@H]2CN1] |
| 8 | 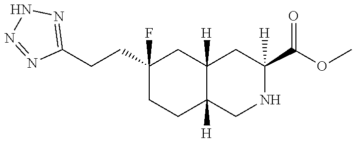 [SMILES: COC(=O)[C@@H]1C[C@H]2C[C@@](F)(CCc3nn[nH]n3)CC[C@H]2CN1] |
| 9 | 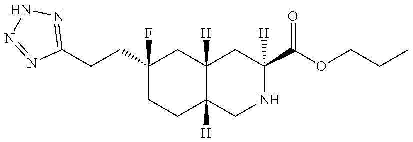 [SMILES: CCCOC(=O)[C@@H]1C[C@H]2C[C@@](F)(CCc3nn[nH]n3)CC[C@H]2CN1] |
| 10 | 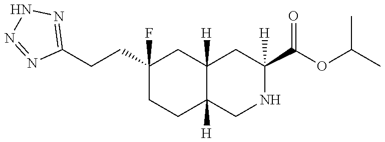 [SMILES: CC(C)OC(=O)[C@@H]1C[C@H]2C[C@@](F)(CCc3nn[nH]n3)CC[C@H]2CN1] |
| 11 | 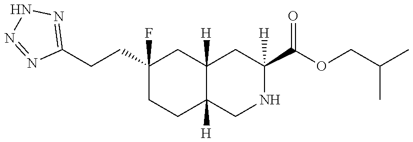 [SMILES: CC(C)COC(=O)[C@@H]1C[C@H]2C[C@@](F)(CCc3nn[nH]n3)CC[C@H]2CN1] |
| 12 | 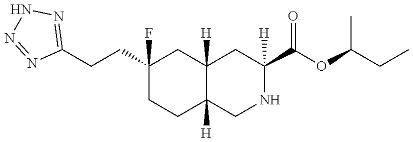 [SMILES: CC[C@H](C)OC(=O)[C@@H]1C[C@H]2C[C@@](F)(CCc3nn[nH]n3)CC[C@H]2CN1] |
| 13 | 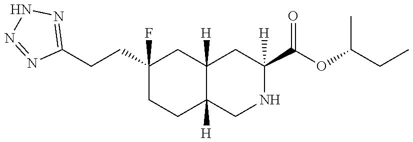 [SMILES: CC[C@@H](C)OC(=O)[C@@H]1C[C@H]2C[C@@](F)(CCc3nn[nH]n3)CC[C@H]2CN1] |
| 14 | 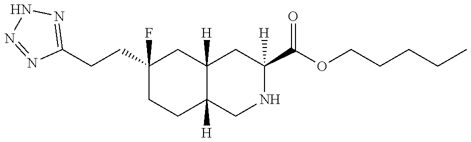 [SMILES: CCCCCOC(=O)[C@@H]1C[C@H]2C[C@@](F)(CCc3nn[nH]n3)CC[C@H]2CN1] |
| 15 | 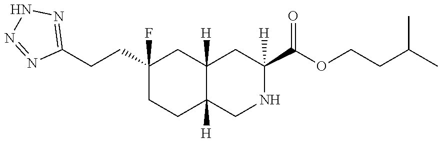 [SMILES: CC(C)CCOC(=O)[C@@H]1C[C@H]2C[C@@](F)(CCc3nn[nH]n3)CC[C@H]2CN1] |
| 16 | 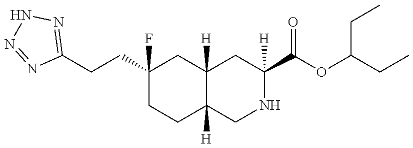 [SMILES: CCC(CC)OC(=O)[C@@H]1C[C@H]2C[C@@](F)(CCc3nn[nH]n3)CC[C@H]2CN1] |
| 17 | 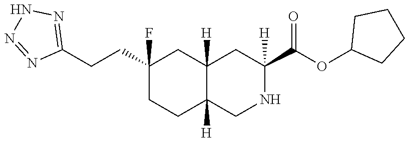 [SMILES: O=C(OC1CCCC1)[C@@H]1C[C@H]2C[C@@](F)(CCc3nn[nH]n3)CC[C@H]2CN1] |
| 18 | 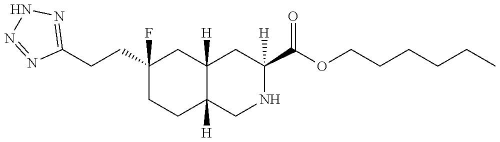 [SMILES: CCCCCCOC(=O)[C@@H]1C[C@H]2C[C@@](F)(CCc3nn[nH]n3)CC[C@H]2CN1] |
| 19 | 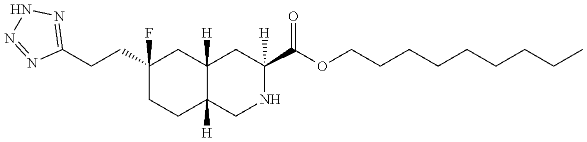 [SMILES: CCCCCCCCCOC(=O)[C@@H]1C[C@H]2C[C@@](F)(CCc3nn[nH]n3)CC[C@H]2CN1] |
| 20 | 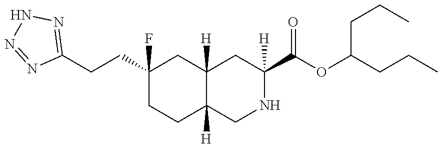 [SMILES: CCCC(CCC)OC(=O)[C@@H]1C[C@H]2C[C@@](F)(CCc3nn[nH]n3)CC[C@H]2CN1] |
| 21 | 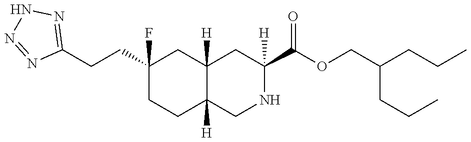 [SMILES: CCCC(CCC)COC(=O)[C@@H]1C[C@H]2C[C@@](F)(CCc3nn[nH]n3)CC[C@H]2CN1] |
| 22 | 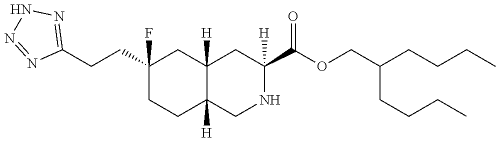 [SMILES: CCCCC(CCCC)COC(=O)[C@@H]1C[C@H]2C[C@@](F)(CCc3nn[nH]n3)CC[C@H]2CN1] |
| 23 | 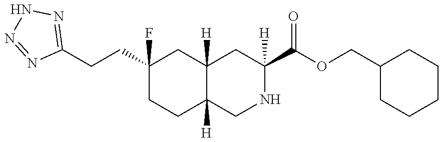 [SMILES: O=C(OCC1CCCCC1)[C@@H]1C[C@H]2C[C@@](F)(CCc3nn[nH]n3)CC[C@H]2CN1] |
| 24 | 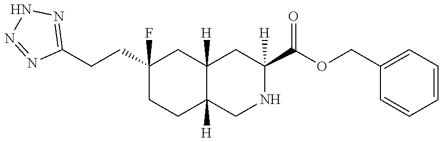 [SMILES: O=C(OCc1ccccc1)[C@@H]1C[C@H]2C[C@@](F)(CCc3nn[nH]n3)CC[C@H]2CN1] |
| 25 | 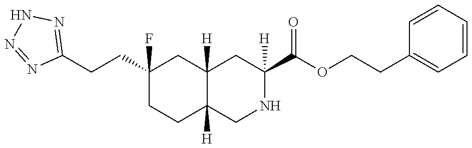 [SMILES: O=C(OCCc1ccccc1)[C@@H]1C[C@H]2C[C@@](F)(CCc3nn[nH]n3)CC[C@H]2CN1] |
| 26 | 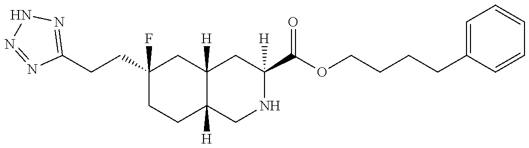 [SMILES: O=C(OCCCCc1ccccc1)[C@@H]1C[C@H]2C[C@@](F)(CCc3nn[nH]n3)CC[C@H]2CN1] |
| 27 | 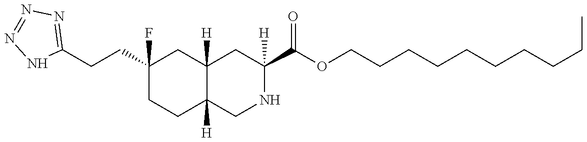 [SMILES: CCCCCCCCCCOC(=O)[C@@H]1C[C@H]2C[C@@](F)(CCc3nnn[nH]3)CC[C@H]2CN1] |
| 28 | 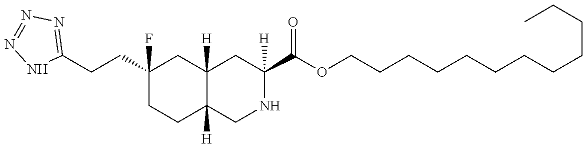 [SMILES: CCCCCCCCCCCCOC(=O)[C@@H]1C[C@H]2C[C@@](F)(CCc3nnn[nH]3)CC[C@H]2CN1] |
| 29 | 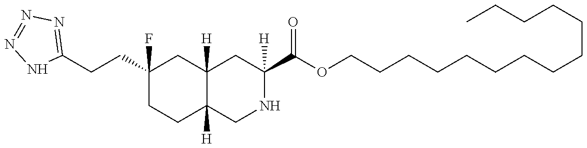 [SMILES: CCCCCCCCCCCCCCOC(=O)[C@@H]1C[C@H]2C[C@@](F)(CCc3nnn[nH]3)CC[C@H]2CN1] |
| 30 | 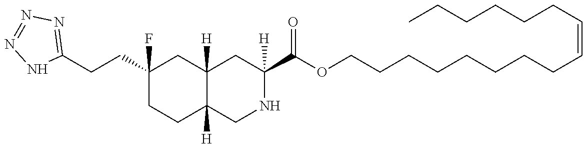 [SMILES: CCCCCC/C=C\CCCCCCCCOC(=O)[C@@H]1C[C@H]2C[C@@](F)(CCc3nnn[nH]3)CC[C@H]2CN1] |
| 31 | 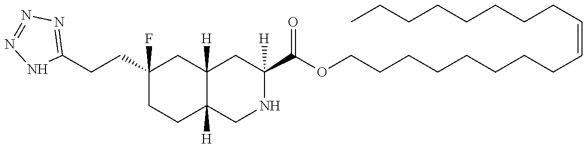 [SMILES: CCCCCCCC/C=C\CCCCCCCCOC(=O)[C@@H]1C[C@H]2C[C@@](F)(CCc3nnn[nH]3)CC[C@H]2CN1] |
| 32 | 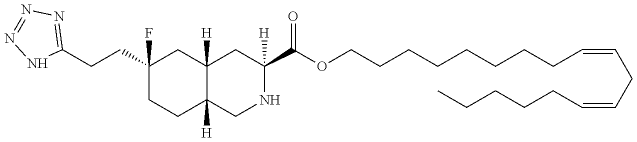 [SMILES: CCCCC/C=C\C/C=C\CCCCCCCCOC(=O)[C@@H]1C[C@H]2C[C@@](F)(CCc3nnn[nH]3)CC[C@H]2CN1] |

### Synthesis of Fluorinated Decahydroisoquinoline Amino Acids XI

### Preparation of (3S,4aS,6S,8aR)-6-fluoro-6-[2-(1H-1,2,3,4-tetrazol-5-yl)ethyl]-decahydroisoquinoline-3-carboxylic acid (1)

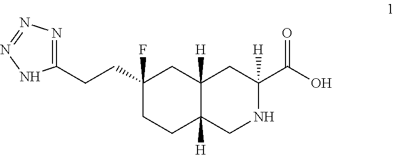 [SMILES: O=C(O)[C@@H]1C[C@H]2C[C@@](F)(CCc3nnn[nH]3)CC[C@H]2CN1]

To a solution of compound 3 (1.29 g, 3.97 mmol) in water (2 mL) was added 2 N NaOH (9 mL, 16.9 mmol) at room temperature under nitrogen atmosphere. The solution was stirred at room temperature for 12 h and then quenched with 1N HCl (18.5 mL) to adjust pH between pH 2-3. The compound was purified by resin catch-and-release, Dowex 50WX8 200-400 mesh resin (Sigma-Aldrich, 1.7 eq/wet mL, 14 mL), which was washed with water until the pH was neutral in a coarse porosity sintered glass funnel. Resin (5 mL) was added to the compound solution and the mixture was stirred for 10 min at room temperature. Fresh resin (9 mL) was placed into a column with a coarse frit and the mixture was gently transferred onto the column (including all the resin). The top of the resin column was covered with cotton to prevent disturbing the layer. The solution was allowed to elute slowly and then the resin was wash with water (3×30 mL) to remove salts, 1:1 THF:water (3×30 mL) to remove organic impurities, water (2×30 mL), and then 6% ammonium hydroxide (300 mL) to release the desired compound. Fractions were collected and desired compound was detected using ninhydrin staining on reverse phase TLC plates (10% acetonitrile-water). Desired fractions were combined and concentrated under vacuum to afford crude product. The crude product was dissolved in water and lyophilized to afford 1 as a white solid (1.02 g, 86% yield). ¹H NMR (299.96 MHz, D₂O) δ 3.58 (dd, J=12.9, 3.9 Hz, 1H), 3.11 (dd, J=19.2, 12.9 Hz, 1H), 3.08 (s, 1H), 2.97 (dd, J=8.8, 7.0 Hz, 2H), 2.35-2.28 (m, 1H), 2.14-1.61 (m, 9H), 1.56-1.47 (m, 1H), 1.38 (dtd, J=42.6, 14.6, 4.6 Hz, 1H) ppm. ¹⁹F NMR (282.22 MHz, D₂O) δ −157.59 (m, uncorrected) ppm. Mass Analysis (ES+)=298.12 [M+H](Formula: C₁₃H₂₀FN₅O₂, Exact Mass: 297.16).

### Preparation of (3S,4aS,6R,8aR)-6-fluoro-6-[2-(1H-1,2,3,4-tetrazol-5-yl)ethyl]-decahydroisoquinoline-3-carboxylic acid (2)

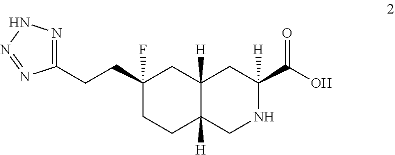 [SMILES: O=C(O)[C@@H]1C[C@H]2C[C@](F)(CCc3nn[nH]n3)CC[C@H]2CN1]

Using a similar protocol to make compound 1, the C6-F-isomer compound 5 was hydrolyzed with NaOH and purified by resin catch-and-release to afford 2. ¹H NMR (299.96 MHz, D₂O) δ 3.75 (dd, J=10.6, 3.4 Hz, 1H), 3.29-3.14 (m, 2H), 2.96 (t, J=7.8 Hz, 2H), 2.22 (dt, J=7.8, 3.6 Hz, 1H), 2.13 (dt, J=7.8, 3.2 Hz, 1H), 2.10-1.86 (m, 3H), 1.84-1.61 (m, 5H) ppm. ¹⁹F NMR (282.22 MHz, D₂O) δ −167.27 (uncorrected, TFA reference −75.69) ppm. Mass Analysis (ES+)=298.26 [M+H](Formula: C₁₃H₂₀FN₅O₂, Exact Mass: 297.16).

### C. Biological Activity

### AMPAR and NMDAR Biological Activity.

The present disclosure relates to chemical composition of matter (molecules) and characterizes biological and pharmacological activity of the molecules as AMPA receptor (AMPAR) antagonists or prodrugs of such molecules. The present disclosure also discloses uses for the treatment of convulsions, seizures, epilepsy, status epilepticus, and other seizure disorders.

Additionally biological activity of compounds as antagonists of AMPA receptors or NMDA receptors (also called ionotropic glutamate-gated ion channels) were conducted using ex vivo functional electrophysiological assays of pyramidal neurons in slices of rat brain prefrontal cortex. Effects of test compounds cf reference compounds were studied by whole cell patch-clamp electrophysiology recordings of s-AMPA-induced currents or NMDA-induced currents, respectively, using rat brain slice prefrontal cortex (layer V) pyramidal neurons.

### Brain Slice Preparation Protocol.

Male Sprague Dawley rats were supplied by Charles River Laboratories (Wilmington, Massachusetts USA) and were housed 4 per cage within a temperature (20.5-23.5° C.) and humidity (30-80%) controlled environment on a 12 hour light/dark cycle with access to food (Teklad Global Soy Protein, Cat. No. T.2920X10, Envigo, Indianapolis, IN, USA) and water ad libitum. At 4-6 weeks of age rats were terminally anaesthetized using isofluorane [(1-chloro-2,2,2-trifluoroethyl difluoromethyl ether) supplied by Baxter Healthcare Corp, Deerfield, Illinois, USA], and decapitated. The brain was removed and 300 m thick coronal prefrontal cortex (PFC) or sagittal hippocampal slices were sectioned using a Vibratome microtome. After brain removal, and throughout slicing, the tissue was submerged in ice cold aqueous cerebrospinal fluid (aCSF). Once slices were cut, they were transferred to a beaker containing aCSF and left at room temperature for a minimum of 1 hour before commencing electrophysiological recordings. After this period, individual slices were transferred to a recording chamber continuously perfused with aCSF at a rate of 4-6 mL/min before commencing experiment protocols. aCSF composition (in mM): NaCl, 127; KCl, 1.9; KH₂PO₄, 1.2; CaCl₂, 2.4; MgCl₂, 1.3; NaHCO₃, 26; D-glucose, 10; in water equilibrated with 95% Oxygen gas-5% CO₂ gas (reagent suppliers listed below). Experiments investigating NMDA currents had aCSF supplemented with 10 μM glycine (reagents suppliers listed below). All experiments were conducted following protocols with the approval of an Institutional Animal Care and Use Committee (IACUC).

### Reagents for Electrophysiology Studies.

Fisher Scientific (Fairlawn, New Jersey, USA) supplied NaCl product #S271; KCl product #P330; CaCl₂) product #C79; MgCl2 product #M33; D-glucose product #D16; HEPES product #BP310; sucrose product #S5, and NaHCO3, product #S233. Millipore-Sigma (St. Louis, Missouri, USA) supplied Mg-ATP product #A9187; CsCl product #C3032; EGTA-Na product #E4378; GTP product #G8877; glycine product #G7126; KOH product #417661; and potassium D-gluconate product #G4500. EMD Chemicals (Gibbstown, New Jersey, USA) supplied KH2PO4 product #PX1565. Tocris and BioTechne (Bristol, United Kingdom and Minneapolis, Minnesota USA) supplied (S)-alpha-amino-3-hydroxy-5-methyl-4-isoxazolepropionic acid, s-AMPA, product #0254; and supplied N-methyl-D-aspartic acid, NMDA, product #0114. Thermo Scientific (Rockford, Illinois USA) supplied DMSO product #20688.

Electrophysiological Recording of s-AMPA or NMDA Induced Currents in Pyramidal Neurons of Brain Slices from Prefrontal Cortex of Rats.

Whole cell patch-clamp recordings were performed from Layer V prefrontal cortex pyramidal neurons at room temperature using the ‘visualized’ version of the patch-clamp technique. Neurons were visualized using a BX51 upright microscope fitted with a 40× LUMPlanFl water immersion objective (Olympus, Richmond Hill, Ontario, Canada). The microscope was connected to a C2400 CCD camera (Hamamatsu Bridgewater, NJ, USA) and images viewed on a VM 5516 B/W monitor (Sanyo, Moriguchi, Osaka Prefecture, Japan). Electrophysiological recordings were obtained using a Multiclamp 700B patch clamp amplifier (Molecular Devices, Sunnyvale, California, USA), with analogue signals digitized on a Digidata 1440a (Molecular Devices, Sunnyvale, California, USA). Patch pipettes were pulled using a P-87 Flaming/Brown micropipette puller (Sutter, Novato, CA, USA), from GC150TF-10 thin-walled borosilicate glass (Harvard Apparatus, Saint-Laurent, Quebec, Canada) which had resistances of between 3 and 8 MΩ when filled with intracellular solution.

Intracellular solutions used for PFC neuron recordings had the following composition (mM): potassium D-gluconate, 140; KCl, 10; EGTA-Na, 1; HEPES, 10; Mg-ATP, 4, 0.3 GTP; with pH and osmolarity compensated with potassium hydroxide and sucrose, respectively in all intracellular solutions (reagent suppliers listed above).

All test compound experiments were carried out in the voltage-clamp recording configuration of the whole cell patch-clamp technique and all recordings performed at a holding potential of −60 mV. Recordings were monitored on a Dell personal computer (PC) running Axon pClamp software (Molecular Devices, Sunnyvale, California, USA) and digitized at 10 kHz.

### AMPA Receptor Biological Activity Assay of Compounds in Brain Slice Neurons.

Compounds were tested in whole-cell patch clamp electrophysiology experiments to measure antagonist effect on s-AMPA induced currents. These experiments examined the effects of a single concentration of each test-compound on 20 μM s-AMPA evoked currents in layer V rat brain slice prefrontal cortex pyramidal neurons. Both 1 μM and 10 μM concentrations of Compounds were tested, respectively in different experiments to the brain slice by bath perfusion from 50 mL syringes arranged in series with the main perfusion line from the aCSF reservoir. 20 μM s-AMPA was pressure-ejected for 100-1000 ms every 1-2 minutes using a NPI PDES-02DX (npi Electronic GmbH, Tamm, Germany) pneumatic picopump connected directly to a microelectrode placed within 200 μm of the recorded neuron. Each protocol was repeated three times. If the peak current was reduced by more than 70% upon first application of a test compound at 10 μM, then a single experiment was conducted at this 10 μM compound concentration. In that case the single 10 μM test of the compound was followed by testing the compound at 1 μM concentration three times.

### NMDA Receptor Biological Activity Assay of Compounds in Brain Slice Neurons.

Compounds are tested in whole-cell patch clamp electrophysiology experiments to measure antagonist effect on NMDA induced currents. NMDA induced currents Experiments examined the effects of a single concentration of each test-compound on 50 μM NMDA-evoked currents in layer V rat brain slice prefrontal cortex pyramidal neurons. A 30 μM concentration of Compound was administered to the slice by bath perfusion from 50 mL syringes arranged in series with the main perfusion line from the aCSF reservoir. 50 μM NMDA was pressure-ejected for 100-1000 ms every 1-2 minutes using a NPI PDES-02DX (npi Electronic GmbH, Tamm, Germany) pneumatic picopump connected directly to a microelectrode placed within 200 μm of the recorded neuron. Compounds were tested at 30 μM concentration in three experiments.

Test Compounds or Reference compounds or inducer s-AMPA or inducer NMDA formulation. Test compounds (Sea Pharmaceuticals LLC, Cambridge Massachusetts USA) were prepared as 10 mM or 30 mM stock solutions in 100% DMSO solvent (solvent supplier listed above). Inducers of currents, s-AMPA or NMDA, were made as a 20 μM or 50 μM stock solution respectively in aCSF. Test compound stock solutions were diluted in the appropriate external recording solution to the final indicated test concentrations immediately prior to use. All compounds were stored at −20° C. prior to use.

### Reference Compound Tests.

Tezampanel (Ref_1) was tested as a reference compound at 30 μM, 10 μM, 3 μM, 1 μM, 0.3 μM, and 0.1 μM concentrations in the s-AMPA electrophysiological assay. Ref_1 showed concentration-dependent inhibition of peak amplitude responses of rat brain slice PFC pyramidal neurons to 20 μM s-AMPA (Ref 1 compound IC₅₀ concentration where 50% of signal is inhibited was observed at 481+/−84 nM in the s-AMPA induced currents in rat brain cortex pyramidal neurons, n=4 to 5 experiments per concentration).

Ref_1 was tested at 30 μM concentration in the NMDA electrophysiological assay. Ref_1 at this concentration shows partial inhibition (40%+/−2%, n=4 experiments) of peak amplitude responses of rat brain slice PFC pyramidal neurons to 50 μM NMDA induced currents in rat brain cortex pyramidal neurons.

### Data and Statistical Analysis of Electrophysiology Experiments.

All data were sampled using pClamp Clampex acquisition software with all offline analysis carried out using Clampfit (MDS Analytical Technologies). Data compilation and figure construction was carried out using Excel (Microsoft). One-way repeated measures analysis of variance (ANOVA, Prism, Graphpad) with Dunnett's post hoc comparison was used for statistical analysis.

TABLE 6
Demonstrates AMPAR or NMDAR antagonist activity of
compounds 1 and 2. Compounds 1 and 2 were tested
as enantiopure samplesTable 6: Summary of In Vitro
Biology Data for Compound # 1 and Compound # 2.

| | Electrophysiological assays for antagonist | | |
|---|---|---|---|
| | activity. % inhibition +/− SEM of s-AMPA or | | |
| | NMDA induced currents in neurons (whole cell | | |
| | patch-clamp recordings in rat brain cortex slices) | | |
| Compound | S-AMPA | S-AMPA | NMDA |
| No. | (Tested at 1 μM) | (Tested at 10 μM) | (Tested at 30 μM) |
| 1 | 55.6 ± 5.8 (n = 3) | >90 (n = 1) | 61.0 ± 6.3 (n = 3) |
| 2 | 21.4 ± 2.1 (n = 3) | NT | NT |

NT = not tested. SEM = Standard Error of the mean. n = number of experiments

### KAINR Antagonist Biological Activity.

### Molecular Biology.

Genetic construction of human recombinant KAINR subunit gene GRIK1 DNA clone for mammalian cell in vitro expression of glutamate gated ion channel subunit GluK1.

A plasmid DNA molecule was constructed for in vitro cell expression of Homo sapiens (human recombinant) Kainate receptor (KAINR) subunit GluK1 in mammalian cells for use by transient transfection of mammalian cells for whole cell patch clamp electrophysiology assays of glutamate gated ion channel KAINR. KAINR is a “ligand activated ion channel” that is expressed on neurons in the Central Nervous System and is activated not only by the endogenous neurotransmitter L-Glutamate but also by 2 natural marine neurotoxins (Kainate and its homolog, Domoate) and by the synthetic molecule ATPA. GluK1 is a protein subunit of KAINR that expresses a functional ligand gated ion channel when expressed in mammalian cells such as human embryonic kidney 293 cells (HEK293) or other mammalian cell lines. GluK1 is encoded by human GRIK1 gene glutamate receptor (ionotropic, kainate 1; Genbank database accession number NP_000821; GluK1 was formerly called GluR5 subunit or EAA3). A commercially available plasmid DNA molecule called hGRIK1/GluR5 VersaClone cDNA plasmid (R-and-D Systems BioTechne catalog number RDC0944) carrying the cloned human hGRIK1 [open reading frame ORF 1-918 version containing Q glutamine at the Q/R editing site (Genbank accession sequence identification NP_000821), along with a Kozak consensus sequence for optimal translation inserted Not1 to Asc1 (restriction enzyme recognition sites)] was purchased from BioTechne Inc., Minneapolis Minnesota, USA. The human GRIK1 cDNA on RDC0944 was previously isolated from a human cDNA library as described by R and D Systems (manufacturer). A commercially available mammalian gene expression plasmid vector pcDNA3.1plus was purchased from Invitrogen LifeTech Inc (Carlsbad, California, USA). Plasmid pcDNA3.1plus is a shuttle vector plasmid DNA molecule that replicates in laboratory cloning strains of E. coli bacteria and contains both an Ampicillin resistance gene (for selection in E. coli bacteria) & a Neomycin antibiotic resistance gene (for selection of mammalian cell transfectants). Plasmid pcDNA3.1plus also contains a strong viral promotor “CMV” (cytomegalovirus) promotor for expression of ORFs in mammalian and a multiple cloning site. A 2.78 kilobase DNA fragment “cassette” containing the human GRIK1 ORF was released from plasmid RDC0944 the hGRIK1/GluR5 VersaClone using restriction enzymes HindIII and XhoI (purchased from New England Biolabs, Massachusetts USA) and was subcloned into the same sites of plasmid pcDNA3.1plus using standard genetic engineering molecular biology techniques. The correct DNA insert and correct DNA orientation were confirmed by Restriction Enzyme digestion analysis by gel electrophoresis followed by ethidium bromide staining and UV visualization of DNA fragments in agarose gels alongside standard DNA size markers. The inserted DNA human GRIK1 and adjoining regions was sequenced by DNA sequencing to confirm the construct. The plasmid DNA mammalian gene expression clone (S0001) containing human recombinant GRIK1 gene encoding human recombinant GluK1 carried in vector pcDNA3.1plus was maintained in E. coli bacteria under ampicillin selection. Plasmid DNA was purified from E. coli bacterial cultures in LB-broth containing ampicillin using commercial extraction kit from Qiagen. Purified DNA concentration was quantified by spectroscopy in TE buffer (standard molecular biology reagent Tris-EDTA solution in DNAase free water) in a quartz cuvette using a spectrometer. Purified DNA was again verified by Restriction Enzyme digestion analysis using agarose gel electrophoresis as described above. Lyophilized aliquots of 50 micrograms per polypropylene plastic-DNAase free microfuge tube were stored at −20 degrees Celsius until use in transfection of mammalian cells. The empty vector pcDNA3.1plus was also handled similarly, DNA purified as preparation of plasmid DNA using Qiagen kit, quantified and stored frozen until use.

### Cell Biology.

Cell culture of HEK293 cells and transient transfection of plasmid DNA expressing GRIK1.

Plasmid DNA (mammalian gene expression clone S0001 containing human recombinant GRIK1 encoding human GluK1 subunit formerly called EAA3 or GluR5) was transfected into human embryonic kidney 293 (HEK293) cells. HEK293 cells were cultured and transfected with standard molecular cell biological techniques previously described for Kainate receptor subunits [rat GluK1 (Sommer B et al 1992), human GluK1 (Korczak B et al 1995) or GluK2 formerly called GluR6 (Jones et al 1997; Wilding T J et al 2008; Lopez M N, Wilding T J, Huettner 2013). HEK293 cells were cultured at 37 degrees C. in atmosphere containing 95% air and 5% CO₂ in a growth medium of MEM (Life Technologies), penicillin, streptomycin and was supplemented with 10% fetal calf serum. Cells were maintained in frozen stocks according to the source of the cells (American Type Culture Collection “ATCC”). Cells were cultured to near confluency and then treated with Protease XXIII and collected, resuspended in MEM medium+Fetal Calf Serum and dispensed into 12-well plates as described (Huettner J E et al 1998). 750 ng S0001 plasmid DNA was transfected into HEK293 cells as described (Huettner J E et al 1998). Growth medium 5 mM Kynurenic Acid (Sigma-Aldrich) to block GluK1 ion channel activity as described (Huettner J E et al 1998). Cell biology culture methods and KAINR ion channel subunit gene cDNA transfection techniques widely familiar to biologists skilled in the art may be used, would yield similar results, and are well described in the literature (Sommer B et al 1992, Korczak B et al 1995, Jones K et al 1997, Huettner J E et al 1998, Wilding T J et al 2008, Lopez M N et al 2013). Control transfections were also conducted with the plasmid DNA “empty vector” (not carrying any ion channel gene) pcDNA3.1 plus which serves as a negative control for electrophysiology studies as HEK293 cells transfected with this “empty vector” DNA do not respond to kainate or glutamate. Cells were cultured 48 hours after transfection as described by Huettner (Jones K et al 1997, Huettner J E et al 1998, Lopez M N et al 2013).

### Electrophysiology.

Kainate receptor (KAINR) ion channel recording of transfected HEK293 cells.

Electrophysiology studies were conducted by whole cell manual patch clamp assay of HEK293 cells transiently transfected with human GRIK1 (clone S0001) expressing ligand activated ion channel human recombinant KAINR subunit GluK1. Electrophysiology techniques followed or slightly modified the peer-reviewed, published methods for HEK293 cells transiently transfected with rat recombinant KAINR subunits GluK1, GluK2 as described by Huettner (Huettner J E et al 1998, Wilding T J et al 2008, Lopez M N et al 2013) with modifications as follows. Kainic Acid (Sigma-Aldrich) was used at 20 μM agonist concentration in KAINR (GluK1) ion channels electrophysiology. Kainate 20 μM currents were recorded in studies of human GluK1 ion channel subunit expressed by HEK293 cells transiently transfected with human GRIK1 (clone S0001). Test articles (reference compounds or experimental compounds) were tested for antagonist effect on Kainate induced GluK1 currents in a range of concentrations from 30 nM, 100 nM, 300 nM, 1 μM, 3 μM, 10 μM in n=3 to 6 experiments per concentration. IC50 values (inhibitory concentration of test article at which 50% inhibition occurred) were calculated in Prism (Graphpad) software (San Diego California USA).

### Pharmacology.

Biological activity of compounds tested in KAINR antagonist bioassay.

Reference compound Tezampanel was custom synthesized, purified, and chemically verified by and supplied by Sea Pharmaceuticals LLC (Cambridge Massachusetts USA). Experimental test compounds were or will be synthesized, purified, and chemically analyzed by Sea Pharmaceuticals LLC.

### Reference Compound Tests.

Table 4 shows results from tests of reference compound Tezampanel (Ref_1) (enantiopure) on Kainate (20 μM)-induced currents in whole cell patch clamp electrophysiology assay of KAINR ion channel (human recombinant GluK1 subunit) expressed in human embryonic kidney 293 (HEK293) cells transiently transfected with GRIK1 DNA expression plasmid S0001.

TABLE 4
Kainate assay.

| | Concentrations | IC50 value + SEM (μM) or | n = |
|---|---|---|---|
| Compound | tested (μM) | % inhib at concentration | experiments |
| Ref_1 | 0.03, 0.1 ,0.3, | 1.1 ± 0.1 | 3 to 4 |
| | 1.0, 3, 10, 30 | | |

### REFERENCES

Sommer B, Burnashev N, Verdoorn T A, Keinanen K, Sakmann B, Seeburg P H. 1992 A glutamate receptor channel with high affinity for domoate and kainate. EMBO J. 11:1651-1656
Korczak B, Nutt S L, Fletcher E J, Hoo K H, Elliott C E, Rampersad V, McWhinnie E A, Kamboj R K 1995. cDNA cloning and functional properties of human glutamate receptor EAA3 (GluR5) in homomeric and heteromeric configuration. Receptors and Channels 3(1):41-49. (Published by Harwood Academic Publishers GmbH, USA; current publisher: Taylor & Francis).
Jones K A, Wilding T J, Huettner J E, Costa A M. 1997. Desensitization of kainate receptors by kainate, glutamate and diastereomers of 4-methylglutamate Neuropharmacology 36:853-863.
Huettner J E, Stack E, Wilding T J. 1998. Antagonism of neuronal kainate receptors by lanthanum and gadolinium. Neuropharmacology 37:1239-1247
Wilding T J, Fulling E, Zhou Y, Huettner J E. 2008. Amino acid substitutions in the pore helix of GluR6 control inhibition by membrane fatty acids. J Gen Physiol. 132:85-99
Lopez M N, Wilding T J, Huettner J E 2013. Q/R site interactions with the M3 helix in GluK2 kainate receptor channels revealed by thermodynamic mutant cycles. J Gen Physiol 142:225-239

### D. In Vivo Pharmacology

In Vivo Pharmacology of Compounds after Treatment of Rodents, Including In Vivo Rodent Epilepsy or Seizure Models and In Vivo Rodent Pain Model.

### Compound Formulation and Preparation for Administration to Animals:

Dose suspensions or solutions of test compounds are formulated as specified in either of two formulations (i) DMSO in aqueous methylcellulose (referred to as DMC) or (ii) saline pH-adjusted (referred to as SPHA). For DMC formulation tests, compounds are dissolved in DMSO and diluted into 0.5% methylcellulose (containing up to 3% DMSO final concentration). 0.5% methylcellulose (0.5% MC, Sigma, Catalog M-0430, St. Louis, Missouri) is prepared in water as per the manufacturer's instructions. DMC mixtures of compounds are homogenized by vortex agitation and stirred on a heated plate (˜40° C.) until either the test compound mixtures is a homogenous suspension or is completely dissolved. SPHA formulation is used for some test articles that are higher aqueous soluble compounds or salts. For SPHA formulation compound powder is mixed directly into physiological (normal) saline (0.9% NaCl), then 0.1 N or 1 N sodium hydroxide solution is added carefully to pH 9 to pH 9.5 and the samples are stirred and agitated with vortex and heating at approximately 40° C. until fully dissolved (up to 15 minutes). Then 0.1 N or 1 N hydrochloric acid solution is then added carefully to adjust the pH to pH 7.1 to pH 7.3. Alternatively 0.5% methylcellulose solution in water can be substituted for normal saline solution. Vehicle solution is prepared using this same protocol without compound present. Dosing mixtures are allowed to equilibrate to room temperature prior to administration. Dosing mixtures are prepared fresh on the day of testing and used in less than 3 hours. All dosing solutions or dosing suspensions are mixed thoroughly prior to administration.

Compounds may be formulated in alternative vehicle formulations not limited to those presented above.

### Animal Handling Protocols and Compound or Vehicle Administration in Rodent Seizure Models (in Mice or Rats).

Adult male Carworth Farms (CF-1) mice (25-35 g) or young male Sprague-Dawley (SD) rats (100-150 g) are obtained from Charles River Laboratories Inc (Wilmington Massachusetts USA). CF1 mice are typically used for the 6 Hz psychomotor seizure model (6 Hz model described in the section below) but could also be used for the Maximal electroshock seizure (MES) model if performed in mice (Maximal electroshock seizure model described in the section below). SD rats Are typically used for MES model but could also be used for 6 Hz psychomotor model if performed in rats. Animals RE allowed free access to food and water, except during testing periods. After delivery from supplier lab to the in vivo pharmacology testing lab, animals are allowed sufficient time to acclimate to housing conditions prior to testing. Animals are housed in plastic cages in rooms with controlled humidity, ventilation, and lighting (12 hours lights on and 12 hours darkness). The animals are housed and fed in a manner consistent with recommendations in the “Guide for Care and Use of Laboratory Animals” (National Research Council), and in accordance with guidelines set by an Institutional Animal Care and Use Committee (IACUC). Animal experiments are conducted in a manner consistent with Animal Research: Reporting of In Vivo Experiments (ARRIVE) guidelines (UK) and are approved by an IACUC. Test compounds or respective vehicle (placebo) are administered using an optimal fluid volume-to-body fluid ratio. Solutions or suspensions of test compounds, reference compounds or vehicles are administered to mice or rats in a volume of 0.01 mL/g body weight (mice) or 0.004 mL/g body weight (rats) by subcutaneous (s.c.) injection, intraperitoneal (i.p.) injection or by oral gavage (p.o.) unless otherwise indicated. Reference drug Levetiracetam or vehicle is administered by intraperitoneal (i.p.) injection.

### Literature Description and In Vivo Pharmacological Validation of the Six Hertz (6 Hz) Psychomotor Seizure Model in Rodents.

Dr. James E. P. Toman first described the 6 Hz psychomotor seizure model in 1951. The 6 Hz psychomotor seizure model was extensively characterized and pharmacologically validated in mice using clinically used anti-epileptic drugs (AED's) by pharmacologist Louis S. Goodman in 1953 who showed certain clinically-used AED's were ineffective in the 6 Hz model in mice vs. the MES model in mice (described in another section below) and was found to resistant to treatment by certain AEDs such as phenytoin. The 6 Hz psychomotor seizure model in mice was little used for the next 50 years until revisited in 2001 by H. Steven White and Harold H. Wolf who pharmacologically compared several classes of AEDs. Toman JE 1951. Neurology 1:444-460. Brown W C et al. 1953. J Pharmacol Exp Ther 107:273-283. Barton M E et al. 2001. Epilepsy Res 47:217-227. Metcalf C S et al. 2017a. Epilepsia 58:484-493. Metcalf C S. 2017b. Epilepsia 58:1073-1084.

### 6 Hz Psychomotor Seizure Test of Compounds for Anti-Seizure Activity.

Mice are given a topical anesthetic on the cornea of each eye prior to placement of corneal electrodes. 6 Hz psychomotor seizures are induced in mice (usually 8 animals per group) using electrical stimulation via corneal electrodes (6 Hz, 0.2 millisecond rectangular pulse, 3 second duration at 22 mA using a Grass 48 stimulator instrument as described in Barton M E et al 2001. Epilepsy Res 47:217-227). Prior to placement of corneal electrodes for electrical stimulation 0.5% tetracaine in saline (Sigma) drops are applied to each eye. Seizures and behaviors that arise from the electrical stimulus in the 6 Hz model may include a minor clonic seizure phase followed by stereotyped automatistic behaviors including stun, forelimb clonus, twitching of the vibrissae and Straub-tail. For a period of 1 minute after stimulation animals are observed for these behaviors by a pharmacologist. If any of these behaviors are observed the animal is considered to have demonstrated a seizure. Animals not presenting any of these behaviors are considered “protected” from seizures. This serves as a screen for in vivo pharmacological activity of test articles or reference anti-epilepsy drugs and defines anti-seizure activity as shown in several publications [Barton M E et al. 2001. Epilepsy Res. 47:217-227; Barton M E et al. 2003 Epilepsy Res. 56:17-26; Brown W C et al. 1953. J Pharmacol Exp Ther 107:273-283. Metcalf C S et al. 2017a. Epilepsia 58:484-493.; Metcalf C S et al. 2017c. Epilepsia 58:239-246.

Unless otherwise indicated pretreatment times of mice or rats are typically 0.5 hours for respective vehicle, and compounds are administered by subcutaneous or intraperitoneal route (unless other times indicated). A pretreatment time of 1 hour is used, though test compounds or respective vehicles may use different pretreatment times. A median effective dose (ED₅₀) and 95% confidence interval (CI) are calculated using a Prism (Graphpad software).

### References for Rodent 6 Hz Psychomotor Seizure Models.

Toman J E 1951. Neurology 1:444-460. “Neuropharmacologic considerations in psychic seizures.”
Brown W C, Schiffman D O, Swinyard E A, Goodman L S. 1953. J Pharmacol Exp Ther 107:273-283. “Comparative Assay of an Antiepileptic Drugs by Psychomotor Seizure Test and Minimal Electroshock Threshold Test.”
Barton M E, Klein B D, Wolf H H, White H S. 2001. Epilepsy Res 47:217-227. “Pharmacological characterization of the 6 Hz psychomotor seizure model of partial epilepsy.”
Metcalf C S, Klein B D, Smith M D, Pruess T, Ceusters M, Lavreysen H, Pype S, Van Osselaer N, Twyman R, White H S. 2017a. Epilepsia 58:484-493. Efficacy of mGlu2 positive allosteric modulators alone and in combination with levetiracetam in the mouse 6 Hz model of psychomotor seizures;
Metcalf C S, West P J, Thomson K E, Edwards S F, Smith M D, White H S, Wilcox K S. 2017b. Epilepsia 58:1073-1084. “Development and pharmacologic characterization of the rat 6 Hz model of partial seizures.”
Metcalf C S, Klein B D, McDougle D R, Zhang L, Kaufmann D, Grzegorz Bulaj G, White H S. 2017c. Epilepsia 58:239-246. “Preclinical Evaluation of Intravenous NAX 810-2, a Novel GalR2-preferring Analog, for Anticonvulsant Efficacy and Pharmacokinetics.”

### Literature Description of MES (Maximum Electroshock Seizure) Model for Testing Molecules for Anti-Convulsant, Anti-Seizure and Anti-Epileptic Activity in Rodents.

Pharmacologist Louis S. Goodman extensively characterized the MES model in vivo pharmacology in treatment studies of experimental anti-convulsant, anti-seizure, anti-epileptic agents compared to anti-epileptic drugs (AEDs) in the 1950s-1970s. Later work 1980s-2010s by H. Steve White and Harold H. Wolf further extended use of the MES seizure model for pharmacological characterization of anti-epileptic compounds.

MES model materials, methods, and validation of several clinically used anti-epileptic drugs are described in Swinyard E A, et al. 1952. J Pharmacol Exp Ther 106:319-330. Goodman's methods for MES model used an electrical stimulus instrument and electrodes described in Woodbury LA, Davenport VD. 1952. Arch Int Pharmacodyn Ther 92:97-107. The MES animal model and this type of MES instrument have been extensively used for in vivo pharmacological characterization of efficacy of anti-convulsive agents and anti-epileptic drugs (AEDs) for the last several decades by the United States National Institute of Health in screening of compounds for anti-convulsant activity (first two references are Chapters in 2 books) White H S, et al. (1995). In Levy R H, Mattson R H, Meldrum B S (Eds) Book title: Antiepileptic Drugs. 4th edition. pp. 99-110; White H S, et al. (2002) In Levy R, Mattson R, Meldrum B, Perucca E (Eds) Book title: Antiepileptic Drugs. 5th edition., pp. 36-48; White H S et al. 1995. Italian Journal Neurological Sciences 16:73-77 White H S, et al. 1998. Advances in Neurol 76:29-39. (Review); White H S, et al. 2008. Epilepsia 49:1213-1220. (Methods: updated MES model description); Barton M E, Peters S C, Shannon H E. 2003 Epilepsy Res. 56:17-26 (detailed methods on mouse 6 Hz and mouse MES models);

### References for Rodent Maximal Electroshock Seizures (MES) Model in Rats or Mice.

Swinyard E A, Brown W C, Goodman L S. 1952. J Pharmacol Exp Ther 106:319-330. “Comparative assays of antiepileptic drugs in mice and rats”
Woodbury L A, Davenport V D. 1952. Arch Int Pharmacodyn Ther 92:97-107. “Design and use of a new electroshock seizure apparatus, and analysis of factors altering seizure threshold and pattern.”
White H S, Woodhead J H, Franklin M R. Swinyard E A, Wolf H H. (1995) General principles: experimental selection, quantification, and evaluation of antiepileptic drugs. In Levy R H, Mattson R H, Meldrum B S (Eds) Book title: Antiepileptic Drugs. 4th edition. Raven, New York, pp. 99-110;
White H S, Woodhead J H, Wilcox K S, Stables J P, Kupferberg H J, Wolf H H. (2002) Discovery and preclinical development of antiepileptic drugs. In Levy R, Mattson R, Meldrum B, Perucca E (Eds) Book title: Antiepileptic Drugs. 5th edition. Lippincott Williams & Wilkins, Philadelphia, pp. 36-48;
White H S, Johnson M, Wolf H H, Kupferberg H J. 1995. Italian Journal Neurological Sciences 16:73-77. “The early identification of anticonvulsant activity: role of the maximal electroshock and subcutaneous pentylenetetrazol seizure models.” (Review);
White H S, Wolf H H, Woodhead J H, Kupferberg H J. 1998. Advances in Neurol 76:29-39. “The national institutes of health anticonvulsant drug development program: screening for efficacy.” (Review);
White H S, Franklin M R, Kupferberg H J, Schmutz M, Stables J P, Wolf H H. 2008. Epilepsia 49:1213-1220. “The anticonvulsant profile of rufinamide (CGP 33101) in rodent seizure models.”
Leander J D, Rathbun R C, Zimmerman D M. 1988. Brain Res. 454:368-732“Anticonvulsant effects of phencyclidine-like drugs: relation to N-methyl-D-aspartic acid antagonism”;
Leander J D. 1989. Epilepsy Res. 4: 28-33 “Evaluation of dextromethorphan and carbetapentane as anti-convulsants and N-methyl-D-aspartic acid antagonists in mice”;
Yamaguchi S, Donevan S D, Rogawski M A. 1993. Epilepsy Res. 15:179-184 “Anticonvulsant activity of AMPA kainate antagonists: comparison of GYKI 52466 and NBOX in maximal electroshock and chemoconvulsant seizure models.”
Barton M E, Peters S C, Shannon H E. 2003 Epilepsy Res. 56:17-26 “Comparison of the effect of glutamate receptor modulators in the 6 Hz and maximal electroshock seizure models
Metcalf C S, West P J, Thomson K E, Edwards S F, Smith M D, White H S, Wilcox K S. 2017c. Epilepsia 58:1073-1084. “Development and pharmacologic characterization of the rat 6 Hz model of partial seizures”.

### Maximal Electroshock Seizure (MES) Model In Vivo Pharmacology Test of Compounds for Anti-Seizure Activity in Rats.

Male Sprague-Dawley rats (body weight 100 to 150 grams at time of testing) are supplied by Charles River Laboratories Wilmington Massachusetts USA (N 8-10 animals per group). Rats are administered test article (experimental compounds, or vehicle, or positive control reference anti-convulsant compounds) by intraperitoneal (i.p.), subcutaneous (s.c.), intravenous (i.v.) or by oral gavage (p.o.) route. Corneas of rats are anesthetized with 0.5% tetracaine in saline at time of dosing and again prior to corneal stimulus. Unless otherwise indicated the pretreatment time for compounds is 30 minutes (pretreatment time for vehicles or other test compounds was 60 min or as indicated) prior to corneal application of stimulus via electrodes (electric stimulus alternating current 60 Hz, 150 mA for 0.2 seconds duration for rats). Note if mice are used the stimulus parameters would follow published procedures of alternating current 60 Hz, 50 mA for 0.2 seconds [Metcalf C S, et al. 2017c. Epilepsia 58:1073-1084; White H S et al. 1995. Italian Journal Neurological Sciences 16:73-77; Barton M E, et al. 2003 Epilepsy Res. 56:17-26 (contains detailed description of MES and 6 Hz models both in mice); Leander J D, Rathbun R C, Zimmerman D M. Brain Res. 1988, 454:68-72; Leander J D 1989 Epilepsy Res. 4:28-33; and Yamaguchi S, Donevan S D, Rogawski M A. 1993 Epilepsy Res. 15:179-184.]. If animals do not show hind limb extension, they are considered protected from the convulsant effect of electroshock. In some cases the effective dose (ED₅₀ and 95% confidence interval) of test compound or reference compound that abolished the tonic-extensor component of the convulsion in 50% of the animals is calculated from dose-response data [Litchfield, JT Jr, Wilcoxon. 1949. J. Pharmacol. Exp. Ther. 96:99-113 “A simplified method of evaluating dose-effect experiments.” ]. One group of animals always receives vehicle treatment (negative control) and one group of animals always receives reference compound treatment (positive control) for each experiment.

### AMPA Receptor Antagonist Activity of Compounds In Vivo.

In vivo activity of AMPA receptor antagonists compounds can be assayed using the Maximum electroshock seizure (MES) model in mice or in rats or the 6 Hz psychomotor seizure model in mice or rats. In vivo efficacy data for AMPA receptor antagonist tezampanel treatment has been shown in mouse MES model in Ornstein P L et al., 1993. J Med. Chem 36:2046-2048 and in both the mouse MES and mouse 6 Hz psychomotor seizure model in Barton M E et al. 2003. Epilepsy Res 56:17-26. In vivo efficacy data for treatment of mice with FDA approved anti-epileptic drug AMPA receptor antagonist (AMPARA) perampanel in both the 6 Hz seizure model and in the maximal electroshock seizure MES model in mice was presented by Hanada T et al. 2011. Epilepsia 52:1331-1340. In vivo efficacy data with experimental AMPA receptor antagonist (AMPARA) compound YM928 tested in MES mice model was shown in Yamashita H, et al. 2004. J Pharmacol Exp Ther 308:127-133. In vivo efficacy data comparing experimental therapeutic AMPARA compound YM928 vs its derivatives cf a clinically studied AMPARA (talampanel) treatment of mice in the MES model was shown in Inami H, et al. 2019. Chem Pharm Bull (Tokyo) 67:699-706.

### References for AMPA Receptor Antagonist Compound or Drug Treatment Efficacy in Rat or Mouse MES or 6 Hz Psychomotor Seizure Models.

Ornstein P L, Arnold M B, Augenstein N K, Lodge D, Leander J D, Schoepp D D. 1993. J Med. Chem 36:2046-2048. “(3SR,4aRS,6RS,8aRS)-6-[2-(1H-tetrazol-5-yl)ethyl]decahydroisoquinoline-3-carboxylic acid: a structurally novel, systemically active, competitive AMPA receptor antagonist” (tezampanel);
Barton M E, Peters S C, Shannon H E. 2003. Epilepsy Res 56:17-26. “Comparison of the effect of glutamate receptor modulators in the 6 Hz and maximal electroshock seizure models” (tezampanel);
Hanada T, Hashizume Y, Tokuhara N, Takenaka O, Kohmura N, Ogasawara A, Hatakeyama S, Ohgoh M, Ueno M, Nishizawa Y. 2011. Epilepsia 52:1331-1340. “Perampanel: A Novel, Orally Active, Noncompetitive AMPA-receptor Antagonist That Reduces Seizure Activity in Rodent Models of Epilepsy” (FDA approved anti-epileptic drug AMPA receptor antagonist perampanel in 6 Hz seizure or MES models in mice);
Yamashita H, Ohno K, Amada Y, Hattori H, Ozawa-Funatsu Y, Toya T, Inami H, Shishikura J-I, Sakamoto S, Okada M, Yamaguchi T. 2004. J Pharmacol Exp Ther 308:127-133. “Effects of 2-[N-(4-chlorophenyl)-N-methylamino]-4H-pyrido[3.2-e]-1,3-thiazin-4-one (YM928), an orally active alpha-amino-3-hydroxy-5-methyl-4-isoxazolepropionic acid receptor antagonist, in models of generalized epileptic seizure in mice and rats” (experimental AMPA receptor antagonist (AMPARA) compound YM928 tested in MES mice model);
Inami H, Shishikura J-I, Yasunaga T, Hirano M, Kimura T, Yamashita H, Ohno K, Sakamoto S. 2019. Chem Pharm Bull (Tokyo) 67:699-706. “Synthesis and pharmacological evaluation of 3-[(4-oxo-4H-pyrido[3,2-e][1,3]thiazin-2-yl)(phenyl)amino]propanenitrile derivatives as orally active AMPA receptor antagonists”. (in vivo efficacy data comparing experimental therapeutic AMPARA compound YM928 vs its derivatives cf a clinically studied AMPARA (talampanel) in the MES model in mice).

### Rotarod Test for Motor Impairment.

Testing mouse performance on the rotarod is conducted in tandem with 6 Hz psychomotor seizure model stimulation to verify whether doses administered produced substantial motor impairment. Evaluation in the rotarod assay is conducted immediately prior to 6 Hz psychomotor seizure model testing. Therefore, each treatment group is subjected to evaluation in the rotarod assay followed immediately by testing in the 6 Hz psychomotor seizure model. When mice are placed on a 1-inch knurled rod rotating at a speed of 6 rpm, the animals can maintain equilibrium for long periods. Motor impairment is assessed by determining whether mice remained on the rotarod during a 1-minute observation period; that is, three falls during a 1 minute period is considered a rotarod failure.

Rat behavior pharmacology observations for signs of motor impairment by trained observer. Rats are monitored after dosing for any signs of impairment by compounds after administration.

### Statistical Analysis.

Seizure protection and rotarod motor impairment data are presented as #(the number of animals protected from seizure)/N (the treatment group size consisting of the number of animals tested by treatment with vehicle or compound at a given dose) and # with motor impairment/N tested, respectively. A Fisher exact test is used to compare motor impairment values for specific treatment groups with VEH-treated animals. For dose-response analyses the dose resulting in 50% efficacy ED₅₀ (and 95% CI) values are calculated using Prism (Graphpad Software) analysis wherein at least three treatment groups (N=6 to 10 animals per group) are used in the calculation. Plasma levels are presented as means±standard error and were compared using a Student's t-test.

### II. Deuterated Acids

### A. Generic Chemical Synthesis

The structures and names of deuterated acids are provided in Table 1, below.

TABLE 1
Deuterated Acids

| # | Structure | Name |
|---|---|---|
| A02 | 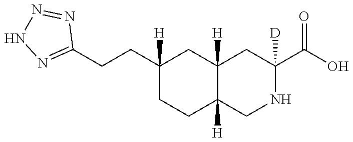 [SMILES: [2H][C@@]1(C(=O)O)C[C@H]2C[C@@H](CCc3nn[nH]n3)CC[C@H]2CN1] | (3S,4aS,6S,8aR)-6-[2-(2H-1,2,3,4- tetrazol-5-yl)ethyl]-decahydro(3- d₁)isoquinoline-3-carboxylic acid |
| A03 | 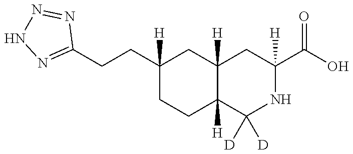 [SMILES: [2H]C1([2H])N[C@H](C(=O)O)C[C@H]2C[C@@H](CCc3nn[nH]n3)CC[C@H]21] | (3S,4aS,6S,8aR)-6-[2-(2H-1,2,3,4- tetrazol-5-yl)ethyl]-decahydro(1,1- d₂)isoquinoline-3-carboxylic acid |
| A04 | 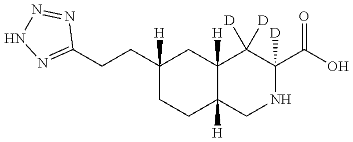 [SMILES: [2H]C1([2H])[C@H]2C[C@@H](CCc3nn[nH]n3)CC[C@H]2CN[C@]1([2H])C(=O)O] | (3S,4aS,6S,8aR)-6-[2-(2H-1,2,3,4- tetrazol-5-yl)ethyl]-decahydro(3,4,4- d₃)isoquinoline-3-carboxylic acid |
| A05 | 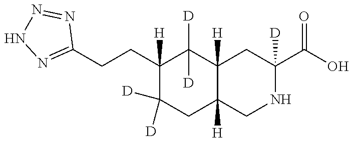 | (3S,4aS,6S,8aR)-6-[2-(2H-1,2,3,4- tetrazol-5-y1)ethyl]-decahydro(3,5,5,7,7- d₅)isoquinoline-3-carboxylic acid |
| A06 | 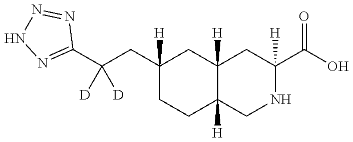 [SMILES: [2H]C([2H])(C[C@H]1CC[C@H]2CN[C@H](C(=O)O)C[C@H]2C1)c1nn[nH]n1] | (3S,4aS,6S,8aR)-6-[2-(2H-1,2,3,4- tetrazol-5-y1)(2,2-d₂)ethyl]- decahydroisoquinoline-3-carboxylic acid |
| A07 | 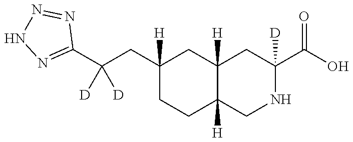 [SMILES: [2H]C([2H])(C[C@H]1CC[C@H]2CN[C@]([2H])(C(=O)O)C[C@H]2C1)c1nn[nH]n1] | (3S,4aS,6S,8aR)-6-[2-(2H-1,2,3,4- tetrazol-5-y1)(2,2-d₂)ethyl]-decahydro(3- d₁)isoquinoline-3-carboxylic acid |
| A08 | 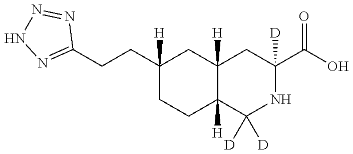 [SMILES: [2H]C1([2H])N[C@]([2H])(C(=O)O)C[C@H]2C[C@@H](CCc3nn[nH]n3)CC[C@H]21] | (3S,4aS,6S,8aR)-6-[2-(2H-1,2,3,4- tetrazol-5-yl)ethyl]-decahydro(1,1,3- d₃)isoquinoline-3-carboxylic acid |
| A09 | 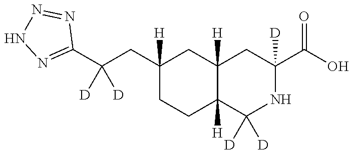 [SMILES: [2H]C([2H])(C[C@H]1CC[C@@H]2[C@H](C1)C[C@@]([2H])(C(=O)O)NC2([2H])[2H])c1nn[nH]n1] | (3S,4aS,6S,8aR)-6-[2-(2H-1,2,3,4- tetrazol-5-y1)(2,2-d₂)ethyl]- decahydro(1,1,3-d₃)isoquinoline-3- carboxylic acid |

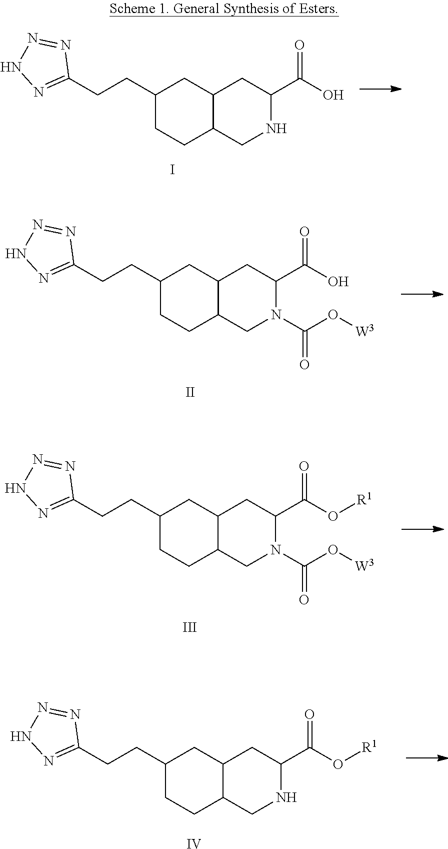

Scheme 1 illustrates the general synthetic scheme to make the desired esters. In step 1, amino acid I is reacted with benzyl chloroformate (Cbz), or similar reagent (e.g., di-tert-butyl dicarbonate (Boc), fluorenylmethyloxycarbonyl chloride (Fmoc), etc.), under basic conditions (e.g., 2N aqueous NaOH, triethylamine, diisopropylethylamine, etc.) in organic solvent (e.g., acetonitrile, DMF, etc.) to form carbamate II (—C(═O)—O—W³). Carbamate II is then coupled to an alcohol starting material (HO-R¹; see Table 3 below) using N,N′-dicyclohexylcarbodiimide (DCC) or related coupling reagent (e.g, EDC, HBTU, HATU, PyBOP, etc.) under basic conditions (e.g., triethylamine, diisopropylethylamine, pyridine, N,N-4-dimethylaminopyridine, etc.) in organic solvent (e.g., dichloromethane, acetonitrile, DMF, etc.) to make ester carbamate III. Ester carbamate III is then deprotected under the appropriate conditions to selectively remove the carbamate protecting group (i.e., hydrogenation, basic, or acidic conditions) and afford the desired prodrugs IV. In some cases, the alcohol (HO-R¹) is a Cbz- or Boc-protected compound, this protecting group is removed at the same time as the carbamate protecting group on compound III, matching the protecting groups is important for this to be possible.

Deuterated analogs of the core acid are made by introducing deuterated reagents at specific steps in the synthesis (see B. Huff “Excitatory amino acid receptor antagonists.” U.S. Pat. No. 5,284,957, 1994; A. M. Brian Arnold, et al. “Process for preparing isoquinoline compounds.” U.S. Pat. No. 5,648,492, 1997; Paul L. Ornstein, et al. “(3SR,4aRS,6RS,8aRS)-6-[2-(1H-tetrazol-5-yI)ethyl]decahydroisoquinoline-3-carboxylic Acid: A Structurally Novel, Systemically Active, Competitive AMPA Receptor Antagonist.” J. Med. Chem., 1993, 36, 2046-2048; and Paul L. Ornstein, et al. “Syntheses of Oxodecahydroisoquinoline-3-carboxylates. Useful Intermediates for the Preparation of Conformationally Defined Excitatory Amino Acid Antagonists.” J. Org. Chem., 1991, 56, 4388-4392). Scheme 2 illustrates the general synthetic scheme to make the deuterated acids. The underlying route is based on established chemistry, while the deuteration operations are novel.

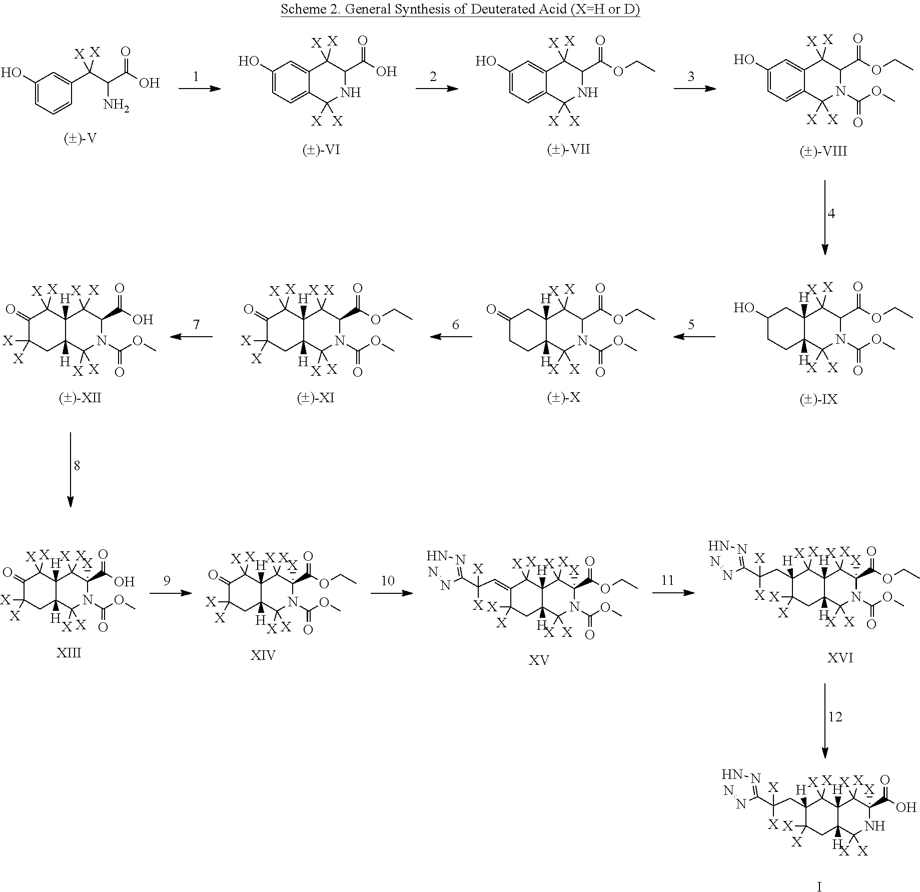

One example of a preparation method can include the following in reference to Scheme 2. In step 1, a Pictet-Spengler reaction of (±)-3-hydroxyphenylalanine V (or 3-hydroxy-L-phenylalanine-α-d₁ (CAS #[1226919-23-8], see Oh, Joong-Suk, et al. “Enantioselective synthesis of α-deuterium labeled chiral α-amino acids via dynamic kinetic resolution of racemic azlactones.” Organic & Biomolecular Chemistry 2011, 9 (23), 7983-7985) and 3-hydroxy-L-phenylalanine-α,β,β-d₃ (CAS #[1226918-52-0], see Gant, Thomas G.; Hodiluk, Craig; Woo, Soon H. PCT Int. Appl., WO 2010054286 A2, 2010, or any other deuterated 3-hydroxyphenylalanine referenced in Gant, Thomas G. et al.) with 37% formaldehyde (or 20% formaldehyde-d₂, CAS #[1664-98-8], Sigma-Aldrich) affords compound VI:
6-hydroxy-1,2,3,4-tetrahydroisoquinoline-3-carboxylic acid
6-hydroxy-1,2,3,4-tetrahydro(3-d₁)isoquinoline-3-carboxylic acid
6-hydroxy-1,2,3,4-tetrahydro(1,1-d₂)isoquinoline-3-carboxylic acid
6-hydroxy-1,2,3,4-tetrahydro(3,4,4-d₃)isoquinoline-3-carboxylic acid

In step 2, esterification of VI with thionyl chloride in ethanol affords ethyl ester VII:
ethyl 6-hydroxy-1,2,3,4-tetrahydroisoquinoline-3-carboxylate
ethyl 6-hydroxy-1,2,3,4-tetrahydro(3-d₁)isoquinoline-3-carboxylate
ethyl 6-hydroxy-1,2,3,4-tetrahydro(1,1-d₂)isoquinoline-3-carboxylate
ethyl 6-hydroxy-1,2,3,4-tetrahydro(3,4,4-d₃)isoquinoline-3-carboxylate

In step 3, reaction of VII with methylchloroformate at 0° C. in the presence of N,N-diisopropylethylamine or similar base (e.g., triethylamine) in dichloromethane or similar solvent (e.g. chloroform, ethyl acetate, THF, 1,2-dichloroethane, toluene, etc.) affords methyl carbamate VIII:
3-ethyl 2-methyl 6-hydroxy-1,2,3,4-tetrahydroisoquinoline-2,3-dicarboxylate
3-ethyl 2-methyl 6-hydroxy-1,2,3,4-tetrahydro(3-d₁)isoquinoline-2,3-dicarboxylate
3-ethyl 2-methyl 6-hydroxy-1,2,3,4-tetrahydro(1,1-d₂)isoquinoline-2,3-dicarboxylate
3-ethyl 2-methyl 6-hydroxy-1,2,3,4-tetrahydro(3,4,4-d₃)isoquinoline-2,3-dicarboxylate

In step 4, high pressure hydrogenation of VIII in absolute ethanol using 5% ruthenium on carbon (Ru/C) at 80° C. under 4.5 MPa of hydrogen atmosphere for 48-96 h affords alcohol IX:
3-ethyl 2-methyl 6-hydroxy-decahydroisoquinoline-2,3-dicarboxylate
3-ethyl 2-methyl 6-hydroxy-decahydro(3-d₁)isoquinoline-2,3-dicarboxylate
3-ethyl 2-methyl 6-hydroxy-decahydro(1,1-d₂)isoquinoline-2,3-dicarboxylate
3-ethyl 2-methyl 6-hydroxy-decahydro(3,4,4-d₃)isoquinoline-2,3-dicarboxylate

In step 5, PCC or PDC oxidation of IX, or alternative oxidation procedure (e.g. Swern conditions), in dichloromethane, or similar solvent (e.g. chloroform, 1,2-dichloroethane, dichlorobenzene, etc.), provides ketone X:
3-ethyl 2-methyl 6-oxo-decahydroisoquinoline-2,3-dicarboxylate
3-ethyl 2-methyl 6-oxo-decahydro(3-d₁)isoquinoline-2,3-dicarboxylate
3-ethyl 2-methyl 6-oxo-decahydro(1,1-d₂)isoquinoline-2,3-dicarboxylate
3-ethyl 2-methyl 6-oxo-decahydro(3,4,4-d₃)isoquinoline-2,3-dicarboxylate

In step 6, epimerization of the C3-position in ketone X is done in ethanol (or ethanol-d) with sodium ethoxide at 50-80° C. for 2 h to afford ketone XI:
3-ethyl 2-methyl 6-oxo-decahydroisoquinoline-2,3-dicarboxylate
3-ethyl 2-methyl 6-oxo-decahydro(3-d₁)isoquinoline-2,3-dicarboxylate
3-ethyl 2-methyl 6-oxo-decahydro(1,1-d₂)isoquinoline-2,3-dicarboxylate
3-ethyl 2-methyl 6-oxo-decahydro(3,4,4-d₃)isoquinoline-2,3-dicarboxylate
3-ethyl 2-methyl 6-oxo-decahydro(3,5,5,7,7-d₅)isoquinoline-2,3-dicarboxylate

If using ethanol-d, the solvent is removed and recharged several times to allow for full proton to deuterium exchange.

In step 7, hydrolysis of XI with 1N aqueous sodium hydroxide in ethanol (or TIF) affords carboxylic acid XII:
2-(methoxycarbonyl)-6-oxo-decahydroisoquinoline-3-carboxylic acid
2-(methoxycarbonyl)-6-oxo-decahydro(3-d₁)isoquinoline-3-carboxylic acid
2-(methoxycarbonyl)-6-oxo-decahydro(1,1-d₂)isoquinoline-3-carboxylic acid
2-(methoxycarbonyl)-6-oxo-decahydro(3,4,4-d₃)isoquinoline-3-carboxylic acid
2-(methoxycarbonyl)-6-oxo-decahydro(3,5,5,7,7-d₅)isoquinoline-3-carboxylic acid

In step 8, chiral resolution of XII into enantiomers is done using (R)-(+)-1-phenyl-ethylamine in THF (or ethyl acetate) followed by salt breaking with Dowex acidic resin in acetonitrile affords chiral ketone carboxylic acid XIII:
(3S,4aS,8aR)-2-(methoxycarbonyl)-6-oxo-decahydroisoquinoline-3-carboxylic acid
(3S,4aS,8aR)-2-(methoxycarbonyl)-6-oxo-decahydro(3-d₁)isoquinoline-3-carboxylic acid
(3S,4aS,8aR)-2-(methoxycarbonyl)-6-oxo-decahydro(1,1-d₂)isoquinoline-3-carboxylic acid
(3S,4aS,8aR)-2-(methoxycarbonyl)-6-oxo-decahydro(3,4,4-d₃)isoquinoline-3-carboxylic acid
(3S,4aS,8aR)-2-(methoxycarbonyl)-6-oxo-decahydro(3,5,5,7,7-d₅)isoquinoline-3-carboxylic acid

In step 9, alkylation of the chiral ketone carboxylate XIII with ethyl iodide in DMF using sodium bicarbonate as base affords chiral ketone ester XIV:
3-ethyl 2-methyl (3S,4aS,8aR)-6-oxo-decahydroisoquinoline-2,3-dicarboxylate
3-ethyl 2-methyl (3S,4aS,8aR)-6-oxo-decahydro(3-d₁)isoquinoline-2,3-dicarboxylate
3-ethyl 2-methyl (3S,4aS,8aR)-6-oxo-decahydro(1,1-d₂)isoquinoline-2,3-dicarboxylate
3-ethyl 2-methyl (3S,4aS,8aR)-6-oxo-decahydro(3,4,4-d₃)isoquinoline-2,3-dicarboxylate
3-ethyl 2-methyl (3S,4aS,8aR)-6-oxo-decahydro(3,5,5,7,7-d₅)isoquinoline-2,3-dicarboxylate

In step 10, the chiral ketone ester XIV is reacted with a tetrazole Wittig reagent (or deuterated tetrazole Wittig A06-3; see below) deprotonated with 1.0 M NaHMDS in THF or similar base (e.g. LDA, LiHMDS, KHMDS) in DMF at 0° C. to afford tetrazole olefin XV:
3-ethyl 2-methyl (3S,4aS,6E,8aR)-6-[2-(2H-1,2,3,4-tetrazole-5-yl)ethylidene]-decahydroisoquinoline-2,3-dicarboxylate
3-ethyl 2-methyl (3S,4aS,6E,8aR)-6-[2-(2H-1,2,3,4-tetrazole-5-yl)ethylidene]-decahydro(3-d₁)isoquinoline-2,3-dicarboxylate
3-ethyl 2-methyl (3S,4aS,6E,8aR)-6-[2-(2H-1,2,3,4-tetrazole-5-yl)ethylidene]-decahydro(1,1-d₂)isoquinoline-2,3-dicarboxylate
3-ethyl 2-methyl (3S,4aS,6E,8aR)-6-[2-(2H-1,2,3,4-tetrazole-5-yl)ethylidene]-decahydro(3,4,4-d₃)isoquinoline-2,3-dicarboxylate
3-ethyl 2-methyl (3S,4aS,6E,8aR)-6-[2-(2H-1,2,3,4-tetrazole-5-yl)ethylidene]-decahydro(3,5,5,7,7-d₅)isoquinoline-2,3-dicarboxylate
3-ethyl 2-methyl (3S,4aS,6E,8aR)-6-[2-(2H-1,2,3,4-tetrazole-5-yl)(2,2-d₂)ethylidene]-decahydroisoquinoline-2,3-dicarboxylate

In step 11, hydrogenation of XV in absolute ethanol, 1:2 acetic acid-ethyl acetate or related solvent (e.g. THF, isopropanol, etc.) using 5% palladium on carbon (Pd/C) or similar catalyst (e.g. PtO₂, 10% Pd/C, etc.) at room temperature to 50° C. under 25-100 psi of hydrogen atmosphere for 12-48 h affords carbamate ester XVI:
3-ethyl 2-methyl (3S,4aS,6S,8aR)-6-[2-(2H-1,2,3,4-tetrazol-5-yl)ethyl]-decahydroisoquinoline-2,3-dicarboxylate
3-ethyl 2-methyl (3S,4aS,6S,8aR)-6-[2-(2H-1,2,3,4-tetrazol-5-yl)ethyl]-decahydro(3-d₁)isoquinoline-2,3-dicarboxylate
3-ethyl 2-methyl (3S,4aS,6S,8aR)-6-[2-(2H-1,2,3,4-tetrazol-5-yl)ethyl]-decahydro(1,1-d₂)isoquinoline-2,3-dicarboxylate
3-ethyl 2-methyl (3S,4aS,6S,8aR)-6-[2-(2H-1,2,3,4-tetrazol-5-yl)ethyl]-decahydro(3,4,4-d₃)isoquinoline-2,3-dicarboxylate
3-ethyl 2-methyl (3S,4aS,6S,8aR)-6-[2-(2H-1,2,3,4-tetrazol-5-yl)ethyl]-decahydro(3,5,5,7,7-d₅)isoquinoline-2,3-dicarboxylate
3-ethyl 2-methyl (3S,4aS,6S,8aR)-6-[2-(2H-1,2,3,4-tetrazol-5-yl)(2,2-d₂)ethyl]-decahydroisoquinoline-2,3-dicarboxylate

In addition, compound XVI is treated with 0.5 M sodium ethoxide in ethanol-d at 40-80° C. to incorporate deuterium into the C3-position and afford carbamate esters XVI:
3-ethyl 2-methyl (3S,4aS,6S,8aR)-6-[2-(2H-1,2,3,4-tetrazol-5-yl)ethyl]-decahydro(3-d₁)isoquinoline-2,3-dicarboxylate
3-ethyl 2-methyl (3S,4aS,6S,8aR)-6-[2-(2H-1,2,3,4-tetrazol-5-yl)(2,2-d₂)ethyl]-decahydro(3-d₁)isoquinoline-2,3-dicarboxylate
3-ethyl 2-methyl (3S,4aS,6S,8aR)-6-[2-(2H-1,2,3,4-tetrazol-5-yl)ethyl]-decahydro(1,1,3-d₃)isoquinoline-2,3-dicarboxylate

In step 12, carbamate ester XVI is dissolved in 6N HCl (or 6N DCl in deuterium oxide) and heated to 90° C. for 2-12 h. Purification by resin catch-and-release and trituration with aprotic organic solvents (e.g. acetonitrile, acetone, ethyl acetate, MTBE, etc.) affords I (see Table 1). These examples are not intended to be limiting, additional deuterated analogs are made using alternative deuterated reagents and these same procedures.

In addition to modifying the established steps using deuterated reagents, other techniques are possible using several known protocols to affect hydrogen-deuterium exchange directly on amines and amino acids (Scheme 3, where X=H or D; and see the following references: Lillian V. A. Hale, et al. “Stereoretentive Deuteration of α-Chiral Amines with D₂O” J. Am. Chem. Soc., 2016, 138 (41), 13489-13492; C. Taglang, et al. “Enantiospecific C—H Activation Using Ruthenium Nanocatalysts.” Angew. Chem. Int. Ed., 2015, 54, 10474-10477; Nkaelang Modutlwa, et al. “Synthesis of deuterium-labelled drugs by hydrogen-deuterium (H-D) exchange using heterogeneous catalysis.” J. Label Compd. Radiopharm., 2010, 53, 686-692; Alessia Michelotti, et al. “Development and Scale-Up of Stereoretentive α-Deuteration of Amines.” Org. Process Res. Dev., 2017, 21, 1741-1744; Oh, Joong-Suk, et al. “Enantioselective synthesis of α-deuterium labeled chiral α-amino acids via dynamic kinetic resolution of racemic azlactones.” Org. Biomol. Chem., 2011, 9 (23), 7983-7985; Gant, Thomas G.; Hodiluk, Craig; Woo, Soon H. “Substituted hydroxyphenylamine compounds.” PCT Int. Appl., WO 2010054286 A2, 2010; Loh, Yong Yao, et al. “Photoredox-catalyzed deuteration and tritiation of pharmaceutical compounds.” Science, 2017, 358(6367), 1182-1187).

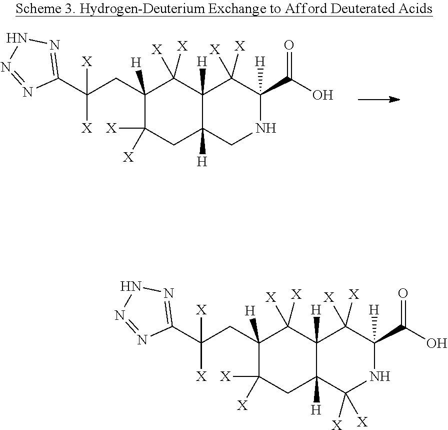

### B. Experimental Chemical Synthesis

Example preparations of the compounds in Table 1 are given below.

### Preparation of A01 (Ref_1)

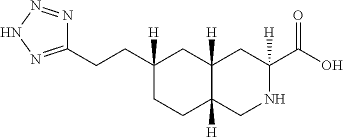 [SMILES: O=C(O)[C@@H]1C[C@H]2C[C@@H](CCc3nn[nH]n3)CC[C@H]2CN1]

This compound was prepared using known methods.

### Preparation of C3-Monodeuterated A02

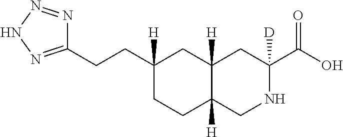 [SMILES: [2H][C@@]1(C(=O)O)C[C@H]2C[C@@H](CCc3nn[nH]n3)CC[C@H]2CN1]

Using the same protocols to synthesis A01, Step 11 is modified by adding an additional step after hydrogenation to deuterate the C3-center under basic conditions, as described below:

Deuteration of the C3-Center: 3-Ethyl 2-methyl (3S,4aS,6S,8aR)-6-[2-(2H-1,2,3,4-tetrazol-5-yl)ethyl]-decahydro(3-d₁)isoquinoline-2,3-dicarboxylate (A02-1).

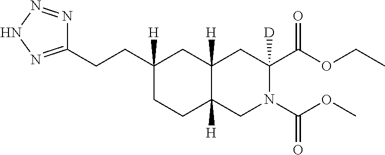 [SMILES: [2H][C@@]1(C(=O)OCC)C[C@H]2C[C@@H](CCc3nn[nH]n3)CC[C@H]2CN1C(=O)OC]

Step 1—3-Ethyl 2-methyl (3S,4aS,6S,8aR)-6-[2-(2H-1,2,3,4-tetrazol-5-yl)ethyl]-decahydroisoquinoline-2,3-dicarboxylate (see structure XVI above) (389 mg, 1.06 mmol) was dissolved in ethanol-d (2.3 mL, 99% D, <6% D₂O, Cambridge Isotope Laboratories, Inc., Catalog #DLM-16-50) and treated with 0.5 M sodium ethoxide in ethanol-d(1.4 eq., 3.0 mL) and stirred at 50° C. for 45 min. The mixture was partially concentrated to remove most of the ethanol-d and charged with fresh ethanol-d (5.3 mL) and stirred at 50° C. for 45 min. The reaction was quenched with 2N HCl in diethyl ether (2.5 eq.) and the mixture was concentrated under vacuum. The residue was purified by silica gel flash column chromatography (0% to 5% methanol-dichloromethane) to obtain A02-1 (300 mg, 77% yield).

### Step 2—Hydrolysis of Ester and Carbamate to Make C3-Monodeuterated A02

Hydrolysis of the ester and carbamate protecting groups in A02-1 was accomplished by dissolving the compound from Step 1 in 6N HCl and heating at 90° C. for 2 h. The reaction was monitored by HPLC. Upon completion the reaction was concentrated under vacuum at 50° C. and then purified by resin catch-and-release using Dowex 50WX8 200-400 resin (eluting with 10% aqueous pyridine or 3N ammonium hydroxide), followed by treatment with decolorizing charcoal in water to afford A02 (124 mg, 54% yield, 98.5% deuterium incorporation assayed by mass spectrometry or ¹H NMR).

### Preparation of C1-Dideuterated A03

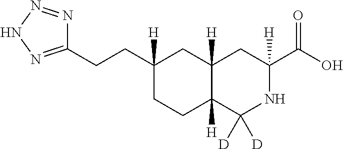 [SMILES: [2H]C1([2H])N[C@H](C(=O)O)C[C@H]2C[C@@H](CCc3nn[nH]n3)CC[C@H]21]

Using the same protocols to synthesis A01, Step 1 is modified by replacing formaldehyde in water with deuterated formaldehyde in deuterium oxide as described below:

Deuteration of the C1-Center: 6-Hydroxy-1,2,3,4-tetrahydro(1,1-d₂)isoquinoline-3-carboxylic acid (A03-1).

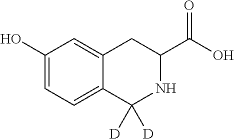 [SMILES: [2H]C1([2H])NC(C(=O)O)Cc2cc(O)ccc21]

A suspension of (±)-3-hydroxyphenylalanine (27.6 mmol) in 20% formaldehyde-d₂ in deuterium oxide (7.2 mL, CAS #[1664-98-8], Sigma-Aldrich) and 0.05 N deuterium chloride in deuterium oxide (40 mL) is heated to 90° C. for 7 h. The mixture is cooled to room temperature and the solid is filtered, washed twice with deuterium oxide (2×4 mL), and dried under vacuum to afford A03-1. This intermediate is incorporated into the established published synthesis to make C1-dideuterated A03.

### Preparation of C4-Trideuterated A04

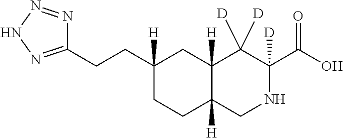 [SMILES: [2H]C1([2H])[C@H]2C[C@@H](CCc3nn[nH]n3)CC[C@H]2CN[C@]1([2H])C(=O)O]

Using the same protocols to synthesis A01, Step 1 is modified by replacing (±)-3-hydroxyphenylalanine with 3-hydroxy-L-phenylalanine-α,β,β-d₃ (CAS #[1226918-52-0]) see Gant, Thomas G.; Hodiluk, Craig; Woo, Soon H. PCT Int. Appl., WO 2010054286 A2, 2010 for preparation).

### Preparation of C3, C5, C7-Pentadeuterated A05

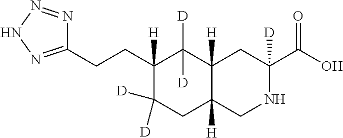

Using the same protocols to synthesis A01, Step 6 is modified by replacing ethanol with ethanol-d.

Deuteration of C3, C5, C7-Centers: 3-Ethyl 2-methyl 6-oxo-decahydro(3,5,5,7,7-d₅)isoquinoline-2,3-dicarboxylate (A05-1).

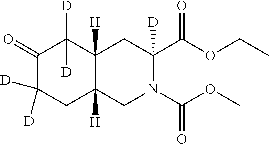

To a 2-dram vial containing 60% sodium hydride dispersion (15 mg, 0.37 mmol), was added ethanol-d (3.0 mL, 99% D, <6% D₂O, Cambridge Isotope Laboratories, Inc., Catalog #DLM-16-50). This sodium ethoxide solution was added to 3-ethyl 2-methyl (3S,4aS,8aR)-6-oxo-decahydroisoquinoline-2,3-dicarboxylate (see structure XIV above, 353 mg, 1.25 mmol) and the mixture was stirred for 18 h at 80° C. Analysis by ¹H NMR and GCMS showed >99% deuterium incorporation at C3 and ˜87% at C5 and C7. The solvent was evaporated, the residue was dissolved in DCM (5 mL), washed with saturated aqueous ammonium chloride solution (1 mL), and the aqueous layer was back-extracted with DCM (3×5 mL). The combined organic layers were dried over anhydrous sodium sulfate and evaporated to afford A05-1 as a pale yellow oil (287 mg, 80% yield). The residue was purified by silica gel flash column chromatography (0% to 50% ethyl acetate/hexanes) and then this intermediate is incorporated into the established published synthesis to make C3, C5, C7-pentadeuterated A05.

### Preparation of C10-Dideuterated A06

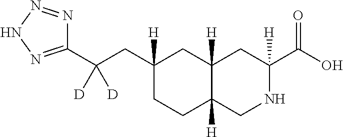 [SMILES: [2H]C([2H])(C[C@H]1CC[C@H]2CN[C@H](C(=O)O)C[C@H]2C1)c1nn[nH]n1]

Using the same protocols to synthesis A01, Step 10 is modified by replacing the tetrazole Wittig reagent with a deuterated tetrazole Wittig reagent (A06-3).

### Preparation of Deuterated Tetrazole Wittig Reagent Triphenyl[2-(2H-1,2,3,4-tetrazol-5-yl)(2,2-d₂)ethyl]phosphonium bromide (A06-3)

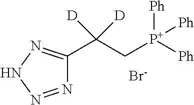 [SMILES: [2H]C([2H])(C[P+](c1ccccc1)(c1ccccc1)c1ccccc1)c1nn[nH]n1 ; [Br-]]

### Step 1—Preparation of 3-Hydroxy(2,2-d₂)-propanenitrile (A06-1)

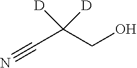

See Steven J. Gould, Junning Lee, John Wityak, “Biosynthesis of streptothricin F7. The fate of the arginine hydrogens” Bioorg. Chem., 1991, 19 (3), 333-350. To a solution of sodium cyanide (1.92 g, 39.18 mmol) in deuterium oxide (150 mL) and methanol-d (90 mL, 99.5% D, Sigma-Aldrich, Catalog #151939) was added 3-hydroxypropionitrile (30 g, 422 mmol), and the resulting solution was heated at reflux for 22-24 h. The solution was then concentrated to 50-60 mL, saturated with solid sodium chloride (˜75 g) and extracted with EtOAc (5×500 mL). The organic layer was dried over sodium sulfate, filtered, and concentrated to obtain A06-1 (22 g, 73% yield, 93% deuteration).

### Step 2—Preparation of 2-(2H-1,2,3,4-Tetrazol-5-yl)(2,2-d₂)ethan-1-ol (A06-2)

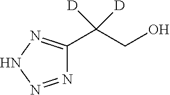

To a mixture of sodium azide (16.08 g, 247.2 mmol) in toluene (80 mL) was added tributyltin chloride (72 mL, 265.32 mmol) and the mixture was stirred for 15 min at room temperature. A06-1 (24 g, 328.8 mmol) was then added and the resulting mixture was heated at 90-95° C. for 20-24 h. The reaction is quenched with 6 M HCl (120 mL, 720 mmol) and the reaction mixture was further heated to reflux for 12 h. The mixture was slowly cooled to room temperature and the layers were separated. The aqueous layer was extracted with 1,2-dichloroethane (4×75 mL) and EtOAc (150 mL). Then the aqueous layer was concentrated under vacuum to obtain a thick residue. This residue was treated with ethanol/IPA (1:1, 200 mL) and filtered to remove inorganic salts (NaCl). The filtrate was concentrated under vacuum and further dried under vacuum over phosphorus pentoxide (P₂O₅) for 24-36 h at room temperature. The residue was treated with ACN (200 mL) at room temperature for 24-36 h with stirring which affords a nice filterable solid. The solid was filtered and treated again with ACN (200 mL) at room temperature for 24 h with stirring. Filtration of the slurry provided the solid, which was further dried under vacuum at room temperature for 24-48 h to obtain the desired product A06-2 (14.23 g, 46% yield, compound purity >99% by HPLC, 93% deuteration by ¹H NMR).

### Step 3—Preparation of Triphenyl[2-(2H-1,2,3,4-tetrazol-5-yl)(2,2-d₂)ethyl]phosphonium bromide (A06-3)

The mixture of alcohol A06-2 (4.6 g, 39.3 mmol) and triphenylphosphine hydrobromide (Ph₃P·HBr, 13.8 g, 40.2 mmol), were ground into fine powder using a mortar and pestle, and poured into a pre-heated (120° C.) reactor which had been purged with nitrogen. The mixture was stirred at 122-127° C. for 3-3.5 h. After completion of the reaction, the mixture was cooled to 100° C. and 1,2-dichloroethane (DCE, 20 mL) was added to the reactor. Stirring was continued at 120° C. for 30-40 min and then cooled to 40° C. After adding ACN (10 mL) the mixture was slowly cooled to room temperature and stirred for 16-18 h. The slurry was filtered and the wet cake was washed with ACN (2×10 mL). The solid obtained was further dried under vacuum at room temperature for 10-12 h to obtain the product as a white solid. The mother liquor was concentrated to ˜60 mL and ACN (15 mL) was added with small amount of salt seed crystals. The mixture was stirred at room temperature for 18-24 h. The slurry was filtrated and the wet cake was washed with ACN (2×10 mL) and dried under vacuum at room temperature for 24-48 h. The first and second crops of solids (total 20.5 g) were combined and heated in ACN (100 mL) at 65-70° C. for 20-30 min and stirred at room temperature for 14-16 h. Filtration provided a solid that was dried under vacuum at room temperature for 24-48 h to obtain the desired deuterated Wittig reagent A06-3 (11.5 g, 66% yield, compound purity 96.5% by HPLC, 85% deuteration by ¹H NMR).

### Preparation of C3, C10-Trideuterated A07

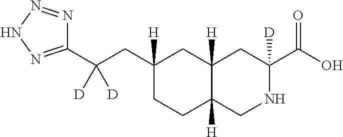 [SMILES: [2H]C([2H])(C[C@H]1CC[C@H]2CN[C@]([2H])(C(=O)O)C[C@H]2C1)c1nn[nH]n1]

Using the same protocols to synthesis A06, Step 11 is modified by adding an additional step after hydrogenation to deuterated the C3-center under basic conditions, as described above for A02.

### Preparation of C1, C3-Trideuterated A08

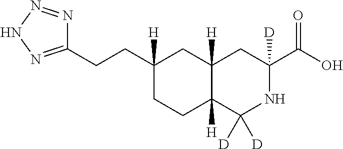 [SMILES: [2H]C1([2H])N[C@]([2H])(C(=O)O)C[C@H]2C[C@@H](CCc3nn[nH]n3)CC[C@H]21]

Using the same protocols to synthesis A03, Step 11 is modified by adding an additional step after hydrogenation to deuterated the C3-center under basic conditions, as described for A02. Alternatively, A08 is made directly from A01 using ruthenium on carbon and deuterium oxide (Alessia Michelotti, et al. “Development and Scale-Up of Stereoretentive α-Deuteration of Amines.” Org. Process Res. Dev., 2017, 21, 1741-1744). A 10-mL round-bottom flask is charged with A01 (1 mmol), Ru/C (5 wt %, 10% w/w), solid sodium hydroxide (3 mmol), and a magnetic stirrer. The flask is capped with a septum and deuterium oxide (2 mL) is added under nitrogen atmosphere. The flask is evacuated and back-filled with hydrogen gas three times and the mixture is stirred at 70° C. under hydrogen atmosphere at balloon pressure. The reaction is monitored by ¹H NMR or MS. Upon completion the solution is cooled to room temperature and filtered through Celite. The filtrate is neutralized with Dowex X-8 [H+] resin (pH of the final solution is between 5 and 6) and then the desired material is eluted from the resin by washing with 25% aqueous ammonium hydroxide (25 mL). The solvent is evaporated under reduced pressure at 45° C. to give trideuterated A08.

### Preparation of C1, C3, C10-Pentadeuterated A09

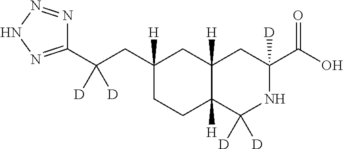 [SMILES: [2H]C([2H])(C[C@H]1CC[C@@H]2[C@H](C1)C[C@@]([2H])(C(=O)O)NC2([2H])[2H])c1nn[nH]n1]

Compound A09 is made directly from A06 using ruthenium on carbon and deuterium oxide (Alessia Michelotti, et al. “Development and Scale-Up of Stereoretentive α-Deuteration of Amines.” Org. Process Res. Dev., 2017, 21, 1741-1744). A 10-mL round-bottom flask is charged with A06 (1 mmol), Ru/C (5 wt %, 10% w/w), solid sodium hydroxide (3 mmol), and a magnetic stirrer. The flask is capped with a septum and deuterium oxide (2 mL) is added under nitrogen atmosphere. The flask is evacuated and back-filled with hydrogen gas three times and the mixture is stirred at 70° C. under hydrogen atmosphere at balloon pressure. The reaction is monitored by ¹H NMR or MS. Upon completion the solution is cooled to room temperature and filtered through Celite. The filtrate is neutralized with Dowex X-8 [H+] resin (pH of the final solution is between 5 and 6) and then the desired material is eluted from the resin by washing with 25% aqueous ammonium hydroxide (25 mL). The solvent is evaporated under reduced pressure at 45° C. to give pentadeuterated A09.

The above compounds of Table 1 include some embodiments of the current disclosure. These examples are not intended to be limiting.

Creating esters from deuterated acids from Table 1, or the parent undeuterated acid A01, may be achieved by preparing carbamate-protected coupling intermediates as illustrated in Table 2. In some embodiments, an example preparation method for carbamate-protected coupling intermediates can include the following method:

### Preparation of Cbz-Protected Intermediate B01

To a slurry of A01 (7.4 g, 26.33 mmol) in THE (60 mL) was added aqueous 2N NaOH (52.5 mL, 105 mmol) at 5-10° C. and stirred for 5-10 min under nitrogen atmosphere. Benzyl chloroformate (7.5 mL, 52.5 mmol) was added slowly at 5-10° C. The mixture was warmed up to room temperature and stirred for 2-3 h (the reaction was monitored by TLC, eluting with ethyl acetate). The mixture was adjusted to pH 3-4 using 3N HCl and diluted with ethyl acetate (200 mL). The layers were separated and the combined organic layers were washed with brine (50 mL), dried over sodium sulfate, and concentrated under vacuum at 40-45° C. The residue was stirred with heptane (100 mL) at room temperature for 16-18 h. The heptane was decanted off and the remaining semi-solid material was dried under high vacuum for to obtain B01 (9.1 g, 83% yield).

Using the above protocol to make B01, starting material A01 can be replaced with the starting materials from Table 1 (e.g., A02-A09) to make alternative Cbz-protected intermediates in Table 2 (e.g., B02-B09).

TABLE 2
Cbz-Protected Core Molecules.

| # | Structure |
|---|---|
| B01 | 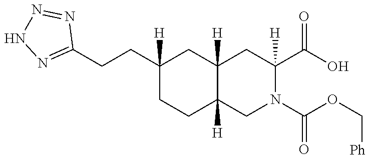 [SMILES: O=C(O)[C@@H]1C[C@H]2C[C@@H](CCc3nn[nH]n3)CC[C@H]2CN1C(=O)OCc1ccccc1] |
| B02 | 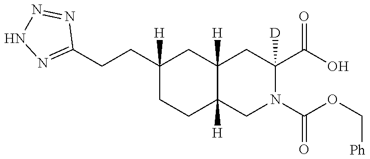 [SMILES: [2H][C@@]1(C(=O)O)C[C@H]2C[C@@H](CCc3nn[nH]n3)CC[C@H]2CN1C(=O)OCc1ccccc1] |
| B03 | 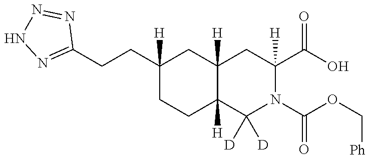 [SMILES: [2H]C1([2H])[C@@H]2CC[C@H](CCc3nn[nH]n3)C[C@@H]2C[C@@H](C(=O)O)N1C(=O)OCc1ccccc1] |
| B04 | 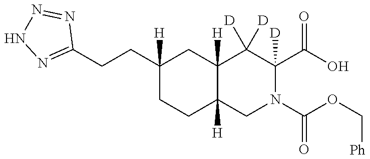 [SMILES: [2H]C1([2H])[C@H]2C[C@@H](CCc3nn[nH]n3)CC[C@H]2CN(C(=O)OCc2ccccc2)[C@]1([2H])C(=O)O] |
| B05 | 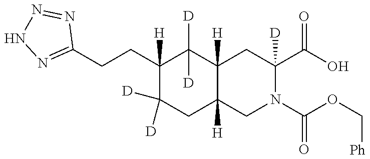 |
| B06 | 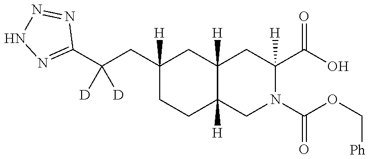 [SMILES: [2H]C([2H])(C[C@H]1CC[C@H]2CN(C(=O)OCc3ccccc3)[C@H](C(=O)O)C[C@H]2C1)c1nn[nH]n1] |
| B07 | 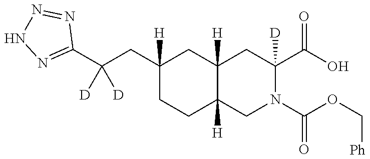 [SMILES: [2H]C([2H])(C[C@H]1CC[C@H]2CN(C(=O)OCc3ccccc3)[C@]([2H])(C(=O)O)C[C@H]2C1)c1nn[nH]n1] |
| B08 | 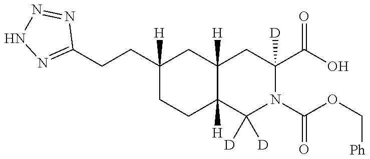 [SMILES: [2H]C1([2H])[C@@H]2CC[C@H](CCc3nn[nH]n3)C[C@@H]2C[C@@]([2H])(C(=O)O)N1C(=O)OCc1ccccc1] |
| B09 | 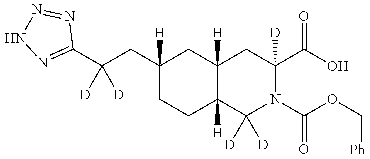 [SMILES: [2H]C([2H])(C[C@H]1CC[C@@H]2[C@H](C1)C[C@@]([2H])(C(=O)O)N(C(=O)OCc1ccccc1)C2([2H])[2H])c1nn[nH]n1] |

Also disclosed is a preparation of alcohol intermediates for attaching to the intermediates of Table 2. These alcohol intermediates may be commercially available or prepared using routine methods known in the art. A non-limiting list of alcohol intermediates is illustrated in Table 3.

TABLE 3
Alcohol Starting Materials.

| # | Structure | [CAS #] | Source and/or Protocol |
|---|---|---|---|
| C001 | 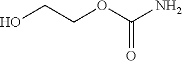 | [5395-01-7] | Enamine Ltd |
| C002 | 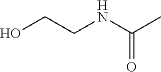 | [142-26-7] | Acros Organics |
| C003 | 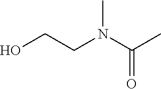 | [15567-95-0] | ChemBridge |
| C004 | 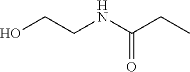 | [18266-55-2] | TCI Chemicals |
| C005 | 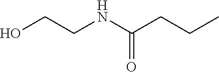 | [23363-91-9] | Ukrorg Synthesis Ltd |
| C006 | 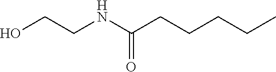 | [7726-06-9] | Ukrorg Synthesis Ltd |
| C007 | 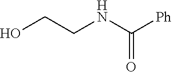 [SMILES: O=C(NCCO)c1ccccc1] | [18838-10-3] | Enamine Ltd |
| C008 | 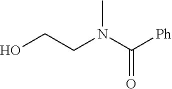 | [57440-15-0] | See Protocol C008 Below |
| C009 | 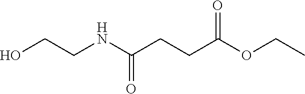 | | See Protocol C009 Below |
| C010 | 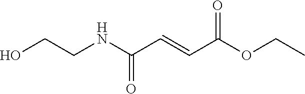 | | See Protocol C010 Below |
| C011 | 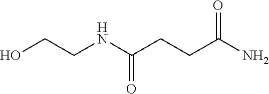 [SMILES: NC(=O)CCC(=O)NCCO] | | See Protocol C011 Below |
| C012 | 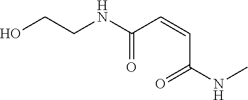 | | See Protocol C012 Below |
| C013 | 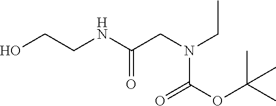 [SMILES: CCN(CC(=O)NCCO)C(=O)OC(C)(C)C] | | See Protocol C013 Below |
| C014 | 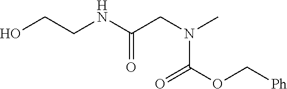 [SMILES: CN(CC(=O)NCCO)C(=O)OCc1ccccc1] | | See Protocol C014 Below |
| C015 | 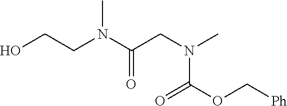 [SMILES: CN(CCO)C(=O)CN(C)C(=O)OCc1ccccc1] | | See Protocol C014 Below |
| C016 | 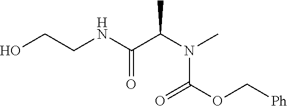 [SMILES: C[C@H](C(=O)NCCO)N(C)C(=O)OCc1ccccc1] | | See Protocol C014 Below |
| C017 | 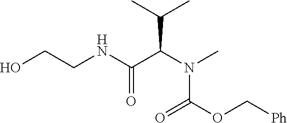 [SMILES: CC(C)[C@H](C(=O)NCCO)N(C)C(=O)OCc1ccccc1] | | See Protocol C014 Below |
| C018 | 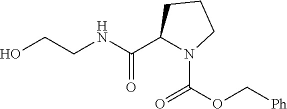 [SMILES: O=C(NCCO)[C@H]1CCCN1C(=O)OCc1ccccc1] | | See Protocol C014 Below |
| C019 | 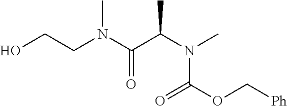 [SMILES: C[C@H](C(=O)N(C)CCO)N(C)C(=O)OCc1ccccc1] | | See Protocol C014 Below |
| C020 | 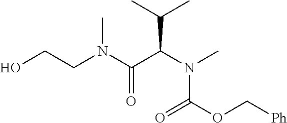 [SMILES: CC(C)[C@H](C(=O)N(C)CCO)N(C)C(=O)OCc1ccccc1] | | See Protocol C014 Below |
| C021 | 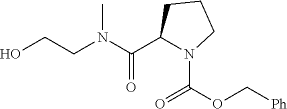 [SMILES: CN(CCO)C(=O)[C@H]1CCCN1C(=O)OCc1ccccc1] | | See Protocol C014 Below |
| C022 | 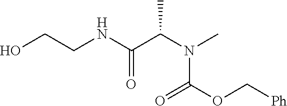 [SMILES: C[C@@H](C(=O)NCCO)N(C)C(=O)OCc1ccccc1] | | See Protocol C014 Below |
| C023 | 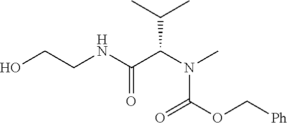 [SMILES: CC(C)[C@@H](C(=O)NCCO)N(C)C(=O)OCc1ccccc1] | | See Protocol C014 Below |
| C024 | 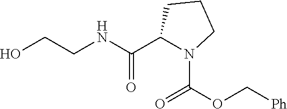 [SMILES: O=C(NCCO)[C@@H]1CCCN1C(=O)OCc1ccccc1] | [95730-14-6] | See Protocol C014 Below |
| C025 | 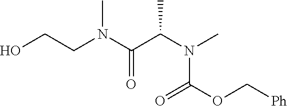 [SMILES: C[C@@H](C(=O)N(C)CCO)N(C)C(=O)OCc1ccccc1] | | See Protocol C014 Below |
| C026 | 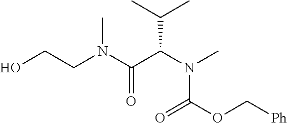 [SMILES: CC(C)[C@@H](C(=O)N(C)CCO)N(C)C(=O)OCc1ccccc1] | | See Protocol C014 Below |
| C027 | 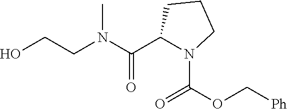 [SMILES: CN(CCO)C(=O)[C@@H]1CCCN1C(=O)OCc1ccccc1] | | See Protocol C014 Below |
| C028 | 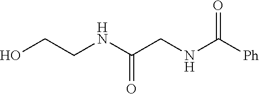 [SMILES: O=C(CNC(=O)c1ccccc1)NCCO] | [72085-01-9] | Matrix Scientific |
| C029 | 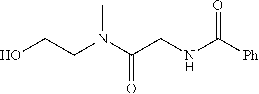 [SMILES: CN(CCO)C(=O)CNC(=O)c1ccccc1] | [1179828-01-3] | See Protocol C014 Below |
| C030 | 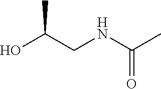 | [1315051-17-2] | See Protocol C031 Below |
| C031 | 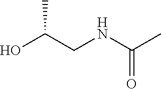 | [116698-06-7] | See Protocol C031 Below |
| C032 | 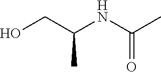 | [35593-62-5] | See Protocol C032 Below |
| C033 | 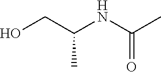 | [145842-51-9] | See Protocol C032 Below |
| C034 | 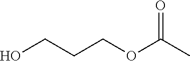 | [36678-05-4] | See Protocol C035 Below |
| C035 | 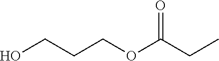 | [155299-59-5] | See Protocol C035 Below |
| C036 | 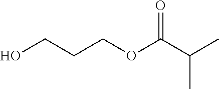 | [126775-65-3] | See Protocol C035 Below |
| C037 | 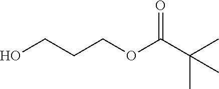 | [267227-69-0] | See Protocol C035 Below |
| C037a | 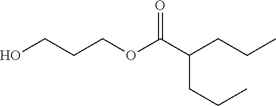 | | See Protocol C035 Below |
| C038 | 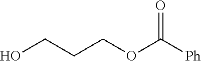 [SMILES: O=C(OCCCO)c1ccccc1] | [6946-99-2] | Manchester Organics and See Protocol C038 Below |
| C039 | 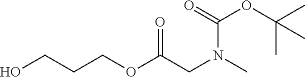 [SMILES: CN(CC(=O)OCCCO)C(=O)OC(C)(C)C] | | See Protocol C039 Below |
| C040 | 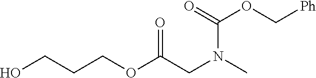 [SMILES: CN(CC(=O)OCCCO)C(=O)OCc1ccccc1] | | See Protocol C039 Below |
| C041 | 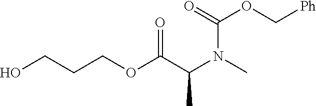 [SMILES: C[C@@H](C(=O)OCCCO)N(C)C(=O)OCc1ccccc1] | | See Protocol C039 Below |
| C042 | 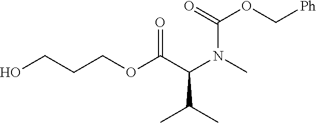 [SMILES: CC(C)[C@@H](C(=O)OCCCO)N(C)C(=O)OCc1ccccc1] | | See Protocol C039 Below |
| C043 | 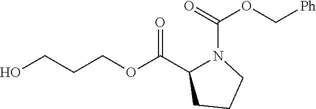 [SMILES: O=C(OCCCO)[C@@H]1CCCN1C(=O)OCc1ccccc1] | | See Protocol C039 Below |
| C044 | 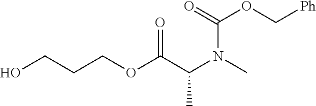 [SMILES: C[C@H](C(=O)OCCCO)N(C)C(=O)OCc1ccccc1] | | See Protocol C039 Below |
| C045 | 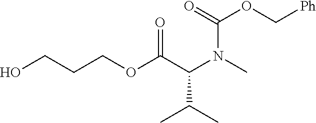 [SMILES: CC(C)[C@H](C(=O)OCCCO)N(C)C(=O)OCc1ccccc1] | | See Protocol C039 Below |
| C046 | 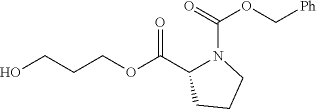 [SMILES: O=C(OCCCO)[C@H]1CCCN1C(=O)OCc1ccccc1] | | See Protocol C039 Below |
| C047 | 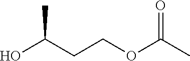 | [128402-22-2] | See Protocol C047 Below |
| C048 | 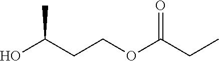 | | See Protocol C047 Below |
| C049 | 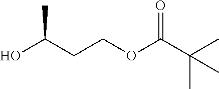 | | See Protocol C047 Below |
| C049a | 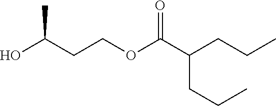 | | See Protocol C047 Below |
| C050 | 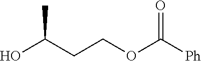 | [82659-86-7] | See Protocol C047 Below |
| C051 | 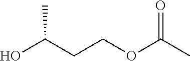 | [342641-02-5] | See Protocol C051 Below |
| C052 | 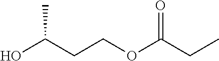 | [138833-38-2] | See Protocol C051 Below |
| C053 | 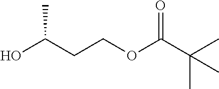 | [432506-90-6] | See Protocol C051 Below |
| C053a | 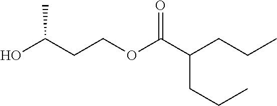 | | See Protocol C051 Below |
| C054 | 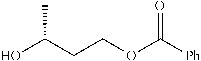 | [82598-19-4] | See Protocol C051 Below |
| C055 | 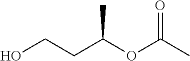 | | See Protocol C055 Below |
| C056 | 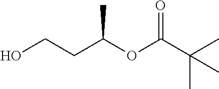 | [1825332-62-4] | See Protocol C056 Below |
| C056a | 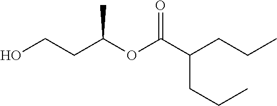 | | See Protocol C056 Below |
| C057 | 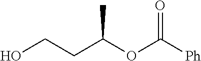 | | See Protocol C055 Below |
| C058 | 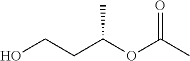 | [128402-23-3] | See Protocol C055 Below |
| C059 | 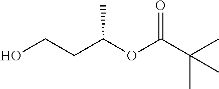 | | See Protocol C056 Below |
| C059a | 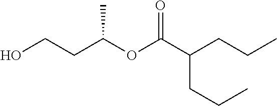 | | See Protocol C056 Below |
| C060 | 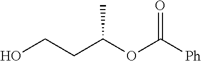 | | See Protocol C055 Below |
| C061 | 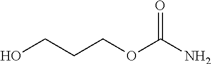 | [60653-88-5] | See Protocol C063 Below |
| C062 | 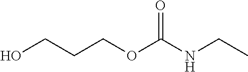 | [1438396-35-0] | See Protocol C063 Below |
| C063 | 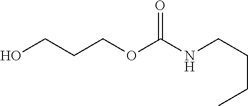 | [117241-48-2] | See Protocol C063 Below |
| C064 | 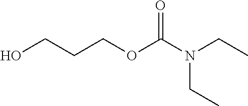 | [243963-73-7] | See Protocol C064 Below |
| C065 | 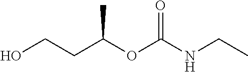 | | See Protocol C065 Below |
| C066 | 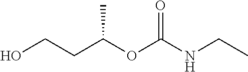 | | See Protocol C065 Below |
| C067 | 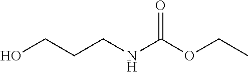 | [74877-62-6] | See Protocol C067 Below |
| C068 | 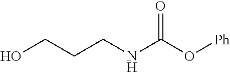 [SMILES: O=C(NCCCO)Oc1ccccc1] | [65935-02-6] | See Protocol C067 Below |
| C069 | 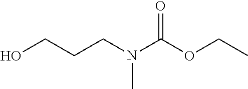 | [1690434-02-6] | See Protocol C067 Below |
| C070 | 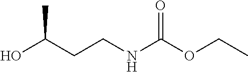 | [1841098-69-8] | See Protocol C067 Below |
| C071 | 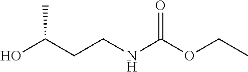 | [1841630-61-2] | See Protocol C067 Below |
| C072 | 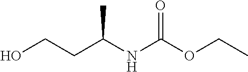 | [1842583-52-1] | See Protocol C067 Below |
| C073 | 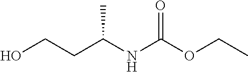 | [1867035-01-5] | See Protocol C067 Below |
| C074 | 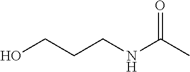 | [10601-73-7] | ChemBridge and See Protocol C074 Below |
| C075 | 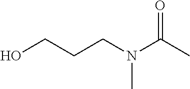 | [118671-64-0] | See Protocol C075 Below |
| C076 | 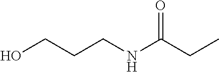 | [10601-74-8] | See Protocol C074 Below |
| C077 | 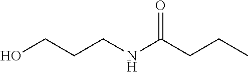 | [137160-71-5] | See Protocol C074 Below |
| C078 | 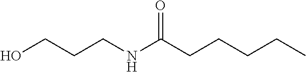 | [68475-65-0] | See Protocol C074 Below |
| C079 | 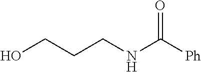 [SMILES: O=C(NCCCO)c1ccccc1] | [13609-09-1] | See Protocol C079 Below |
| C080 | 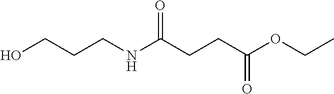 | [1155050-12-6] | See Protocol C009 Below |
| C081 | 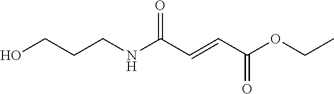 | | See Protocol C010 Below |
| C082 | 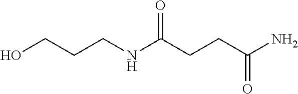 [SMILES: NC(=O)CCC(=O)NCCCO] | [1181455-01-5] | See Protocol C011 Below |
| C083 | 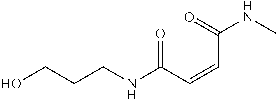 | | See Protocol C012 Below |
| C084 | 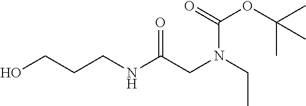 [SMILES: CCN(CC(=O)NCCCO)C(=O)OC(C)(C)C] | | See Protocol C013 Below |
| C085 | 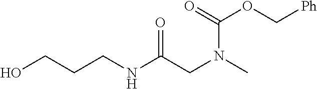 [SMILES: CN(CC(=O)NCCCO)C(=O)OCc1ccccc1] | | See Protocol C014 Below |
| C086 | 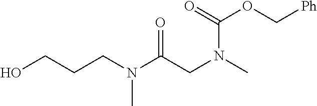 [SMILES: CN(CCCO)C(=O)CN(C)C(=O)OCc1ccccc1] | | See Protocol C014 Below |
| C087 | 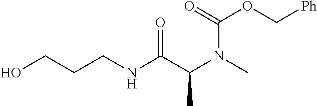 [SMILES: C[C@@H](C(=O)NCCCO)N(C)C(=O)OCc1ccccc1] | | See Protocol C014 Below |
| C088 | 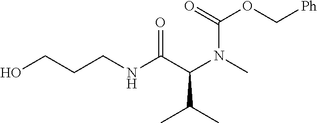 [SMILES: CC(C)[C@@H](C(=O)NCCCO)N(C)C(=O)OCc1ccccc1] | | See Protocol C014 Below |
| C089 | 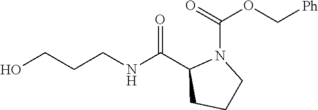 [SMILES: O=C(NCCCO)[C@@H]1CCCN1C(=O)OCc1ccccc1] | | See Protocol C014 Below |
| C090 | 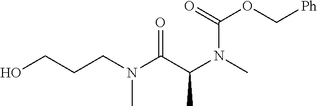 [SMILES: C[C@@H](C(=O)N(C)CCCO)N(C)C(=O)OCc1ccccc1] | | See Protocol C014 Below |
| C091 | 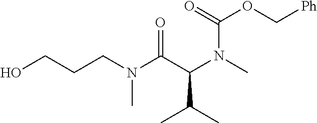 [SMILES: CC(C)[C@@H](C(=O)N(C)CCCO)N(C)C(=O)OCc1ccccc1] | | See Protocol C014 Below |
| C092 | 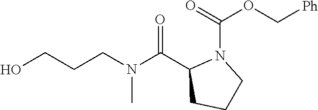 [SMILES: CN(CCCO)C(=O)[C@@H]1CCCN1C(=O)OCc1ccccc1] | | See Protocol C014 Below |
| C093 | 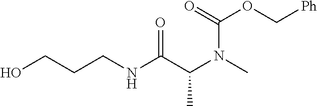 [SMILES: C[C@H](C(=O)NCCCO)N(C)C(=O)OCc1ccccc1] | | See Protocol C014 Below |
| C094 | 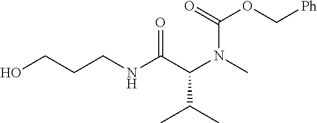 [SMILES: CC(C)[C@H](C(=O)NCCCO)N(C)C(=O)OCc1ccccc1] | | See Protocol C014 Below |
| C095 | 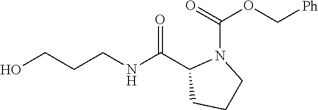 [SMILES: O=C(NCCCO)[C@H]1CCCN1C(=O)OCc1ccccc1] | | See Protocol C014 Below |
| C096 | 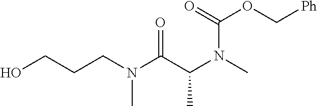 [SMILES: C[C@H](C(=O)N(C)CCCO)N(C)C(=O)OCc1ccccc1] | | See Protocol C014 Below |
| C097 | 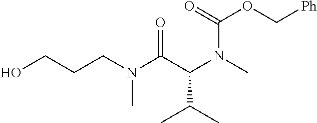 [SMILES: CC(C)[C@H](C(=O)N(C)CCCO)N(C)C(=O)OCc1ccccc1] | | See Protocol C014 Below |
| C098 | 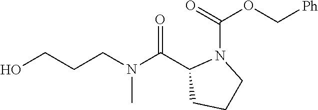 [SMILES: CN(CCCO)C(=O)[C@H]1CCCN1C(=O)OCc1ccccc1] | | See Protocol C014 Below |
| C099 | 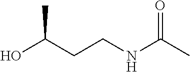 | [1841088-45-6] | See Protocol C074 Below |
| C100 | 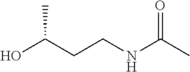 | [1841134-19-7] | See Protocol C074 Below |
| C101 | 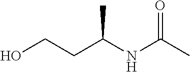 | [1841458-79-4] | See Protocol C074 Below |
| C102 | 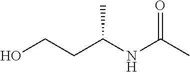 | [1841120-59-9] | See Protocol C074 Below |
| C103 | 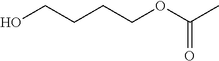 | [35435-68-8] | See Protocol C103 Below |
| C104 | 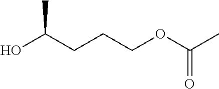 | [131864-74-9] | See Protocol C104 Below |
| C105 | 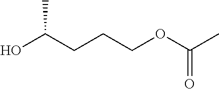 | [1187785-15-4] | See Protocol C105 Below |
| C106 | 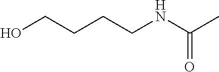 | [75694-83-6] | See Protocol C074 Below |
| C107 | 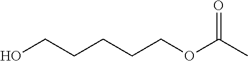 | [68750-23-2] | See Protocol C107 Below |
| C108 | 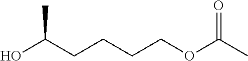 | [2102554-31-2] | See Protocol C108 Below |
| C109 | 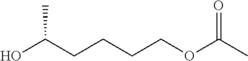 | | See Protocol C108 Below |
| C110 | 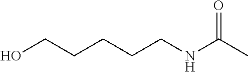 | [40447-15-2] | See Protocol C110 Below |
| C111 | 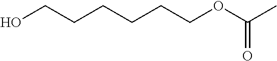 | [68750-24-3] | See Protocol C103 Below |
| C112 | 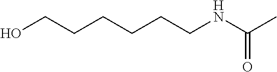 | [23363-92-0] | See Protocol C074 Below |
| C113 | 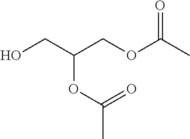 | [102-62-5] | TRC Canada |
| C114 | 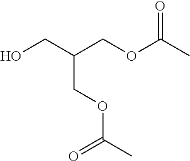 | [138435-86-6] | See Protocol C114 Below |
| C115 | 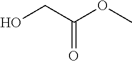 | [96-35-5] | Sigma-Aldrich |
| C116 | 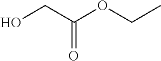 | [623-50-7] | Sigma-Aldrich |
| C117 | 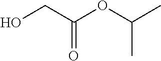 | [623-61-0] | Combi-Blocks |
| C118 | 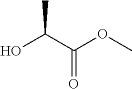 | [27871-49-4] | Sigma-Aldrich |
| C119 | 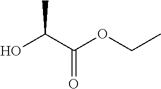 | [687-47-8] | Sigma-Aldrich |
| C120 | 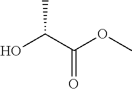 | [17392-83-5] | Sigma-Aldrich |
| C121 | 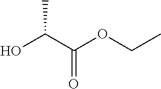 | [7699-00-5] | Sigma-Aldrich |
| C122 | 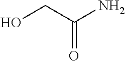 | [598-42-5] | Sigma-Aldrich |
| C123 | 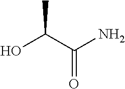 | [89673-71-2] | Sigma-Aldrich |
| C124 | 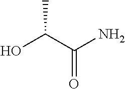 | [598-81-2] | Sigma-Aldrich |
| C125 | 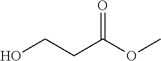 | [6149-41-3] | Enamine Ltd |
| C126 | 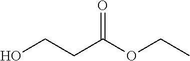 | [623-72-3] | Sigma-Aldrich |
| C127 | 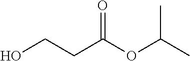 | [84098-45-3] | See Protocol C127 Below |
| C128 | 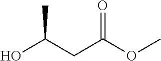 | [53562-86-0] | Sigma-Aldrich |
| C129 | 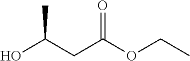 | [56816-01-4] | Sigma-Aldrich |
| C130 | 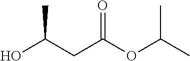 | [111970-49-1] | See Protocol C130 Below |
| C131 | 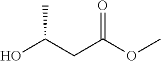 | [3976-69-0] | Sigma-Aldrich |
| C132 | 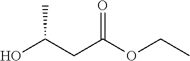 | [24915-95-5] | Sigma-Aldrich |
| C133 | 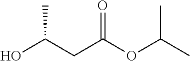 | [87128-49-2] | See Protocol C130 Below |
| C134 | 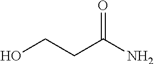 | 2651-43-6] | Enamine Ltd |
| C135 | 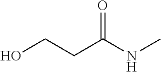 | [6830-81-5] | See Protocol C139 Below |
| C136 | 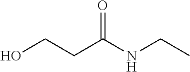 | [38052-79-8] | Enamine Ltd |
| C137 | 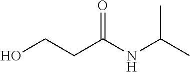 | [7399-84-0] | Enamine Ltd |
| C138 | 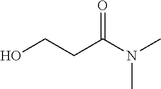 | [29164-29-2] | Enamine Ltd |
| C139 | 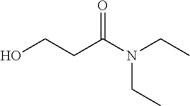 | [64558-46-9] | See Protocol C139 Below |
| C140 | 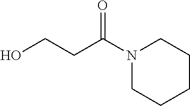 [SMILES: O=C(CCO)N1CCCCC1] | [86452-58-6] | See Protocol C139 Below |
| C141 | 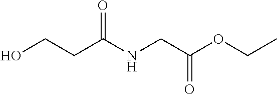 | [2121102-72-3] | See Protocol C141 Below |
| C142 | 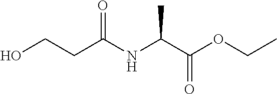 | | See Protocol C141 Below |
| C143 | 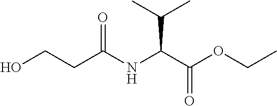 | | See Protocol C141 Below |
| C144 | 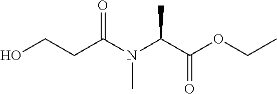 | | See Protocol C141 Below |
| C145 | 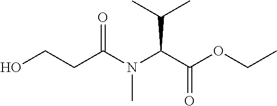 [SMILES: CCOC(=O)[C@H](C(C)C)N(C)C(=O)CCO] | | See Protocol C141 Below |
| C146 | 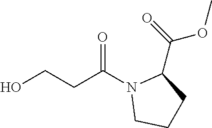 | | See Protocol C141 Below |
| C147 | 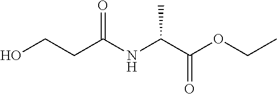 | | See Protocol C141 Below |
| C148 | 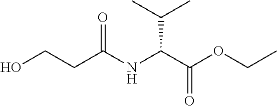 | | See Protocol C141 Below |
| C149 | 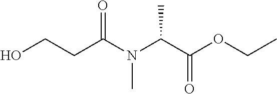 | | See Protocol C141 Below |
| C150 | 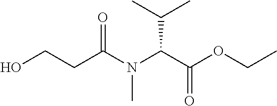 [SMILES: CCOC(=O)[C@@H](C(C)C)N(C)C(=O)CCO] | | See Protocol C141 Below |
| C151 | 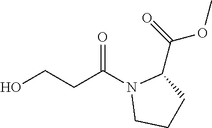 | [1349415-38-8] | See Protocol C141 Below |
| C152 | 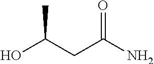 | [197852-03-2] | See Protocol C152 Below |
| C153 | 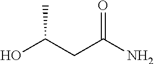 | [223540-10-1] | See Protocol C153 Below |
| C154 | 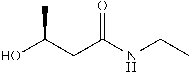 | [118988-13-9] | See Protocol C154 Below |
| C155 | 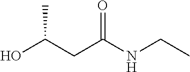 | [1315000-24-8] | See Protocol C154 Below |
| C156 | 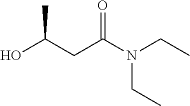 | [884594-11-0] | See Protocol C156 Below |
| C157 | 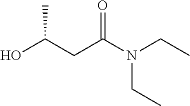 | [884594-12-1] | See Protocol C156 Below |
| C158 | 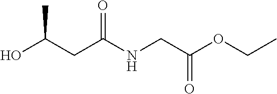 | | See Protocol C158 Below |
| C159 | 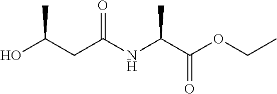 | | See Protocol C158 Below |
| C160 | 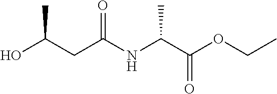 | | See Protocol C158 Below |
| C161 | 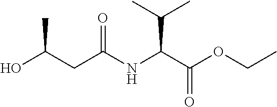 [SMILES: CCOC(=O)[C@@H](NC(=O)C[C@H](C)O)C(C)C] | | See Protocol C158 Below |
| C162 | 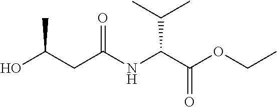 [SMILES: CCOC(=O)[C@H](NC(=O)C[C@H](C)O)C(C)C] | | See Protocol C158 Below |
| C163 | 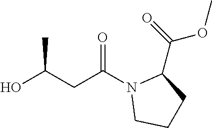 | | See Protocol C158 Below |
| C164 | 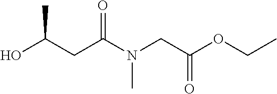 | | See Protocol C158 Below |
| C165 | 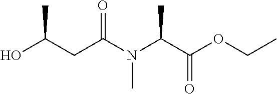 | | See Protocol C158 Below |
| C166 | 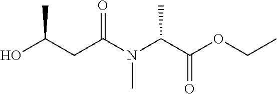 | | See Protocol C158 Below |
| C167 | 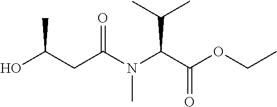 [SMILES: CCOC(=O)[C@H](C(C)C)N(C)C(=O)C[C@H](C)O] | | See Protocol C158 Below |
| C168 | 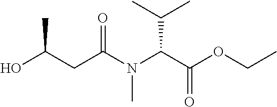 [SMILES: CCOC(=O)[C@@H](C(C)C)N(C)C(=O)C[C@H](C)O] | | See Protocol C158 Below |
| C169 | 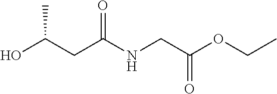 | | See Protocol C158 Below |
| C170 | 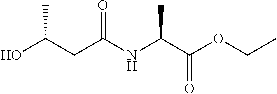 | | See Protocol C158 Below |
| C171 | 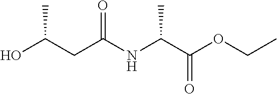 | | See Protocol C158 Below |
| C172 | 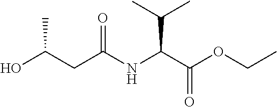 [SMILES: CCOC(=O)[C@@H](NC(=O)C[C@@H](C)O)C(C)C] | | See Protocol C158 Below |
| C173 | 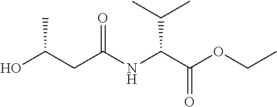 [SMILES: CCOC(=O)[C@H](NC(=O)C[C@@H](C)O)C(C)C] | | See Protocol C158 Below |
| C174 | 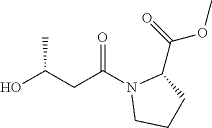 | | See Protocol C158 Below |
| C175 | 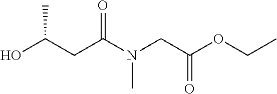 | | See Protocol C158 Below |
| C176 | 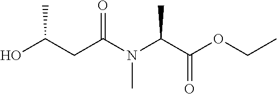 | | See Protocol C158 Below |
| C177 | 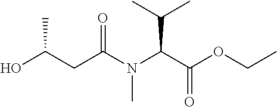 [SMILES: CCOC(=O)[C@H](C(C)C)N(C)C(=O)C[C@@H](C)O] | | See Protocol C158 Below |
| C178 | 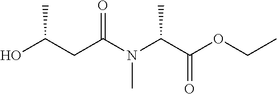 | | See Protocol C158 Below |
| C179 | 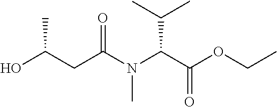 [SMILES: CCOC(=O)[C@@H](C(C)C)N(C)C(=O)C[C@@H](C)O] | | See Protocol C158 Below |
| C180 | 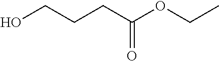 | [999-10-0] | Matrix Scientific |
| C181 | 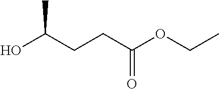 | [112789-84-1] | See Protocol C108 Below |
| C182 | 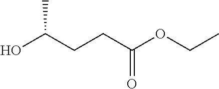 | [99631-16-0] | See Protocol C108 Below |
| C183 | 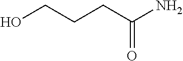 | [927-60-6] | See Protocol C183 Below |
| C184 | 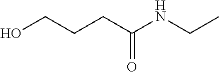 | [42042-64-8] | See Protocol C184 Below |
| C185 | 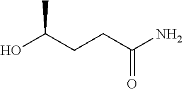 | | See Protocol C183 or C189 Below |
| C186 | 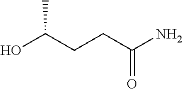 | | See Protocol C183 or C189 Below |
| C187 | 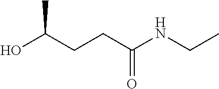 | | See Protocol C184 Below |
| C188 | 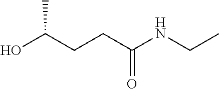 | | See Protocol C184 Below |
| C189 | 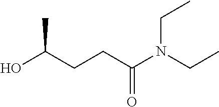 | | See Protocol C189 Below |
| C190 | 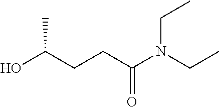 | | See Protocol C189 Below |
| C191 | 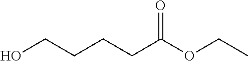 | [13107-53-4] | Matrix Scientific |
| C192 | 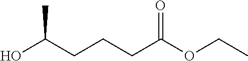 | [118627-53-5] | See Protocol C192 Below |
| C193 | 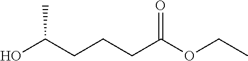 | [195875-25-3] | See Protocol C192 Below |
| C194 | 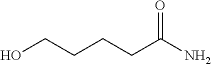 | [29686-12-2] | Sigma-Aldrich |
| C195 | 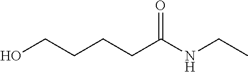 | [1226291-91-3] | See Protocol C184 or C197 Below |
| C196 | 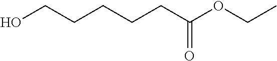 | [5299-60-5] | Sigma-Aldrich |
| C197 | 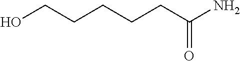 | [4547-52-8] | See Protocol C197 Below |
| C198 | 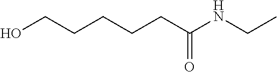 | [57021-66-6] | See Protocol C197 Below |
| C199 | 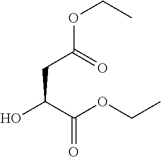 | [691-84-9] | TCI Chemicals |
| C200 | 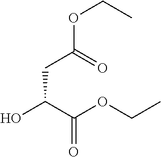 | [7554-28-1] | TCI Chemicals |
| C201 | 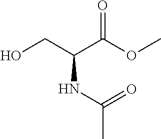 | [54322-41-7] | See Protocol C202 Below |
| C202 | 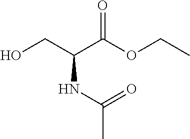 | [2743-56-8] | See Protocol C202 Below |
| C203 | 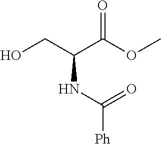 [SMILES: COC(=O)[C@H](CO)NC(=O)c1ccccc1] | [20989-42-8] | See Protocol C204 Below |
| C204 | 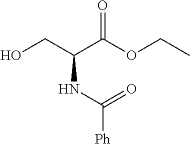 [SMILES: CCOC(=O)[C@H](CO)NC(=O)c1ccccc1] | [6332-41-8] | See Protocol C204 Below |
| C205 | 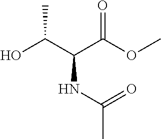 | [2458-78-8] | See Protocol C202 Below |
| C206 | 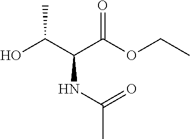 | [98633-04-6] | See Protocol C206 Below |
| C207 | 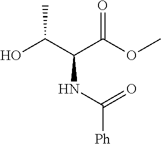 [SMILES: COC(=O)[C@@H](NC(=O)c1ccccc1)[C@@H](C)O] | [79893-89-3] | Sigma-Aldrich and See Protocol C207 Below |
| C208 | 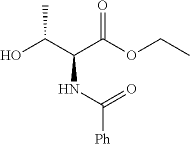 [SMILES: CCOC(=O)[C@@H](NC(=O)c1ccccc1)[C@@H](C)O] | [23161-25-3] | See Protocol C204 Below |
| C209 | 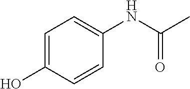 | [103-90-2] | Sigma-Aldrich |
| C210 | 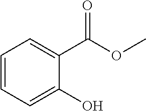 | [119-36-8] | Sigma-Aldrich |
| C211 | 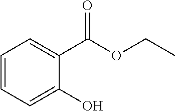 | [118-61-6] | Sigma-Aldrich |
| C212 | 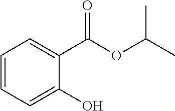 | [607-85-2] | TCI Chemicals |
| C213 | 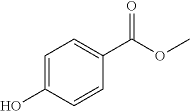 | [99-76-3] | Sigma-Aldrich |
| C214 | 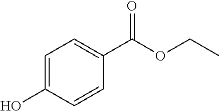 | [120-47-8] | Sigma-Aldrich |
| C215 | 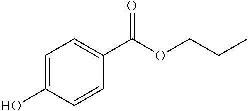 | [94-13-3] | Sigma-Aldrich |
| C216 | 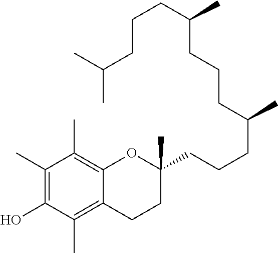 [SMILES: Cc1c(C)c2c(c(C)c1O)CC[C@@](C)(CCC[C@H](C)CCC[C@H](C)CCCC(C)C)O2] | [59-02-9] | Sigma-Aldrich |
| C217 | 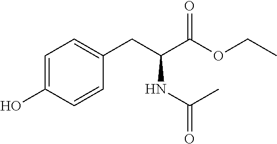 [SMILES: CCOC(=O)[C@H](Cc1ccc(O)cc1)NC(C)=O] | [36546-50-6] | Sigma-Aldrich |

### Preparation of Alcohol Intermediates:

The alcohol intermediates of Table 3 may be prepared using the following reactions:

### Preparation of N-(2-Hydroxyethyl)-N-methylbenzamide (C008)

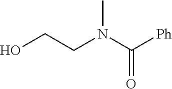

Compound C008 is prepared according to Nishii, Yuji et al. Eur. J. Org. Chem., 2017, 34, 5010-5014 and Nishii, Yuji et al. Synlett, 2015, 26 (13), 1831-1834.

### Preparation of Ethyl 3-[(2-Hydroxyethyl)carbamoyl]propanoate (C009)

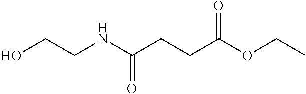

To a solution of 2-aminoethanol (1.0 mmol) in dichloromethane (5 mL) is added triethylamine (1.0 mmol) and the mixture is cooled to 0° C. After stirring at 0° C. for 10 min ethyl 4-chloro-4-oxobutanoate (1.0 mmol) is added slowly to the solution at 0° C. and the mixture is stirred at room temperature for 1 h. The reaction is diluted with water (50 mL), and extracted with dichloromethane (2×50 mL). The dichloromethane layers are washed with 0.1 N HCl (10 mL), water (50 mL), brine (25 mL), dried over sodium sulfate, and concentrated under vacuum. The crude material is purified by silica gel flash column chromatography to obtain the desired product C009. Compound C080 is prepared using this same protocol but replacing 2-aminoethanol with 3-aminopropan-1-ol.

### Preparation of Ethyl (2E)-3-[(2-Hydroxyethyl)carbamoyl]prop-2-enoate (C0010)

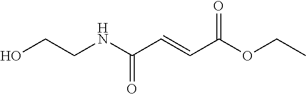

Compound C010 is prepared according to the protocol for C009 but using ethyl (2E)-4-chloro-4-oxobut-2-enoate (CAS #[26367-48-6]) instead of 4-chloro-4-oxobutanoate. Compound C081 is prepared using this same protocol but replacing 2-aminoethanol with 3-aminopropan-1-ol.

### Preparation of N-(2-Hydroxyethyl)butanediamide (C011)

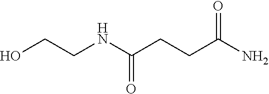 [SMILES: NC(=O)CCC(=O)NCCO]

To a solution of 2-aminoethanol (1.0 mmol) and 3-carbamoylpropanoic acid (1 mmol, CAS #[638-32-4]) in dichloromethane (5 mL) is added triethylamine (2.0 mmol) and the mixture is cooled to 0° C. After stirring at 0° C. for 10 min, 1-(3-dimethylaminopropyl)-3-ethylcarbodiimide hydrochloride (EDC, 1.0 mmol) is added as a solid and the solution is stirred at 0° C. for 2 h and then overnight at room temperature. The reaction is diluted with 0.1 N HCl (10 mL), and extracted with dichloromethane (3×25 mL). The dichloromethane layers are washed with brine (25 mL), dried over sodium sulfate, and concentrated under vacuum. The crude material is purified by silica gel flash column chromatography to obtain the desired product C011. Compound C082 is prepared using this same protocol but replacing 2-aminoethanol with 3-aminopropan-1-ol.

### Preparation of (2Z)—N′-(2-Hydroxyethyl)-N-methylbut-2-enediamide (C012)

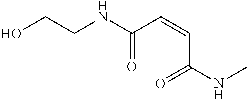

Compound C012 is prepared according to the protocol for C011 but using N-methylmaleamic acid (CAS #[6936-48-7]) instead of 3-carbamoylpropanoic acid. Compound C083 is prepared using this same protocol but replacing 2-aminoethanol with 3-aminopropan-1-ol.

### Preparation of tert-Butyl N-ethyl-N-{[(2-hydroxyethyl)carbamoyl]methyl}carbamate (C013)

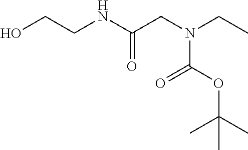 [SMILES: CCN(CC(=O)NCCO)C(=O)OC(C)(C)C]

To a solution of 2-aminoethanol (1.0 mmol) and 2-{[(tert-butoxy)carbonyl](ethyl)amino}acetic acid (1 mmol, CAS #[149794-10-5]) in dichloromethane (5 mL) is added triethylamine (2.0 mmol) and the mixture is cooled to 0° C. After stirring at 0° C. for 10 min, 1-(3-dimethylaminopropyl)-3-ethylcarbodiimide hydrochloride (1.0 mmol) is added as a solid and the solution is stirred at 0° C. for 2 h and then overnight at room temperature. The reaction is diluted with 0.1 N HCl (10 mL), and extracted with dichloromethane (3×25 mL). The dichloromethane layers are washed with brine (25 mL), dried over sodium sulfate, and concentrated under vacuum. The crude material is purified by silica gel flash column chromatography to obtain the desired product C013. After coupling to the active core molecules, the Boc-group is removed using 1:4 triflouroacetic acid/dichloromethane as the final step. Compound C084 is prepared using this same protocol but replacing 2-aminoethanol with 3-aminopropan-1-ol.

### Preparation of Benzyl N-{[(2-hydroxyethyl)carbamoyl]methyl}-N-methylcarbamate (C014)

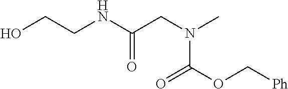 [SMILES: CN(CC(=O)NCCO)C(=O)OCc1ccccc1]

To a solution of 2-aminoethanol (1.0 mmol) and 2-{[(benzyloxy)carbonyl](methyl)amino}acetic acid (1 mmol, N-carbobenzoxy-N-methylglycine, CAS #[39608-31-6]) in acetonitrile (5 mL) is added diisopropylethylamine (2.0 mmol) and the mixture is cooled to 0° C. After stirring at 0° C. for 10 min, TBTU (1.0 mmol, 2-(1H-benzotriazole-1-yl)-1,1,3,3-tetramethylaminium tetrafluoroborate) is added portion-wise as a solid and the solution is stirred at 0° C. for 2 h and then overnight at room temperature. The solvent is removed under vacuum and the residue is dissolved in ethyl acetate (25 mL). The mixture is washed with 1 N HCl (2×25 mL), saturated aqueous sodium bicarbonate (2×25 mL) and brine (25 mL). The organic layer is dried over sodium sulfate, and concentrated under vacuum. The crude material is purified by silica gel flash column chromatography to obtain the desired product C014. Alternatively, amines are coupled to Cbz-protected amino acids by making mixed anhydrides as demonstrated by Thomasset, Amelia et al. Synthesis, 2014, 46 (2), 242-250. Compounds C015-C027 are prepared using this same protocol replacing 2-aminoethanol with 2-(methylamino)ethan-1-ol if needed and switching from glycine-based carboxylic acids to alanine-, valine-, and proline-based carboxylic acids accordingly. Compound C029 is prepared by replacing 2-aminoethanol with 2-(methylamino)ethan-1-ol and coupling to hippuric acid (CAS #[495-69-2]). Compounds C085-C098 are prepared using this same protocol replacing 2-aminoethanol with 3-aminopropan-1-ol or 3-(methylamino)propan-1-ol as appropriate and switching from glycine-based carboxylic acids to alanine-, valine-, and proline-based carboxylic acids accordingly.

### Preparation of N-[(2R)-2-Hydroxypropyl]acetamide (C031)

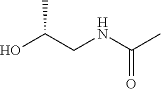

Compound C031 is prepared according to Gotor, Vicente et al. J. Chem. Soc., Chem. Comm., 1988, (14), 957-958. Compound C030 is prepared using this same protocol but replacing (2R)-1-aminopropan-2-ol with (2S)-1-aminopropan-2-ol.

### Preparation of N-[(2S)-1-Hydroxypropan-2-yl]acetamide (C032)

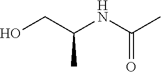

Compound C032 is prepared according to Bartolucci, Silvia et al. Tetrahedron 2016, 72 (18), 2233-2238. Compound C033 is prepared using this same protocol but replacing (2S)-2-aminopropan-1-ol with (2R)-2-aminopropan-1-ol.

### Preparation of 3-Hydroxypropyl Propionate (C035)

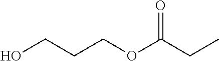

See Ishihara, k. et al. J. Org. Chem., 1993, 58, 3791-3793 & Ibe, Kouta et al. Tetrahedron Lett., 2014, 55 (51), 7039-7042 for a related protocol. To a solution of 3-benzyloxy-1-propanol (2 g, 12.0 mmol) in dichloromethane (15 mL) was added diisopropylethylamine (8.3 mL, 48.12 mmol) at 0° C. After stirring at 0° C. for 10 min propionyl chloride (2.1 mL, 24.06 mmol) was added slowly to the solution at 0° C. and the mixture was stirred at room temperature for 18-20 h. The reaction was diluted with water (50 mL), and the layers were separated. The dichloromethane layer was washed with dilute HCl (10 mL), water (3×50 mL), dried over sodium sulfate, and concentrated under vacuum at 40-45° C. The crude material was used for next reaction without further purification. To a mixture of benzyl-protected derivative in isopropanol-THF (1:1, 20 mL) was added 10% Pd/C (1 g). This mixture was de-gassed using three cycles of vacuum and hydrogen purging. The reaction was stirred under balloon pressure of hydrogen (˜15 psi) for 2-3 h. Then the reaction mixture was filtered through Celite, washed with THE (20 mL) and the filtrate was concentrated under vacuum. The residue was purified by distillation at 100-120° C. under atmospheric pressure to afford C035 (1.5 g, 96% yield). Compounds C034, C036, and C037 are prepared using this same protocol replacing propionyl chloride with acetyl chloride, 2-methylpropanoyl chloride, and trimethylacetyl chloride respectively.

### Preparation of 3-Hydroxypropyl benzoate (C038)

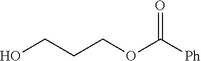 [SMILES: O=C(OCCCO)c1ccccc1]

Compound C038 is prepared according to Chen, Y. and Wang, P. G. Tetrahedron Lett., 2001, 42 (30), 4955-4958 and Wakita, Natsumi and Hara, Shoji Tetrahedron 2010, 66 (40), 7939-7945. Compounds C050, C054, C057, and C060 are prepared using this same protocol replacing 1,2-propanediol with (R)- and (S)-(−)-1,3-butanediols.

### Preparation of 3-Hydroxypropyl 2-{[(tert-butoxy)carbonyl](methyl)amino}acetate (C039)

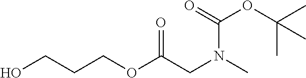 [SMILES: CN(CC(=O)OCCCO)C(=O)OC(C)(C)C]

Compound C039 is prepared according to the protocol for C013 but using ethyl 2-{[(tert-butoxy)carbonyl](methyl)amino}acetic acid (Boc-Sarcosine, CAS #[13734-36-6]) instead of 2-{[(tert-butoxy)carbonyl](ethyl)amino}acetic acid, and 1,3-propanediol instead of 2-aminoethanol. Compounds C040-C046 are prepared using this same protocol replacing glycine-based carboxylic acids to alanine-, valine-, and proline-based carboxylic acids accordingly.

### Preparation of (S)-3-Hydroxybutyl Acetate (C047)

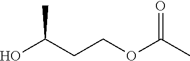

See Ishihara, k. et al. J. Org. Chem., 1993, 58, 3791-3793 & Ibe, Kouta et al. Tetrahedron Lett., 2014, 55 (51), 7039-7042 for a related protocol. To a solution of (S)-(+)-1,3-butanediol (500 mg, 5.55 mmol) and diisopropylethylamine (1.44 g, 11.10 mmol) in dichloromethane (10 mL) was added acetyl chloride (435 mg, 5.55 mmol) at −78° C. drop-wise. The solution was stirred at −78° C. for 3 h. The reaction mixture was then poured into 1M HCl (20 mL), and extracted with ethyl acetate (3×50 mL). The combined organic layers were dried over sodium sulfate and concentrated under vacuum. The crude material was purified by silica gel flash column chromatography (0% to 50% ethyl acetate/heptane) to obtain the desired product C047 (480 mg, 66% yield). Compounds C048-C050 are prepared using this same protocol replacing acetyl chloride with propionyl chloride, trimethylacetyl chloride, and benzoyl chloride, respectively.

### Preparation of (R)-3-Hydroxybutyl Acetate (C051)

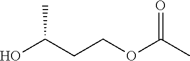

See Ishihara, k. et al. J. Org. Chem., 1993, 58, 3791-3793 & Ibe, Kouta et al. Tetrahedron Lett., 2014, 55 (51), 7039-7042 for a related protocol. To a solution of (R)-(−)-1,3-butanediol (500 mg, 5.55 mmol) and diisopropylethylamine (1.44 g, 11.10 mmol) in dichloromethane (10 mL) was added acetyl chloride (435 mg, 5.55 mmol) at −78° C. drop-wise. The solution was stirred at −78° C. for 3 h. The reaction mixture was then poured into 1M HCl (20 mL), and extracted with ethyl acetate (3×50 mL). The combined organic layers were dried over sodium sulfate and concentrated under vacuum. The crude material was purified by silica gel flash column chromatography (0% to 50% ethyl acetate/heptane) to obtain the desired product C051 (600 mg, 82% yield). Compounds C052-C054 are prepared using this same protocol replacing acetyl chloride with propionyl chloride, trimethylacetyl chloride, and benzoyl chloride, respectively.

### Preparation of (2R)-4-Hydroxybutan-2-yl acetate (C055)

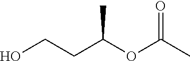

### Step 1: (2R)-4-[(tert-Butyldimethylsilyl)oxy]butan-2-yl acetate (C055-1)

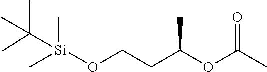 [SMILES: CC(=O)O[C@H](C)CCO[Si](C)(C)C(C)(C)C]

See Gary H. Posner, et al. J. Org. Chem., 1995, 60, 4617-4628 for a related protocol. To a solution of (2R)-4-[(tert-butyldimethylsilyl)oxy]butan-2-ol (1.0 mmol) and diisopropylethylamine (1.1 mmol) in dichloromethane (5 mL) is added 2-methylpropanoyl chloride (1.0 mmol) at −78° C. drop-wise. The solution is stirred and warmed to room temperature over 3 h. The reaction mixture is poured into 0.2 M HCl (20 mL), and extracted with dichloromethane (3×50 mL). The combined organic layers are dried over sodium sulfate and concentrated under vacuum. The crude material is purified by silica gel flash column chromatography to afford (2R)-4-[(tert-butyldimethylsilyl)oxy]butan-2-yl acetate (C055-1).

### Step 2: (2R)-4-Hydroxybutan-2-yl acetate (C055)

To C055-1 (0.5 mmol) is added 5% hydrofluoric acid in acetonitrile (3.0 mL) at room temperature. After 8 min, the reaction mixture is neutralized with aqueous sodium bicarbonate solution, extracted with chloroform (2×10 mL), dried over anhydrous sodium sulfate, concentrated in under vacuum, and purified by silica gel flash column chromatography. The alcohol C055 is used in the next reaction without a complete characterization due to its instability.

Compounds C057, C058, and C060 are prepared using this same protocol replacing acetyl chloride with benzoyl chloride and (2R)-4-[(tert-butyldimethylsilyl)oxy]butan-2-ol with (2S)-4-[(tert-butyldimethylsilyl)oxy]butan-2-ol, accordingly.

### Preparation of (2R)-4-Hydroxybutan-2-yl 2,2-dimethylpropanoate (C056)

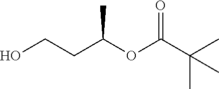

Compound C056 is prepared according to Rival, Nicolas et al. Tetrahedron Lett., 2015, 56 (49), 6823-6826. Compound C059 is prepared using this same protocol replacing (R)-(−)-1,3-butanediol with (S)-(+)-1,3-butanediol.

### Preparation of 3-Hydroxypropyl N-butylcarbamate (C063)

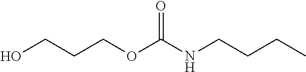

Compound C063 is prepared according to J. Am. Chem. Soc., 2015, 137 (44), 14019-14022. Compounds C061 and C062 are prepared using this same protocol replacing butylamine with ammonia and ethylamine, respectively.

### Preparation of 3-Hydroxypropyl N,N-diethylcarbamate (C064)

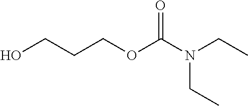

Compound C064 is prepared according to Synth. Commun., 1999, 29 (18), 3207-3214.

### Preparation of (2R)-4-Hydroxybutan-2-yl N-ethylcarbamate (C065)

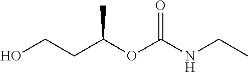

### Step 1: (2R)-4-[(tert-Butyldimethylsilyl)oxy]butan-2-yl N-ethylcarbamate (C065-1)

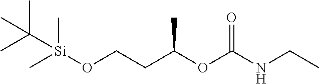 [SMILES: CCNC(=O)O[C@H](C)CCO[Si](C)(C)C(C)(C)C]

See Gary H. Posner, et al. J. Org. Chem., 1995, 60, 4617-4628 for a related protocol. To a solution of (2R)-4-[(tert-butyldimethylsilyl)oxy]butan-2-ol (1.0 mmol) and 4-N,N-dimethylpyridine (1.1 mmol) in dichloromethane (5 mL) is added ethylisocyanate (1.0 mmol) at room temperature. The solution is stirred overnight. The reaction mixture is poured into 0.2 M HCl (20 mL), and extracted with dichloromethane (3×50 mL). The combined organic layers are dried over sodium sulfate and concentrated under vacuum. The crude material is purified by silica gel flash column chromatography to afford (2R)-4-[(tert-butyldimethylsilyl)oxy]butan-2-yl acetate (C065-1).

### Step 2: (2R)-4-Hydroxybutan-2-yl N-ethylcarbamate (C065)

To C065-1 (0.5 mmol) is added 5% hydrofluoric acid in acetonitrile (3.0 mL) at room temperature. After 8 min, the reaction mixture is neutralized with aqueous sodium bicarbonate solution, extracted with chloroform (2×10 mL), dried over anhydrous sodium sulfate, concentrated in under vacuum, and purified by silica gel flash column chromatography to afford C065.

Compound C066 is prepared using this same protocol replacing (2R)-4-[(tert-butyldimethylsilyl)oxy]butan-2-ol with (2S)-4-[(tert-butyldimethylsilyl)oxy]butan-2-ol.

### Preparation of Ethyl N-(3-Hydroxypropyl)carbamate (C067)

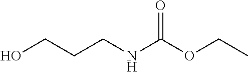

Compound C067 is prepared according to Xu, Guozhang et al. J. Med. Chem., 2001, 44 (24), 4092-4113. Using this same protocol ethyl chloroformate is replaced with phenyl chloroformate to make C068 and 3-aminopropan-1-ol is replaced with 3-(methylamino)propan-1-ol to make C069. Compounds C070-C073 are prepared using this same protocol replacing 3-aminopropan-1-ol with chiral 3-aminobutan-1-ols and 4-aminobutan-2-ols, accordingly.

### Preparation of N-(3-Hydroxypropyl)acetamide (C074)

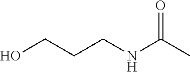

Compound C074 is prepared according to Yadav, Veejendra K. and Babu, K. Ganesh J. Org. Chem., 2004, 69 (2), 577-580 and Vokkaliga, Smitha et al. Tetrahedron Lett., 2011, 52 (21), 2722-2724. Compounds C076-C078 are prepared using this same protocol replacing acetyl chloride with propanoyl chloride, butanoyl chloride, and hexanoyl chloride, respectively. Compounds C099-C102 are prepared using this same protocol replacing 3-aminopropan-1-ol with chiral 3-aminobutan-1-ols and 4-aminobutan-2-ols, accordingly. Compounds C106 and C112 are prepared by replacing 3-aminopropan-1-ol with 4-aminobutan-1-ol and 6-aminohexan-1-ol, respectively.

### Preparation of N-(3-Hydroxypropyl)acetamide (C075)

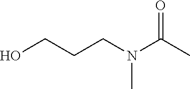

Compound C075 is prepared according to Gracias, Vijaya et al. J. Am. Chem. Soc., 1995, 117 (30), 8047-8.

### Preparation of N-(3-Hydroxypropyl)benzamide (C079)

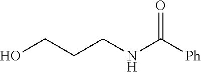 [SMILES: O=C(NCCCO)c1ccccc1]

Compound C079 is prepared according to Mollo, Maria C. and Orelli, Liliana R. Org. Lett., 2016, 18 (23), 6116-6119.

### Preparation of 4-Hydroxybutyl acetate (C103)

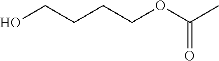

Compound C103 is prepared according to Desrat, Sandy et al. Org. Biomol. Chem., 2015, 13 (19), 5520-5531. Compound C111 is prepared using this protocol replacing 1,4-butanediol with 1,6-hexanediol.

### Preparation of (4S)-4-Hydroxypentyl acetate (C104)

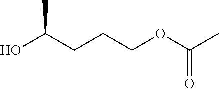

Compound C104 is prepared according to Koh, P.-F. and Loh, T.-P. Green Chemistry 2015, 17 (7), 3746-3750, Larson, Gerald L. and Fry, James L. Organic Reactions (Hoboken, NJ, United States), 2008, 71, 1-737 and Diaz-Rodriguez, Alba et al. ACS Catalysis, 2014, 4(2), 386-393.

### Preparation of (4R)-4-Hydroxypentyl acetate (C105)

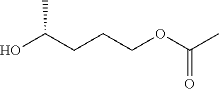

Compound C105 is prepared according to Chakraborty, Tushar Kanti et al. Tetrahedron 2009, 65 (34), 6925-6931.

### Preparation of 5-Hydroxypentyl acetate (C107)

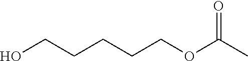

Compound C107 is prepared according to Rival, Nicolas et al. Tetrahedron Lett., 2015, 56 (49), 6823-6826.

### Preparation of (5S)-5-Hydroxyhexyl acetate (C108)

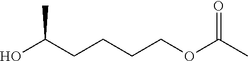

Compound C108 is prepared according to Smith, James R. et al. J. Am. Chem. Soc., 2017, 139 (27), 9148-9151. Compound C109 is prepared using this same protocol but with the opposite enantiomer of the catalyst. Compounds C181 and C182 are prepared by replacing hex-5-en-1-yl acetate with ethyl pent-4-enoate.

### Preparation of N-(5-Hydroxypentyl)acetamide (C110)

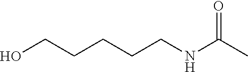

Compound C110 is prepared according to Temperini, Andrea et al. Synth. Commun., 2010, 40 (2), 295-302.

### Preparation of 2-[(Acetyloxy)methyl]-3-hydroxypropyl acetate (C114)

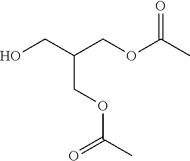

Compound C114 is prepared according to Marcuccio, Sebastian Mario and Jarvis, Karen Elizabeth PCT Int. Appl., 9912927, 1999.

### Preparation of Propan-2-yl 3-Hydroxypropanoate (C127)

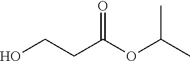

Compound C127 is prepared according to Parsy, Christophe Claude et al. PCT Int. Appl., 2013177195, 2013.

### Preparation of Propan-2-yl (3S)-3-Hydroxybutanoate (C130)

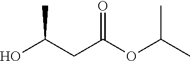

Compound C130 is prepared according to Noyori, Ryoji et al. J. Am. Chem. Soc., 1987, 109 (19), 5856-8 and Osawa, Tsutomu et al. Tetrahedron: Asymm., 2014, 25 (24), 1630-1633. Compound C133 is made using a similar protocol.

### Preparation of N,N-Diethyl-3-hydroxypropanamide (C139)

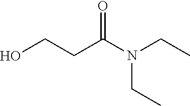

See T. L. Gresham, J. E. Jensen, F. W. Shaver, R. A. Bankert, F. T. Fiedorex J. Am. Chem. Soc., 1951, 73, 3168-3171. To a cold solution (0-5° C.) of diethylamine (1.64 mL, 15.82 mmol) in diethyl ether (10 mL) was added β-propiolactone (1.14 g, 15.82 mmol) slowly using a syringe over 5 min. After stirring at room temperature for 20-30 min the reaction mixture was concentrated under vacuum. The residue was purified by silica gel flash column chromatography (0-100% ethyl acetate/heptane) to afford the desired product C139 (300 mg, 13% yield). Compounds C135 and C140 are prepared using this same protocol replacing diethylamine with methylamine and piperdine, respectively.

### Preparation of Ethyl 2-(3-Hydroxypropanamido)acetate (C141)

Compound C141 is prepared from β-propiolactone and ethyl glycine using a protocol adapted from T. L. Gresham, J. E. Jensen, F. W. Shaver, R. A. Bankert, F. T. Fiedorex J. Am. Chem. Soc., 1951, 73, 3168-3171 and Lienard, Benoit M. R. et al Org. Biomol. Chem., 2008, 6 (13), 2282-2294. Compounds C142-C151 are prepared using this same protocol replacing ethyl glycine with alanine-, valine-, and proline-based amino esters, accordingly.

### Preparation of (3S)-3-Hydroxybutanamide (C152)

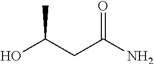

Compound C152 is prepared according to Boyer, Stephen James et al. PCT Int. Appl. 2011071716, 2011 and Rozzell, J. David, Jr. U.S. Pat. No. 5,834,261, 1998.

### Preparation of (3R)-3-Hydroxybutanamide (C153)

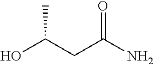

Compound C153 is prepared according to Ma, Da-You et al. J. Org. Chem., 2008, 73 (11), 4087-4091 and Yamano, Toru et al. Tetrahedron Lett., 1999, 40 (13), 2577-2580.

### Preparation of (3S)—N-Ethyl-3-hydroxybutanamide (C154)

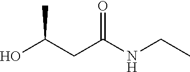

Compound C154 is prepared according to Kawai, Masao et al. Bull. Chem. Soc. Japan, 1988, 61 (8), 3014-16. Compounds C154 and C155 are also prepared from C129 and C132, respectively, using a protocol adapted from Just, George et al. PCT Int. Appl., 2000000499, 2000.

### Preparation of (3S)—N,N-Diethyl-3-hydroxybutanamide (C156)

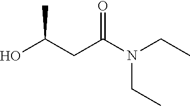

Compound C156 is prepared using enzymatic methods according to Gupta, Antje et al. PCT Int. Appl., 2006045598, 2006. Alternatively, compounds C156 and C157 are also prepared using catalytic asymmetric hydrogenation according to Li, Wanfang et al. Org. Lett., 2011, 13 (15), 3876-3879.

### Preparation of Ethyl 2-[(3S)-3-Hydroxybutanamido]acetate (C158)

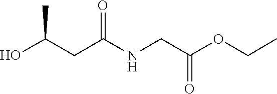

Compound C158 is prepared by coupling (3S)-3-hydroxybutanoic acid to ethyl glycine under EDC coupling conditions based on protocols described in Angehrn, Peter et al. J. Med. Chem., 2004, 47 (6), 1487-1513. Compounds C159-C179 are prepared using this same protocol replacing ethyl glycine with alanine-, valine-, and proline-based amino esters and (3S)-3-hydroxybutanoic with (3R)-3-hydroxybutanoic, accordingly.

### Preparation of 4-Hydroxybutanamide (C183)

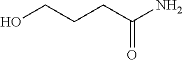

Compound C183 is prepared according to Bruice, Thomas C. and Marquardt, Fritz Hans J. Am. Chem. Soc., 1962, 84, 365-370. Compounds C185 and C186 are prepared using this same protocol replacing 7-butyrolactone with chiral (S)-γ-valerolactone (Sigma-Aldrich, CAS #[19041-15-7]) and (R)-γ-valerolactone (Sigma-Aldrich, CAS #[58917-25-2]), respectively.

### Preparation of N-Ethyl-4-hydroxybutanamide (C184)

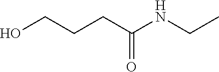

Compound C184 is prepared according to Cacciaglia, Roberto et al. PCT Int. Appl., 9806690, 1998. Compounds C187 and C188 are prepared using this same protocol replacing γ-butyrolactone with chiral (S)-γ-valerolactone (Sigma-Aldrich, CAS #[19041-15-7]) and (R)-γ-valerolactone (Sigma-Aldrich, CAS #[58917-25-2]), respectively. Compound C195 is prepared by replacing γ-butyrolactone with δ-valerolactone.

### Preparation of (4S)—N,N-Diethyl-4-hydroxypentanamide (C189)

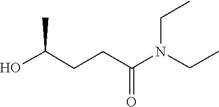

Compound C189 is prepared according to Matsumoto, Kiyoshi et al. Chem. Lett., 1987, (5), 803-804 replacing racemic γ-valerolactone with chiral (S)-γ-valerolactone (Sigma-Aldrich, CAS #[19041-15-7]). Compound C190 is prepared using this same protocol with (R)-γ-valerolactone (Sigma-Aldrich, CAS #[58917-25-2]). Compounds C185 and C186 are prepared by replacing diethylamine with ammonia. Compounds C187 and C188 are prepared by replacing diethylamine with ethylamine.

### Preparation of Ethyl (5S)-5-Hydroxyhexanoate (C192)

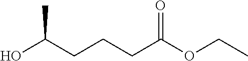

Compound C192 is prepared according to Araldi, Gian Luca et al. PCT Int. Appl., 2003103604, 2003 and Diaz-Rodriguez, Alba et al. ACS Catalysis, 2014, 4(2), 386-393. Compound C193 is prepared using a similar protocol.

### Preparation of 6-Hydroxyhexanamide (C197)

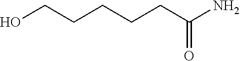

Compound C197 is prepared according to Zhou, Jianrong and Fu, Gregory C. J. Am. Chem. Soc., 2003, 125 (41), 12527-12530 and Shi, Yuan et al. J. Am. Chem. Soc., 2003, 135 (38), 14313-14320. Compound C198 is prepared by replacing ammonia with ethylamine. Compound C195 is prepared by replacing both ammonia with ethylamine and F-caprolactone with 6-valerolactone.

### Preparation of N—Ac-L-Serine Ethyl Ester (C202)

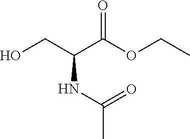

See J. Am. Chem. Soc., 1993, 115 (5), 1629-1631 for a related protocol. To a mixture of L-serine ethyl ester hydrochloride salt (170 mg, 1 mmol) in dry ethyl acetate (8 mL) was added triethylamine (0.28 mL, 0.78 mmol) and the mixture was stirred at room temperature for 10-15 min. The mixture was cooled to 0° C. and then acetyl chloride (0.07 mL, 1 mmol) was added slowly and stirred between 0-15° C. for 2 h (the reaction was monitored by TLC, eluting with 100% ethyl acetate and stained by KMnO₄ solution). The solid triethylamine hydrochloride salt was removed by filtration, washed with ethyl acetate (8 mL), and the filtrate was concentrated under vacuum at 40-45° C. The crude material was purified by silica gel flash column chromatography (0% to 70% ethyl acetate/heptane) to obtain the desired product C202 (80 mg, 46% yield). Compounds C201 and C205 are prepared using this same protocol replacing L-serine ethyl ester hydrochloride salt with L-serine methyl ester hydrochloride salt and L-threonine methyl ester hydrochloride, respectively.

### Preparation of N-Bz-L-Serine Ethyl Ester (C204)

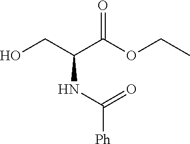 [SMILES: CCOC(=O)[C@H](CO)NC(=O)c1ccccc1]

See Brandstatter, Marco et al. J. Org. Chem., 2015, 80 (1), 40-51 and Matthies, Stefan et al. From J. Am. Chem. Soc., 2015, 137 (8), 2848-2851. To a mixture of L-serine ethyl ester hydrochloride salt (910 mg, 5.35 mmol) in dry ethyl acetate (40 mL) was added triethylamine (1.5 mL, 10.74 mmol) and the mixture was stirred at room temperature for 10-15 min. The mixture was cooled to 0° C. and then benzoyl chloride (752 mg, 5.35 mmol) was added slowly and stirred at 0-15° C. for 2-3 h (the reaction was monitored by TLC eluting with 60% ethyl acetate/heptane and stained by KMnO₄ solution). The solid triethylamine hydrochloride salt was removed by filtration, washed with ethyl acetate (8 mL), and the filtrate was concentrated under vacuum at 40-45° C. The residue was purified by silica gel flash column chromatography (0-40% ethyl acetate/heptane) to afford C204 (750 mg, 59% yield). Compounds C203 and C208 are prepared using this same protocol replacing L-serine ethyl ester hydrochloride salt with L-serine methyl ester hydrochloride salt and L-threonine methyl ester hydrochloride salt, respectively.

### Preparation of N—Ac-L-Threonine Ethyl Ester (C206)

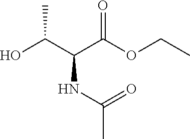

Compound C206 is prepared according to Genet, J. P.; Pinel, C.; Mallart, S.; Juge, S.; Thorimbert, S.; Laffitte, J. A. Tetrahedron: Asymm., 1991, 2 (7), 555-67.

### Preparation of N-Bz-L-Threonine Methyl Ester (C207)

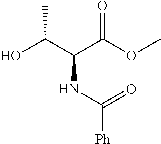 [SMILES: COC(=O)[C@@H](NC(=O)c1ccccc1)[C@@H](C)O]

To a mixture of L-threonine methyl ester hydrochloride salt (210 mg, 1.24 mmol) in dry ethyl acetate (10 mL) was added triethylamine (0.35 mL, 2.48 mmol) and the mixture was stirred at room temperature for 10-15 min. After cooling the mixture to 0° C., benzoyl chloride (0.14 mL, 1.24 mmol) was added slowly and stirred at 0-15° C. for 2-3 h (the reaction was monitored by TLC, eluent ethyl acetate and stained by KMnO₄ solution). The solid triethylamine hydrochloride salt was removed by filtration, washed with ethyl acetate (8 mL), and the filtrate was concentrated under vacuum at 40-45° C. The crude material was purified by silica gel flash column chromatography (0% to 70% ethyl acetate/heptane) to obtain C207 (60 mg, 20% yield).

Using the protocols to make the alcohols above, one skilled in the art can synthesize various other alcohols as shown in Table 3. These examples are not intended to be limiting, additional alcohol are made using alternative reagents (e.g. methylated vs ethylated vs propylated intermediates, etc.) and these same procedures.

The alcohol intermediates of Table 3 can be coupled to the intermediates of Table 2, in one example, by a method including adding DCC (229 mg, 1.1 mmol) and catalytic 4-dimethylaminopyridine (20-30 mg) to a mixture of B01 (351 mg, 0.83 mmol) and C035 (140 mg, 1 mmol) in dichloromethane (13 mL) under nitrogen atmosphere. The mixture was stirred at room temperature for 16-18 h and monitored by TLC (4:1 ethyl acetate/heptane) or HPLC. Upon completion, acetonitrile (15 mL) was added and the mixture was stirred for 5-10 min. The solid precipitate was removed by filtration through a sintered glass-funnel and the solid was washed with acetonitrile (10-15 mL). The filtrate was concentrated under vacuum at 40-45° C. and purified by silica gel flash column chromatography (0% to 100% ethyl acetate/heptane) to obtain the Cbz-protected coupled product D01 (190 mg, 44% yield).

Following coupling, the Cbz protecting group can be removed. In one example, this removal can take place, and the resulting molecule be purified by a method including:

To a mixture of D01 (190 mg, 0.58 mmol) in isopropanol-TIF (1:1, 10 mL) was added 10% Pd/C (dry) (100 mg). This mixture was degassed and hydrogen purged by three cycles of vacuum, release, and placed under hydrogen atmosphere. The mixture was stirred under hydrogen for 1.5-2 h and monitored by HPLC/TLC. The reaction mixture was filtered through Celite, washed with THF (15-20 mL), and the filtrate was concentrated under vacuum at 40-45° C. The residue was further dried under high vacuum for 10-12 h and treated with heptane (15-20 mL) at room temperature for 16-18 h and decanted. The solid was further dried under high vacuum 10-12 h to obtain crude 4 (135 mg, 93% yield). The residue was purified using a Waters Oasis HLB Cartridge (20 cc, 1 g Sorbent, 30 m Particle Size), which was previously washed with methanol (3 mL), acetonitrile (3 mL), and water (3×5 mL). The residue (˜50 mg) was dissolved in 0.2 N HCl (1 mL), loaded onto the column, and water was used to fully transfer the compound to the column. The column was flushed with water, and then the compound was eluted with 4%, 8%, 10%, 15%, and 20% acetonitrile-water. Desired fractions were determined using reverse phase TLC plates (25% acetonitrile-water) and concentrated to afford 4 as the hydrochloride salt (multiple purifications were combined to afford 28.1 mg).

While example reactions are given above, it should be understood that similar methods can be used for any core compound shown in Table 1 to create the intermediates of Table 2, which can be coupled with any of the alcohols shown in Table 3. The resulting molecule can include any of the compounds illustrated below in Table 4.

TABLE 4
Compounds.

| # | Structure | Core | Alcohol |
|---|---|---|---|
| 1 | 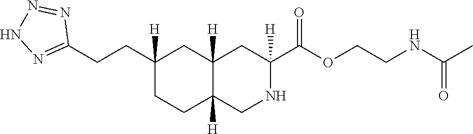 [SMILES: CC(=O)NCCOC(=O)[C@@H]1C[C@H]2C[C@@H](CCc3nn[nH]n3)CC[C@H]2CN1] | A01 | C002 |
| 2 | 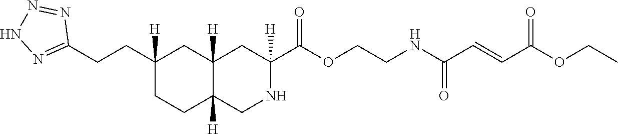 [SMILES: CCOC(=O)/C=C/C(=O)NCCOC(=O)[C@@H]1C[C@H]2C[C@@H](CCc3nn[nH]n3)CC[C@H]2CN1] | A01 | C010 |
| 3 | 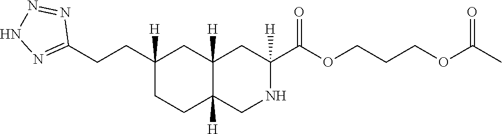 [SMILES: CC(=O)OCCCOC(=O)[C@@H]1C[C@H]2C[C@@H](CCc3nn[nH]n3)CC[C@H]2CN1] | A01 | C034 |
| 4 | 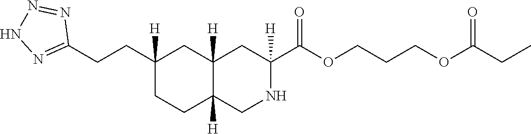 [SMILES: CCC(=O)OCCCOC(=O)[C@@H]1C[C@H]2C[C@@H](CCc3nn[nH]n3)CC[C@H]2CN1] | A01 | C035 |
| 5 | 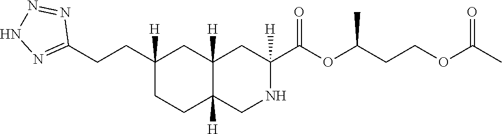 [SMILES: CC(=O)OCC[C@H](C)OC(=O)[C@@H]1C[C@H]2C[C@@H](CCc3nn[nH]n3)CC[C@H]2CN1] | A01 | C047 |
| 6 | 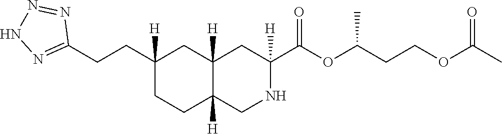 [SMILES: CC(=O)OCC[C@@H](C)OC(=O)[C@@H]1C[C@H]2C[C@@H](CCc3nn[nH]n3)CC[C@H]2CN1] | A01 | C051 |
| 7 | 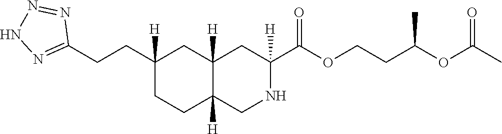 [SMILES: CC(=O)O[C@H](C)CCOC(=O)[C@@H]1C[C@H]2C[C@@H](CCc3nn[nH]n3)CC[C@H]2CN1] | A01 | C055 |
| 8 | 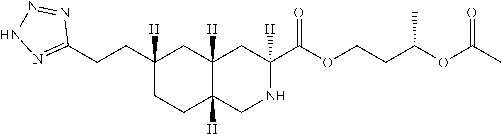 [SMILES: CC(=O)O[C@@H](C)CCOC(=O)[C@@H]1C[C@H]2C[C@@H](CCc3nn[nH]n3)CC[C@H]2CN1] | A01 | C058 |
| 9 | 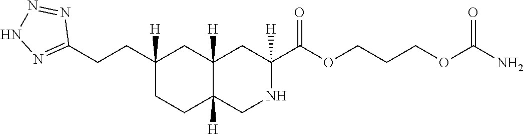 [SMILES: NC(=O)OCCCOC(=O)[C@@H]1C[C@H]2C[C@@H](CCc3nn[nH]n3)CC[C@H]2CN1] | A01 | C061 |
| 10 | 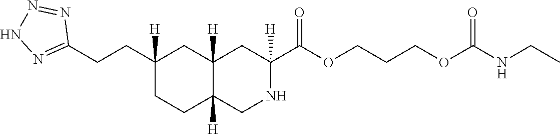 [SMILES: CCNC(=O)OCCCOC(=O)[C@@H]1C[C@H]2C[C@@H](CCc3nn[nH]n3)CC[C@H]2CN1] | A01 | C062 |
| 11 | 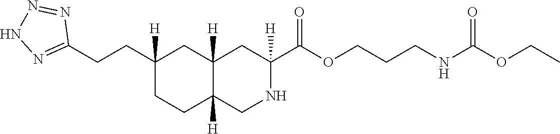 [SMILES: CCOC(=O)NCCCOC(=O)[C@@H]1C[C@H]2C[C@@H](CCc3nn[nH]n3)CC[C@H]2CN1] | A01 | C067 |
| 12 | 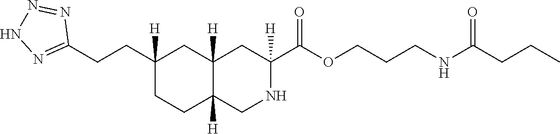 [SMILES: CCCC(=O)NCCCOC(=O)[C@@H]1C[C@H]2C[C@@H](CCc3nn[nH]n3)CC[C@H]2CN1] | A01 | C077 |
| 13 | 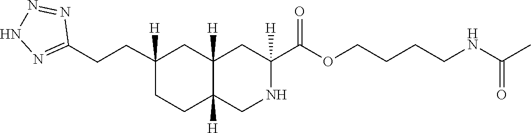 [SMILES: CC(=O)NCCCCOC(=O)[C@@H]1C[C@H]2C[C@@H](CCc3nn[nH]n3)CC[C@H]2CN1] | A01 | C106 |
| 14 | 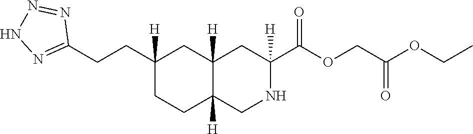 [SMILES: CCOC(=O)COC(=O)[C@@H]1C[C@H]2C[C@@H](CCc3nn[nH]n3)CC[C@H]2CN1] | A01 | C116 |
| 15 | 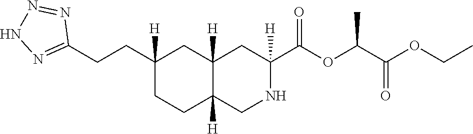 [SMILES: CCOC(=O)[C@H](C)OC(=O)[C@@H]1C[C@H]2C[C@@H](CCc3nn[nH]n3)CC[C@H]2CN1] | A01 | C119 |
| 16 | 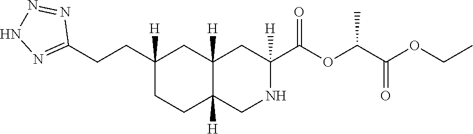 [SMILES: CCOC(=O)[C@@H](C)OC(=O)[C@@H]1C[C@H]2C[C@@H](CCc3nn[nH]n3)CC[C@H]2CN1] | A01 | C121 |
| 17 | 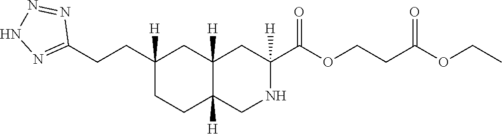 [SMILES: CCOC(=O)CCOC(=O)[C@@H]1C[C@H]2C[C@@H](CCc3nn[nH]n3)CC[C@H]2CN1] | A01 | C126 |
| 18 | 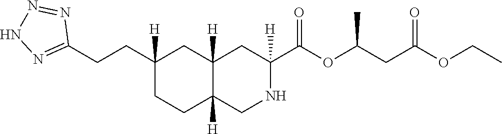 [SMILES: CCOC(=O)C[C@H](C)OC(=O)[C@@H]1C[C@H]2C[C@@H](CCc3nn[nH]n3)CC[C@H]2CN1] | A01 | C129 |
| 19 | 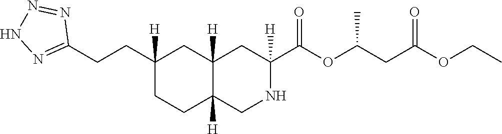 [SMILES: CCOC(=O)C[C@@H](C)OC(=O)[C@@H]1C[C@H]2C[C@@H](CCc3nn[nH]n3)CC[C@H]2CN1] | A01 | C132 |
| 20 | 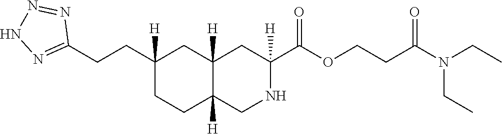 [SMILES: CCN(CC)C(=O)CCOC(=O)[C@@H]1C[C@H]2C[C@@H](CCc3nn[nH]n3)CC[C@H]2CN1] | A01 | C139 |
| 21 | 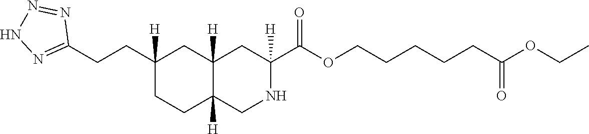 [SMILES: CCOC(=O)CCCCCOC(=O)[C@@H]1C[C@H]2C[C@@H](CCc3nn[nH]n3)CC[C@H]2CN1] | A01 | C196 |
| 22 | 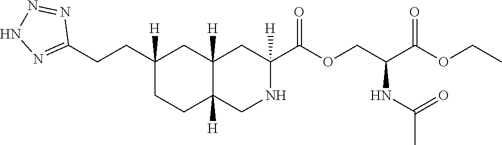 [SMILES: CCOC(=O)[C@H](COC(=O)[C@@H]1C[C@H]2C[C@@H](CCc3nn[nH]n3)CC[C@H]2CN1)NC(C)=O] | A01 | C202 |
| 23 | 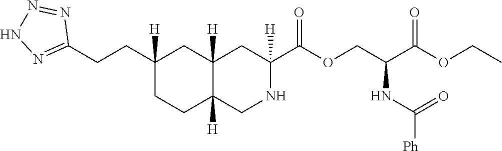 [SMILES: CCOC(=O)[C@H](COC(=O)[C@@H]1C[C@H]2C[C@@H](CCc3nn[nH]n3)CC[C@H]2CN1)NC(=O)c1ccccc1] | A01 | C204 |
| 24 | 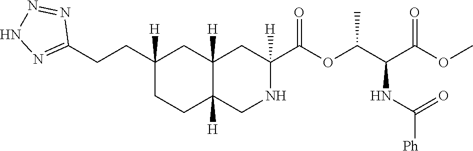 [SMILES: COC(=O)[C@@H](NC(=O)c1ccccc1)[C@@H](C)OC(=O)[C@@H]1C[C@H]2C[C@@H](CCc3nn[nH]n3)CC[C@H]2CN1] | A01 | C207 |
| 25 | 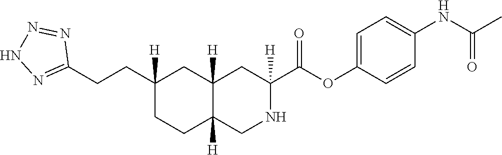 [SMILES: CC(=O)Nc1ccc(OC(=O)[C@@H]2C[C@H]3C[C@@H](CCc4nn[nH]n4)CC[C@H]3CN2)cc1] | A01 | C209 |
| 26 | 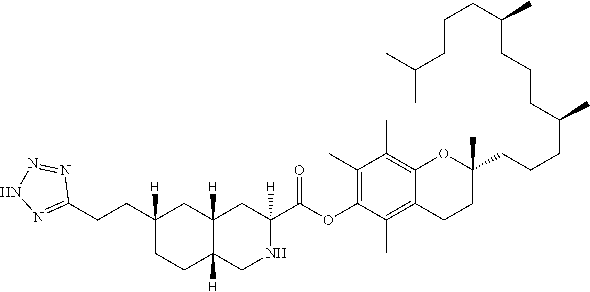 [SMILES: Cc1c(C)c2c(c(C)c1OC(=O)[C@@H]1C[C@H]3C[C@@H](CCc4nn[nH]n4)CC[C@H]3CN1)CC[C@@](C)(CCC[C@H](C)CCC[C@H](C)CCCC(C)C)O2] | A01 | C216 |
| 27 | 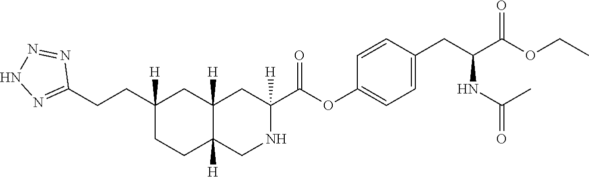 [SMILES: CCOC(=O)[C@H](Cc1ccc(OC(=O)[C@@H]2C[C@H]3C[C@@H](CCc4nn[nH]n4)CC[C@H]3CN2)cc1)NC(C)=O] | A01 | C217 |
| 28 | 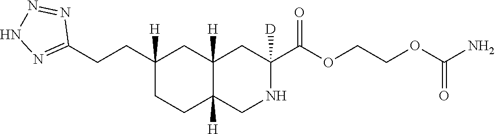 [SMILES: [2H][C@@]1(C(=O)OCCOC(N)=O)C[C@H]2C[C@@H](CCc3nn[nH]n3)CC[C@H]2CN1] | A02 | C001 |
| 29 | 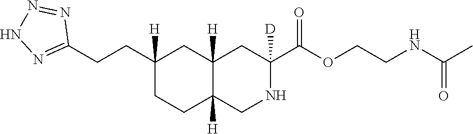 [SMILES: [2H][C@@]1(C(=O)OCCNC(C)=O)C[C@H]2C[C@@H](CCc3nn[nH]n3)CC[C@H]2CN1] | A02 | C002 |
| 30 | 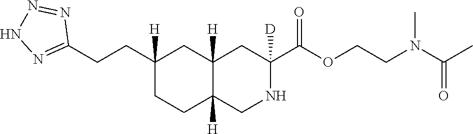 [SMILES: [2H][C@@]1(C(=O)OCCN(C)C(C)=O)C[C@H]2C[C@@H](CCc3nn[nH]n3)CC[C@H]2CN1] | A02 | C003 |
| 31 | 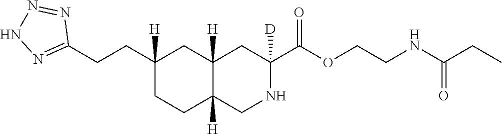 [SMILES: [2H][C@@]1(C(=O)OCCNC(=O)CC)C[C@H]2C[C@@H](CCc3nn[nH]n3)CC[C@H]2CN1] | A02 | C004 |
| 32 | 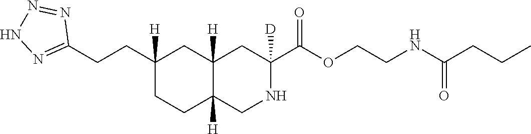 [SMILES: [2H][C@@]1(C(=O)OCCNC(=O)CCC)C[C@H]2C[C@@H](CCc3nn[nH]n3)CC[C@H]2CN1] | A02 | C005 |
| 33 | 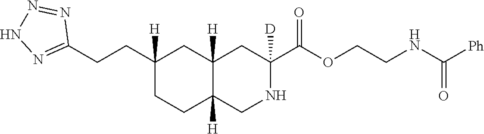 [SMILES: [2H][C@@]1(C(=O)OCCNC(=O)c2ccccc2)C[C@H]2C[C@@H](CCc3nn[nH]n3)CC[C@H]2CN1] | A02 | C007 |
| 34 | 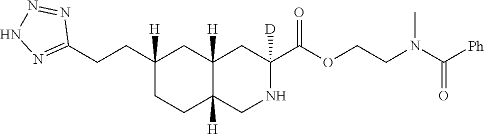 [SMILES: [2H][C@@]1(C(=O)OCCN(C)C(=O)c2ccccc2)C[C@H]2C[C@@H](CCc3nn[nH]n3)CC[C@H]2CN1] | A02 | C008 |
| 35 | 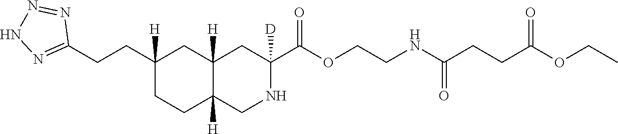 [SMILES: [2H][C@@]1(C(=O)OCCNC(=O)CCC(=O)OCC)C[C@H]2C[C@@H](CCc3nn[nH]n3)CC[C@H]2CN1] | A02 | C009 |
| 36 | 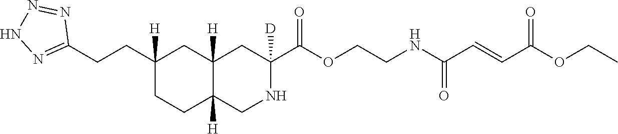 [SMILES: [2H][C@@]1(C(=O)OCCNC(=O)/C=C/C(=O)OCC)C[C@H]2C[C@@H](CCc3nn[nH]n3)CC[C@H]2CN1] | A02 | C010 |
| 37 | 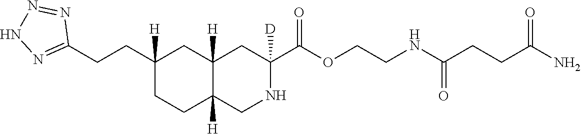 [SMILES: [2H][C@@]1(C(=O)OCCNC(=O)CCC(N)=O)C[C@H]2C[C@@H](CCc3nn[nH]n3)CC[C@H]2CN1] | A02 | C011 |
| 38 | 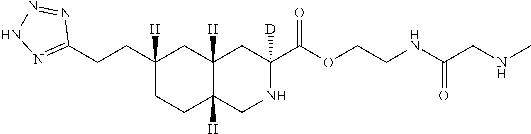 [SMILES: [2H][C@@]1(C(=O)OCCNC(=O)CNC)C[C@H]2C[C@@H](CCc3nn[nH]n3)CC[C@H]2CN1] | A02 | C014 |
| 39 | 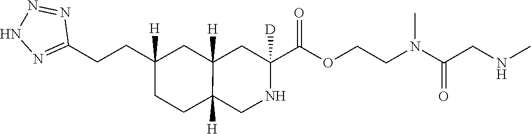 [SMILES: [2H][C@@]1(C(=O)OCCN(C)C(=O)CNC)C[C@H]2C[C@@H](CCc3nn[nH]n3)CC[C@H]2CN1] | A02 | C015 |
| 40 | 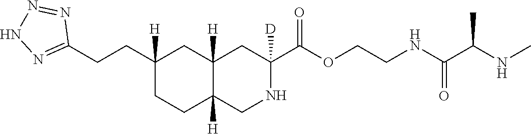 [SMILES: [2H][C@@]1(C(=O)OCCNC(=O)[C@@H](C)NC)C[C@H]2C[C@@H](CCc3nn[nH]n3)CC[C@H]2CN1] | A02 | C016 |
| 41 | 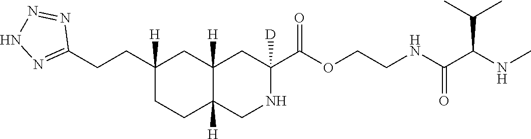 [SMILES: [2H][C@@]1(C(=O)OCCNC(=O)[C@H](NC)C(C)C)C[C@H]2C[C@@H](CCc3nn[nH]n3)CC[C@H]2CN1] | A02 | C017 |
| 42 | 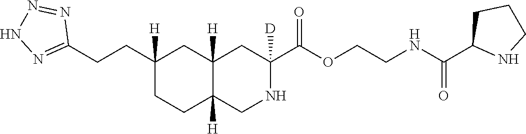 [SMILES: [2H][C@@]1(C(=O)OCCNC(=O)[C@H]2CCCN2)C[C@H]2C[C@@H](CCc3nn[nH]n3)CC[C@H]2CN1] | A02 | C018 |
| 43 | 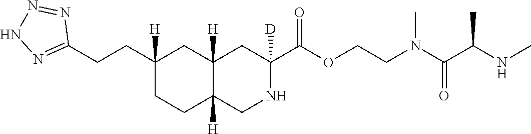 [SMILES: [2H][C@@]1(C(=O)OCCN(C)C(=O)[C@@H](C)NC)C[C@H]2C[C@@H](CCc3nn[nH]n3)CC[C@H]2CN1] | A02 | C019 |
| 44 | 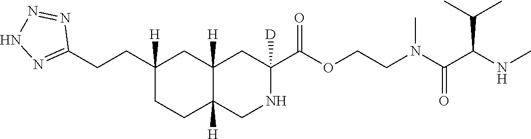 [SMILES: [2H][C@@]1(C(=O)OCCN(C)C(=O)[C@H](NC)C(C)C)C[C@H]2C[C@@H](CCc3nn[nH]n3)CC[C@H]2CN1] | A02 | C020 |
| 45 | 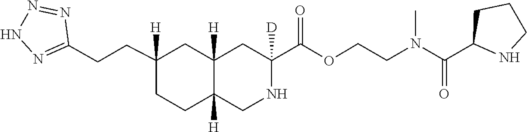 [SMILES: [2H][C@@]1(C(=O)OCCN(C)C(=O)[C@H]2CCCN2)C[C@H]2C[C@@H](CCc3nn[nH]n3)CC[C@H]2CN1] | A02 | C021 |
| 46 | 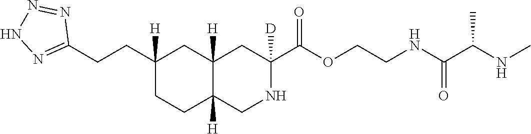 [SMILES: [2H][C@@]1(C(=O)OCCNC(=O)[C@H](C)NC)C[C@H]2C[C@@H](CCc3nn[nH]n3)CC[C@H]2CN1] | A02 | C022 |
| 47 | 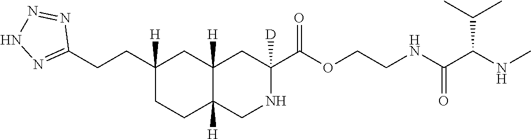 [SMILES: [2H][C@@]1(C(=O)OCCNC(=O)[C@@H](NC)C(C)C)C[C@H]2C[C@@H](CCc3nn[nH]n3)CC[C@H]2CN1] | A02 | C023 |
| 48 | 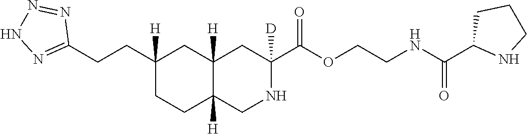 [SMILES: [2H][C@@]1(C(=O)OCCNC(=O)[C@@H]2CCCN2)C[C@H]2C[C@@H](CCc3nn[nH]n3)CC[C@H]2CN1] | A02 | C024 |
| 49 | 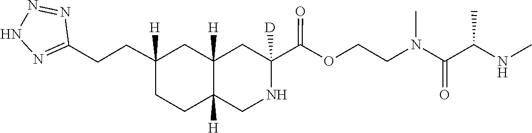 [SMILES: [2H][C@@]1(C(=O)OCCN(C)C(=O)[C@H](C)NC)C[C@H]2C[C@@H](CCc3nn[nH]n3)CC[C@H]2CN1] | A02 | C025 |
| 50 | 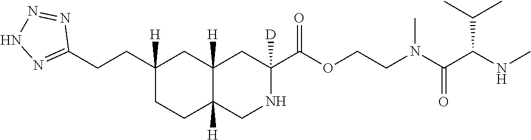 [SMILES: [2H][C@@]1(C(=O)OCCN(C)C(=O)[C@@H](NC)C(C)C)C[C@H]2C[C@@H](CCc3nn[nH]n3)CC[C@H]2CN1] | A02 | C026 |
| 51 | 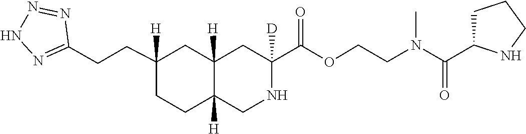 [SMILES: [2H][C@@]1(C(=O)OCCN(C)C(=O)[C@@H]2CCCN2)C[C@H]2C[C@@H](CCc3nn[nH]n3)CC[C@H]2CN1] | A02 | C027 |
| 52 | 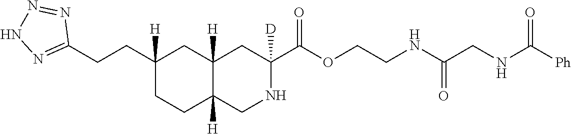 [SMILES: [2H][C@@]1(C(=O)OCCNC(=O)CNC(=O)c2ccccc2)C[C@H]2C[C@@H](CCc3nn[nH]n3)CC[C@H]2CN1] | A02 | C028 |
| 53 | 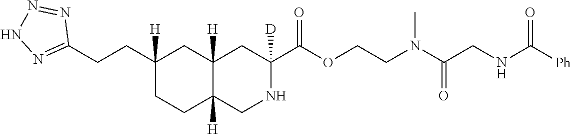 [SMILES: [2H][C@@]1(C(=O)OCCN(C)C(=O)CNC(=O)c2ccccc2)C[C@H]2C[C@@H](CCc3nn[nH]n3)CC[C@H]2CN1] | A02 | C029 |
| 54 | 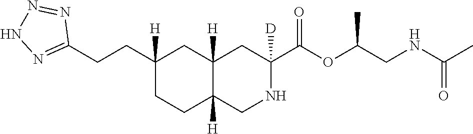 [SMILES: [2H][C@@]1(C(=O)O[C@@H](C)CNC(C)=O)C[C@H]2C[C@@H](CCc3nn[nH]n3)CC[C@H]2CN1] | A02 | C030 |
| 55 | 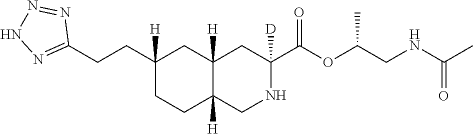 [SMILES: [2H][C@@]1(C(=O)O[C@H](C)CNC(C)=O)C[C@H]2C[C@@H](CCc3nn[nH]n3)CC[C@H]2CN1] | A02 | C031 |
| 56 | 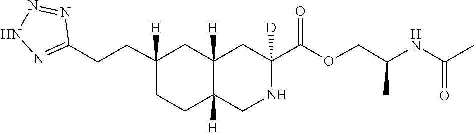 [SMILES: [2H][C@@]1(C(=O)OC[C@H](C)NC(C)=O)C[C@H]2C[C@@H](CCc3nn[nH]n3)CC[C@H]2CN1] | A02 | C032 |
| 57 | 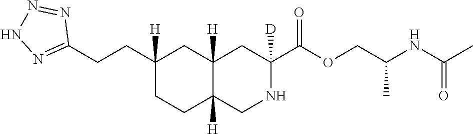 [SMILES: [2H][C@@]1(C(=O)OC[C@@H](C)NC(C)=O)C[C@H]2C[C@@H](CCc3nn[nH]n3)CC[C@H]2CN1] | A02 | C033 |
| 58 | 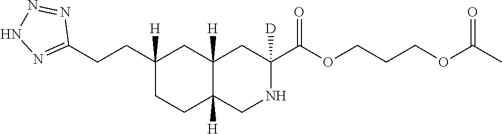 [SMILES: [2H][C@@]1(C(=O)OCCCOC(C)=O)C[C@H]2C[C@@H](CCc3nn[nH]n3)CC[C@H]2CN1] | A02 | C034 |
| 59 | 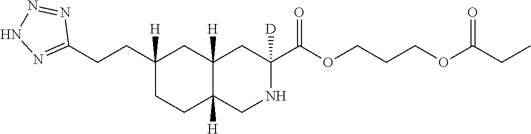 [SMILES: [2H][C@@]1(C(=O)OCCCOC(=O)CC)C[C@H]2C[C@@H](CCc3nn[nH]n3)CC[C@H]2CN1] | A02 | C035 |
| 60 | 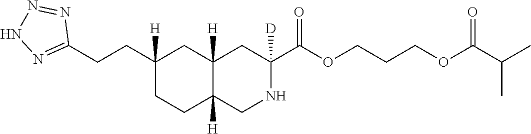 [SMILES: [2H][C@@]1(C(=O)OCCCOC(=O)C(C)C)C[C@H]2C[C@@H](CCc3nn[nH]n3)CC[C@H]2CN1] | A02 | C036 |
| 61 | 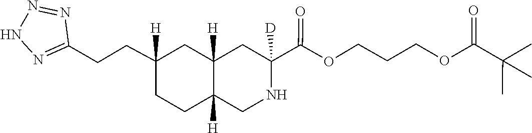 [SMILES: [2H][C@@]1(C(=O)OCCCOC(=O)C(C)(C)C)C[C@H]2C[C@@H](CCc3nn[nH]n3)CC[C@H]2CN1] | A02 | C037 |
| 62 | 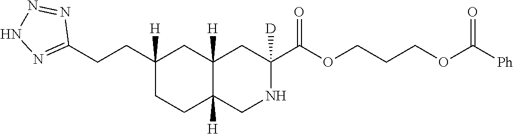 [SMILES: [2H][C@@]1(C(=O)OCCCOC(=O)c2ccccc2)C[C@H]2C[C@@H](CCc3nn[nH]n3)CC[C@H]2CN1] | A02 | C038 |
| 63 | 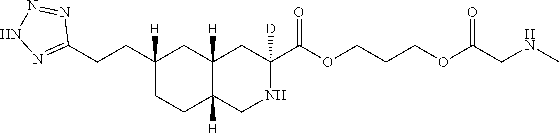 [SMILES: [2H][C@@]1(C(=O)OCCCOC(=O)CNC)C[C@H]2C[C@@H](CCc3nn[nH]n3)CC[C@H]2CN1] | A02 | C040 |
| 64 | 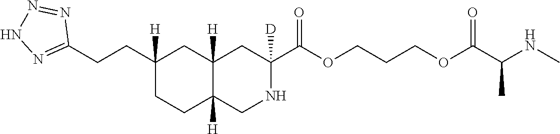 [SMILES: [2H][C@@]1(C(=O)OCCCOC(=O)[C@H](C)NC)C[C@H]2C[C@@H](CCc3nn[nH]n3)CC[C@H]2CN1] | A02 | C041 |
| 65 | 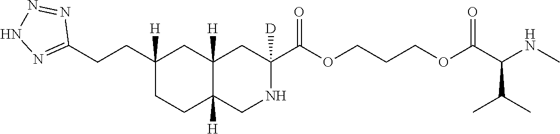 [SMILES: [2H][C@@]1(C(=O)OCCCOC(=O)[C@@H](NC)C(C)C)C[C@H]2C[C@@H](CCc3nn[nH]n3)CC[C@H]2CN1] | A02 | C042 |
| 66 | 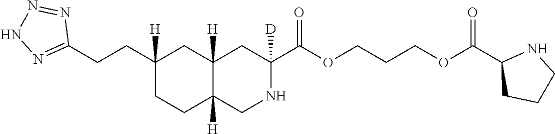 [SMILES: [2H][C@@]1(C(=O)OCCCOC(=O)[C@@H]2CCCN2)C[C@H]2C[C@@H](CCc3nn[nH]n3)CC[C@H]2CN1] | A02 | C043 |
| 67 | 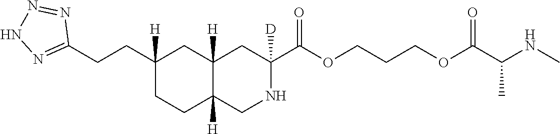 [SMILES: [2H][C@@]1(C(=O)OCCCOC(=O)[C@@H](C)NC)C[C@H]2C[C@@H](CCc3nn[nH]n3)CC[C@H]2CN1] | A02 | C044 |
| 68 | 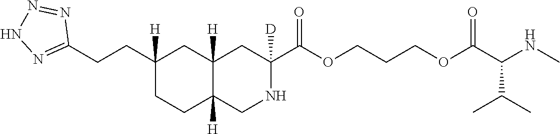 [SMILES: [2H][C@@]1(C(=O)OCCCOC(=O)[C@H](NC)C(C)C)C[C@H]2C[C@@H](CCc3nn[nH]n3)CC[C@H]2CN1] | A02 | C045 |
| 69 | 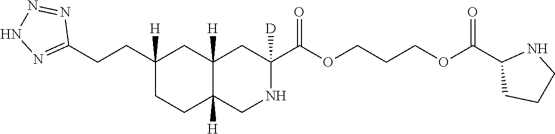 [SMILES: [2H][C@@]1(C(=O)OCCCOC(=O)[C@H]2CCCN2)C[C@H]2C[C@@H](CCc3nn[nH]n3)CC[C@H]2CN1] | A02 | C046 |
| 70 | 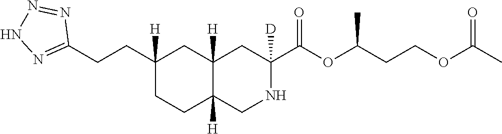 [SMILES: [2H][C@@]1(C(=O)O[C@@H](C)CCOC(C)=O)C[C@H]2C[C@@H](CCc3nn[nH]n3)CC[C@H]2CN1] | A02 | C047 |
| 71 | 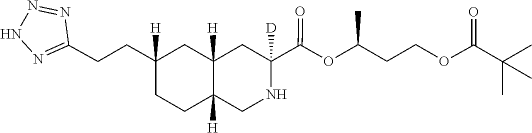 [SMILES: [2H][C@@]1(C(=O)O[C@@H](C)CCOC(=O)C(C)(C)C)C[C@H]2C[C@@H](CCc3nn[nH]n3)CC[C@H]2CN1] | A02 | C049 |
| 72 | 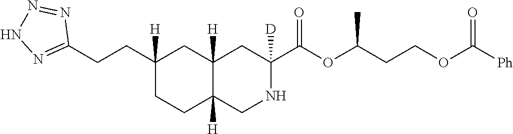 [SMILES: [2H][C@@]1(C(=O)O[C@@H](C)CCOC(=O)c2ccccc2)C[C@H]2C[C@@H](CCc3nn[nH]n3)CC[C@H]2CN1] | A02 | C050 |
| 73 | 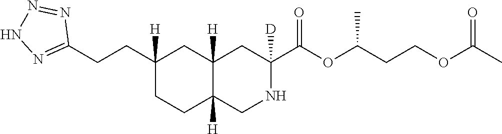 [SMILES: [2H][C@@]1(C(=O)O[C@H](C)CCOC(C)=O)C[C@H]2C[C@@H](CCc3nn[nH]n3)CC[C@H]2CN1] | A02 | C051 |
| 74 | 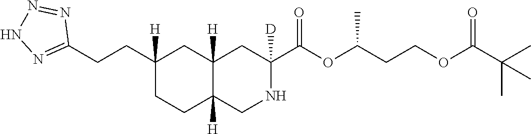 [SMILES: [2H][C@@]1(C(=O)O[C@H](C)CCOC(=O)C(C)(C)C)C[C@H]2C[C@@H](CCc3nn[nH]n3)CC[C@H]2CN1] | A02 | C053 |
| 75 | 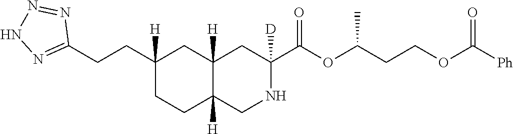 [SMILES: [2H][C@@]1(C(=O)O[C@H](C)CCOC(=O)c2ccccc2)C[C@H]2C[C@@H](CCc3nn[nH]n3)CC[C@H]2CN1] | A02 | C054 |
| 76 | 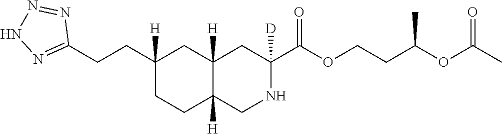 [SMILES: [2H][C@@]1(C(=O)OCC[C@@H](C)OC(C)=O)C[C@H]2C[C@@H](CCc3nn[nH]n3)CC[C@H]2CN1] | A02 | C055 |
| 77 | 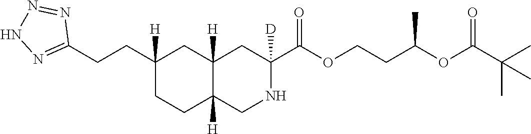 [SMILES: [2H][C@@]1(C(=O)OCC[C@@H](C)OC(=O)C(C)(C)C)C[C@H]2C[C@@H](CCc3nn[nH]n3)CC[C@H]2CN1] | A02 | C056 |
| 78 | 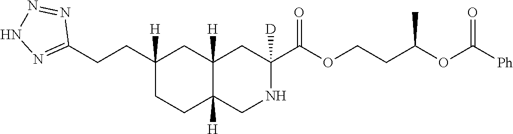 [SMILES: [2H][C@@]1(C(=O)OCC[C@@H](C)OC(=O)c2ccccc2)C[C@H]2C[C@@H](CCc3nn[nH]n3)CC[C@H]2CN1] | A02 | C057 |
| 79 | 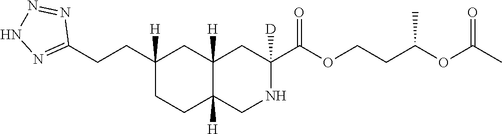 [SMILES: [2H][C@@]1(C(=O)OCC[C@H](C)OC(C)=O)C[C@H]2C[C@@H](CCc3nn[nH]n3)CC[C@H]2CN1] | A02 | C058 |
| 80 | 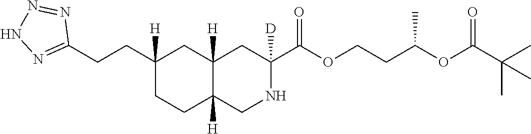 [SMILES: [2H][C@@]1(C(=O)OCC[C@H](C)OC(=O)C(C)(C)C)C[C@H]2C[C@@H](CCc3nn[nH]n3)CC[C@H]2CN1] | A02 | C059 |
| 81 | 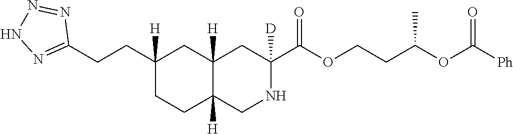 [SMILES: [2H][C@@]1(C(=O)OCC[C@H](C)OC(=O)c2ccccc2)C[C@H]2C[C@@H](CCc3nn[nH]n3)CC[C@H]2CN1] | A02 | C060 |
| 82 | 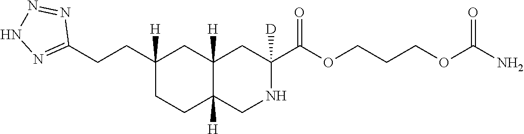 [SMILES: [2H][C@@]1(C(=O)OCCCOC(N)=O)C[C@H]2C[C@@H](CCc3nn[nH]n3)CC[C@H]2CN1] | A02 | C061 |
| 83 | 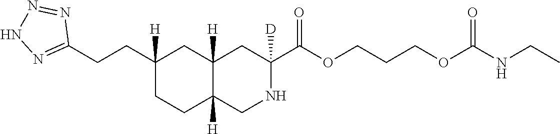 [SMILES: [2H][C@@]1(C(=O)OCCCOC(=O)NCC)C[C@H]2C[C@@H](CCc3nn[nH]n3)CC[C@H]2CN1] | A02 | C062 |
| 84 | 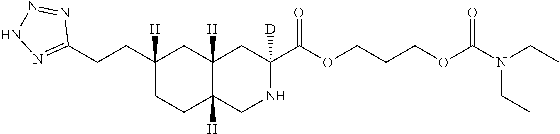 [SMILES: [2H][C@@]1(C(=O)OCCCOC(=O)N(CC)CC)C[C@H]2C[C@@H](CCc3nn[nH]n3)CC[C@H]2CN1] | A02 | C064 |
| 85 | 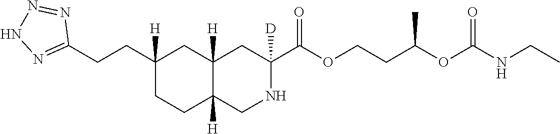 [SMILES: [2H][C@@]1(C(=O)OCC[C@@H](C)OC(=O)NCC)C[C@H]2C[C@@H](CCc3nn[nH]n3)CC[C@H]2CN1] | A02 | C065 |
| 86 | 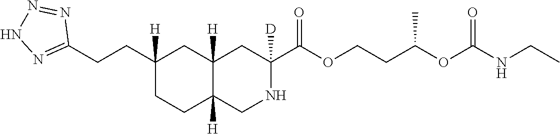 [SMILES: [2H][C@@]1(C(=O)OCC[C@H](C)OC(=O)NCC)C[C@H]2C[C@@H](CCc3nn[nH]n3)CC[C@H]2CN1] | A02 | C066 |
| 87 | 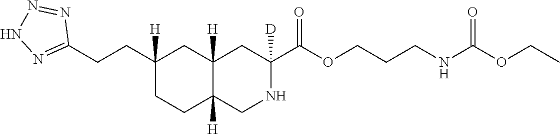 [SMILES: [2H][C@@]1(C(=O)OCCCNC(=O)OCC)C[C@H]2C[C@@H](CCc3nn[nH]n3)CC[C@H]2CN1] | A02 | C067 |
| 88 | 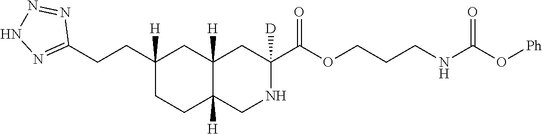 [SMILES: [2H][C@@]1(C(=O)OCCCNC(=O)Oc2ccccc2)C[C@H]2C[C@@H](CCc3nn[nH]n3)CC[C@H]2CN1] | A02 | C068 |
| 89 | 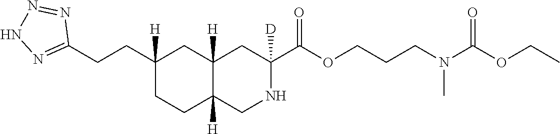 [SMILES: [2H][C@@]1(C(=O)OCCCN(C)C(=O)OCC)C[C@H]2C[C@@H](CCc3nn[nH]n3)CC[C@H]2CN1] | A02 | C069 |
| 90 | 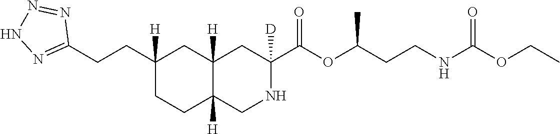 [SMILES: [2H][C@@]1(C(=O)O[C@@H](C)CCNC(=O)OCC)C[C@H]2C[C@@H](CCc3nn[nH]n3)CC[C@H]2CN1] | A02 | C070 |
| 91 | 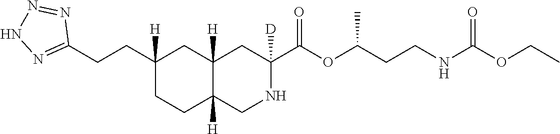 [SMILES: [2H][C@@]1(C(=O)O[C@H](C)CCNC(=O)OCC)C[C@H]2C[C@@H](CCc3nn[nH]n3)CC[C@H]2CN1] | A02 | C071 |
| 92 | 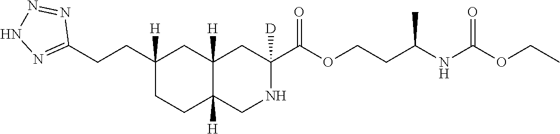 [SMILES: [2H][C@@]1(C(=O)OCC[C@@H](C)NC(=O)OCC)C[C@H]2C[C@@H](CCc3nn[nH]n3)CC[C@H]2CN1] | A02 | C072 |
| 93 | 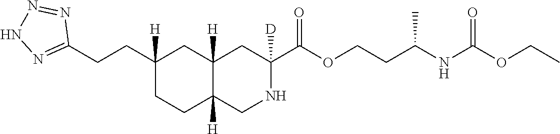 [SMILES: [2H][C@@]1(C(=O)OCC[C@H](C)NC(=O)OCC)C[C@H]2C[C@@H](CCc3nn[nH]n3)CC[C@H]2CN1] | A02 | C073 |
| 94 | 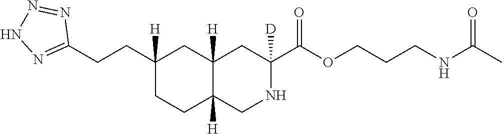 [SMILES: [2H][C@@]1(C(=O)OCCCNC(C)=O)C[C@H]2C[C@@H](CCc3nn[nH]n3)CC[C@H]2CN1] | A02 | C074 |
| 95 | 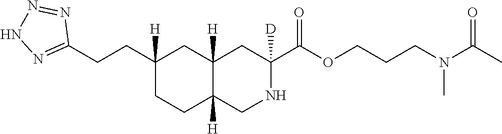 [SMILES: [2H][C@@]1(C(=O)OCCCN(C)C(C)=O)C[C@H]2C[C@@H](CCc3nn[nH]n3)CC[C@H]2CN1] | A02 | C075 |
| 96 | 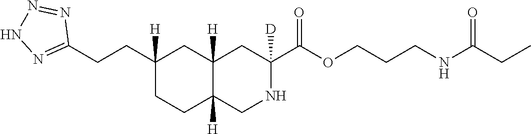 [SMILES: [2H][C@@]1(C(=O)OCCCNC(=O)CC)C[C@H]2C[C@@H](CCc3nn[nH]n3)CC[C@H]2CN1] | A02 | C076 |
| 97 | 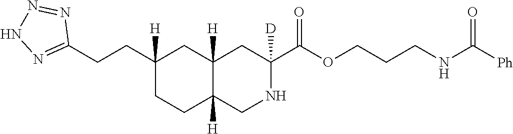 [SMILES: [2H][C@@]1(C(=O)OCCCNC(=O)c2ccccc2)C[C@H]2C[C@@H](CCc3nn[nH]n3)CC[C@H]2CN1] | A02 | C079 |
| 98 | 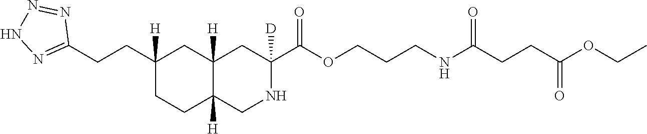 [SMILES: [2H][C@@]1(C(=O)OCCCNC(=O)CCC(=O)OCC)C[C@H]2C[C@@H](CCc3nn[nH]n3)CC[C@H]2CN1] | A02 | C080 |
| 99 | 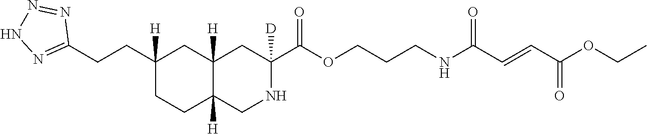 [SMILES: [2H][C@@]1(C(=O)OCCCNC(=O)/C=C/C(=O)OCC)C[C@H]2C[C@@H](CCc3nn[nH]n3)CC[C@H]2CN1] | A02 | C081 |
| 100 | 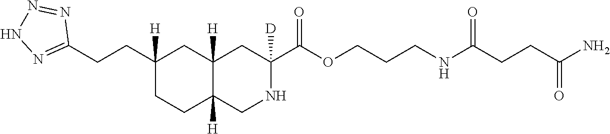 [SMILES: [2H][C@@]1(C(=O)OCCCNC(=O)CCC(N)=O)C[C@H]2C[C@@H](CCc3nn[nH]n3)CC[C@H]2CN1] | A02 | C082 |
| 101 | 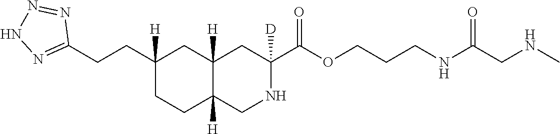 [SMILES: [2H][C@@]1(C(=O)OCCCNC(=O)CNC)C[C@H]2C[C@@H](CCc3nn[nH]n3)CC[C@H]2CN1] | A02 | C085 |
| 102 | 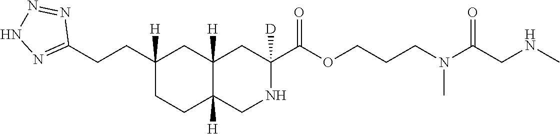 [SMILES: [2H][C@@]1(C(=O)OCCCN(C)C(=O)CNC)C[C@H]2C[C@@H](CCc3nn[nH]n3)CC[C@H]2CN1] | A02 | C086 |
| 103 | 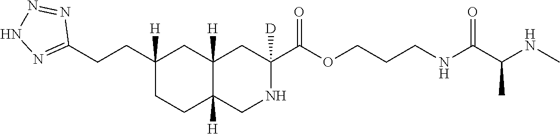 [SMILES: [2H][C@@]1(C(=O)OCCCNC(=O)[C@H](C)NC)C[C@H]2C[C@@H](CCc3nn[nH]n3)CC[C@H]2CN1] | A02 | C087 |
| 104 | 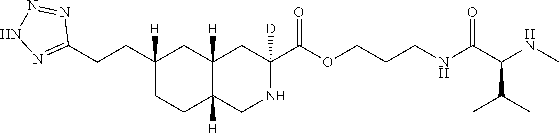 [SMILES: [2H][C@@]1(C(=O)OCCCNC(=O)[C@@H](NC)C(C)C)C[C@H]2C[C@@H](CCc3nn[nH]n3)CC[C@H]2CN1] | A02 | C088 |
| 105 | 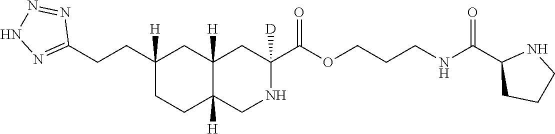 [SMILES: [2H][C@@]1(C(=O)OCCCNC(=O)[C@@H]2CCCN2)C[C@H]2C[C@@H](CCc3nn[nH]n3)CC[C@H]2CN1] | A02 | C089 |
| 106 | 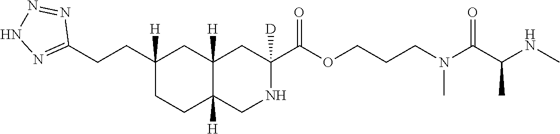 [SMILES: [2H][C@@]1(C(=O)OCCCN(C)C(=O)[C@H](C)NC)C[C@H]2C[C@@H](CCc3nn[nH]n3)CC[C@H]2CN1] | A02 | C090 |
| 107 | 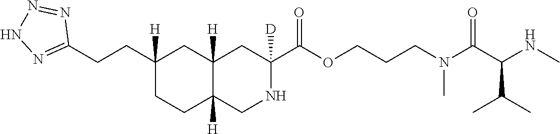 [SMILES: [2H][C@@]1(C(=O)OCCCN(C)C(=O)[C@@H](NC)C(C)C)C[C@H]2C[C@@H](CCc3nn[nH]n3)CC[C@H]2CN1] | A02 | C091 |
| 108 | 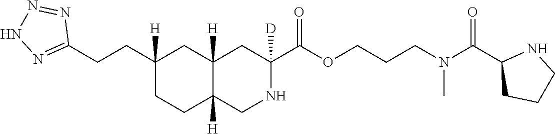 [SMILES: [2H][C@@]1(C(=O)OCCCN(C)C(=O)[C@@H]2CCCN2)C[C@H]2C[C@@H](CCc3nn[nH]n3)CC[C@H]2CN1] | A02 | C092 |
| 109 | 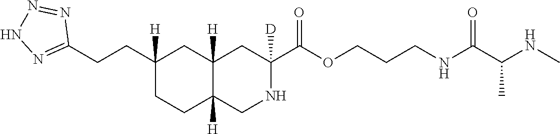 [SMILES: [2H][C@@]1(C(=O)OCCCNC(=O)[C@@H](C)NC)C[C@H]2C[C@@H](CCc3nn[nH]n3)CC[C@H]2CN1] | A02 | C093 |
| 110 | 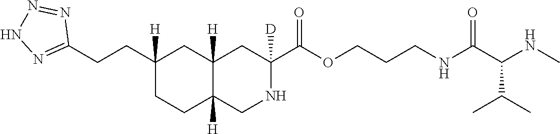 [SMILES: [2H][C@@]1(C(=O)OCCCNC(=O)[C@H](NC)C(C)C)C[C@H]2C[C@@H](CCc3nn[nH]n3)CC[C@H]2CN1] | A02 | C094 |
| 111 | 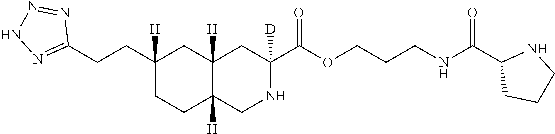 [SMILES: [2H][C@@]1(C(=O)OCCCNC(=O)[C@H]2CCCN2)C[C@H]2C[C@@H](CCc3nn[nH]n3)CC[C@H]2CN1] | A02 | C095 |
| 112 | 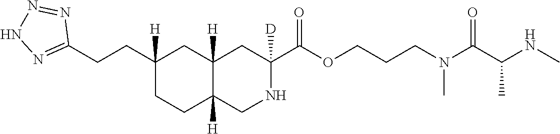 [SMILES: [2H][C@@]1(C(=O)OCCCN(C)C(=O)[C@@H](C)NC)C[C@H]2C[C@@H](CCc3nn[nH]n3)CC[C@H]2CN1] | A02 | C096 |
| 113 | 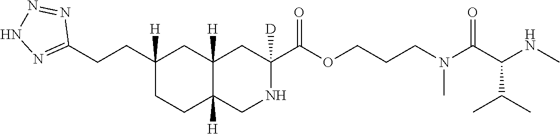 [SMILES: [2H][C@@]1(C(=O)OCCCN(C)C(=O)[C@H](NC)C(C)C)C[C@H]2C[C@@H](CCc3nn[nH]n3)CC[C@H]2CN1] | A02 | C097 |
| 114 | 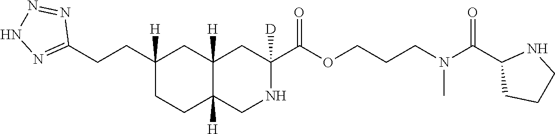 [SMILES: [2H][C@@]1(C(=O)OCCCN(C)C(=O)[C@H]2CCCN2)C[C@H]2C[C@@H](CCc3nn[nH]n3)CC[C@H]2CN1] | A02 | C098 |
| 115 | 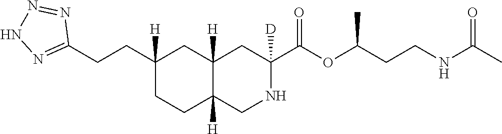 [SMILES: [2H][C@@]1(C(=O)O[C@@H](C)CCNC(C)=O)C[C@H]2C[C@@H](CCc3nn[nH]n3)CC[C@H]2CN1] | A02 | C099 |
| 116 | 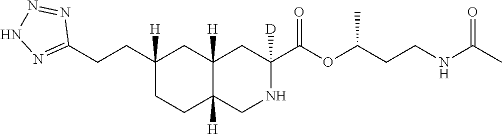 [SMILES: [2H][C@@]1(C(=O)O[C@H](C)CCNC(C)=O)C[C@H]2C[C@@H](CCc3nn[nH]n3)CC[C@H]2CN1] | A02 | C100 |
| 117 | 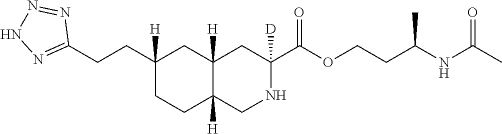 [SMILES: [2H][C@@]1(C(=O)OCC[C@@H](C)NC(C)=O)C[C@H]2C[C@@H](CCc3nn[nH]n3)CC[C@H]2CN1] | A02 | C101 |
| 118 | 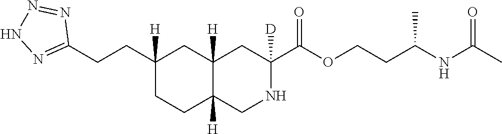 [SMILES: [2H][C@@]1(C(=O)OCC[C@H](C)NC(C)=O)C[C@H]2C[C@@H](CCc3nn[nH]n3)CC[C@H]2CN1] | A02 | C102 |
| 119 | 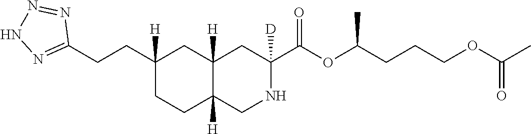 [SMILES: [2H][C@@]1(C(=O)O[C@@H](C)CCCOC(C)=O)C[C@H]2C[C@@H](CCc3nn[nH]n3)CC[C@H]2CN1] | A02 | C104 |
| 120 | 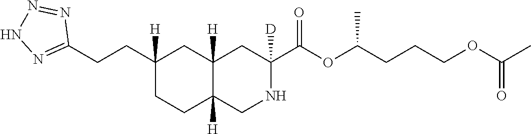 [SMILES: [2H][C@@]1(C(=O)O[C@H](C)CCCOC(C)=O)C[C@H]2C[C@@H](CCc3nn[nH]n3)CC[C@H]2CN1] | A02 | C105 |
| 121 | 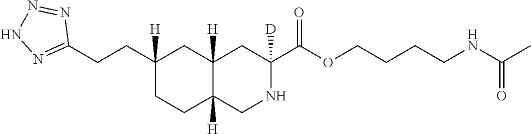 [SMILES: [2H][C@@]1(C(=O)OCCCCNC(C)=O)C[C@H]2C[C@@H](CCc3nn[nH]n3)CC[C@H]2CN1] | A02 | C106 |
| 122 | 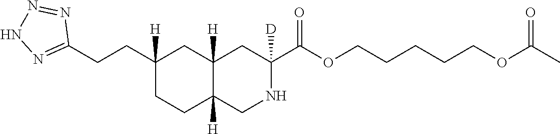 [SMILES: [2H][C@@]1(C(=O)OCCCCCOC(C)=O)C[C@H]2C[C@@H](CCc3nn[nH]n3)CC[C@H]2CN1] | A02 | C107 |
| 123 | 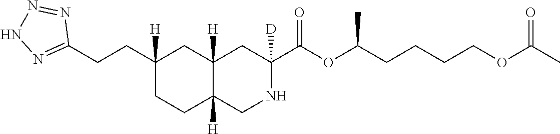 [SMILES: [2H][C@@]1(C(=O)O[C@@H](C)CCCCOC(C)=O)C[C@H]2C[C@@H](CCc3nn[nH]n3)CC[C@H]2CN1] | A02 | C108 |
| 124 | 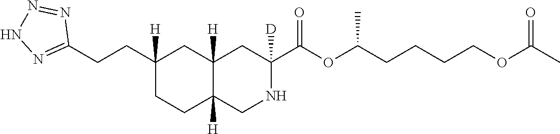 [SMILES: [2H][C@@]1(C(=O)O[C@H](C)CCCCOC(C)=O)C[C@H]2C[C@@H](CCc3nn[nH]n3)CC[C@H]2CN1] | A02 | C109 |
| 125 | 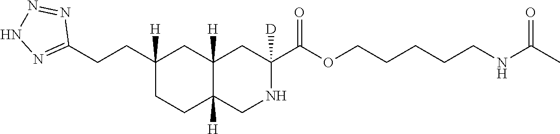 [SMILES: [2H][C@@]1(C(=O)OCCCCCNC(C)=O)C[C@H]2C[C@@H](CCc3nn[nH]n3)CC[C@H]2CN1] | A02 | C110 |
| 126 | 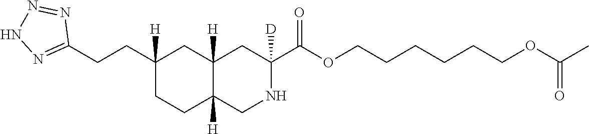 [SMILES: [2H][C@@]1(C(=O)OCCCCCCOC(C)=O)C[C@H]2C[C@@H](CCc3nn[nH]n3)CC[C@H]2CN1] | A02 | C111 |
| 127 | 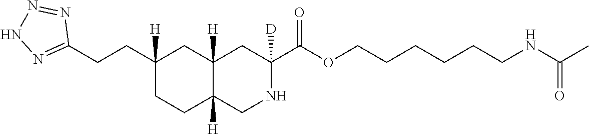 [SMILES: [2H][C@@]1(C(=O)OCCCCCCNC(C)=O)C[C@H]2C[C@@H](CCc3nn[nH]n3)CC[C@H]2CN1] | A02 | C112 |
| 128 | 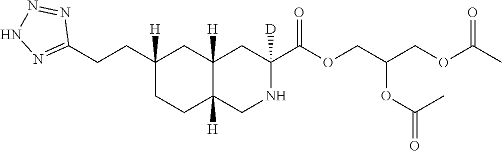 [SMILES: [2H][C@@]1(C(=O)OCC(COC(C)=O)OC(C)=O)C[C@H]2C[C@@H](CCc3nn[nH]n3)CC[C@H]2CN1] | A02 | C113 |
| 129 | 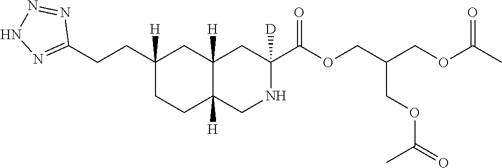 [SMILES: [2H][C@@]1(C(=O)OCC(COC(C)=O)COC(C)=O)C[C@H]2C[C@@H](CCc3nn[nH]n3)CC[C@H]2CN1] | A02 | C114 |
| 130 | 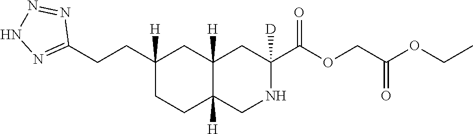 [SMILES: [2H][C@@]1(C(=O)OCC(=O)OCC)C[C@H]2C[C@@H](CCc3nn[nH]n3)CC[C@H]2CN1] | A02 | C116 |
| 131 | 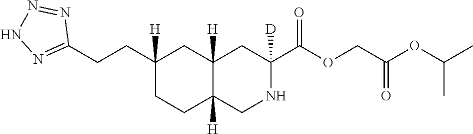 [SMILES: [2H][C@@]1(C(=O)OCC(=O)OC(C)C)C[C@H]2C[C@@H](CCc3nn[nH]n3)CC[C@H]2CN1] | A02 | C117 |
| 132 | 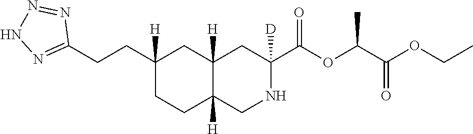 [SMILES: [2H][C@@]1(C(=O)O[C@@H](C)C(=O)OCC)C[C@H]2C[C@@H](CCc3nn[nH]n3)CC[C@H]2CN1] | A02 | C119 |
| 133 | 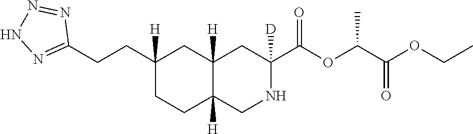 [SMILES: [2H][C@@]1(C(=O)O[C@H](C)C(=O)OCC)C[C@H]2C[C@@H](CCc3nn[nH]n3)CC[C@H]2CN1] | A02 | C121 |
| 134 | 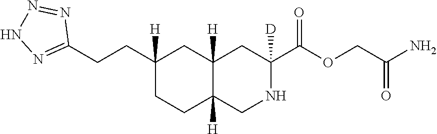 [SMILES: [2H][C@@]1(C(=O)OCC(N)=O)C[C@H]2C[C@@H](CCc3nn[nH]n3)CC[C@H]2CN1] | A02 | C122 |
| 135 | 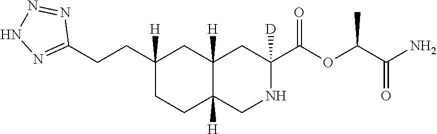 [SMILES: [2H][C@@]1(C(=O)O[C@@H](C)C(N)=O)C[C@H]2C[C@@H](CCc3nn[nH]n3)CC[C@H]2CN1] | A02 | C123 |
| 136 | 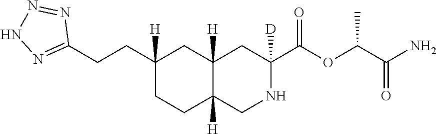 [SMILES: [2H][C@@]1(C(=O)O[C@H](C)C(N)=O)C[C@H]2C[C@@H](CCc3nn[nH]n3)CC[C@H]2CN1] | A02 | C124 |
| 137 | 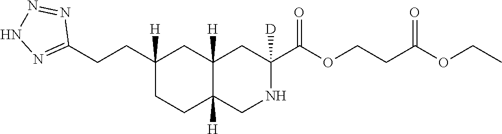 [SMILES: [2H][C@@]1(C(=O)OCCC(=O)OCC)C[C@H]2C[C@@H](CCc3nn[nH]n3)CC[C@H]2CN1] | A02 | C126 |
| 138 | 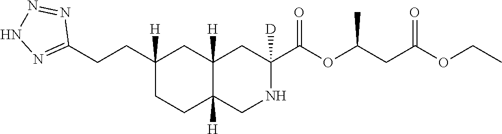 [SMILES: [2H][C@@]1(C(=O)O[C@@H](C)CC(=O)OCC)C[C@H]2C[C@@H](CCc3nn[nH]n3)CC[C@H]2CN1] | A02 | C129 |
| 139 | 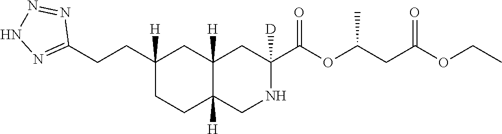 [SMILES: [2H][C@@]1(C(=O)O[C@H](C)CC(=O)OCC)C[C@H]2C[C@@H](CCc3nn[nH]n3)CC[C@H]2CN1] | A02 | C132 |
| 140 | 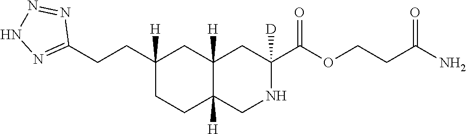 [SMILES: [2H][C@@]1(C(=O)OCCC(N)=O)C[C@H]2C[C@@H](CCc3nn[nH]n3)CC[C@H]2CN1] | A02 | C134 |
| 141 | 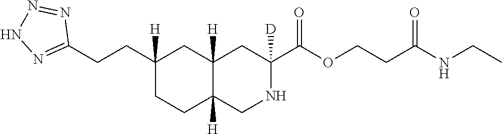 [SMILES: [2H][C@@]1(C(=O)OCCC(=O)NCC)C[C@H]2C[C@@H](CCc3nn[nH]n3)CC[C@H]2CN1] | A02 | C136 |
| 142 | 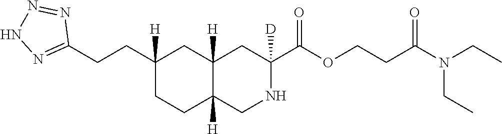 [SMILES: [2H][C@@]1(C(=O)OCCC(=O)N(CC)CC)C[C@H]2C[C@@H](CCc3nn[nH]n3)CC[C@H]2CN1] | A02 | C139 |
| 143 | 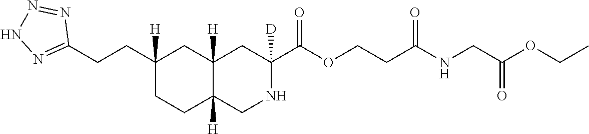 [SMILES: [2H][C@@]1(C(=O)OCCC(=O)NCC(=O)OCC)C[C@H]2C[C@@H](CCc3nn[nH]n3)CC[C@H]2CN1] | A02 | C141 |
| 144 | 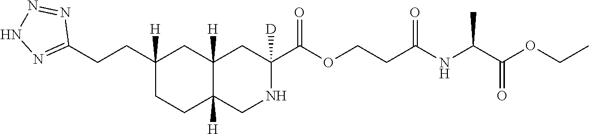 [SMILES: [2H][C@@]1(C(=O)OCCC(=O)N[C@@H](C)C(=O)OCC)C[C@H]2C[C@@H](CCc3nn[nH]n3)CC[C@H]2CN1] | A02 | C142 |
| 145 | 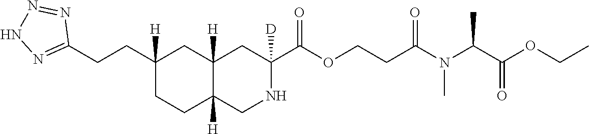 [SMILES: [2H][C@@]1(C(=O)OCCC(=O)N(C)[C@@H](C)C(=O)OCC)C[C@H]2C[C@@H](CCc3nn[nH]n3)CC[C@H]2CN1] | A02 | C144 |
| 146 | 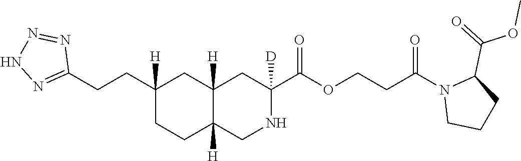 [SMILES: [2H][C@@]1(C(=O)OCCC(=O)N2CCC[C@@H]2C(=O)OC)C[C@H]2C[C@@H](CCc3nn[nH]n3)CC[C@H]2CN1] | A02 | C146 |
| 147 | 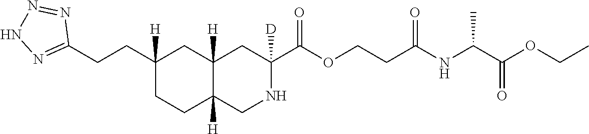 [SMILES: [2H][C@@]1(C(=O)OCCC(=O)N[C@H](C)C(=O)OCC)C[C@H]2C[C@@H](CCc3nn[nH]n3)CC[C@H]2CN1] | A02 | C147 |
| 148 | 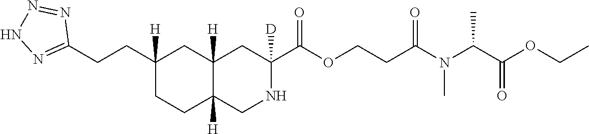 [SMILES: [2H][C@@]1(C(=O)OCCC(=O)N(C)[C@H](C)C(=O)OCC)C[C@H]2C[C@@H](CCc3nn[nH]n3)CC[C@H]2CN1] | A02 | C149 |
| 149 | 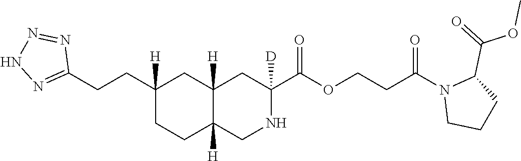 [SMILES: [2H][C@@]1(C(=O)OCCC(=O)N2CCC[C@H]2C(=O)OC)C[C@H]2C[C@@H](CCc3nn[nH]n3)CC[C@H]2CN1] | A02 | C151 |
| 150 | 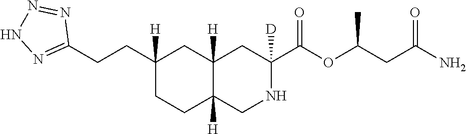 [SMILES: [2H][C@@]1(C(=O)O[C@@H](C)CC(N)=O)C[C@H]2C[C@@H](CCc3nn[nH]n3)CC[C@H]2CN1] | A02 | C152 |
| 151 | 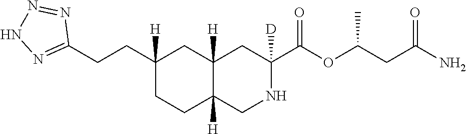 [SMILES: [2H][C@@]1(C(=O)O[C@H](C)CC(N)=O)C[C@H]2C[C@@H](CCc3nn[nH]n3)CC[C@H]2CN1] | A02 | C153 |
| 152 | 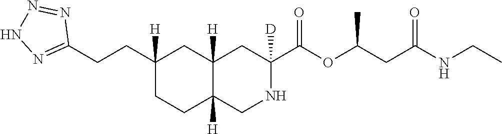 [SMILES: [2H][C@@]1(C(=O)O[C@@H](C)CC(=O)NCC)C[C@H]2C[C@@H](CCc3nn[nH]n3)CC[C@H]2CN1] | A02 | C154 |
| 153 | 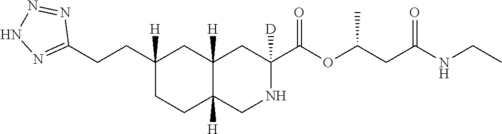 [SMILES: [2H][C@@]1(C(=O)O[C@H](C)CC(=O)NCC)C[C@H]2C[C@@H](CCc3nn[nH]n3)CC[C@H]2CN1] | A02 | C155 |
| 154 | 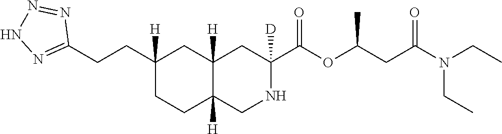 [SMILES: [2H][C@@]1(C(=O)O[C@@H](C)CC(=O)N(CC)CC)C[C@H]2C[C@@H](CCc3nn[nH]n3)CC[C@H]2CN1] | A02 | C156 |
| 155 | 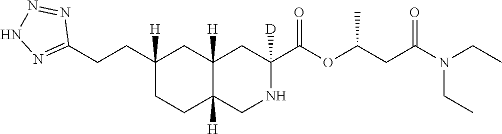 [SMILES: [2H][C@@]1(C(=O)O[C@H](C)CC(=O)N(CC)CC)C[C@H]2C[C@@H](CCc3nn[nH]n3)CC[C@H]2CN1] | A02 | C157 |
| 156 | 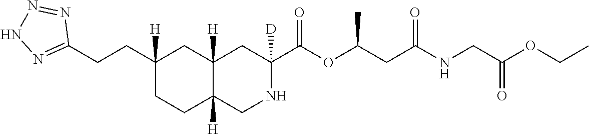 [SMILES: [2H][C@@]1(C(=O)O[C@@H](C)CC(=O)NCC(=O)OCC)C[C@H]2C[C@@H](CCc3nn[nH]n3)CC[C@H]2CN1] | A02 | C158 |
| 157 | 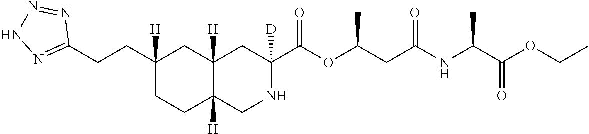 [SMILES: [2H][C@@]1(C(=O)O[C@@H](C)CC(=O)N[C@@H](C)C(=O)OCC)C[C@H]2C[C@@H](CCc3nn[nH]n3)CC[C@H]2CN1] | A02 | C159 |
| 158 | 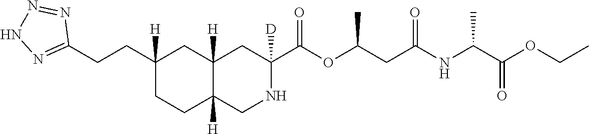 [SMILES: [2H][C@@]1(C(=O)O[C@@H](C)CC(=O)N[C@H](C)C(=O)OCC)C[C@H]2C[C@@H](CCc3nn[nH]n3)CC[C@H]2CN1] | A02 | C160 |
| 159 | 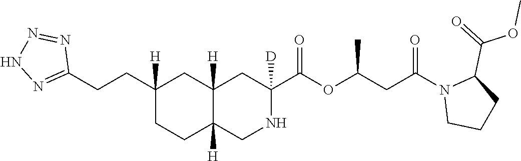 [SMILES: [2H][C@@]1(C(=O)O[C@@H](C)CC(=O)N2CCC[C@@H]2C(=O)OC)C[C@H]2C[C@@H](CCc3nn[nH]n3)CC[C@H]2CN1] | A02 | C163 |
| 160 | 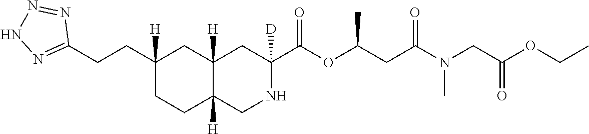 [SMILES: [2H][C@@]1(C(=O)O[C@@H](C)CC(=O)N(C)CC(=O)OCC)C[C@H]2C[C@@H](CCc3nn[nH]n3)CC[C@H]2CN1] | A02 | C164 |
| 161 | 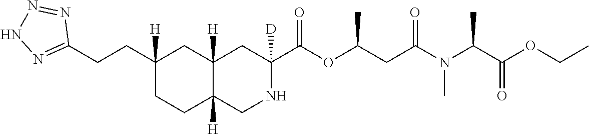 [SMILES: [2H][C@@]1(C(=O)O[C@@H](C)CC(=O)N(C)[C@@H](C)C(=O)OCC)C[C@H]2C[C@@H](CCc3nn[nH]n3)CC[C@H]2CN1] | A02 | C165 |
| 162 | 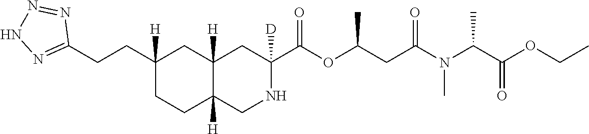 [SMILES: [2H][C@@]1(C(=O)O[C@@H](C)CC(=O)N(C)[C@H](C)C(=O)OCC)C[C@H]2C[C@@H](CCc3nn[nH]n3)CC[C@H]2CN1] | A02 | C166 |
| 163 | 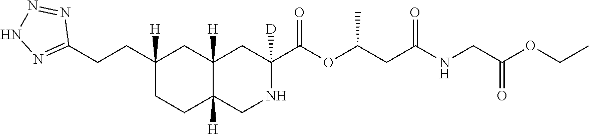 [SMILES: [2H][C@@]1(C(=O)O[C@H](C)CC(=O)NCC(=O)OCC)C[C@H]2C[C@@H](CCc3nn[nH]n3)CC[C@H]2CN1] | A02 | C169 |
| 164 | 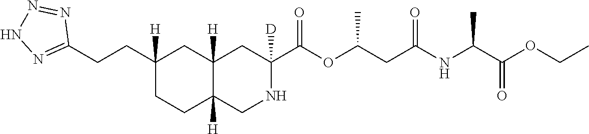 [SMILES: [2H][C@@]1(C(=O)O[C@H](C)CC(=O)N[C@@H](C)C(=O)OCC)C[C@H]2C[C@@H](CCc3nn[nH]n3)CC[C@H]2CN1] | A02 | C170 |
| 165 | 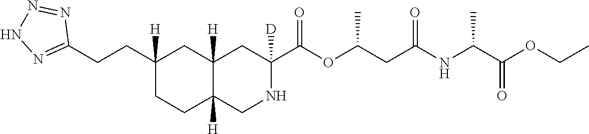 [SMILES: [2H][C@@]1(C(=O)O[C@H](C)CC(=O)N[C@H](C)C(=O)OCC)C[C@H]2C[C@@H](CCc3nn[nH]n3)CC[C@H]2CN1] | A02 | C171 |
| 166 | 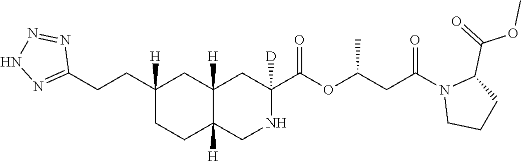 [SMILES: [2H][C@@]1(C(=O)O[C@H](C)CC(=O)N2CCC[C@H]2C(=O)OC)C[C@H]2C[C@@H](CCc3nn[nH]n3)CC[C@H]2CN1] | A02 | C174 |
| 167 | 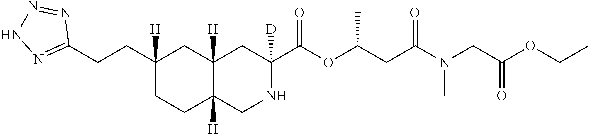 [SMILES: [2H][C@@]1(C(=O)O[C@H](C)CC(=O)N(C)CC(=O)OCC)C[C@H]2C[C@@H](CCc3nn[nH]n3)CC[C@H]2CN1] | A02 | C175 |
| 168 | 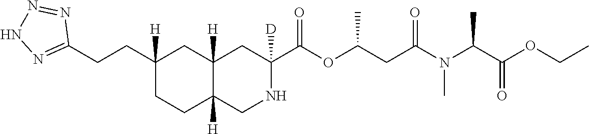 [SMILES: [2H][C@@]1(C(=O)O[C@H](C)CC(=O)N(C)[C@@H](C)C(=O)OCC)C[C@H]2C[C@@H](CCc3nn[nH]n3)CC[C@H]2CN1] | A02 | C176 |
| 169 | 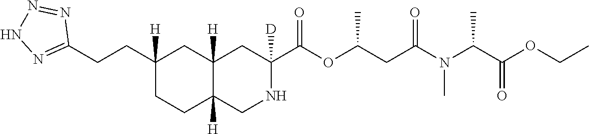 [SMILES: [2H][C@@]1(C(=O)O[C@H](C)CC(=O)N(C)[C@H](C)C(=O)OCC)C[C@H]2C[C@@H](CCc3nn[nH]n3)CC[C@H]2CN1] | A02 | C178 |
| 170 | 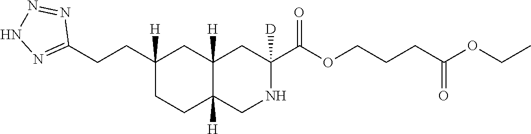 [SMILES: [2H][C@@]1(C(=O)OCCCC(=O)OCC)C[C@H]2C[C@@H](CCc3nn[nH]n3)CC[C@H]2CN1] | A02 | C180 |
| 171 | 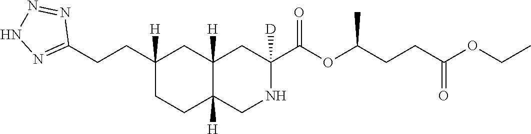 [SMILES: [2H][C@@]1(C(=O)O[C@@H](C)CCC(=O)OCC)C[C@H]2C[C@@H](CCc3nn[nH]n3)CC[C@H]2CN1] | A02 | C181 |
| 172 | 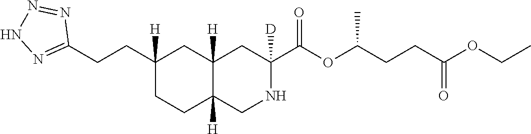 [SMILES: [2H][C@@]1(C(=O)O[C@H](C)CCC(=O)OCC)C[C@H]2C[C@@H](CCc3nn[nH]n3)CC[C@H]2CN1] | A02 | C182 |
| 173 | 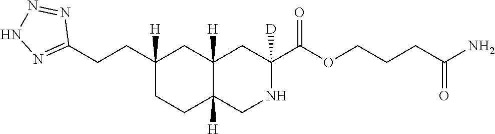 [SMILES: [2H][C@@]1(C(=O)OCCCC(N)=O)C[C@H]2C[C@@H](CCc3nn[nH]n3)CC[C@H]2CN1] | A02 | C183 |
| 174 | 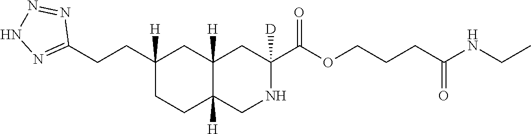 [SMILES: [2H][C@@]1(C(=O)OCCCC(=O)NCC)C[C@H]2C[C@@H](CCc3nn[nH]n3)CC[C@H]2CN1] | A02 | C184 |
| 175 | 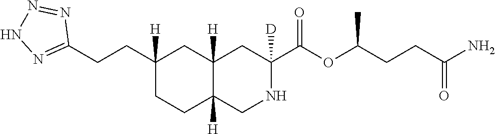 [SMILES: [2H][C@@]1(C(=O)O[C@@H](C)CCC(N)=O)C[C@H]2C[C@@H](CCc3nn[nH]n3)CC[C@H]2CN1] | A02 | C185 |
| 176 | 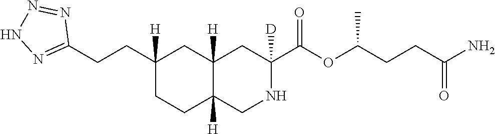 [SMILES: [2H][C@@]1(C(=O)O[C@H](C)CCC(N)=O)C[C@H]2C[C@@H](CCc3nn[nH]n3)CC[C@H]2CN1] | A02 | C186 |
| 177 | 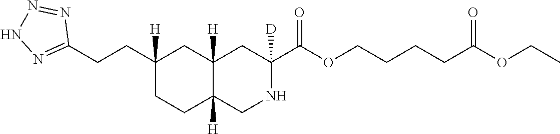 [SMILES: [2H][C@@]1(C(=O)OCCCCC(=O)OCC)C[C@H]2C[C@@H](CCc3nn[nH]n3)CC[C@H]2CN1] | A02 | C191 |
| 178 | 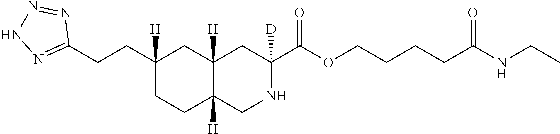 [SMILES: [2H][C@@]1(C(=O)OCCCCC(=O)NCC)C[C@H]2C[C@@H](CCc3nn[nH]n3)CC[C@H]2CN1] | A02 | C195 |
| 179 | 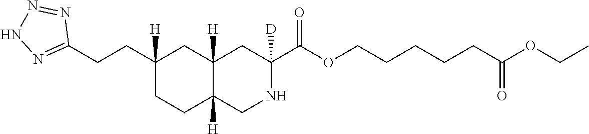 [SMILES: [2H][C@@]1(C(=O)OCCCCCC(=O)OCC)C[C@H]2C[C@@H](CCc3nn[nH]n3)CC[C@H]2CN1] | A02 | C196 |
| 180 | 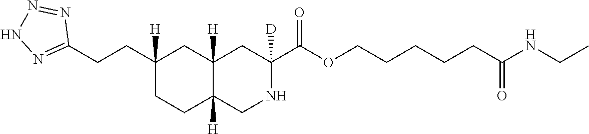 [SMILES: [2H][C@@]1(C(=O)OCCCCCC(=O)NCC)C[C@H]2C[C@@H](CCc3nn[nH]n3)CC[C@H]2CN1] | A02 | C198 |
| 181 | 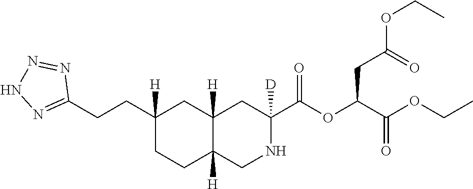 [SMILES: [2H][C@@]1(C(=O)O[C@@H](CC(=O)OCC)C(=O)OCC)C[C@H]2C[C@@H](CCc3nn[nH]n3)CC[C@H]2CN1] | A02 | C199 |
| 182 | 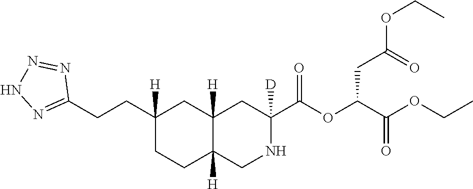 [SMILES: [2H][C@@]1(C(=O)O[C@H](CC(=O)OCC)C(=O)OCC)C[C@H]2C[C@@H](CCc3nn[nH]n3)CC[C@H]2CN1] | A02 | C200 |
| 183 | 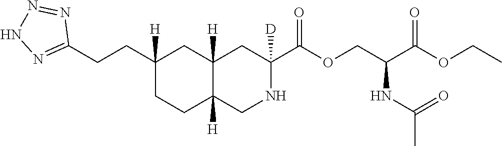 [SMILES: [2H][C@@]1(C(=O)OC[C@H](NC(C)=O)C(=O)OCC)C[C@H]2C[C@@H](CCc3nn[nH]n3)CC[C@H]2CN1] | A02 | C202 |
| 184 | 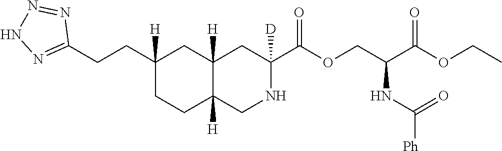 [SMILES: [2H][C@@]1(C(=O)OC[C@H](NC(=O)c2ccccc2)C(=O)OCC)C[C@H]2C[C@@H](CCc3nn[nH]n3)CC[C@H]2CN1] | A02 | C204 |
| 185 | 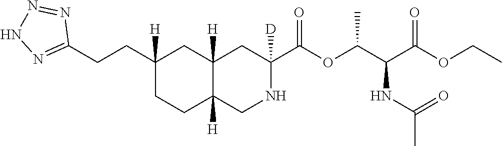 [SMILES: [2H][C@@]1(C(=O)O[C@H](C)[C@H](NC(C)=O)C(=O)OCC)C[C@H]2C[C@@H](CCc3nn[nH]n3)CC[C@H]2CN1] | A02 | C206 |
| 186 | 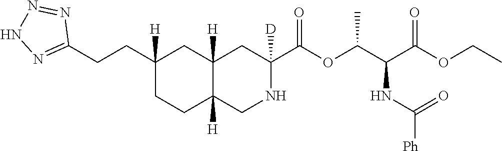 [SMILES: [2H][C@@]1(C(=O)O[C@H](C)[C@H](NC(=O)c2ccccc2)C(=O)OCC)C[C@H]2C[C@@H](CCc3nn[nH]n3)CC[C@H]2CN1] | A02 | C207 |
| 187 | 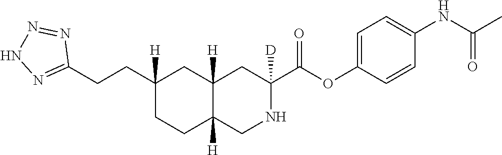 [SMILES: [2H][C@@]1(C(=O)Oc2ccc(NC(C)=O)cc2)C[C@H]2C[C@@H](CCc3nn[nH]n3)CC[C@H]2CN1] | A02 | C209 |
| 188 | 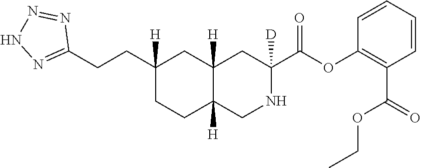 [SMILES: [2H][C@@]1(C(=O)Oc2ccccc2C(=O)OCC)C[C@H]2C[C@@H](CCc3nn[nH]n3)CC[C@H]2CN1] | A02 | C211 |
| 189 | 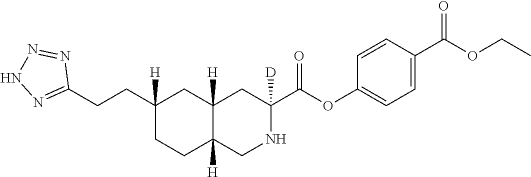 [SMILES: [2H][C@@]1(C(=O)Oc2ccc(C(=O)OCC)cc2)C[C@H]2C[C@@H](CCc3nn[nH]n3)CC[C@H]2CN1] | A02 | C214 |
| 190 | 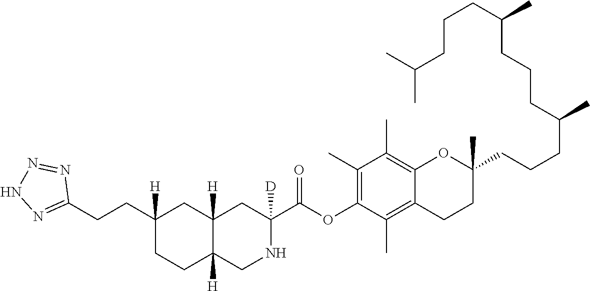 [SMILES: [2H][C@@]1(C(=O)Oc2c(C)c(C)c3c(c2C)CC[C@@](C)(CCC[C@H](C)CCC[C@H](C)CCCC(C)C)O3)C[C@H]2C[C@@H](CCc3nn[nH]n3)CC[C@H]2CN1] | A02 | C216 |
| 191 | 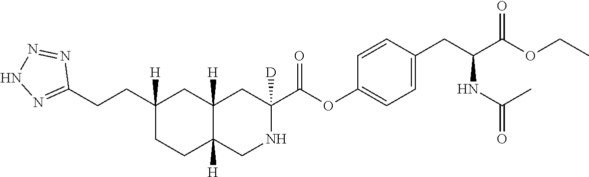 [SMILES: [2H][C@@]1(C(=O)Oc2ccc(C[C@H](NC(C)=O)C(=O)OCC)cc2)C[C@H]2C[C@@H](CCc3nn[nH]n3)CC[C@H]2CN1] | A02 | C217 |
| 192 | 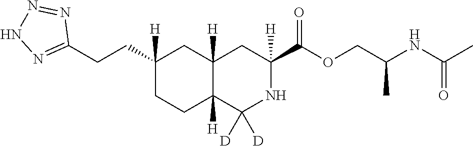 [SMILES: [2H]C1([2H])N[C@H](C(=O)OC[C@H](C)NC(C)=O)C[C@H]2C[C@@H](CCc3nn[nH]n3)CC[C@H]21] | A03 | C032 |
| 193 | 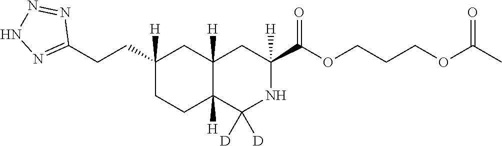 [SMILES: [2H]C1([2H])N[C@H](C(=O)OCCCOC(C)=O)C[C@H]2C[C@@H](CCc3nn[nH]n3)CC[C@H]21] | A03 | C034 |
| 194 | 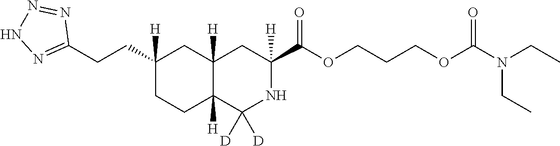 [SMILES: [2H]C1([2H])N[C@H](C(=O)OCCCOC(=O)N(CC)CC)C[C@H]2C[C@@H](CCc3nn[nH]n3)CC[C@H]21] | A03 | C064 |
| 195 | 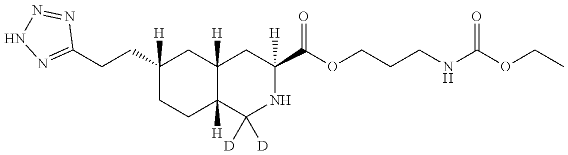 [SMILES: [2H]C1([2H])N[C@H](C(=O)OCCCNC(=O)OCC)C[C@H]2C[C@@H](CCc3nn[nH]n3)CC[C@H]21] | A03 | C067 |
| 196 | 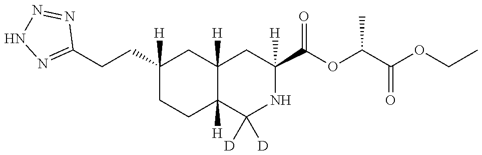 [SMILES: [2H]C1([2H])N[C@H](C(=O)O[C@H](C)C(=O)OCC)C[C@H]2C[C@@H](CCc3nn[nH]n3)CC[C@H]21] | A03 | C121 |
| 197 | 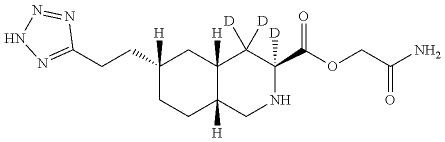 [SMILES: [2H]C1([2H])[C@H]2C[C@@H](CCc3nn[nH]n3)CC[C@H]2CN[C@]1([2H])C(=O)OCC(N)=O] | A04 | C122 |
| 198 | 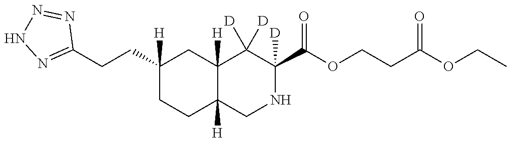 [SMILES: [2H]C1([2H])[C@H]2C[C@@H](CCc3nn[nH]n3)CC[C@H]2CN[C@]1([2H])C(=O)OCCC(=O)OCC] | A04 | C126 |
| 199 | 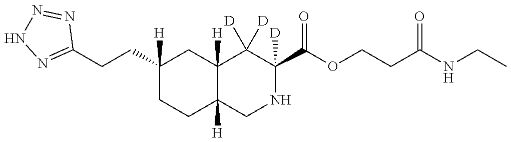 [SMILES: [2H]C1([2H])[C@H]2C[C@@H](CCc3nn[nH]n3)CC[C@H]2CN[C@]1([2H])C(=O)OCCC(=O)NCC] | A04 | C136 |
| 200 | 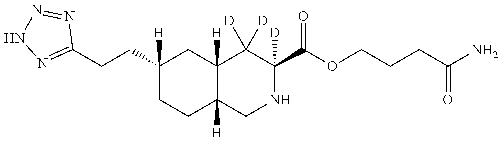 [SMILES: [2H]C1([2H])[C@H]2C[C@@H](CCc3nn[nH]n3)CC[C@H]2CN[C@]1([2H])C(=O)OCCCC(N)=O] | A04 | C183 |
| 201 | 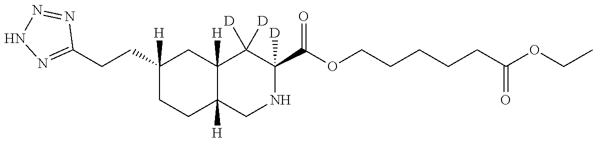 [SMILES: [2H]C1([2H])[C@H]2C[C@@H](CCc3nn[nH]n3)CC[C@H]2CN[C@]1([2H])C(=O)OCCCCCC(=O)OCC] | A04 | C196 |
| 202 | 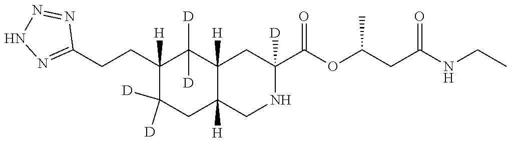 | A05 | C155 |
| 203 | 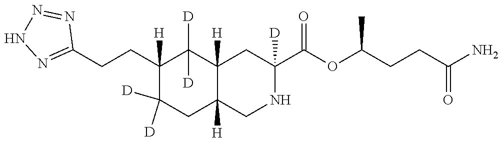 | A05 | C185 |
| 204 | 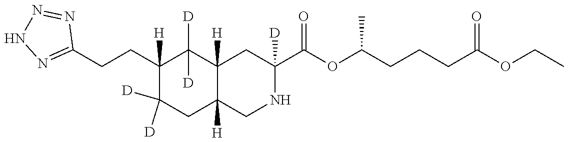 | A05 | C193 |
| 205 | 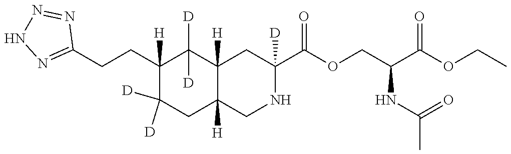 | A05 | C202 |
| 206 | 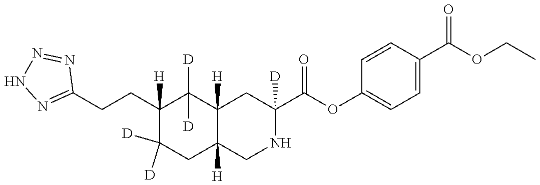 | A05 | C214 |
| 207 | 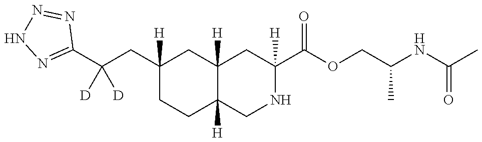 [SMILES: [2H]C([2H])(C[C@H]1CC[C@H]2CN[C@H](C(=O)OC[C@@H](C)NC(C)=O)C[C@H]2C1)c1nn[nH]n1] | A06 | C033 |
| 208 | 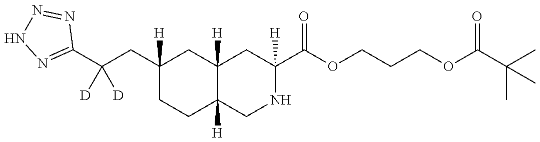 [SMILES: [2H]C([2H])(C[C@H]1CC[C@H]2CN[C@H](C(=O)OCCCOC(=O)C(C)(C)C)C[C@H]2C1)c1nn[nH]n1] | A06 | C037 |
| 209 | 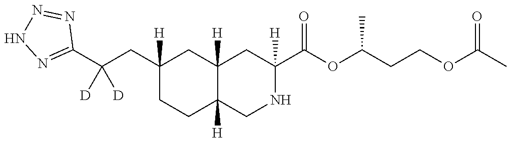 [SMILES: [2H]C([2H])(C[C@H]1CC[C@H]2CN[C@H](C(=O)O[C@H](C)CCOC(C)=O)C[C@H]2C1)c1nn[nH]n1] | A06 | C051 |
| 210 | 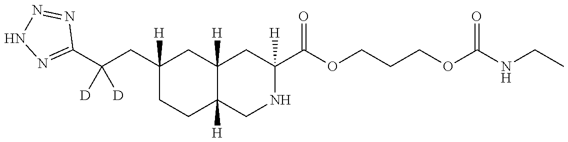 [SMILES: [2H]C([2H])(C[C@H]1CC[C@H]2CN[C@H](C(=O)OCCCOC(=O)NCC)C[C@H]2C1)c1nn[nH]n1] | A06 | C062 |
| 211 | 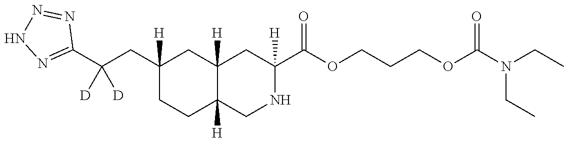 [SMILES: [2H]C([2H])(C[C@H]1CC[C@H]2CN[C@H](C(=O)OCCCOC(=O)N(CC)CC)C[C@H]2C1)c1nn[nH]n1] | A06 | C064 |
| 212 | 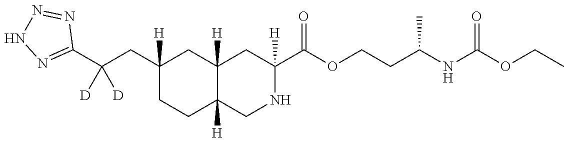 [SMILES: [2H]C([2H])(C[C@H]1CC[C@H]2CN[C@H](C(=O)OCC[C@H](C)NC(=O)OCC)C[C@H]2C1)c1nn[nH]n1] | A06 | C073 |
| 213 | 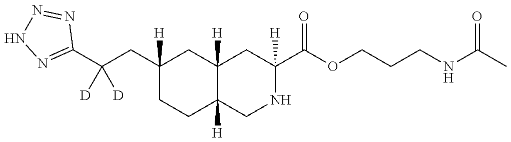 [SMILES: [2H]C([2H])(C[C@H]1CC[C@H]2CN[C@H](C(=O)OCCCNC(C)=O)C[C@H]2C1)c1nn[nH]n1] | A06 | C075 |
| 214 | 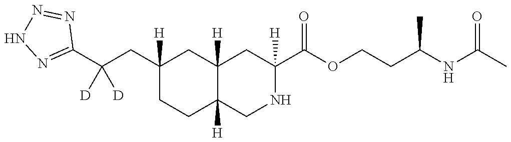 [SMILES: [2H]C([2H])(C[C@H]1CC[C@H]2CN[C@H](C(=O)OCC[C@@H](C)NC(C)=O)C[C@H]2C1)c1nn[nH]n1] | A06 | C101 |
| 215 | 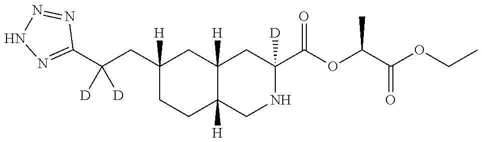 [SMILES: [2H]C([2H])(C[C@H]1CC[C@H]2CN[C@]([2H])(C(=O)O[C@@H](C)C(=O)OCC)C[C@H]2C1)c1nn[nH]n1] | A07 | C119 |
| 216 | 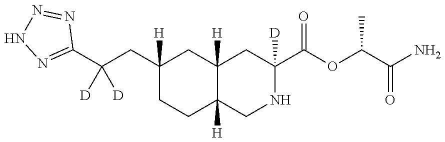 [SMILES: [2H]C([2H])(C[C@H]1CC[C@H]2CN[C@]([2H])(C(=O)O[C@H](C)C(N)=O)C[C@H]2C1)c1nn[nH]n1] | A07 | C124 |
| 217 | 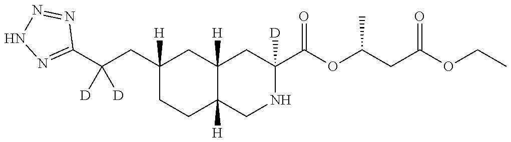 [SMILES: [2H]C([2H])(C[C@H]1CC[C@H]2CN[C@]([2H])(C(=O)O[C@H](C)CC(=O)OCC)C[C@H]2C1)c1nn[nH]n1] | A07 | C132 |
| 218 | 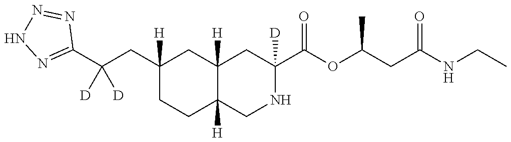 [SMILES: [2H]C([2H])(C[C@H]1CC[C@H]2CN[C@]([2H])(C(=O)O[C@@H](C)CC(=O)NCC)C[C@H]2C1)c1nn[nH]n1] | A07 | C154 |
| 219 | 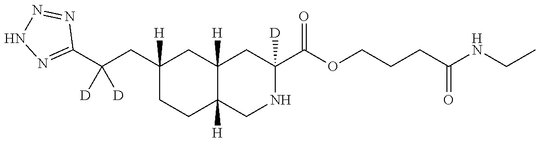 [SMILES: [2H]C([2H])(C[C@H]1CC[C@H]2CN[C@]([2H])(C(=O)OCCCC(=O)NCC)C[C@H]2C1)c1nn[nH]n1] | A07 | C184 |
| 220 | 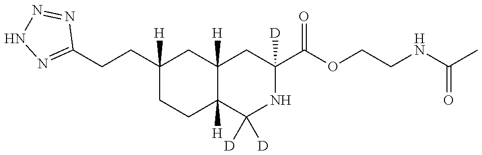 [SMILES: [2H]C1([2H])N[C@]([2H])(C(=O)OCCNC(C)=O)C[C@H]2C[C@@H](CCc3nn[nH]n3)CC[C@H]21] | A08 | C002 |
| 221 | 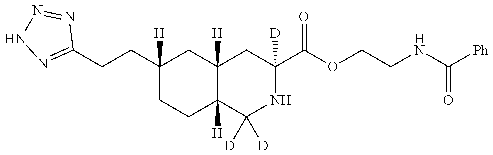 [SMILES: [2H]C1([2H])N[C@]([2H])(C(=O)OCCNC(=O)c2ccccc2)C[C@H]2C[C@@H](CCc3nn[nH]n3)CC[C@H]21] | A08 | C007 |
| 222 | 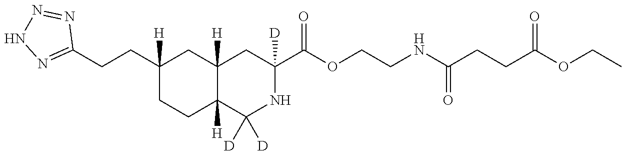 [SMILES: [2H]C1([2H])N[C@]([2H])(C(=O)OCCNC(=O)CCC(=O)OCC)C[C@H]2C[C@@H](CCc3nn[nH]n3)CC[C@H]21] | A08 | C009 |
| 223 | 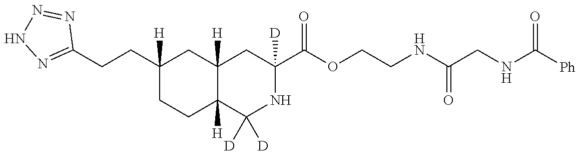 [SMILES: [2H]C1([2H])N[C@]([2H])(C(=O)OCCNC(=O)CNC(=O)c2ccccc2)C[C@H]2C[C@@H](CCc3nn[nH]n3)CC[C@H]21] | A08 | C028 |
| 224 | 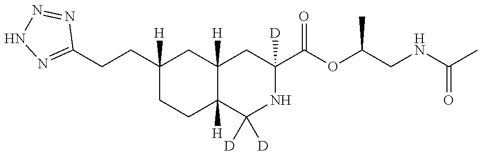 [SMILES: [2H]C1([2H])N[C@]([2H])(C(=O)O[C@@H](C)CNC(C)=O)C[C@H]2C[C@@H](CCc3nn[nH]n3)CC[C@H]21] | A08 | C030 |
| 225 | 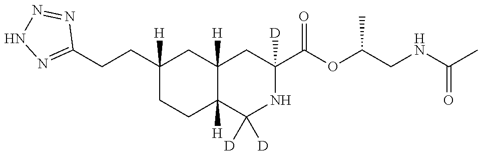 [SMILES: [2H]C1([2H])N[C@]([2H])(C(=O)O[C@H](C)CNC(C)=O)C[C@H]2C[C@@H](CCc3nn[nH]n3)CC[C@H]21] | A08 | C031 |
| 226 | 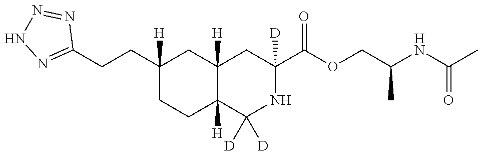 [SMILES: [2H]C1([2H])N[C@]([2H])(C(=O)OC[C@H](C)NC(C)=O)C[C@H]2C[C@@H](CCc3nn[nH]n3)CC[C@H]21] | A08 | C032 |
| 227 | 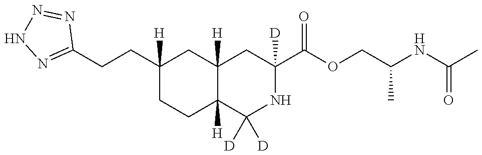 [SMILES: [2H]C1([2H])N[C@]([2H])(C(=O)OC[C@@H](C)NC(C)=O)C[C@H]2C[C@@H](CCc3nn[nH]n3)CC[C@H]21] | A08 | C033 |
| 228 | 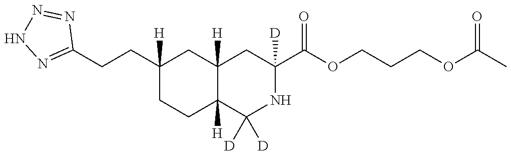 [SMILES: [2H]C1([2H])N[C@]([2H])(C(=O)OCCCOC(C)=O)C[C@H]2C[C@@H](CCc3nn[nH]n3)CC[C@H]21] | A08 | C034 |
| 229 | 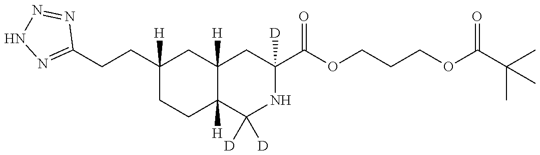 [SMILES: [2H]C1([2H])N[C@]([2H])(C(=O)OCCCOC(=O)C(C)(C)C)C[C@H]2C[C@@H](CCc3nn[nH]n3)CC[C@H]21] | A08 | C037 |
| 230 | 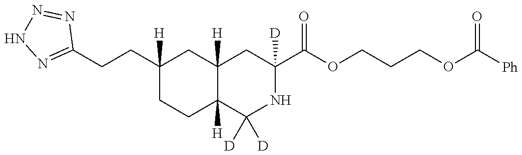 [SMILES: [2H]C1([2H])N[C@]([2H])(C(=O)OCCCOC(=O)c2ccccc2)C[C@H]2C[C@@H](CCc3nn[nH]n3)CC[C@H]21] | A08 | C038 |
| 231 | 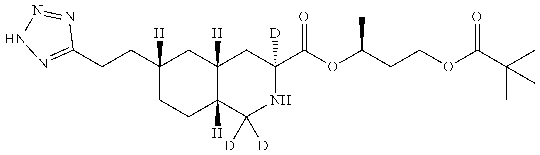 [SMILES: [2H]C1([2H])N[C@]([2H])(C(=O)O[C@@H](C)CCOC(=O)C(C)(C)C)C[C@H]2C[C@@H](CCc3nn[nH]n3)CC[C@H]21] | A08 | C049 |
| 232 | 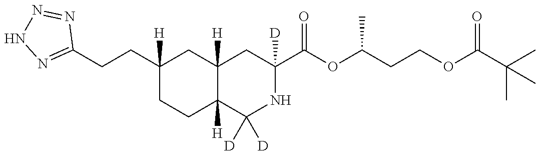 [SMILES: [2H]C1([2H])N[C@]([2H])(C(=O)O[C@H](C)CCOC(=O)C(C)(C)C)C[C@H]2C[C@@H](CCc3nn[nH]n3)CC[C@H]21] | A08 | C053 |
| 233 | 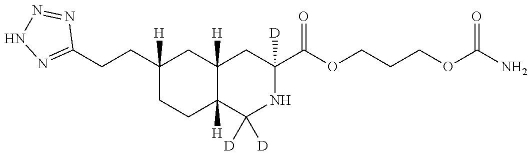 [SMILES: [2H]C1([2H])N[C@]([2H])(C(=O)OCCCOC(N)=O)C[C@H]2C[C@@H](CCc3nn[nH]n3)CC[C@H]21] | A08 | C061 |
| 234 | 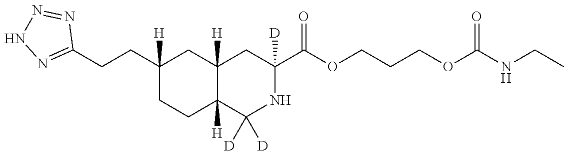 [SMILES: [2H]C1([2H])N[C@]([2H])(C(=O)OCCCOC(=O)NCC)C[C@H]2C[C@@H](CCc3nn[nH]n3)CC[C@H]21] | A08 | C062 |
| 235 | 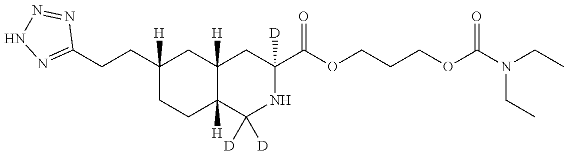 [SMILES: [2H]C1([2H])N[C@]([2H])(C(=O)OCCCOC(=O)N(CC)CC)C[C@H]2C[C@@H](CCc3nn[nH]n3)CC[C@H]21] | A08 | C064 |
| 236 | 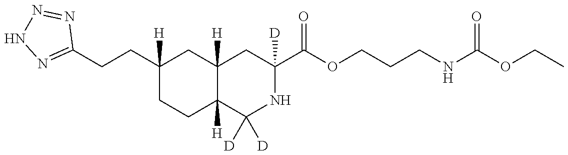 [SMILES: [2H]C1([2H])N[C@]([2H])(C(=O)OCCCNC(=O)OCC)C[C@H]2C[C@@H](CCc3nn[nH]n3)CC[C@H]21] | A08 | C067 |
| 237 | 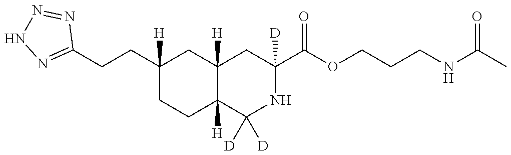 [SMILES: [2H]C1([2H])N[C@]([2H])(C(=O)OCCCNC(C)=O)C[C@H]2C[C@@H](CCc3nn[nH]n3)CC[C@H]21] | A08 | C075 |
| 238 | 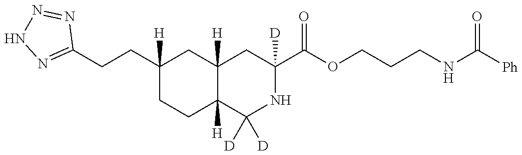 [SMILES: [2H]C1([2H])N[C@]([2H])(C(=O)OCCCNC(=O)c2ccccc2)C[C@H]2C[C@@H](CCc3nn[nH]n3)CC[C@H]21] | A08 | C079 |
| 239 | 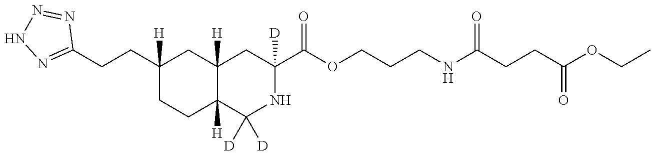 [SMILES: [2H]C1([2H])N[C@]([2H])(C(=O)OCCCNC(=O)CCC(=O)OCC)C[C@H]2C[C@@H](CCc3nn[nH]n3)CC[C@H]21] | A08 | C080 |
| 240 | 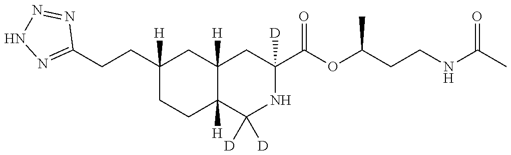 [SMILES: [2H]C1([2H])N[C@]([2H])(C(=O)O[C@@H](C)CCNC(C)=O)C[C@H]2C[C@@H](CCc3nn[nH]n3)CC[C@H]21] | A08 | C099 |
| 241 | 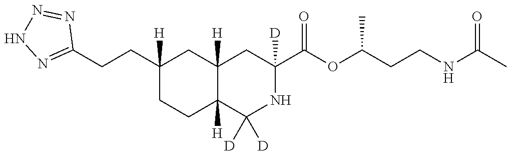 [SMILES: [2H]C1([2H])N[C@]([2H])(C(=O)O[C@H](C)CCNC(C)=O)C[C@H]2C[C@@H](CCc3nn[nH]n3)CC[C@H]21] | A08 | C100 |
| 242 | 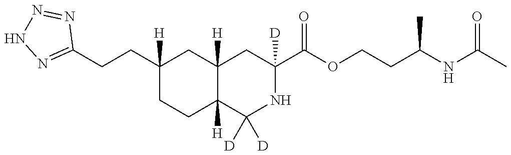 [SMILES: [2H]C1([2H])N[C@]([2H])(C(=O)OCC[C@@H](C)NC(C)=O)C[C@H]2C[C@@H](CCc3nn[nH]n3)CC[C@H]21] | A08 | C101 |
| 243 | 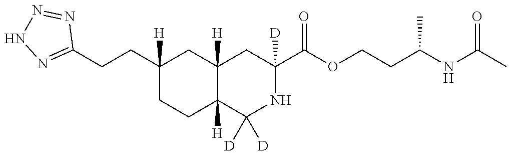 [SMILES: [2H]C1([2H])N[C@]([2H])(C(=O)OCC[C@H](C)NC(C)=O)C[C@H]2C[C@@H](CCc3nn[nH]n3)CC[C@H]21] | A08 | C102 |
| 244 | 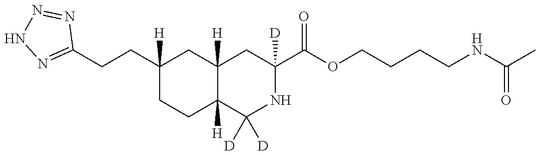 [SMILES: [2H]C1([2H])N[C@]([2H])(C(=O)OCCCCNC(C)=O)C[C@H]2C[C@@H](CCc3nn[nH]n3)CC[C@H]21] | A08 | C106 |
| 245 | 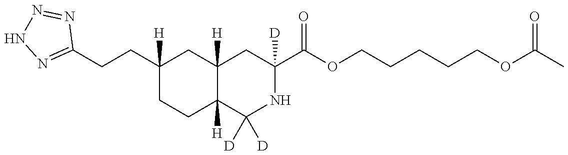 [SMILES: [2H]C1([2H])N[C@]([2H])(C(=O)OCCCCCOC(C)=O)C[C@H]2C[C@@H](CCc3nn[nH]n3)CC[C@H]21] | A08 | C107 |
| 246 | 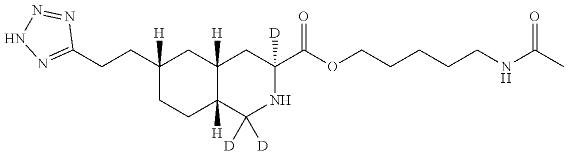 [SMILES: [2H]C1([2H])N[C@]([2H])(C(=O)OCCCCCNC(C)=O)C[C@H]2C[C@@H](CCc3nn[nH]n3)CC[C@H]21] | A08 | C110 |
| 247 | 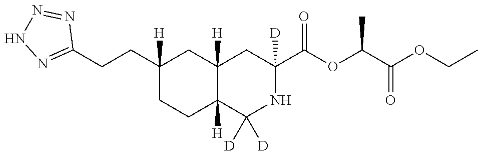 [SMILES: [2H]C1([2H])N[C@]([2H])(C(=O)O[C@@H](C)C(=O)OCC)C[C@H]2C[C@@H](CCc3nn[nH]n3)CC[C@H]21] | A08 | C119 |
| 248 | 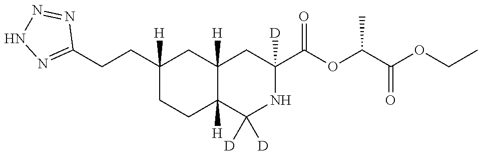 [SMILES: [2H]C1([2H])N[C@]([2H])(C(=O)O[C@H](C)C(=O)OCC)C[C@H]2C[C@@H](CCc3nn[nH]n3)CC[C@H]21] | A08 | C121 |
| 249 | 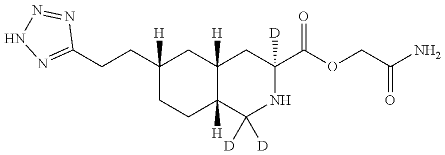 [SMILES: [2H]C1([2H])N[C@]([2H])(C(=O)OCC(N)=O)C[C@H]2C[C@@H](CCc3nn[nH]n3)CC[C@H]21] | A08 | C122 |
| 250 | 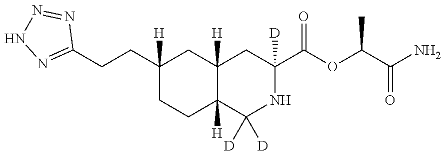 [SMILES: [2H]C1([2H])N[C@]([2H])(C(=O)O[C@@H](C)C(N)=O)C[C@H]2C[C@@H](CCc3nn[nH]n3)CC[C@H]21] | A08 | C123 |
| 251 | 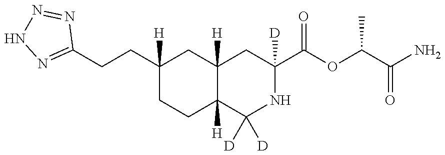 [SMILES: [2H]C1([2H])N[C@]([2H])(C(=O)O[C@H](C)C(N)=O)C[C@H]2C[C@@H](CCc3nn[nH]n3)CC[C@H]21] | A08 | C124 |
| 252 | 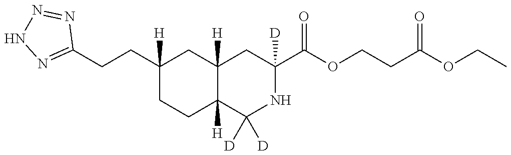 [SMILES: [2H]C1([2H])N[C@]([2H])(C(=O)OCCC(=O)OCC)C[C@H]2C[C@@H](CCc3nn[nH]n3)CC[C@H]21] | A08 | C126 |
| 253 | 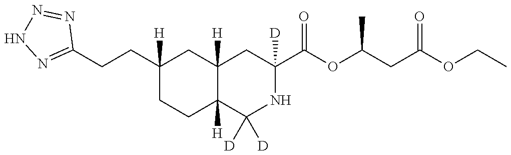 [SMILES: [2H]C1([2H])N[C@]([2H])(C(=O)O[C@@H](C)CC(=O)OCC)C[C@H]2C[C@@H](CCc3nn[nH]n3)CC[C@H]21] | A08 | C129 |
| 254 | 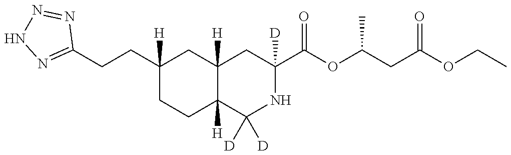 [SMILES: [2H]C1([2H])N[C@]([2H])(C(=O)O[C@H](C)CC(=O)OCC)C[C@H]2C[C@@H](CCc3nn[nH]n3)CC[C@H]21] | A08 | C132 |
| 255 | 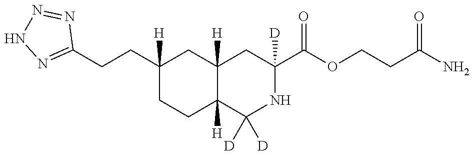 [SMILES: [2H]C1([2H])N[C@]([2H])(C(=O)OCCC(N)=O)C[C@H]2C[C@@H](CCc3nn[nH]n3)CC[C@H]21] | A08 | C134 |
| 256 | 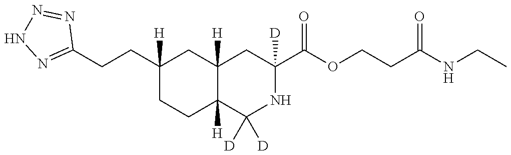 [SMILES: [2H]C1([2H])N[C@]([2H])(C(=O)OCCC(=O)NCC)C[C@H]2C[C@@H](CCc3nn[nH]n3)CC[C@H]21] | A08 | C136 |
| 257 | 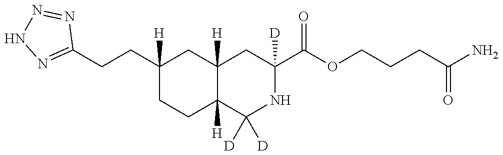 [SMILES: [2H]C1([2H])N[C@]([2H])(C(=O)OCCCC(N)=O)C[C@H]2C[C@@H](CCc3nn[nH]n3)CC[C@H]21] | A08 | C183 |
| 258 | 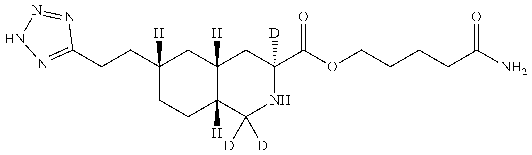 [SMILES: [2H]C1([2H])N[C@]([2H])(C(=O)OCCCCC(N)=O)C[C@H]2C[C@@H](CCc3nn[nH]n3)CC[C@H]21] | A08 | C194 |
| 259 | 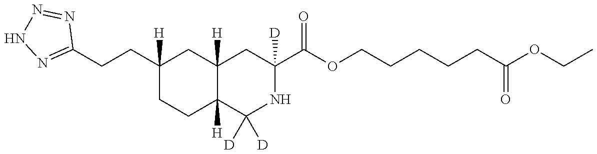 [SMILES: [2H]C1([2H])N[C@]([2H])(C(=O)OCCCCCC(=O)OCC)C[C@H]2C[C@@H](CCc3nn[nH]n3)CC[C@H]21] | A08 | C196 |
| 260 | 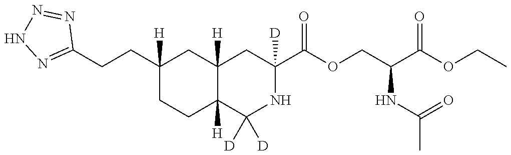 [SMILES: [2H]C1([2H])N[C@]([2H])(C(=O)OC[C@H](NC(C)=O)C(=O)OCC)C[C@H]2C[C@@H](CCc3nn[nH]n3)CC[C@H]21] | A08 | C202 |
| 261 | 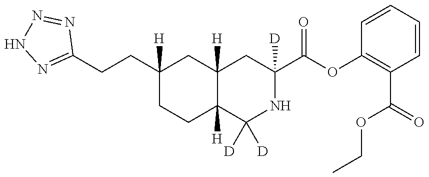 [SMILES: [2H]C1([2H])N[C@]([2H])(C(=O)Oc2ccccc2C(=O)OCC)C[C@H]2C[C@@H](CCc3nn[nH]n3)CC[C@H]21] | A08 | C211 |
| 262 | 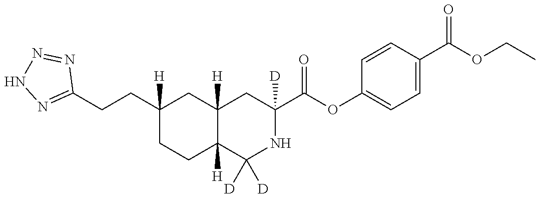 [SMILES: [2H]C1([2H])N[C@]([2H])(C(=O)Oc2ccc(C(=O)OCC)cc2)C[C@H]2C[C@@H](CCc3nn[nH]n3)CC[C@H]21] | A08 | C214 |
| 263 | 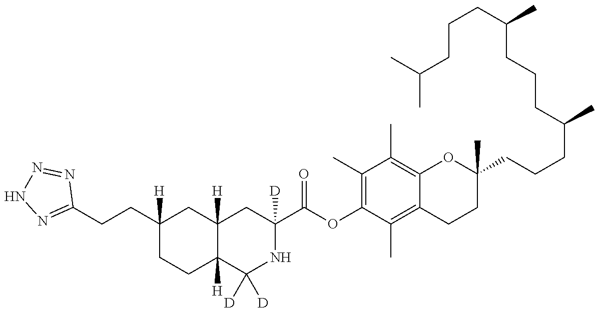 [SMILES: [2H]C1([2H])N[C@]([2H])(C(=O)Oc2c(C)c(C)c3c(c2C)CC[C@@](C)(CCC[C@H](C)CCC[C@H](C)CCCC(C)C)O3)C[C@H]2C[C@@H](CCc3nn[nH]n3)CC[C@H]21] | A08 | C216 |
| 264 | 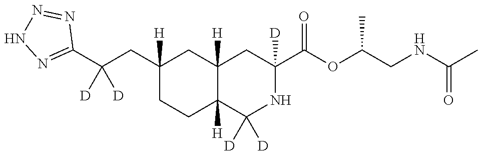 [SMILES: [2H]C([2H])(C[C@H]1CC[C@@H]2[C@H](C1)C[C@@]([2H])(C(=O)O[C@H](C)CNC(C)=O)NC2([2H])[2H])c1nn[nH]n1] | A09 | C031 |
| 265 | 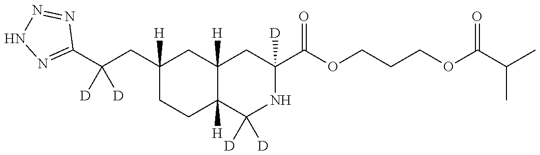 [SMILES: [2H]C([2H])(C[C@H]1CC[C@@H]2[C@H](C1)C[C@@]([2H])(C(=O)OCCCOC(=O)C(C)C)NC2([2H])[2H])c1nn[nH]n1] | A09 | C036 |
| 266 | 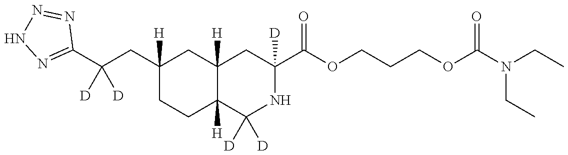 [SMILES: [2H]C([2H])(C[C@H]1CC[C@@H]2[C@H](C1)C[C@@]([2H])(C(=O)OCCCOC(=O)N(CC)CC)NC2([2H])[2H])c1nn[nH]n1] | A09 | C064 |
| 267 | 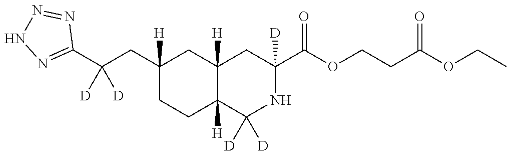 [SMILES: [2H]C([2H])(C[C@H]1CC[C@@H]2[C@H](C1)C[C@@]([2H])(C(=O)OCCC(=O)OCC)NC2([2H])[2H])c1nn[nH]n1] | A09 | C126 |
| 268 | 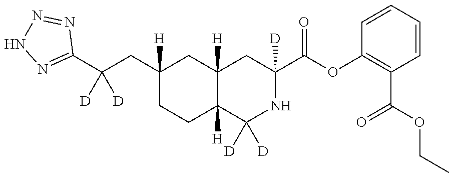 [SMILES: [2H]C([2H])(C[C@H]1CC[C@@H]2[C@H](C1)C[C@@]([2H])(C(=O)Oc1ccccc1C(=O)OCC)NC2([2H])[2H])c1nn[nH]n1] | A09 | C211 |
| 269 | 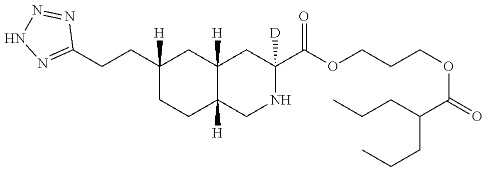 [SMILES: [2H][C@@]1(C(=O)OCCCOC(=O)C(CCC)CCC)C[C@H]2C[C@@H](CCc3nn[nH]n3)CC[C@H]2CN1] | A02 | C37a |

### C. In Vitro Biology

The present disclosure includes esters that are enzymatically activated in vivo to produce active acids (for instance, those of Table 1). Compounds were analyzed, after incubation in rat and human plasma, or S9 liver microsomes fractions, for the disappearance of the ester species and appearance of the acid species. Compounds (1 μM) were incubated in 0.5 mL volume reactions, in duplicate, in plasma or S9 liver microsomes fractions (rat or human) at 37° C. for between 1 minute and 60 minutes, reactions were quenched on ice and by addition of 3 volumes acetonitrile containing 0.1% formic acid, and samples were analyzed by LC/MS/MS. Standard reverse phase HPLC and API 4000 triple quadrupole mass spectrometry were used for analysis.

First, in vitro biochemical assays of prodrug activation were studied for release of the core molecules in Table 1. Test compounds were studied in vitro in test reactions in the presence and absence of rodent or human esterase enzymes to measure the biochemical release of core molecules from compounds. Briefly, compound stock solutions in DMSO solvent were diluted into test reactions (compound final reaction concentration 1 μM and assay final DMSO concentration 1%) and were incubated at 37° C. for 30 minutes in vitro in a reaction buffer consisting of potassium phosphate (20 mM, pH 7.3) and albumin protein 1 mg/mL [(BSA) essentially fatty acid free, purified by electrophoresis from bovine serum; Sigma catalog A0281] either with or without protein extracts containing either rat or human esterase enzyme activity. Liver extract (S9 fraction) from rats (Xenotech LLC catalog number R1000.S9) or from healthy human donors (Xenotech LLC, Lexena Kansas catalog number H0620.59) were used as sources of esterase activity included in the reactions at 1 mg/mL liver S9 protein concentration for rat assays or 3 mg/mL liver S9 protein concentration for human assays. After incubation at 37° C. for 30 minutes the reactions were placed on ice and extracted by adding 3 volumes of acetonitrile (ACN) containing 0.1% formic acid and internal standard (tolbutamide). One volume of sample (reaction mixture with acidified ACN) was diluted in an equal volume of deionized water containing 0.1% formic acid and then injected using an Acquity Sample Manager into a Acquity Liquid Chromatagraph (LC, Waters Corp Milford Massachusetts) with Mass spectrometry (MS) detection using an model 5500 Triple Quad instrument (AB Sciex Corp., Framingham Massachusetts). Samples were eluted on an HPLC Kinetex C₁₈ column (Phenomenex Corp, Torrance California 2.1×50 mm column dimensions; 2.6 micron particle size) and were eluted at 0.6 mL/min using two mobile phases (mobile phase A consisting of 0.1% formic acid in water and mobile phase B consisting of 0.1% formic acid in acetonitrile). Compound A01 (Ref_1) levels were quantified each time by preparing a standard curve of A01 spiked into matrix-matched buffer and extracts prepared as described above.

For quantification of core molecule levels in reactions not containing animal or human esterase activity a standard curve of compounds from Table 1 (e.g. A01 or Ref_1) was prepared with known concentrations of the compound (1, 5, 10, 50, 100, 500, and 1000 mg/mL) in buffer consisting of potassium phosphate (20 mM, pH 7.3) containing albumin 1 mg/mL [essentially fatty acid free, purified by electrophoresis from bovine serum (BSA), Sigma catalog A0281].

For quantification of A01 (Ref_1) levels in reactions containing Liver extract (S9 fraction from rat or human), a standard curve of A01 was generated by preparing samples of reaction buffer containing human liver S9 fraction at known concentrations of A01 (1, 5, 10, 50, 100, 500, and 1000 ng/mL) in same buffer containing heat-inactivated, MAFP-treated human liver S9 fraction (prepared as described above). Extracts were prepared as above for each reaction.

For reactions incubated for 0 minutes at 37° C. the following procedure was followed. Esterase enzyme sources (rat or human; natural or recombinant) were first heat-inactivated by incubation at 65° C. for 15 minutes, then cooled on ice, followed by treatment with esterase inhibitor methyl arachidonyl fluorophosphonate (MAFP, Sigma catalog M2939) at 10 μM at room temperature for 15 minutes. This heat-inactivated, MAFP-treated esterase source material was then mixed with compound 1 μM at 37° C. and organic solvent extracts were prepared immediately for the time 0 minutes 37° C. samples. Extracts of reactions were subjected to liquid chromatography mass spectrometry (LCMS) to quantify A01 (Ref_1) levels compared to standard curves. The limit of quantitation of A01 was measured at 1 mg/mL for samples in human liver S9 or in reaction buffer standard curves, as measured by this LCMS assay.

The above assay conditions with slight modifications were used to study reactions at 37° C. using other esterase enzyme sources including human recombinant carboxylesterase 1 (CES1, Biotechne Inc Minneapolis MN catalog number 4920-CE), human recombinant carboxylesterase 2 (CES2, Biotechne Inc Minneapolis MN catalog number 5657-CE) or plasma from humans or animals (rodent or nonrodent). The standard curve of the A01, or other core molecule analytes, is prepared in matrix-matched buffer (either reaction buffer with BSA or in plasma).

Additionally, ex vivo ionotropic glutamate receptor biological activity assays were conducted. Whole cell patch clamp assays of s-AMPA-induced currents in rat brain slice prefrontal cortex neurons and NMDA-induced currents in rat brain slice prefrontal cortex neurons were examined.

### Slice Preparation.

Male Sprague Dawley rats were supplied by Charles River (Saint Constant, Quebec, Canada) housed 4 per cage within a temperature (20.5-23.5° C.) and humidity (30-80%) controlled environment on a 12 hour light/dark cycle with access to food (Teklad Global Soy Protein, #T.2920X10, Envigo, Indianapolis, IN, USA) and water ad libitum. At 5-6 weeks of age rats were terminally anaesthetised using isofluorane, and decapitated. The brain was removed and 250-400 μm thick coronal prefrontal cortex (PFC) or sagittal hippocampal slices were sectioned using a Vibratome microtome. After brain removal, and throughout slicing, the tissue was submerged in ice cold cerebrospinal fluid (aCSF). Once slices were cut, they were transferred to a beaker containing aCSF and left at room temperature for a minimum of 1 hour before commencing electrophysiological recordings. After this period, individual slices were transferred to a recording chamber continuously perfused with aCSF at a rate of 4-6 mL/min before commencing experiment protocols. An aCSF composition (in mM): NaCl, 127; KCl, 1.9; KH₂PO₄, 1.2; CaCl₂), 2.4; MgCl₂, 1.3; NaHCO₃, 26; D-glucose, 10; equilibrated with 95% Oxygen gas-5% CO₂ gas. Experiments investigating NMDA currents had aCSF supplemented with 10 μM glycine (see appendix for details of all reagent suppliers). All experiments were conducted with the approval of Animal Use Protocol.

### Electrophysiological Recording.

Whole cell patch-clamp recordings were performed from Layer V prefrontal cortex pyramidal neurons, hippocampal CA1 pyramidal and hippocampal stratum radiatum (SR) interneurons at room temperature using the ‘visualised’ version of the patch-clamp technique. Neurons were visualized using a BX51 upright microscope fitted with a 40× LUMPlanFl water immersion objective (Olympus, Richmond Hill, Ontario, Canada). The microscope was connected to a C2400 CCD camera (Hamamatsu Bridgewater, NJ, USA) and images viewed on a VM 5516 B/W monitor (Sanyo, Moriguchi, Osaka Prefecture, Japan). Electrophysiological recordings were obtained using a Multiclamp 700B patch clamp amplifier (Molecular Devices, Sunnyvale, California, USA), with analogue signals digitized on a Digidata 1440a (Molecular Devices, Sunnyvale, California, USA). Patch pipettes were pulled using a P-87 Flaming/Brown micropipette puller (Sutter, Novato, CA, USA), from GC150TF-10 thin-walled borosilicate glass (Harvard Apparatus, Saint-Laurent, Quebec, Canada) which had resistances of between 3 and 8 MΩ when filled with intracellular solution.

Intracellular solutions used for PFC neuron recordings had the following composition (mM): Potassium gluconate, 140; KCl, 10; EGTA-Na, 1; HEPES, 10; Mg-ATP, 4, 0.3 GTP, and for hippocampal recordings had the following composition (mM): KCL, 140; CsCl, 20; EGTA-Na, 1; HEPES, 10; Mg-ATP, 4; GTP, 0.3; QX-314-Br, 5; with pH and osmolarity compensated with potassium hydroxide and sucrose, respectively in all intracellular solutions (see appendix for details of all reagent suppliers).

All test compound experiments were carried out in the voltage clamp recording configuration of the whole cell patch-clamp technique and all recordings performed at a holding potential of −60 mV. Recordings were monitored on a Dell PC running Axon pClamp software (Molecular Devices, Sunnyvale, California, USA) and digitized at 10 kHz.

### Compound Screening.

Experiments were performed to examine the effects of a single concentration of each test-compound on 20 μM s-AMPA-evoked currents in layer V rat prefrontal cortex pyramidal neurons. 10 μM or 30 μM test-compound was administered to the slice by bath perfusion from 50 mL syringes arranged in series with the main perfusion line from the aCSF reservoir. 20 μM s-AMPA or 50 μM NMDA was pressure-ejected for 100-1000 ms every 1-2 minutes using a NPI PDES-02DX (npi Electronic GmbH, Tamm, Germany) pneumatic picopump connected directly to a microelectrode placed within 200 μm of the recorded neuron. Each protocol was repeated three times. If the peak s-AMPA/NMDA current was reduced by more than 70% upon first application of a test-compound, a single concentration of 1 μM test-compound was then used and repeated three times.

Test Compound or s-AMPA Formulation.

Test compounds were prepared as 10 mM or 30 mM stock solutions in 100% DMSO solvent. s-AMPA or NMDA were made as a 20 μM or 50 μM stock solution respectively in aCSF. Test compound stock solutions were diluted in the appropriate external recording solution to the final indicated test concentrations immediately prior to use. All compounds were stored at −20° C. prior to use.

### Reference Compound Tests.

Compound A01 (Ref_1) was tested at 30 μM, 10 μM, 3 μM, 1 μM, 0.3 μM, 0.1 μM concentrations in the s-AMPA assay. Compound A01 (Ref_1) showed concentration-dependent inhibition of peak amplitude responses of rat brain slice PFC pyramidal neurons to 20 μM s-AMPA. A01 (Ref_1) IC₅₀ concentration (where 50% of signal is inhibited) was observed at 481+/−84 nM in the s-AMPA induced currents in cortex pyramidal neurons, n=4 to 5 experiments per concentration.

Compound A01 (Ref_1) was tested at 30 μM concentration in the NMDA assay. Compound A01 at this concentration shows partial inhibition (40%+/−2%, n=4 experiments) of peak amplitude responses of rat brain slice PFC pyramidal neurons to 50 μM NMDA induced currents in cortex pyramidal neurons.

### Data and Statistical Analysis.

All data were sampled using pClamp Clampex acquisition software with all offline analysis carried out using Clampfit (MDS Analytical Technologies). Data compilation and figure construction was carried out using Excel (Microsoft). One-way repeated measures analysis of variance (ANOVA, Prism, Graphpad) with Dunnett's post hoc comparison was used for statistical analysis.

Also carried out were in vitro radioligand binding assays to rat brain membrane receptors, including AMPA receptor assays using ³H-radioligand and NMDA receptor assays using ³H-radioligand.

### Brain Tissue Membrane Preparation for AMPA Receptor Binding Assays.

Crude membrane preparations are prepared from rat brain cortex tissue based on modification of two methods [Enna S J and Snyder S H. “Properties of gamma-aminobutyric acid (GABA) receptor binding in rat brain synaptic membrane fractions.” Brain Res. 1975, 100, 81-97; Murphy D E, Snowhill E W, Williams M. “Characterization of quisqualate recognition sites in rat brain tissue using DL-[³H]alpha-amino-3-hydroxy-5-methylisoxazole-4-propionic acid (AMPA) and a filtration assay.” Neurochem. Res. 1987, 12, 775-781]. Briefly, male Wistar rats are housed in a laboratory with standard animal care and use husbandry procedures, are fed a standard chow diet, and are given access to food and water ad libidum. After 5 days acclimation in above conditions rats are killed by inhalation of carbon dioxide gas. Brains are removed and cortex tissue is rapidly collected on ice. Cortex tissue (˜250 g) is cut in pieces and mechanically homogenized on ice in 200 mL of ice-cold buffer solution [50 mM Tris-HCl, 2.5 mM CaCl₂, 100 mM KSCN (pH 7.1) in water] using an electric-powered tissue homogenizer instrument (Polytron). The homogenate is centrifuged (30,000×g 15 min at 4° C.). The supernatant is discarded. The pellet is washed twice [each time by re-suspension in ice-cold buffer with the Polytron, the volume is adjusted to 320 mL with ice-cold buffer, and the homogenate is centrifuged as described above; the supernatant is discarded and the pellet is re-suspended and centrifuged again]. The resulting pellet is re-suspended in buffer using the Polytron, the volume is adjusted to 320 mL, and the homogenate is incubated 30 min at 37° C. followed by centrifugation (30,000×g 15 min at 4° C.). The supernatant is discarded and the resulting pellet is washed once as described above using re-suspension and centrifugation. The supernatant is discarded and the final pellet is re-suspended in ice-cold buffer using a 21-gauge needle and a syringe. Protein concentration of the rat brain cortex tissue membrane homogenate is determined by the Bradford method compared to a standard curve measured with gamma-globulin protein (Bradford MM. “A rapid and sensitive method for the quantitation of microgram quantities of protein utilizing the principle of protein-dye binding.” Anal. Biochemistry 1976, 72, 248-254). Aliquots of the rat brain cortex tissue membrane homogenates are stored at −80° C. until use.

### AMPA Receptor Radioligand Binding Assay.

Radioligand DL-[³H]-AMPA binding to AMPA receptor is assayed based on modification of the filtration assay method of Murphy et al. [Murphy D E, Snowhill E W, Williams M. “Characterization of quisqualate recognition sites in rat brain tissue using DL-[³H]alpha-amino-3-hydroxy-5-methylisoxazole-4-propionic acid (AMPA) and a filtration assay.” Neurochem. Res. 1987, 12, 775-781]. Briefly, rat brain cortex tissue membrane homogenate (prepared as described above) is thawed on ice and 200 micrograms of protein is suspended in assay buffer solution [50 mM Tris-HCl, 2.5 mM CaCl₂, 100 mM KSCN (pH 7.1) in water]. Test compounds are dissolved and diluted in DMSO then added as 1:100 dilutions into assay reactions (0.2 mL final assay reaction volume, maximum final DMSO concentration in the reactions was 1%). The radioligand amino-3-hydroxy-5-methylisoxazole-4-propionic acid-DL-alpha-[5-methyl-³H] also called DL-[³H]-AMPA is added to reactions at a final assay concentration of 8 nM. Radiochemical purity of DL-[³H]-AMPA is >97% by HPLC analysis; specific activity range of batches used is 40-70 Ci/mmol (1480-2590 GBq/mmol). The radioligand is stored at a concentration of 1.0 mCi/ml (37 MBq/mL) as a solution in ethanol:water (1:1) under argon gas at −20° C. (product number NET833, manufacturer Perkin Elmer Inc; Boston, Massachusetts USA; references for the synthesis and characterization of DL-[3H]-AMPA: Lauridsen J Honore T. “Preparation of deuterium labelled alpha-amino-3-hydroxy-5-methylisoxazole-4-propionic acid (AMPA)” J Labelled Comp. Radiopharm. 1981, 18, 1479-1484; Honore T, Lauridsen J, Krogsgaard-Larsen P. “The binding of [³H]AMPA, a structural analogue of glutamic acid, to rat brain membranes” J. Neurochem. 1982, 38, 173-178). Rat brain cortex tissue membrane homogenates are incubated with DL-[3H]-AMPA in the presence or absence of test compound for 60 min at 4° C. Nonspecific binding is determined in the presence of 1 mM L-glutamate (Sigma, St. Louis Missouri, USA). After incubation reaction samples are filtered rapidly under vacuum through glass fiber filters (GF/B, Packard manufacturer Perkin-Elmer; Waltham, Massachusetts USA) which are presoaked in assay buffer) and rinsed several times with ice-cold assay buffer using a 96-sample cell harvester [Unifilter, Packard instrument, manufacturer Perkin-Elmer; Waltham, Massachusetts USA]. The filters are dried then counted for radioactivity in a scintillation counter (Topcount Packard instrument; manufacturer Perkin-Elmer; Waltham, Massachusetts USA) using scintillation cocktail (MicroScint-O, Packard (product number 6013611, manufacturer Perkin-Elmer; Waltham, Massachusetts USA). Percent nonspecific binding is noted. Data are expressed as percent inhibition of the control radioligand specific binding. Data are analyzed in Graphpad Prism Software (San Diego, California USA) and when possible IC₅₀ values calculated. The AMPA binding constant is K_{d} 82 nM in these assays and the reference ligand L-glutamate IC₅₀ 535 nM.

### Brain Membrane Preparation for Use in NMDA Receptor Radioligand Binding Assays.

Crude membrane preparations are prepared from rat brain cortex tissue based on modification of a published method (See Enna S J 1975 above). Dissections and most steps other than centrifugations performed on ice and all other steps are at 4 degrees C. or using ice-cold buffers. Cortex tissue (250 grams) from Wistar rats (housed and cared for as described above in section above on Brain membrane preparation for AMPA receptor radioligand binding assays) is collected (Cerebellum is discarded), cut in small pieces and pooled in 650 ml of buffer A (5 mM Tris-Base buffer pH 8.0) and homogenized using an electric-powered tissue homogenizer instrument (Polytron). The homogenate is centrifuged (40,000×g 15 min at 4° C.) and supernatant discarded. The pellet is resuspended in 650 ml buffer B (5 mM Tris-Base pH 8.0, 10 mM EDTA) using a 21 gauge needle. The suspension is centrifuged (40,000×g 15 min at 4° C.) and supernatant discarded. The pellet is resuspended in buffer B using a 21 gauge needle and through and 26 gauge needle on ice. Protein concentration is determined by Bradford assay (see Bradford 1976 above). Aliquots are prepared and frozen at −80° C. until use in NMDA receptor radioligand binding assay.

### NMDA Receptor Radioligand Binding Assay.

The NMDA receptor radioligand binding assay was based on modification of the methods of Sills M A, Fagg G, Pozza M, Angst C, Brundish D E, Hurt S D, Wilusz E J, Williams M. “[³H] CGP 39653: a new N-methyl-D-aspartate antagonist radioligand with low nanomolar affinity in rat brain.” Eur. J. Pharmacol. 1991, 192, 19-24. Briefly, membrane homogenates of rat brain cerebral cortex tissue prepared above under (NMDA receptor brain preparation) (1 mg protein) are incubated for 60 min at 4° C. with 5 nM [³H]CGP 39653 [NMDA receptor radioligand; CGP 39653 [propyl-2,3-3H] Perkin-Elmer product number NET1050; radiochemical purity of >97% by HPLC analysis; specific activity range of batches used is 20-50 Ci/mmol (740-1850 GBq/mmol); radioligand stored at a concentration of 1.0 mCi/ml (37 MBq/mL) as a solution in ethanol:water (1:9) under argon gas at −20° C.; manufacturer Perkin Elmer Inc; Boston, Massachusetts USA] in the absence or presence of the test compound in a buffer containing 5 mM Tris-HCl (pH 7.7) and 10 mM EDTA-Tris. Nonspecific binding is determined in the presence of 100 μM L-glutamate. Following incubation, the samples are filtered rapidly under vacuum through glass fiber filters (GF/B, Whatman) and rinsed several times with ice-cold incubation buffer using a 48-sample cell harvester (Brandel). The filters are dried then counted for radioactivity in a scintillation counter (LS series, Beckman) using scintillation cocktail (Formula 989, Packard). The results are expressed as a percent inhibition of the control radioligand specific binding. The standard reference compound is CGS 19755, which is tested in each experiment at several concentrations to obtain a competition curve from which its IC50 is calculated (Tocris catalog number 1241, CAS #110347-85-8, cis-4-[phosphomethyl]-piperidine-2-carboxylic acid, Biotechne Minneapolis Minnesota; see Lehmann et al. “CGS 19755, a selective and competitive N-methyl-D-aspartate-type excitatory amino acid receptor antagonist.” J. Pharmacol. Exp. Ther. 1988, 246-265).

### D. In Vivo Pharmacology

In vivo pharmacology in rodents, including in vivo rodent epilepsy or seizure models and in vivo rodent pain models.

### Compound Preparation for Administration to Animals.

Dosing solutions were prepared by dissolving test compounds in DMSO and diluting into 0.5% methylcellulose (1% DMSO final concentration). 0.5% Methylcellulose (0.5% MC, Sigma, Catalog M-0430, St. Louis, Missouri) was prepared in water as per the manufacturer's instructions. Solutions were vortexed stirred on a heated plate (˜40° C.) until complete dissolution was achieved. Dosing solutions were prepared fresh on the day of testing. All solutions were mixed thoroughly prior to administration.

Compound A01 (Ref_1) was formulated by mixing compound powder directly into 0.5% MC, then 0.1 N or 1 N sodium hydroxide solution was added carefully to pH 9 to pH 10 and the samples were stirred and vortexed with heating to 45° C. for 15-30 min until fully dissolved. Then 0.1 N or 1 N hydrochloric acid solution was then added carefully to pH 7.3. Vehicle solution was prepared using this same protocol without compound present. Levetiracetam (TCI America, Portland Oregon) dosing solution was prepared in a 0.5% MC. Perampanel dosing solution was prepared in 0.5% MC with sonication and heating at 40° C. followed by stirring for at least 2 hours or longer at 40° C.

### Animals and Compound Administration.

Adult male Carworth Farms (CF-1) mice (20-30 g), used for the 6 Hz psychomotor seizure assays, were obtained from Charles River Laboratories (Kingston NY). Animals were allowed free access to food and water, except during testing periods. After delivery, animals were allowed sufficient time to acclimate to housing conditions prior to testing. All mice were housed in plastic cages in rooms with controlled humidity, ventilation, and lighting (12 hours on/12 hours off). The animals were housed and fed in a manner consistent with the recommendations in the “Guide for Care and Use of Laboratory Animals” (National Research Council), and in accordance with guidelines set by the Institutional Animal Care and Use Committee of the University of Utah. Animal experiments were conducted in a manner consistent with Animal Research: Reporting of In Vivo Experiments (ARRIVE) guidelines (UK). Test compounds or respective vehicle (placebo) were administered using an optimal fluid volume-to-body fluid ratio. Solutions were administered to mice in a volume of 0.01 mL/g body weight by subcutaneous (s.c.) injection unless otherwise indicated. Levetiracetam was administered by intraperitoneal (i.p.) injection. Test compounds, perampanel, or vehicle could also be administered by oral route of administration.

### Mouse 6 Hz Psychomotor Seizure Model of Partial Epilepsy.

Focal seizures were induced in mice via corneal stimulation (6 Hz, 0.2 msec rectangular pulse, 3 second duration at 22 mA [Barton M E, Klein B D, Wolf H H, White H S. “Pharmacological characterization of the 6 Hz psychomotor seizure model of partial epilepsy.” Epilepsy Res. 2001, 47(3), 217-27; Barton M E, Peters S C, Shannon H E. “Comparison of the effect of glutamate receptor modulators in the 6 Hz and maximal electroshock seizure models.” Epilepsy Res. 2003, 56 (1), 17-26; Metcalf C S, Klein B D, McDougle D R, Zhang L, Kaufmann D, Bulaj G, White H S. “Preclinical evaluation of intravenous NAX 810-2, a novel GalR2-preferring analog, for anticonvulsant efficacy and pharmacokinetics.” Epilepsia. 2017, 58 (2), 239-246; and Metcalf C S, West P J, Thomson K E, Edwards S F, Smith M D, White H S, Wilcox KS. “Development and pharmacologic characterization of the rat 6 Hz model of partial seizures.” Epilepsia. 2017, 58 (6), 1073-1084]). Mice were tested at 22 mA stimulus intensity unless otherwise indicated (32 mA or 44 mA stimulus intensities could be tested as well). Prior to stimulation, drops of 0.5% tetracaine were applied to each eye. The seizures that arise from corneal stimulation in this assay are characterized by a minimal clonic phase followed by stereotyped automatistic behaviors including stun, forelimb clonus, twitching of the vibrissae, and Straub-tail. If any of these behaviors occurred during a 1-minute observation period following stimulation, the animal was considered as having demonstrated a seizure. Animals not displaying any of the behaviors noted above were considered “protected.” Separate studies were conducted to evaluate dose-response relationships for each compound using 22 mA stimulus intensities (32 mA or 44 mA could be used as well). Initial time-course studies were conducted to determine pretreatment times for each compounds. Unless otherwise indicated pretreatment times were 0.5 hours for respective vehicle, compound A01 (Ref_1), perampanel, and test compounds, and 1 hour for levetiracetam. Test compounds or respective vehicles were tested in some cases at other pretreatment times as indicated. A median effective dose (ED₅₀) and 95% confidence interval (CI) were calculated using a Probit analysis. For combination studies, compounds were administered using the same pretreatment time and route used for single administration studies.

### Rotarod Test for Motor Impairment.

Testing mouse performance on the rotarod was conducted in tandem with 6 Hz psychomotor seizure model stimulation to verify whether doses administered produced substantial motor impairment. Evaluation in the rotarod assay was conducted immediately prior to 6 Hz psychomotor seizure model testing. Therefore, each treatment group was subjected to evaluation in the rotarod assay followed immediately by testing in the 6 Hz psychomotor seizure model. When mice are placed on a 1-inch knurled rod rotating at a speed of 6 rpm, the animals can maintain equilibrium for long periods. Motor impairment was assessed by determining whether mice remained on the rotarod during a 1-minute observation period; that is, three falls during a 1 minute period was considered a rotarod failure. Test compounds administered to CF-1 mice at 13 mg/kg by s.c. route were comparable to vehicle treatment and did not impair rotarod performance.

### Serum Collection for Analysis of Plasma Drug Levels.

In various tests, animals were sacrificed following testing, and trunk blood was collected for quantification of drug levels. Immediately after testing, animals were decapitated and trunk blood was collected into a BD Vacutainer tube containing dipotassium ethylenediaminetetraacetic acid (K₂EDTA) and chilled on ice until centrifugation. Following centrifugation (10,000 g, 5-7 min), the plasma was removed and transferred to a labeled microcentrifuge tube and stored at −80° C. until shipment to the sponsor. The frozen sample was placed in a labeled centrifuge tube and stored at −80° C. until analysis of samples.

### Statistical Analysis.

Seizure protection and rotarod motor impairment data are presented as # protected/N tested and # with motor impairment/N tested, respectively. A Fisher exact test was used to compare motor impairment values for specific treatment groups with VEH-treated animals. For dose-response analyses, ED₅₀ (and 95% CI) values were calculated using a Probit analysis wherein at least four treatment groups (N=8 per group) were used in the calculation. Plasma levels are presented as means±standard error and were compared using a Student's t-test.

MES (Maximum Electroshock induced Seizure) In Vivo Model of Anti-convulsant Activity in Rodents.

In vivo AMPA antagonist activity of compounds can be assayed using the Maximum electroshock-induced seizure (MES) model in mice. This test measures anti-convulsant effects of pharmacological treatments on tonic convulsions of hind-extremities (tonic hind-limb extension). Male CF-1 mice (obtainable from Charles River Laboratories) (n=10 or more mice group) are administered either test articles (experimental compounds, or vehicle, or positive control reference anti-convulsant compounds) by intraperitoneal (i.p.), subcutaneous (s.c.), intravenous (i.v.) or by oral gavage (p.o.) route. The treatments are either 15 to 120 min or as indicated prior to corneal application of electric shock (alternating current typically 60 Hz and 40 mA for 0.1 sec; or 60 Hz at 50 mA for 0.2 sec; or 50 Hz and 18 mA for 0.2 sec or as previously described using corneal electrodes [Schmutz M, Portet C, Jeker A, Klebs K, Vassout A, Allgeier H, Heckendorn R, Fagg G E, Olpe H R, van Riezen H. “The competitive NMDA receptor antagonists CGP 37849 and CGP 39551 are potent, orally-active anticonvulsants in rodents.” Naunyn Schmiedebergs Arch Pharmacol. 1990, 342 (1), 61-6; Leander J D, Rathbun R C, Zimmerman D M. “Anticonvulsant effects of phencyclidine-like drugs: relation to N-methyl-D-aspartic acid antagonism.” Brain Res. 1988, 454 (1-2), 368-72; Leander J D. “Evaluation of dextromethorphan and carbetapentane as anticonvulsants and N-methyl-D-aspartic acid antagonists in mice.” Epilepsy Res. 1989, 4 (1), 28-33; and Yamaguchi S, Donevan S D, Rogawski M A. “Anticonvulsant activity of AMPA/kainate antagonists: comparison of GYKI 52466 and NBOX in maximal electroshock and chemoconvulsant seizure models.” Epilepsy Res. 1993, 15 (3), 179-84.)]. If animals do not show hind limb extension, they are considered protected from the convulsant effect of electroshock. In some cases the effective dose (ED₅₀ and 95% confidence interval) of test compound or reference compound that abolished the tonic-extensor component of the convulsion in 50% of the animals is calculated from dose-response data [Litchfield, JT Jr, Wilcoxon. “A simplified method of evaluating dose-effect experiments.” J. Pharmacol. Exp. Ther. 1949, 96 (2), 99-113]. One group of animals always receives vehicle treatment (negative control) and one group of animals always receives reference compound treatment (positive control) for each experiment.

### Lithium-Pilocarpine Induced Status Epilepticus Rat Model.

Either Long-Evans or Sprague-Dawley rats can be used following published procedures to induce status epilepticus in animals (see Metcalf C S, Radwanski P B, Bealer S L. “Status epilepticus produces chronic alterations in cardiac sympathovagal balance.” Epilepsia. 2009, 50 (4), 747-54; Clifford, D. B., Olney, J. W., Maniotis, A., Collins, R. C., Zorumski, C. F. “The functional anatomy and pathology of lithium-pilocarpine and high-dose pilocarpine seizures.” Neuroscience, 1987, 23, 953-968; Hanada T, Ido K, Kosasa T. “Effect of perampanel, a novel AMPA antagonist, on benzodiazepine-resistant status epilepticus in a lithium-pilocarpine rat model.” Pharmacol. Res. Perspect. 2014, 2 (5), e00063; Wu T, Ido K, Osada Y, Kotani S, Tamaoka A, Hanada T “The neuroprotective effect of perampanel in lithium-pilocarpine rat seizure model.” Epilepsy Res. 2017, 137, 152-158). The effect of test compounds vs respective vehicle is compared to reference compounds perampanel or other reference compounds.

Although the invention has been described with reference to a specific embodiment this description is not meant to be construed in a limiting sense. The invention being thus described, it is apparent that the same can be varied in many ways. Such variations are not to be regarded as a departure from the spirit and scope of the present invention, and all such modifications, alternatives, and equivalents as would be obvious to one skilled in the art are intended to be included within the scope of the following claims.

Results from the in vitro assay using extracts from both human and rat liver shows that compound A01 (Table 1) was efficiently released from the esters tested. Table 5 shows the results of selected compounds from Table 4 in an in vivo seizure protection study in mice.

TABLE 5
Prodrug In Vivo Data.

| | Protection from Psychomotor Seizures | | | |
|---|---|---|---|---|
| | Mice 6 Hz In Vivo Model (values are | | | |
| Compound | number mice protected/number of mice tested) | | | |
| ID | Test 1* | Test 2* | Test 3\hat{ } | Test 4^{#} |
| 4 | 8/8 | 8/8 | | |
| 5 | 6/8 | 7/8 | 4/4 | 4/4 |
| 6 | 7/8 | 4/8 | 4/4 | 3/4 |
| 14 | 7/8 | | | |
| 15 | 6/8 | | | |
| 16 | 3/8 | | | |
| 17 | 8/8 | 6/8 | 4/4 | 3/4 |
| 18 | 3/8 | | | |
| 19 | 7/8 | 5/8 | 4/4 | 4/4 |
| 20 | 5/8 | | | |
| 22 | 5/8 | | | |
| 23 | 1/8 | | | |
| 24 | 5/8 | | | |

*Tests 1 and 2 were conducted by administering compound or vehicle treatment 30 minutes prior to 22 mA stimulus in the 6 Hz model.
\hat{ }Test 3 was conducted by administering compound or vehicle treatment 1 hour prior to 22 mA stimulus in the 6 Hz model.
^{#}Test 4 conducted by first administering compound or vehicle treatment 2 hours prior to 22 mA stimulus in the 6 Hz model.

### Clauses

1. A compound of formula Ia or Ib:

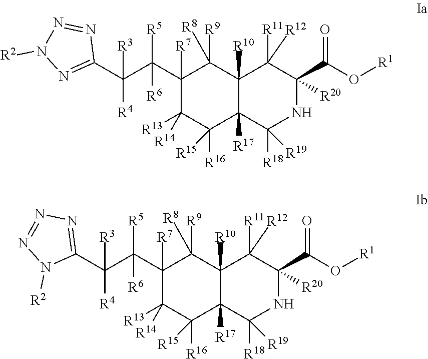

wherein:
R¹ is selected from H, (C₁-C₂₀)hydrocarbyl, and —CH(R^{a})R^{b};
R^{a} is selected from H, —(C₁-C₆)alkyl, and —C(═O)—O—(C₁-C₆)alkyl;
R^{b} is selected from —(C₁-C₁₀)hydrocarbyl-O—C(═O)—R^{c}, —(C₁-C₁₀)hydrocarbyl[-OC(═O)—R^{c}]₂, -direct bond-C(═O)—R^{c} or —(C₁-C₁₀)hydrocarbyl-C(═O)—R^{c}, —CH(NHR^{d})C(═O)—R^{c}, and —(C₁-C₁₀)hydrocarbyl-NR^{e}—C(═O)—R^{f};
R^{c} is selected from —O—(C₁-C₁₀)hydrocarbyl, —NH—(C₁-C₁₀)hydrocarbyl, —N—[(C₁-C₁₀)hydrocarbyl]₂, where the hydrocarbyl groups can be combined to form a (C₄-C₆) nitrogenous aliphatic heterocycle, —(C₁-C₁₀)hydrocarbyl, —NH₂, —(C₁-C₁₀)hydrocarbyl-NH—R^{e}, where the hydrocarbyl group can be combined with R^{e} to form a (C₄-C₆) nitrogenous aliphatic heterocycle, and —NR^{e}—(C₁-C₁₀)hydrocarbyl-C(═O)—O—(C₁-C₁₀)hydrocarbyl, where the hydrocarbyl group and R^{e} can be combined to form a (C₄-C₆) nitrogenous aliphatic heterocycle;
R^{d} is selected from H, —(C₁-C₁₀)hydrocarbyl, and —C(═O)(C₁-C₁₀)hydrocarbyl;
R^{e} is selected from H, and —(C₁-C₆)alkyl;
R^{f} is selected from —O—(C₁-C₁₀)hydrocarbyl, —(C₁-C₁₀)hydrocarbyl, -direct bond-C(═O)—O—(C₁-C₁₀)hydrocarbyl or —(C₁-C₄)hydrocarbyl-C(═O)—O—(C₁-C₁₀)hydrocarbyl, -direct bond-C(═O)—NH₂ or —(C₁-C₄)hydrocarbyl-C(═O)—NH₂, -direct bond-C(═O)—NH—(C₁-C₁₀)hydrocarbyl or —(C₁-C₄)hydrocarbyl-C(═O)—NH—(C₁-C₁₀)hydrocarbyl, —(C₁-C₁₀)hydrocarbyl-NH—R^{e}, where the hydrocarbyl linker can be combined with R^{e} to form a (C₄-C₆) nitrogenous aliphatic heterocycle, and —(C₁-C₁₀)hydrocarbyl-NH—C(═O)(C₁-C₁₀)hydrocarbyl;
R² is selected from H and optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂;
R⁷ is selected from hydrogen, deuterium, and fluorine;
R³-R⁶ and R⁸-R²⁰ are independently selected from hydrogen and deuterium; and with the provisos:
   (i) when R¹ is C₁-C₆ alkyl or aryl and R² is a nitrogen protecting group, wherein said nitrogen protecting group is selected from trityl, benzyl, t-butyl, t-butyldimethylsilyl, and triphenylsilyl, then either (a) R⁷ is fluorine or (b) at least one of R³-R²⁰ is deuterium;
   (ii) when R¹ is H, C₁-C₆ alkyl, substituted alkyl, cycloalkyl, or arylalkyl and R² is H, then either (a) R⁷ is fluorine or at (b) least one of R³-R²⁰ is deuterium; and
   (iii) when R⁷ is fluorine, then R² is not H.

2. A compound according to claim 1, with the structure of formula IIa, IIb, IIIa, or IIb:

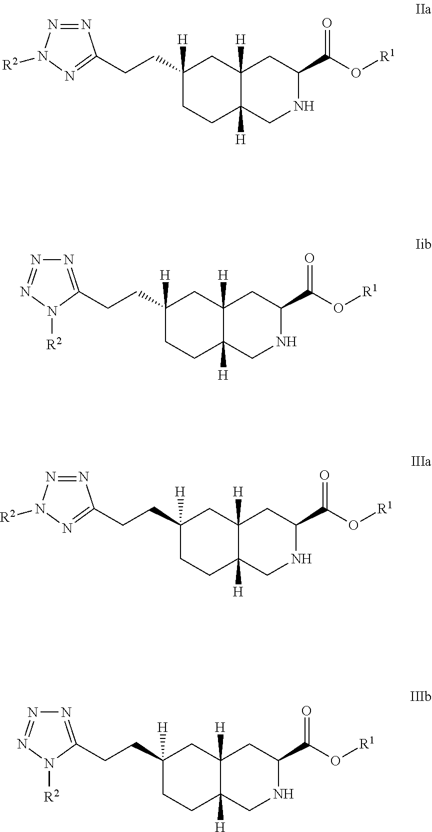

wherein:
R¹ is selected from H and (C₁-C₂₀)hydrocarbyl;
R² is optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂; and
R³-R²⁰ are each H.

3. A compound according to claim 1, with the structure of formula IVa, IVb, Va, or Vb:

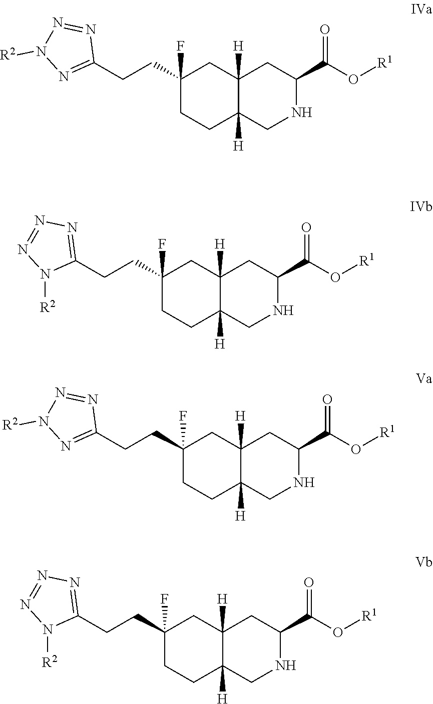

wherein:
R¹ is selected from H and (C₁-C₂₀)hydrocarbyl;
R³-R⁶ and R⁸-R²⁰ are each H;
R⁷ is fluorine; and
R² is optionally substituted (C₁-C₇)hydrocarbyl, wherein said optionally substituted (C₁-C₇)hydrocarbyl may be substituted with one or more of —F, —CN, —OH, —O—C(═O)(C₁-C₄)hydrocarbyl, —C(═O)—OH, —C(═O)—O—CH₃, —C(═O)—NH—CH₃, —NH(C₁-C₄)hydrocarbyl, —N[(C₁-C₄)hydrocarbyl)]₂, or —C(═O)—N[(C₁-C₃)hydrocarbyl]₂;

4. A compound according to claim 3, with the structure of formula IVa or IVb:

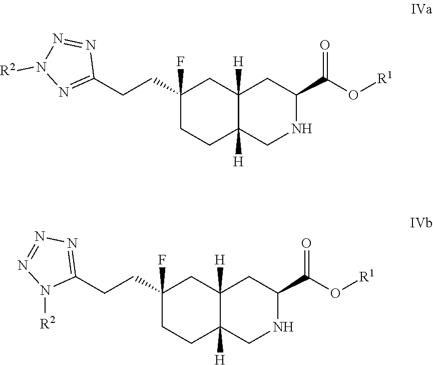

5. A compound according to claim 3, with the structure of formula V:

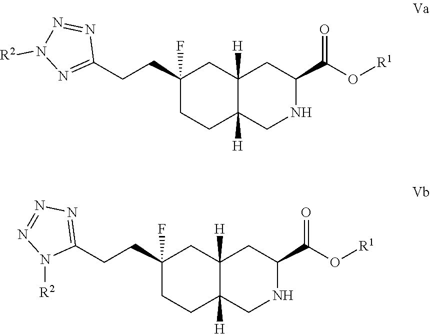

6. A compound according to any one of claims 1-5, wherein R¹ is selected from H and aliphatic (C₁-C₂₀)hydrocarbyl optionally substituted with one or two phenyl groups with the proviso that R¹ contains twenty carbons or less.

7. A compound according to any one of claims 1-6, wherein R¹ is selected from H and (C₁-C₂₀)alkyl optionally substituted with one or two phenyl groups with the proviso that R¹ contains twenty carbons or less.

8. A compound according to any one of claims 1-5, wherein R¹ is H or (C₁-C₁₃)hydrocarbyl.

9. A compound according to any one of claims 1-6 and 8, wherein R¹ is H or aliphatic (C₁-C₁₃)hydrocarbyl optionally substituted with one or two phenyl groups with the proviso that R¹ contains twenty carbons or less.

10. A compound according to any one of claims 1-9, wherein R¹ is H or (C₁-C₁₃)alkyl optionally substituted with one or two phenyl groups with the proviso that R¹ contains twenty carbons or less.

11. A compound according to any one of claims 1-10, wherein R¹ is H or (C₁-C₁₀)alkyl optionally substituted with one or two phenyl groups with the proviso that R¹ contains twenty carbons or less.

12. A compound according to any one of claims 1-5 wherein R is H or C_{q}Hᵣ, and wherein:
q is 1 and r is 3;
q is 2 and r is 5;
q is 3 and r is 3, 5, or 7;
q is 4 and r is 5, 7, or 9;
q is 5 and r is 7, 9, or 11;
q is 6 and r is 5, 7, 9, 11, or 13.
q is 7 and r is 7, 9, 11, 13, or 15;
q is 8 and r is 5, 7, 9, 11, 13, 15, or 17;
q is 9 and r is 7, 9, 11, 13, 15, 17, or 19;
q is 10 and r is 7, 9, 11, 13, 15, 17, 19, or 21;
q is 11 and r is 9, 11, 13, 15, 17, 19, 21, or 23;
q is 12 and r is 7, 9, 11, 13, 15, 17, 19, 21, 23, or 25;
q is 13 and r is 9, 11, 13, 15, 17, 19, 21, 23, 25, or 27;
q is 14 and r is 9, 11, 13, 15, 17, 19, 21, 23, 25, 27, or 29;
q is 15 and r is 11, 13, 15, 17, 19, 21, 23, 25, 27, 29, or 31;
q is 16 and r is 9, 11, 13, 15, 17, 19, 21, 23, 25, 27, 29, 31, or 33;
q is 17 and r is 11, 15, 17, 19, 21, 23, 25, 27, 29, 31, 33, or 35;
q is 18 and r is 11, 13, 15, 17, 19, 21, 23, 25, 27, 29, 31, 33, 35, or 37;
q is 19 and r is 13, 15, 17, 19, 21, 23, 25, 27, 29, 31, 33, 35, 37, or 39; or
q is 20 and r is 11, 13, 15, 17, 19, 21, 23, 25, 27, 29, 31, 33, 35, 37, 39, or 41.

13. A compound according to any one of claims 1-5 and 8, wherein R¹ is H, methyl, ethyl, n-propyl, isopropyl, cyclopropyl, n-butyl, 1-methylpropyl, 1-methyl-2-ethylbutyl, 2-ethylbutyl, 2-methylpropyl, tert-butyl, 2-methylcyclopropyl, 1-methylcyclopropyl, cyclobutyl, cyclopropylmethyl (i.e.,

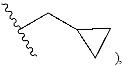 [SMILES: *CC1CC1]

n-pentyl, 1-methylbutyl, 2-methylbutyl, 3-methylbutyl, 1-ethylpropyl, 1,2-dimethylpropyl, 1,1-dimethylpropyl, 2,2-dimethylpropyl, cyclobutylmethyl (i.e.,

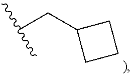 [SMILES: *CC1CCC1]

2-(cyclopropyl)ethyl (i.e.,

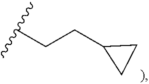 [SMILES: *CCC1CC1]

cyclopentyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,3-dimethylbutyl, 2,2-dimethylbutyl, 3,3-dimethylbutyl, 1,1,2-trimethylpropyl, 1,2,2-trimethylpropyl, 3-(cyclopropyl)propyl (i.e.,

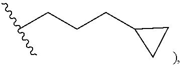 [SMILES: *CCCC1CC1]

2-(cyclobutyl)ethyl (i.e.,

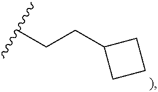 [SMILES: *CCC1CCC1]

cyclopentylmethyl (i.e.,

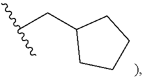 [SMILES: *CC1CCCC1]

cyclohexyl, cyclohexylmethyl, 2-cyclohexylethyl, dicyclohexylmethyl, n-octyl, benzyl, diphenylmethyl, decyl, dodecyl, tetradecyl, hexadecyl, 237ctadic-9-enyl, octadecyl, 237ctadic-9-enyl, 237ctadic-9,12-dienyl, 2-propylpentyl, 2-butylhexyl, 2-pentylheptyl, or 2-hexyloctyl.

14. A compound according to any one of claims 1-5, 8, 9, and 12, wherein R¹ is H, methyl, ethyl, n-propyl, isopropyl, n-butyl, tert-butyl, 2-methylbutyl, 2-ethylbutyl, or cyclohexyl.

15. A compound according to any one of claims 1-14, wherein R¹ is H.

16. A compound according to any one of claims 1-14, wherein R¹ is ethyl.

17. A compound according to any one of claims 1-14, wherein R¹ is isopropyl.

18. A compound according to any one of claims 1-14, wherein R¹ is 2-ethylbutyl.

19. A compound according to any one of claims 1-5, 8, 9, 13, and 14, wherein R¹ is cyclohexyl.

20. A compound according to any one of claims 1-19, wherein R² is optionally substituted (C₁-C₇)hydrocarbyl, and wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂.

21. A compound according to claim 20, wherein R² is optionally substituted benzyl, and wherein said optionally substituted benzyl is optionally substituted with one or more of —C(═O)—OH, —C(═O)—O—CH₃, or —C(═O)—NH—CH₃.

22. A compound according to claim 20, wherein R² is optionally substituted (C₁-C₃)hydrocarbyl, and wherein said optionally substituted (C₁-C₃)hydrocarbyl is substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂.

23. A compound according to claim 22, wherein R² is methyl optionally substituted with one or more of —F, —O—C(═O)(C₁-C₄)hydrocarbyl, —C(═O)—OH, —C(═O)—O—CH₃, or —C(═O)—NH—CH₃.
24. A compound according to claim 23, wherein R² is methyl.
25. A compound according to claim 23, wherein R² is difluoromethyl.
26. A compound according to claim 23, wherein R² is CH₂—O—C(═O)(C₁-C₄)hydrocarbyl.
27. A compound according to claim 26, wherein R² is CH₂—O—C(═O)—CH₃.
28. A compound according to claim 23, wherein R² is CH₂—C(═O)—OH.
29. A compound according to claim 23, wherein R² is CH₂—C(═O)—O—CH₃.
30. A compound according to claim 23, wherein R² is CH₂—C(═O)—NH—CH₃.
31. A compound according to claim 22, wherein R² is ethyl optionally substituted with one or more of —OH, —O—C(═O)(C₁-C₄)hydrocarbyl, —NH(C₁-C₄)hydrocarbyl, or —N[(C₁-C₄)hydrocarbyl)]₂.

32. A compound according to claim 30, wherein R² is ethyl.

33. A compound according to claim 30, wherein R² is ethanol.

34. A compound according to claim 30, wherein R² is (CH₂)₂—O—C(═O)—CH₃.

35. A compound according to claim 30, wherein R² is (CH₂)₂—N[(C₁-C₄)hydrocarbyl)]₂.

36. A compound according to claim 22, wherein R² is propyl optionally substituted with one or more of —CN.

37. A compound according to claim 1, with the structure of formula VIa or VIb:

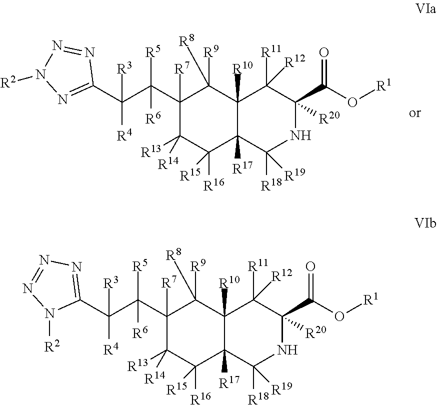

wherein:
R¹ is selected from —CH(R^{a})R^{b} and aromatic (C₁-C₂₀)hydrocarbyl;
R^{a} is selected from H, —(C₁-C₆)alkyl, and —C(═O)—O—(C₁-C₆)alkyl;
R^{b} is selected from —(C₁-C₁₀)hydrocarbyl-O—C(═O)—R^{c}, —(C₁-C₁₀)hydrocarbyl[-OC(═O)—R^{c}]₂, -direct bond-C(═O)—R^{c} or —(C₁-C₁₀)hydrocarbyl-C(═O)—R^{c}, —CH(NHR^{d})C(═O)—R^{c}, and —(C₁-C₁₀)hydrocarbyl-NR^{e}—C(═O)—R^{f};
R^{c} is selected from —O—(C₁-C₁₀)hydrocarbyl, —NH—(C₁-C₁₀)hydrocarbyl, —N—[(C₁-C₁₀)hydrocarbyl]₂, where the hydrocarbyl groups can be combined to form a (C₄-C₆) nitrogenous aliphatic heterocycle, —(C₁-C₁₀)hydrocarbyl, —NH₂, —(C₁-C₁₀)hydrocarbyl-NH—R^{e}, where the hydrocarbyl group can be combined with R^{e} to form a (C₄-C₆) nitrogenous aliphatic heterocycle, and —NR^{e}—(C₁-C₁₀)hydrocarbyl-C(═O)—O—(C₁-C₁₀)hydrocarbyl, where the hydrocarbyl group and R^{e} can be combined to form a (C₄-C₆) nitrogenous aliphatic heterocycle;
R^{d} is selected from H, —(C₁-C₁₀)hydrocarbyl, and —C(═O)(C₁-C₁₀)hydrocarbyl;
R^{e} is selected from H, and —(C₁-C₆)alkyl;
R^{f} is selected from —O—(C₁-C₁₀)hydrocarbyl, —(C₁-C₁₀)hydrocarbyl, -direct bond-C(═O)—O—(C₁-C₁₀)hydrocarbyl or —(C₁-C₄)hydrocarbyl-C(═O)—O—(C₁-C₁₀)hydrocarbyl, -direct bond-C(═O)—NH₂ or —(C₁-C₄)hydrocarbyl-C(═O)—NH₂, -direct bond-C(═O)—NH—(C₁-C₁₀)hydrocarbyl or —(C₁-C₄)hydrocarbyl-C(═O)—NH—(C₁-C₁₀)hydrocarbyl, —(C₁-C₁₀)hydrocarbyl-NH—R^{e}, where the hydrocarbyl linker can be combined with R^{e} to form a (C₄-C₆) nitrogenous aliphatic heterocycle, and —(C₁-C₁₀)hydrocarbyl-NH—C(═O)(C₁-C₁₀)hydrocarbyl;
R² is selected from H and optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂;
R³-R²⁰ are each independently selected from hydrogen and deuterium; and
wherein at least one of R³-R²⁰ is deuterium.

38. A compound according to claim 37, with the structure of formula VIIa or VIIb:

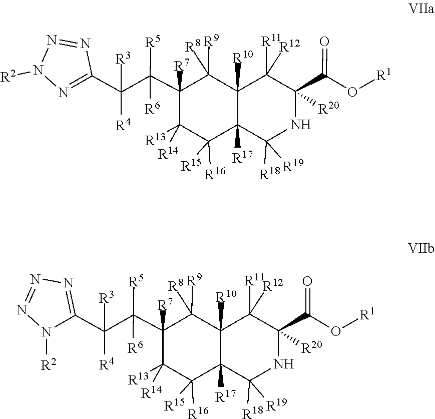

39. A compound according to any one of claims 37-38, wherein R¹ is —CH(R^{a})R^{b}.

40. A compound according to claim 39, wherein R^{b} is —(C₁-C₁₀)hydrocarbyl-O—C(═O)—R^{c}.

41. A compound according to claim 40, wherein R^{b} is —(C₁-C₅)alkyl-O—C(═O)—R^{c}.

42. A compound according to claim 39, wherein R^{b} is -direct bond-(CH(CH₃))—O—C(═O)—R^{c} or —(C₁-C₄)alkyl-(CH(CH₃))—O—C(═O)—R^{c}.

43. A compound according to any one of claims 40-42, wherein R^{c} is —O—(C₁-C₆)alkyl or —O— phenyl.

44. A compound according to any one of claims 40-42, wherein R^{c} is —NH₂ or —NH—(C₁-C₆)alkyl.

45. A compound according to any one of claims 40-42, wherein R^{c} is —N—[(C₁-C₆)alkyl]₂, where the alkyl groups can be combined to form a (C₄-C₆) nitrogenous aliphatic heterocycle.

46. A compound according to any one of claims 40-42, wherein R^{c} is —(C₁-C₇)alkyl.

47. A compound according to any one of claims 40-42, wherein R^{c} is -phenyl.

48. A compound according to any one of claims 40-42, wherein R^{c} is —(C₁-C₆)alkyl-NH—R^{e}.

49. A compound according to claim 48, wherein R^{e} is (C₁-C₆)alkyl.

50. A compound according to claim 49, wherein R^{e} is methyl or ethyl.

51. A compound according to claim 48, wherein the alkyl linker and R^{e} are combined to form a (C₄-C₆) nitrogenous aliphatic heterocycle.

52. A compound according to claim 51, wherein said (C₄-C₆) nitrogenous aliphatic heterocycle is azetidine, pyrrolidine, or piperidine.

53. A compound according to claim 52, wherein said (C₄-C₆) nitrogenous aliphatic heterocycle is pyrrolidine.

54. A compound according to any one of claims 40-53, wherein R^{a} is H.

55. A compound according to any one of claims 40-53, wherein R^{a} is methyl.

56. A compound according to claim 39, wherein R^{b} is —(C₁-C₁₀)hydrocarbyl[-OC(═O)—R^{c}]₂.

57. A compound according to claim 56, wherein R^{b} is —(C₁-C₄)alkyl[-OC(═O)—R^{c}]₂.

58. A compound according to claim 57, wherein R^{c} is —(C₁-C₇)alkyl.

59. A compound according to claim 57, wherein R^{c} is -phenyl.

60. A compound according to any one of claims 56-59, wherein R^{a} is H.

61. A compound according to any one of claims 56-59, wherein R^{a} is methyl.

62. A compound according to claim 39, wherein R^{b} is —(C₁-C₁₀)hydrocarbyl-NR^{e}—C(═O)—R^{f}.

63. A compound according to claim 62, wherein R^{b} is —(C₁-C₅)alkyl-NR^{e}—C(═O)—R^{f}.

64. A compound according to claim 62, wherein R^{b} is -direct bond-(CH(CH₃))—NR^{e}—C(═O)—R^{f} or —(C₁-C₄)alkyl-(CH(CH₃))—NR^{e}—C(═O)—R^{f}.

65. A compound according to any one of claims 62-64, wherein R^{e} is H.

66. A compound according to any one of claims 62-64, wherein R^{e} is methyl or ethyl.

67. A compound according to any one of claims 62-64, wherein R^{f} is —O—(C₁-C₆)alkyl.

68. A compound according to any one of claims 62-64, wherein R^{f} is —O-phenyl.

69. A compound according to any one of claims 62-64, wherein R^{f} is —(C₁-C₆)alkyl.

70. A compound according to any one of claims 62-64, wherein R^{f} is -phenyl.

71. A compound according to any one of claims 62-64, wherein R^{f} is -direct bond-C(═O)—O—(C₁-C₆)alkyl or —(C₁-C₄)hydrocarbyl-C(═O)—O—(C₁-C₆)alkyl.

72. A compound according to claim 71, wherein R^{f} is —CH═CH—C(═O)—O—(C₁-C₆)alkyl.

73. A compound according to any one of claims 62-64, wherein R^{f} is -direct bond-C(═O)—NH₂ or —(C₁-C₄)hydrocarbyl-C(═O)—NH₂.

74. A compound according to claim 73, wherein R^{f} is —CH═CH—C(═O)—NH₂.

75. A compound according to any one of claims 62-64, wherein R^{f} is -direct bond-C(═O)—NH—(C₁-C₆)alkyl or —(C₁-C₄)hydrocarbyl-C(═O)—NH—(C₁-C₆)alkyl.

76. A compound according to claim 75, wherein R^{f} is —CH═CH—C(═O)—NH—(C₁-C₆)alkyl.

77. A compound according to any one of claims 62-64, wherein R^{f} is —(C₁-C₆)alkyl-NH—R^{e}, where the alkyl linker can be combined with R^{e} to form a (C₄-C₆) nitrogenous aliphatic heterocycle.

78. A compound according to claim 77, wherein said (C₄-C₆) nitrogenous aliphatic heterocycle is azetidine, pyrrolidine, or piperidine.

79. A compound according to claim 78, wherein said (C₄-C₆) nitrogenous aliphatic heterocycle is pyrrolidine.

80. A compound according to any one of claims 62-64, wherein R^{f} is —(C₁-C₆)alkyl-NH—C(═O)(C₁-C₁₀)hydrocarbyl.

81. A compound according to claim 80 wherein R^{f} is —(C₁-C₆)alkyl-NH—C(═O)(C₁-C₆)alkyl.

82. A compound according to claim 80 wherein R^{f} is —(C₁-C₆)alkyl-NH—C(═O)phenyl.

83. A compound according to any one of claims 62-82 wherein R^{a} is H.

84. A compound according to any one of claims 62-82 wherein R^{a} is methyl.

85. A compound according to claim 39, wherein R^{b} is -direct bond-C(═O)—R^{c}.

86. A compound according to claim 39, wherein R^{b} is —(C₁-C₅)alkyl-C(═O)—R^{c}.

87. A compound according to any one of claims 85-86, wherein R^{c} is —O—(C₁-C₆)alkyl.

88. A compound according to any one of claims 85-86, wherein R^{c} is —NH₂ or —NH—(C₁-C₆)alkyl.

89. A compound according to any one of claims 85-87, wherein R^{c} is —N—[(C₁-C₆)alkyl]₂, where the alkyl groups can be combined to form a (C₄-C₆) nitrogenous aliphatic heterocycle.

90. A compound according to claim 89, wherein said (C₄-C₆) nitrogenous aliphatic heterocycle is azetidine, pyrrolidine, or piperidine.

91. A compound according to claim 85-86, wherein R^{c} is —NR^{e}—(C₁-C₆)alkyl-C(═O)—O—(C₁-C₆)alkyl.

92. A compound according to claim 91, wherein R^{e} is H.

93. A compound according to claim 91, wherein R^{e} is methyl.

94. A compound according to claim 91, wherein the alkyl group is combined with R^{e} to form a (C₄-C₆) nitrogenous aliphatic heterocycle.

95. A compound according to claim 94, wherein said (C₄-C₆) nitrogenous aliphatic heterocycle is azetidine, pyrrolidine, or piperidine.

96. A compound according to any one of claims 85-95 wherein R^{a} is H.

97. A compound according to any one of claims 85-95 wherein R^{a} is methyl.

98. A compound according to any one of claims 85-87 wherein R^{a} is —C(═O)—O—(C₁-C₄)alkyl.

99. A compound according to claim 39 wherein R^{b} is —CH(NHR^{d})C(═O)—R^{c}.

100. A compound according to claim 99 wherein R^{c} is —O—(C₁-C₆)alkyl.

101. A compound according to claim 100 wherein R^{d} is —(C₁-C₆)alkyl.

102. A compound according to claim 100 wherein R^{d} is —C(═O)(C₁-C₆)alkyl.

103. A compound according to claim 100 wherein R^{d} is —C(═O)phenyl.

104. A compound according to any one of claims 39-103 wherein R^{a} is H.

105. A compound according to any one of claims 39-103 wherein R^{a} is methyl.

106. A compound according to any one of claims 37-38 wherein R¹ is aromatic (C₁-C₂₀)hydrocarbyl.

107. A compound according to claim 106 wherein R¹ is a substituted or unsubstituted benzoate ester.

108. A compound according to claim 107 wherein R¹ is an ethyl meta-benzoate, ethyl ortho-benzoate, or ethyl para-benzoate.

109. A compound according to claim 107 wherein R¹ is a tyrosine derivative.

110. A compound according to claim 107 wherein R¹ is α-tocopherol.

111. A compound according to claim 107 wherein R¹ is an anilide.

112. A compound according to claim 111 wherein R¹ is acetaminophen.

113. A compound according to any of claims 37-112, wherein each of R³-R²⁰ is hydrogen.

114. A compound according to any of claims 37-112, wherein at least one of R³-R²⁰ is deuterium.

115. A compound according to claim 114, wherein R³⁻⁴ is deuterium.

116. A compound according to claim 114, wherein R²⁰ is deuterium.

117. A compound according to claim 114, wherein R¹⁸⁻²⁰ is deuterium.

118. A compound according to any one of claims 37-38 wherein said compound is selected from any compound in Table 4.

119. A compound according to any one of claims 37-118, wherein R² is H.

120. A compound according to any one of claims 37-118, wherein R² is optionally substituted (C₁-C₇)hydrocarbyl, and wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂.

121. A compound according to claim 120, wherein R² is optionally substituted benzyl, and wherein said optionally substituted benzyl is optionally substituted with one or more of —C(═O)—OH, —C(═O)—O—CH₃, or —C(═O)—NH—CH₃.

122. A compound according to claim 120, wherein R² is optionally substituted (C₁-C₃)hydrocarbyl, and wherein said optionally substituted (C₁-C₃)hydrocarbyl is substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂.

123. A compound according to claim 122, wherein R² is methyl optionally substituted with one or more of —F, —O—C(═O)(C₁-C₄)hydrocarbyl, —C(═O)—OH, —C(═O)—O—CH₃, or —C(═O)—NH—CH₃.

124. A compound according to claim 123, wherein R² is methyl.

125. A compound according to claim 123, wherein R² is difluoro methyl.

126. A compound according to claim 123, wherein R² is CH₂—O—C(═O)(C₁-C₄)hydrocarbyl.

127. A compound according to claim 126, wherein R² is CH₂—O—C(═O)—CH₃.

128. A compound according to claim 123, wherein R² is CH₂—C(═O)—OH.

129. A compound according to claim 123, wherein R² is CH₂—C(═O)—O—CH₃.

130. A compound according to claim 123, wherein R² is CH₂—C(═O)—NH—CH₃.

131. A compound according to claim 122, wherein R² is ethyl optionally substituted with one or more of —OH, —O—C(═O)(C₁-C₄)hydrocarbyl, —NH(C₁-C₄)hydrocarbyl, or —N[(C₁-C₄)hydrocarbyl)]₂.

132. A compound according to claim 131, wherein R² is ethyl.

133. A compound according to claim 131, wherein R² is ethanol.

134. A compound according to claim 131, wherein R² is (CH₂)₂—O—C(═O)—CH₃.

135. A compound according to claim 122, wherein R² is propyl optionally substituted with one or more of —CN.

136. A compound according to any one of claims 37-38, with the structure of formula VIII:

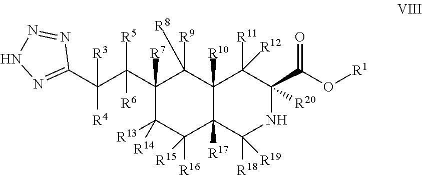

wherein:
at least one of R³-R²⁰ is deuterium; and
R¹ is chosen from H and (C₁-C₁₀) hydrocarbyl.

137. A pharmaceutical composition comprising a pharmaceutically acceptable carrier and a compound according to any one of claims 1-136, or a pharmaceutically acceptable salt thereof.

138. A method of treating a seizure disorder, status epilepticus, partial-onset seizures, primary generalized tonic-clonic seizures, or convulsions, comprising administering to a subject a therapeutically or prophylactically effective amount of the compound of any one of claims 1-136.

139. The method according to claim 138, wherein said seizure disorder is epilepsy.

140. The method according to claim 138, wherein said seizure-disorder is a heritable genetic seizure disorder.

141. The method according to claim 138, wherein said seizure-disorder may result from a brain tumor, traumatic brain injury (either concussive or penetrating), or head injury.

142. The method according to claim 138, wherein said seizure-disorder may result from brain injury during or after viral infection, bacterial infection, parasitic infection, prion disease or idiopathic causes.

143. The method according to claim 138, wherein said seizure-disorder may result from hemorrhagic stroke or ischemic stroke as post-stroke seizures or recurrent epilepsy after stroke.

144. The method according to claim 138, wherein said seizure-disorder may result from systemic autoimmune disorders (autoimmune epilepsy), autoimmune encephalitis, Rasmussen's encephalitis (RE), febrile-seizures, febrile-related epilepsies, fever induced refractory epilepsy syndrome (FIRES), or neuroinflammation.

What is claimed is:

1. A compound of formula Ia or Ib: 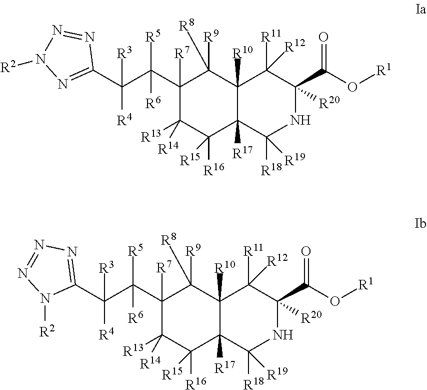
wherein:
R¹ is selected from H, (C₁-C₂₀)hydrocarbyl, and —CH(R^{a})R^{b};
R^{a} is selected from H, —(C₁-C₆)alkyl, and —C(═O)—O—(C₁-C₆)alkyl;
R^{b} is selected from —(C₁-C₁₀)hydrocarbyl-O—C(═O)—R^{c}, —(C₁-C₁₀)hydrocarbyl[-OC(═O)—R^{c}]₂, -direct bond-C(═O)—R^{c} or —(C₁-C₁₀)hydrocarbyl-C(═O)—R^{c}, —CH(NHR^{d})C(═O)—R^{c}, and —(C₁-C₁₀)hydrocarbyl-NR^{e}—C(═O)—R^{f};
R^{c} is selected from —O—(C₁-C₁₀)hydrocarbyl, —NH—(C₁-C₁₀)hydrocarbyl, —N—[(C₁-C₁₀)hydrocarbyl]₂, where the hydrocarbyl groups can be combined to form a (C₄-C₆) nitrogenous aliphatic heterocycle, —(C₁-C₁₀)hydrocarbyl, —NH₂, —(C₁-C₁₀)hydrocarbyl-NH—R^{e}, where the hydrocarbyl group can be combined with R^{e} to form a (C₄-C₆) nitrogenous aliphatic heterocycle, and —NR^{e}—(C₁-C₁₀)hydrocarbyl-C(═O)—O—(C₁-C₁₀)hydrocarbyl, where the hydrocarbyl group and R^{e} can be combined to form a (C₄-C₆) nitrogenous aliphatic heterocycle;
R^{d} is selected from H, —(C₁-C₁₀)hydrocarbyl, and —C(═O)(C₁-C₁₀)hydrocarbyl;
R^{e} is selected from H, and —(C₁-C₆)alkyl;
R^{f} is selected from —O—(C₁-C₁₀)hydrocarbyl, —(C₁-C₁₀)hydrocarbyl, -direct bond-C(═O)—O—(C₁-C₁₀)hydrocarbyl or —(C₁-C₄)hydrocarbyl-C(═O)—O—(C₁-C₁₀)hydrocarbyl, -direct bond-C(═O)—NH₂ or —(C₁-C₄)hydrocarbyl-C(═O)—NH₂, -direct bond-C(═O)—NH—(C₁-C₁₀)hydrocarbyl or —(C₁-C₄)hydrocarbyl-C(═O)—NH—(C₁-C₁₀)hydrocarbyl, —(C₁-C₁₀)hydrocarbyl-NH—R^{e}, where the hydrocarbyl linker can be combined with R^{e} to form a (C₄-C₆) nitrogenous aliphatic heterocycle, and —(C₁-C₁₀)hydrocarbyl-NH—C(═O)(C₁-C₁₀)hydrocarbyl;
R² is selected from H and optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂;
R⁷ is selected from hydrogen, deuterium, and fluorine;
R³-R⁶ and R⁸-R²⁰ are independently selected from hydrogen and deuterium; and
with the provisos:
(i) when R¹ is C₁-C₆ alkyl or aryl and R² is a nitrogen protecting group, wherein said nitrogen protecting group is selected from trityl, benzyl, t-butyl, t-butyldimethylsilyl, and triphenylsilyl, then either (a) R⁷ is fluorine or (b) at least one of R³-R²⁰ is deuterium;
(ii) when R¹ is H, C₁-C₆ alkyl, substituted alkyl, cycloalkyl, or arylalkyl and R² is H, then either (a) R⁷ is fluorine or at (b) least one of R³-R²⁰ is deuterium; and
(iii) when R⁷ is fluorine, then R² is not H.

2. A compound according to claim 1, with the structure of formula IIa, IIb, IIIa, or IIb: 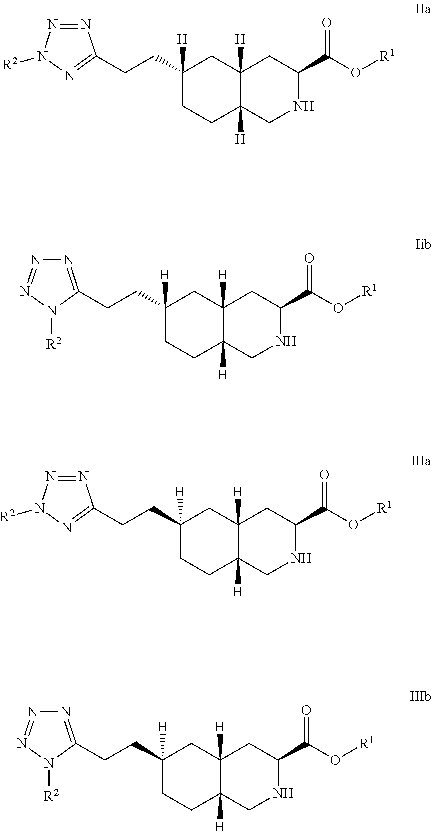
wherein:
R¹ is selected from H and (C₁-C₂₀)hydrocarbyl;
R² is optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂; and
R³-R²⁰ are each H.

3. A compound according to claim 1, with the structure of formula IVa, IVb, Va, or Vb: 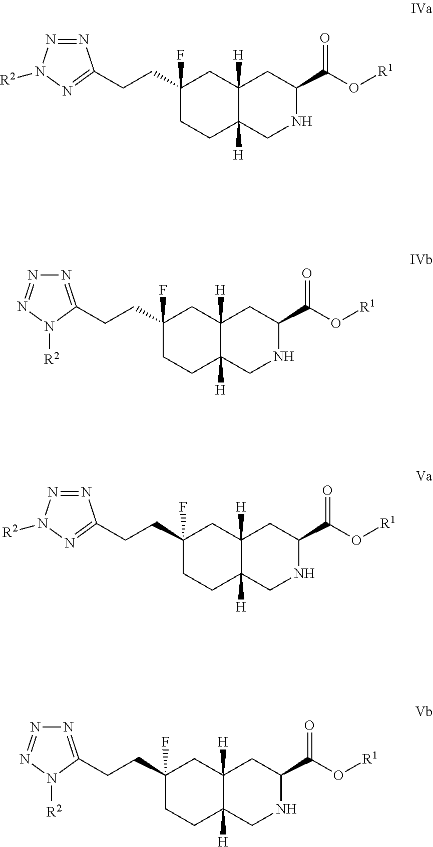
wherein:
R¹ is selected from H and (C₁-C₂₀)hydrocarbyl;
R³-R⁶ and R⁸-R²⁰ are each H;
R⁷ is fluorine; and
R² is optionally substituted (C₁-C₇)hydrocarbyl, wherein said optionally substituted (C₁-C₇)hydrocarbyl may be substituted with one or more of —F, —CN, —OH, —O—C(═O)(C₁-C₄)hydrocarbyl, —C(═O)—OH, —C(═O)—O—CH₃, —C(═O)—NH—CH₃, —NH(C₁-C₄)hydrocarbyl, —N[(C₁-C₄)hydrocarbyl)]₂, or —C(═O)—N[(C₁-C₃)hydrocarbyl]₂;

4. A compound according to claim 3, with the structure of formula IVa or IVb:

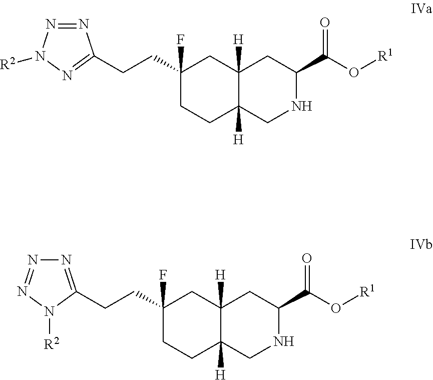

5. A compound according to claim 3, with the structure of formula V:

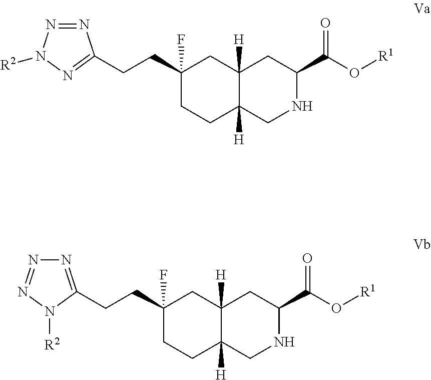

6. A compound according to any one of claims 1-5, wherein R¹ is selected from H and aliphatic (C₁-C₂₀)hydrocarbyl optionally substituted with one or two phenyl groups with the proviso that R¹ contains twenty carbons or less.

7. A compound according to any one of claims 1-6, wherein R¹ is selected from H and (C₁-C₂₀)alkyl optionally substituted with one or two phenyl groups with the proviso that R¹ contains twenty carbons or less.

8. A compound according to any one of claims 1-5, wherein R¹ is H or (C₁-C₁₃)hydrocarbyl.

9. A compound according to any one of claims 1-6 and 8, wherein R¹ is H or aliphatic (C₁-C₁₃)hydrocarbyl optionally substituted with one or two phenyl groups with the proviso that R¹ contains twenty carbons or less.

10. A compound according to any one of claims 1-9, wherein R¹ is H or (C₁-C₁₃)alkyl optionally substituted with one or two phenyl groups with the proviso that R¹ contains twenty carbons or less.

11. A compound according to any one of claims 1-10, wherein R¹ is H or (C₁-C₁₀)alkyl optionally substituted with one or two phenyl groups with the proviso that R¹ contains twenty carbons or less.

12. A compound according to any one of claims 1-5 wherein R is H or C_{q}Hᵣ, and wherein:
q is 1 and r is 3;
q is 2 and r is 5;
q is 3 and r is 3, 5, or 7;
q is 4 and r is 5, 7, or 9;
g is 5 and r is 7, 9, or 11;
g is 6 and r is 5, 7, 9, 11, or 13.
q is 7 and r is 7, 9, 11, 13, or 15;
q is 8 and r is 5, 7, 9, 11, 13, 15, or 17;
q is 9 and r is 7, 9, 11, 13, 15, 17, or 19;
q is 10 and r is 7, 9, 11, 13, 15, 17, 19, or 21;
q is 11 and r is 9, 11, 13, 15, 17, 19, 21, or 23;
q is 12 and r is 7, 9, 11, 13, 15, 17, 19, 21, 23, or 25;
q is 13 and r is 9, 11, 13, 15, 17, 19, 21, 23, 25, or 27;
q is 14 and r is 9, 11, 13, 15, 17, 19, 21, 23, 25, 27, or 29;
q is 15 and r is 11, 13, 15, 17, 19, 21, 23, 25, 27, 29, or 31;
q is 16 and r is 9, 11, 13, 15, 17, 19, 21, 23, 25, 27, 29, 31, or 33;
q is 17 and r is 11, 15, 17, 19, 21, 23, 25, 27, 29, 31, 33, or 35;
q is 18 and r is 11, 13, 15, 17, 19, 21, 23, 25, 27, 29, 31, 33, 35, or 37;
q is 19 and r is 13, 15, 17, 19, 21, 23, 25, 27, 29, 31, 33, 35, 37, or 39; or
q is 20 and r is 11, 13, 15, 17, 19, 21, 23, 25, 27, 29, 31, 33, 35, 37, 39, or 41.

13. A compound according to any one of claims 1-5 and 8, wherein R¹ is H, methyl, ethyl, n-propyl, isopropyl, cyclopropyl, n-butyl, 1-methylpropyl, 1-methyl-2-ethylbutyl, 2-ethylbutyl, 2-methylpropyl, tert-butyl, 2-methylcyclopropyl, 1-methylcyclopropyl, cyclobutyl, cyclopropylmethyl (i.e., 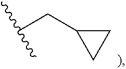 [SMILES: *CC1CC1]
n-pentyl, 1-methylbutyl, 2-methylbutyl, 3-methylbutyl, 1-ethylpropyl, 1,2-dimethylpropyl, 1,1-dimethylpropyl, 2,2-dimethylpropyl, cyclobutylmethyl (i.e., 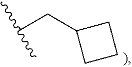 [SMILES: *CC1CCC1]
2-(cyclopropyl)ethyl (i.e., 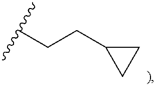 [SMILES: *CCC1CC1]
cyclopentyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,3-dimethylbutyl, 2,2-dimethylbutyl, 3,3-dimethylbutyl, 1,1,2-trimethylpropyl, 1,2,2-trimethylpropyl, 3-(cyclopropyl)propyl (i.e., 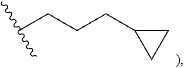 [SMILES: *CCCC1CC1]
2-(cyclobutyl)ethyl (i.e., 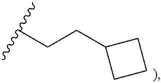 [SMILES: *CCC1CCC1]
cyclopentylmethyl (i.e., 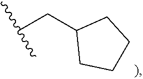 [SMILES: *CC1CCCC1]
cyclohexyl, cyclohexylmethyl, 2-cyclohexylethyl, dicyclohexylmethyl, n-octyl, benzyl, diphenylmethyl, decyl, dodecyl, tetradecyl, hexadecyl, 253ctadic-9-enyl, octadecyl, 253ctadic-9-enyl, 253ctadic-9,12-dienyl, 2-propylpentyl, 2-butylhexyl, 2-pentylheptyl, or 2-hexyloctyl.

14. A compound according to any one of claims 1-5, 8, 9, and 12, wherein R¹ is H, methyl, ethyl, n-propyl, isopropyl, n-butyl, tert-butyl, 2-methylbutyl, 2-ethylbutyl, or cyclohexyl.

15. A compound according to any one of claims 1-14, wherein R¹ is H.

16. A compound according to any one of claims 1-14, wherein R¹ is ethyl.

17. A compound according to any one of claims 1-14, wherein R¹ is isopropyl.

18. A compound according to any one of claims 1-14, wherein R¹ is 2-ethylbutyl.

19. A compound according to any one of claims 1-5, 8, 9, 13, and 14, wherein R¹ is cyclohexyl.

20. A compound according to any one of claims 1-19, wherein R² is optionally substituted (C₁-C₇)hydrocarbyl, and wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂.

21. A compound according to claim 20, wherein R² is optionally substituted benzyl, and wherein said optionally substituted benzyl is optionally substituted with one or more of —C(═O)—OH, —C(═O)—O—CH₃, or —C(═O)—NH—CH₃.

22. A compound according to claim 20, wherein R² is optionally substituted (C₁-C₃)hydrocarbyl, and wherein said optionally substituted (C₁-C₃)hydrocarbyl is substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂.

23. A compound according to claim 22, wherein R² is methyl optionally substituted with one or more of —F, —O—C(═O)(C₁-C₄)hydrocarbyl, —C(═O)—OH, —C(═O)—O—CH₃, or —C(═O)—NH—CH₃.

24. A compound according to claim 23, wherein R² is methyl.

25. A compound according to claim 23, wherein R² is difluoromethyl.

26. A compound according to claim 23, wherein R² is CH₂—O—C(═O)(C₁-C₄)hydrocarbyl.

27. A compound according to claim 26, wherein R² is CH₂—O—C(═O)—CH₃.

28. A compound according to claim 23, wherein R² is CH₂—C(═O)—OH.

29. A compound according to claim 23, wherein R² is CH₂—C(═O)—O—CH₃.

30. A compound according to claim 23, wherein R² is CH₂—C(═O)—NH—CH₃.

31. A compound according to claim 22, wherein R² is ethyl optionally substituted with one or more of —OH, —O—C(═O)(C₁-C₄)hydrocarbyl, —NH(C₁-C₄)hydrocarbyl, or —N[(C₁-C₄)hydrocarbyl)]₂.

32. A compound according to claim 30, wherein R² is ethyl.

33. A compound according to claim 30, wherein R² is ethanol.

34. A compound according to claim 30, wherein R² is (CH₂)₂—O—C(═O)—CH₃.

35. A compound according to claim 30, wherein R² is (CH₂)₂—N[(C₁-C₄)hydrocarbyl)]₂.

36. A compound according to claim 22, wherein R² is propyl optionally substituted with one or more of —CN.

37. A compound according to claim 1, with the structure of formula VIa or VIb: 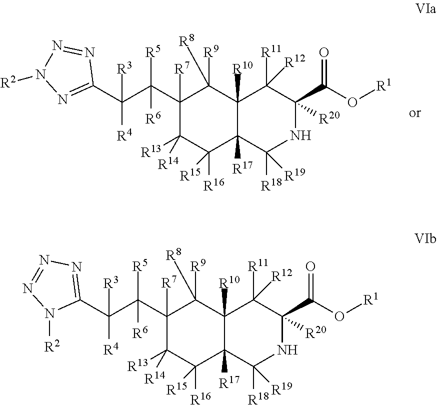
wherein:
R¹ is selected from —CH(R^{a})R^{b} and aromatic (C₁-C₂₀)hydrocarbyl;
R^{a} is selected from H, —(C₁-C₆)alkyl, and —C(═O)—O—(C₁-C₆)alkyl;
R^{b} is selected from —(C₁-C₁₀)hydrocarbyl-O—C(═O)—R^{c}, —(C₁-C₁₀)hydrocarbyl[-OC(═O)—R^{c}]₂, -direct bond-C(═O)—R^{c} or —(C₁-C₁₀)hydrocarbyl-C(═O)—R^{c}, —CH(NHR^{d})C(═O)—R^{c}, and —(C₁-C₁₀)hydrocarbyl-NR^{e}—C(═O)—R^{f};
R^{c} is selected from —O—(C₁-C₁₀)hydrocarbyl, —NH—(C₁-C₁₀)hydrocarbyl, —N—[(C₁-C₁₀)hydrocarbyl]₂, where the hydrocarbyl groups can be combined to form a (C₄-C₆) nitrogenous aliphatic heterocycle, —(C₁-C₁₀)hydrocarbyl, —NH₂, —(C₁-C₁₀)hydrocarbyl-NH—R^{e}, where the hydrocarbyl group can be combined with R^{e} to form a (C₄-C₆) nitrogenous aliphatic heterocycle, and —NR^{e}—(C₁-C₁₀)hydrocarbyl-C(═O)—O—(C₁-C₁₀)hydrocarbyl, where the hydrocarbyl group and R^{e} can be combined to form a (C₄-C₆) nitrogenous aliphatic heterocycle;
R^{d} is selected from H, —(C₁-C₁₀)hydrocarbyl, and —C(═O)(C₁-C₁₀)hydrocarbyl;
R^{e} is selected from H, and —(C₁-C₆)alkyl;
R^{f} is selected from —O—(C₁-C₁₀)hydrocarbyl, —(C₁-C₁₀)hydrocarbyl, -direct bond-C(═O)—O—(C₁-C₁₀)hydrocarbyl or —(C₁-C₄)hydrocarbyl-C(═O)—O—(C₁-C₁₀)hydrocarbyl, -direct bond-C(═O)—NH₂ or —(C₁-C₄)hydrocarbyl-C(═O)—NH₂, -direct bond-C(═O)—NH—(C₁-C₁₀)hydrocarbyl or —(C₁-C₄)hydrocarbyl-C(═O)—NH—(C₁-C₁₀)hydrocarbyl, —(C₁-C₁₀)hydrocarbyl-NH—R^{e}, where the hydrocarbyl linker can be combined with R^{e} to form a (C₄-C₆) nitrogenous aliphatic heterocycle, and —(C₁-C₁₀)hydrocarbyl-NH—C(═O)(C₁-C₁₀)hydrocarbyl;
R² is selected from H and optionally substituted (C₁-C₇)hydrocarbyl, wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂;
R³-R²⁰ are each independently selected from hydrogen and deuterium; and
wherein at least one of R³-R²⁰ is deuterium.

38. A compound according to claim 37, with the structure of formula VIIa or VIIb:

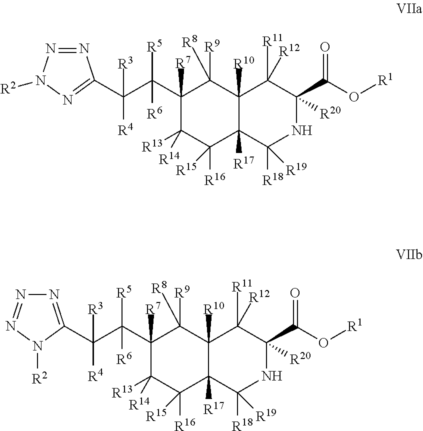

39. A compound according to any one of claims 37-38, wherein R¹ is —CH(R^{a})R^{b}.

40. A compound according to claim 39, wherein R^{b} is —(C₁-C₁₀)hydrocarbyl-O—C(═O)—R^{c}.

41. A compound according to claim 40, wherein R^{b} is —(C₁-C₅)alkyl-O—C(═O)—R^{c}.

42. A compound according to claim 39, wherein R^{b} is -direct bond-(CH(CH₃))—O—C(═O)—R^{c} or —(C₁-C₄)alkyl-(CH(CH₃))—O—C(═O)—R^{c}.

43. A compound according to any one of claims 40-42, wherein R^{c} is —O—(C₁-C₆)alkyl or —O— phenyl.

44. A compound according to any one of claims 40-42, wherein R^{c} is —NH₂ or —NH—(C₁-C₆)alkyl.

45. A compound according to any one of claims 40-42, wherein R^{c} is —N—[(C₁-C₆)alkyl]₂, where the alkyl groups can be combined to form a (C₄-C₆) nitrogenous aliphatic heterocycle.

46. A compound according to any one of claims 40-42, wherein R^{c} is —(C₁-C₇)alkyl.

47. A compound according to any one of claims 40-42, wherein R^{c} is -phenyl.

48. A compound according to any one of claims 40-42, wherein R^{c} is —(C₁-C₆)alkyl-NH—R^{e}.

49. A compound according to claim 48, wherein R^{e} is (C₁-C₆)alkyl.

50. A compound according to claim 49, wherein R^{e} is methyl or ethyl.

51. A compound according to claim 48, wherein the alkyl linker and R^{e} are combined to form a (C₄-C₆) nitrogenous aliphatic heterocycle.

52. A compound according to claim 51, wherein said (C₄-C₆) nitrogenous aliphatic heterocycle is azetidine, pyrrolidine, or piperidine.

53. A compound according to claim 52, wherein said (C₄-C₆) nitrogenous aliphatic heterocycle is pyrrolidine.

54. A compound according to any one of claims 40-53, wherein R^{a} is H.

55. A compound according to any one of claims 40-53, wherein R^{a} is methyl.

56. A compound according to claim 39, wherein R^{b} is —(C₁-C₁₀)hydrocarbyl[-OC(═O)—R^{c}]₂.

57. A compound according to claim 56, wherein R^{b} is —(C₁-C₄)alkyl[-OC(═O)—R^{c}]₂.

58. A compound according to claim 57, wherein R^{c} is —(C₁-C₇)alkyl.

59. A compound according to claim 57, wherein R^{c} is -phenyl.

60. A compound according to any one of claims 56-59, wherein R^{a} is H.

61. A compound according to any one of claims 56-59, wherein R^{a} is methyl.

62. A compound according to claim 39, wherein R^{b} is —(C₁-C₁₀)hydrocarbyl-NR^{e}—C(═O)—R^{f}.

63. A compound according to claim 62, wherein R^{b} is —(C₁-C₅)alkyl-NR^{e}—C(═O)—R^{f}.

64. A compound according to claim 62, wherein R^{b} is -direct bond-(CH(CH₃))—NR^{e}—C(═O)—R^{f} or —(C₁-C₄)alkyl-(CH(CH₃))—NR^{e}—C(═O)—R^{f}.

65. A compound according to any one of claims 62-64, wherein R^{e} is H.

66. A compound according to any one of claims 62-64, wherein R^{e} is methyl or ethyl.

67. A compound according to any one of claims 62-64, wherein R^{f} is —O—(C₁-C₆)alkyl.

68. A compound according to any one of claims 62-64, wherein R^{f} is —O-phenyl.

69. A compound according to any one of claims 62-64, wherein R^{f} is —(C₁-C₆)alkyl.

70. A compound according to any one of claims 62-64, wherein R^{f} is -phenyl.

71. A compound according to any one of claims 62-64, wherein R^{f} is -direct bond-C(═O)—O—(C₁-C₆)alkyl or —(C₁-C₄)hydrocarbyl-C(═O)—O—(C₁-C₆)alkyl.

72. A compound according to claim 71, wherein R^{f} is —CH═CH—C(═O)—O—(C₁-C₆)alkyl.

73. A compound according to any one of claims 62-64, wherein R^{f} is -direct bond-C(═O)—NH₂ or —(C₁-C₄)hydrocarbyl-C(═O)—NH₂.

74. A compound according to claim 73, wherein R^{f} is —CH═CH—C(═O)—NH₂.

75. A compound according to any one of claims 62-64, wherein R^{f} is -direct bond-C(═O)—NH—(C₁-C₆)alkyl or —(C₁-C₄)hydrocarbyl-C(═O)—NH—(C₁-C₆)alkyl.

76. A compound according to claim 75, wherein R^{f} is —CH═CH—C(═O)—NH—(C₁-C₆)alkyl.

77. A compound according to any one of claims 62-64, wherein R^{f} is —(C₁-C₆)alkyl-NH—R^{e}, where the alkyl linker can be combined with R^{e} to form a (C₄-C₆) nitrogenous aliphatic heterocycle.

78. A compound according to claim 77, wherein said (C₄-C₆) nitrogenous aliphatic heterocycle is azetidine, pyrrolidine, or piperidine.

79. A compound according to claim 78, wherein said (C₄-C₆) nitrogenous aliphatic heterocycle is pyrrolidine.

80. A compound according to any one of claims 62-64, wherein R^{f} is —(C₁-C₆)alkyl-NH—C(═O)(C₁-C₁₀)hydrocarbyl.

81. A compound according to claim 80 wherein R^{f} is —(C₁-C₆)alkyl-NH—C(═O)(C₁-C₆)alkyl.

82. A compound according to claim 80 wherein R^{f} is —(C₁-C₆)alkyl-NH—C(═O)phenyl.

83. A compound according to any one of claims 62-82 wherein R^{a} is H.

84. A compound according to any one of claims 62-82 wherein R^{a} is methyl.

85. A compound according to claim 39, wherein R^{b} is -direct bond-C(═O)—R^{c}.

86. A compound according to claim 39, wherein R^{b} is —(C₁-C₅)alkyl-C(═O)—R^{c}.

87. A compound according to any one of claims 85-86, wherein R^{c} is —O—(C₁-C₆)alkyl.

88. A compound according to any one of claims 85-86, wherein R^{c} is —NH₂ or —NH—(C₁-C₆)alkyl.

89. A compound according to any one of claims 85-87, wherein R^{c} is —N—[(C₁-C₆)alkyl]₂, where the alkyl groups can be combined to form a (C₄-C₆) nitrogenous aliphatic heterocycle.

90. A compound according to claim 89, wherein said (C₄-C₆) nitrogenous aliphatic heterocycle is azetidine, pyrrolidine, or piperidine.

91. A compound according to claim 85-86, wherein R^{c} is —NR^{e}—(C₁-C₆)alkyl-C(═O)—O—(C₁-C₆)alkyl.

92. A compound according to claim 91, wherein R^{e} is H.

93. A compound according to claim 91, wherein R^{e} is methyl.

94. A compound according to claim 91, wherein the alkyl group is combined with R^{e} to form a (C₄-C₆) nitrogenous aliphatic heterocycle.

95. A compound according to claim 94, wherein said (C₄-C₆) nitrogenous aliphatic heterocycle is azetidine, pyrrolidine, or piperidine.

96. A compound according to any one of claims 85-95 wherein R^{a} is H.

97. A compound according to any one of claims 85-95 wherein R^{a} is methyl.

98. A compound according to any one of claims 85-87 wherein R^{a} is —C(═O)—O—(C₁-C₄)alkyl.

99. A compound according to claim 39 wherein R^{b} is —CH(NHR^{d})C(═O)—R^{c}.

100. A compound according to claim 99 wherein R^{c} is —O—(C₁-C₆)alkyl.

101. A compound according to claim 100 wherein R^{d} is —(C₁-C₆)alkyl.

102. A compound according to claim 100 wherein R^{d} is —C(═O)(C₁-C₆)alkyl.

103. A compound according to claim 100 wherein R^{d} is —C(═O)phenyl.

104. A compound according to any one of claims 39-103 wherein R^{a} is H.

105. A compound according to any one of claims 39-103 wherein R^{a} is methyl.

106. A compound according to any one of claims 37-38 wherein R¹ is aromatic (C₁-C₂₀)hydrocarbyl.

107. A compound according to claim 106 wherein R¹ is a substituted or unsubstituted benzoate ester.

108. A compound according to claim 107 wherein R¹ is an ethyl meta-benzoate, ethyl ortho-benzoate, or ethyl para-benzoate.

109. A compound according to claim 107 wherein R¹ is a tyrosine derivative.

110. A compound according to claim 107 wherein R¹ is α-tocopherol.

111. A compound according to claim 107 wherein R¹ is an anilide.

112. A compound according to claim 111 wherein R¹ is acetaminophen.

113. A compound according to any of claims 37-112, wherein each of R³-R²⁰ is hydrogen.

114. A compound according to any of claims 37-112, wherein at least one of R³-R²⁰ is deuterium.

115. A compound according to claim 114, wherein R³⁻⁴ is deuterium.

116. A compound according to claim 114, wherein R²⁰ is deuterium.

117. A compound according to claim 114, wherein R¹⁸⁻²⁰ is deuterium.

118. A compound according to any one of claims 37-38 wherein said compound is selected from any compound in Table 4.

119. A compound according to any one of claims 37-118, wherein R² is H.

120. A compound according to any one of claims 37-118, wherein R² is optionally substituted (C₁-C₇)hydrocarbyl, and wherein said optionally substituted (C₁-C₇)hydrocarbyl is optionally substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂.

121. A compound according to claim 120, wherein R² is optionally substituted benzyl, and wherein said optionally substituted benzyl is optionally substituted with one or more of —C(═O)—OH, —C(═O)—O—CH₃, or —C(═O)—NH—CH₃.

122. A compound according to claim 120, wherein R² is optionally substituted (C₁-C₃)hydrocarbyl, and wherein said optionally substituted (C₁-C₃)hydrocarbyl is substituted with one or more of —F, —CN, —OH, —O—C(═O)(C₁-C₄)hydrocarbyl, —C(═O)—OH, —C(═O)—O—CH₃, —C(═O)—NH—CH₃, —C(═O)NH₂, —NH(C₁-C₄)hydrocarbyl, —N[(C₁-C₄)hydrocarbyl)]₂, or —C(═O)—N[(C₁-C₃)hydrocarbyl]₂.

123. A compound according to claim 122, wherein R² is methyl optionally substituted with one or more of —F, —O—C(═O)(C₁-C₄)hydrocarbyl, —C(═O)—OH, —C(═O)—O—CH₃, or —C(═O)—NH—CH₃.

124. A compound according to claim 123, wherein R² is methyl.

125. A compound according to claim 123, wherein R² is difluoro methyl.

126. A compound according to claim 123, wherein R² is CH₂—O—C(═O)(C₁-C₄)hydrocarbyl.

127. A compound according to claim 126, wherein R² is CH₂—O—C(═O)—CH₃.

128. A compound according to claim 123, wherein R² is CH₂—C(═O)—OH.

129. A compound according to claim 123, wherein R² is CH₂—C(═O)—O—CH₃.

130. A compound according to claim 123, wherein R² is CH₂—C(═O)—NH—CH₃.

131. A compound according to claim 122, wherein R² is ethyl optionally substituted with one or more of —OH, —O—C(═O)(C₁-C₄)hydrocarbyl, —NH(C₁-C₄)hydrocarbyl, or —N[(C₁-C₄)hydrocarbyl)]₂.

132. A compound according to claim 131, wherein R² is ethyl.

133. A compound according to claim 131, wherein R² is ethanol.

134. A compound according to claim 131, wherein R² is (CH₂)₂—O—C(═O)—CH₃.

135. A compound according to claim 122, wherein R² is propyl optionally substituted with one or more of —CN.

136. A compound according to any one of claims 37-38, with the structure of formula VIII: 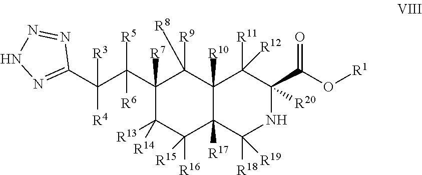
wherein:
at least one of R³-R²⁰ is deuterium; and
R¹ is chosen from H and (C₁-C₁₀) hydrocarbyl.

137. A pharmaceutical composition comprising a pharmaceutically acceptable carrier and a compound according to any one of claims 1-136, or a pharmaceutically acceptable salt thereof.

138. A method of treating a seizure disorder, status epilepticus, partial-onset seizures, primary generalized tonic-clonic seizures, or convulsions, comprising administering to a subject a therapeutically or prophylactically effective amount of the compound of any one of claims 1-136.

139. The method according to claim 138, wherein said seizure disorder is epilepsy.

140. The method according to claim 138, wherein said seizure-disorder is a heritable genetic seizure disorder.

141. The method according to claim 138, wherein said seizure-disorder may result from a brain tumor, traumatic brain injury (either concussive or penetrating), or head injury.

142. The method according to claim 138, wherein said seizure-disorder may result from brain injury during or after viral infection, bacterial infection, parasitic infection, prion disease or idiopathic causes.

143. The method according to claim 138, wherein said seizure-disorder may result from hemorrhagic stroke or ischemic stroke as post-stroke seizures or recurrent epilepsy after stroke.

144. The method according to claim 138, wherein said seizure-disorder may result from systemic autoimmune disorders (autoimmune epilepsy), autoimmune encephalitis, Rasmussen's encephalitis (RE), febrile-seizures, febrile-related epilepsies, fever induced refractory epilepsy syndrome (FIRES), or neuroinflammation.
